(19) United States
(12) Patent Application Publication
(10) Pub. No.: US 2005/0043533 A1
(43) Pub. Date: Feb. 24, 2005

(54) Process for making alpha-substituted hydroxamic acids

(76) Inventors: Kevin A. Babiak, Evanston, IL (US); Terri L. Boehm, Ballwin, MO (US); Pierre-Jean Colson, Skokie, IL (US); Payman N. Farid, Vernon Hills, IL (US); Donald J. Gallagher, Vista, CA (US); Christian H. Huber, Farmington Hills, MI (US); Russell J. Linderman, Old Lyme, CT (US); Chin Liu, Vernon Hills, IL (US); Eduardo K. Mar, Northbrook, IL (US); Murad Newaz, Waterford, CT (US); Daniel J. Pippel, Del Mar, CA (US); Claire A. Przybyla, Oakdale, CT (US); Christopher N. Robb, Mundelein, IL (US); Glenn L. Stahl, Buffalo Grove, IL (US); Ravindra S. Topgi, Palatine, IL (US); Gerald A. Weisenburger, Groton, CT (US); Edward E. Yonan, Carol Stream, IL (US)

Correspondence Address: HARNESS, DICKEY, & PIERCE, P.L.C, 7700 BONHOMME, STE 400, ST. LOUIS, MO, 63105, US

(21) Appl. No.: 10/807,884

(22) Filed: Mar. 24, 2004

Related U.S. Application Data
(60) Provisional application No. 60/457,649, filed on Mar. 25, 2003.

(51) Int. Cl. C07D 43/02

(52) U.S. Cl. 544/360; 546/188

(57) ABSTRACT

This invention is directed generally to a process for making α-substituted hydroxamic acids (including salts thereof) generally corresponding in structure to Formula (I): 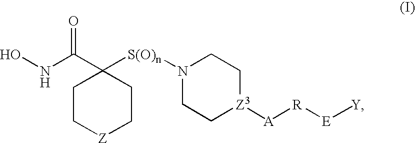
wherein n, Z, Z³, A, R, E, and Y are as defined in this patent. This invention also is directed to compounds that may, for example, be used as intermediates in such a process, as well as processes for making such compounds.

### PRIORITY CLAIM TO RELATED PATENT APPLICATION

This patent claims priority to U.S. Provisional Patent Application Ser. No. 60/457,649 (filed Mar. 25, 2003). The entire text of U.S. Provisional Patent Application Ser. No. 60/457,649 is incorporated by reference into this patent.

### FIELD OF THE INVENTION

This invention is directed generally to a process for making α-substituted hydroxamic acids (including α-substituted hydroxamic acid salts), and particularly to hydroxamic acids wherein the hydroxamic acid α-carbon is substituted with a piperidinylthio, piperidinylsulfoxido, piperidinylsulfonyl, piperazinylthio, piperazinylsulfoxido, or piperazinylsulfonyl substituent. This invention also is directed to compounds that may, for example, be used as intermediates in such a process, as well as processes for making such compounds.

### BACKGROUND OF THE INVENTION

Connective tissue is a required component of all mammals. It provides rigidity, differentiation, attachments, and, in some cases, elasticity. Connective tissue components include, for example, collagen, elastin, proteoglycans, fibronectin, and laminin. These biochemicals make up (or are components of) structures, such as skin, bone, teeth, tendon, cartilage, basement membrane, blood vessels, cornea, and vitreous humor.

Under normal conditions, connective tissue turnover and/or repair processes are in equilibrium with connective tissue production. Degradation of connective tissue is carried out by the action of proteinases released from resident tissue cells and/or invading inflammatory or tumor cells.

Matrix metalloproteinases, a family of zinc-dependent proteinases, make up a major class of enzymes involved in degrading connective tissue. Matrix metalloproteinases are divided into classes, with some members having several different names in common use. Examples are: MMP-1 (also known as collagenase 1, fibroblast collagenase, or EC 3.4.24.3); MMP-2 (also known as gelatinase A, 72kDa gelatinase, basement membrane collagenase, or EC 3.4.24.24), MMP-3 (also known as stromelysin 1 or EC 3.4.24.17), proteoglycanase, MMP-7 (also known as matrilysin), MMP-8 (also known as collagenase II, neutrophil collagenase, or EC 3.4.24.34), MMP-9 (also known as gelatinase B, 92kDa gelatinase, or EC 3.4.24.35), MMP-10 (also known as stromelysin 2 or EC 3.4.24.22), MMP-11 (also known as stromelysin 3), MMP-12 (also known as metalloelastase, human macrophage elastase or HME), MMP-13 (also known as collagenase 111), and MMP-14 (also known as MT1-MMP or membrane MMP). See, generally, Woessner, J. F., “The Matrix Metalloprotease Family” in Matrix Metalloproteinases, pp. 1-14 (Edited by Parks, W. C. & Mecham, R. P., Academic Press, San Diego, Calif. 1998).

Excessive breakdown of connective tissue by MMPs is a feature of many pathological conditions. Inhibition of MMPs therefore provides a control mechanism for tissue decomposition to treat these pathological conditions. Such pathological conditions generally include, for example, tissue destruction, fibrotic diseases, pathological matrix weakening, defective injury repair, cardiovascular diseases, pulmonary diseases, kidney diseases, liver diseases, ophthalmologic diseases, and diseases of the central nervous system. Specific examples of such conditions include rheumatoid arthritis, osteoarthritis, septic arthritis, multiple sclerosis, a decubitis ulcer, corneal ulceration, epidermal ulceration, gastric ulceration, tumor metastasis, tumor invasion, tumor angiogenesis, periodontal disease, liver cirrhosis, fibrotic lung disease, emphysema, otosclerosis, atherosclerosis, proteinuria, coronary thrombosis, dilated cardiomyopathy, congestive heart failure, aortic aneurysm, epidermolysis bullosa, bone disease, Alzheimer's disease, defective injury repair (e.g., weak repairs, adhesions such as post-surgical adhesions, and scarring), post-myocardial infarction, bone disease, and chronic obstructive pulmonary disease. MMPs (particularly MMP-9) also have been reported to be associated with pathological conditions related to nitrosative and oxidative stress. See Gu, Zezong et al., “S-Nitrosylation of Matrix Metalloproteinases: Signaling Pathway to Neuronal Cell Death,” Science, vol. 297, pp. 1186-90 (2002).

Matrix metalloproteinases also are involved in the biosynthesis of tumor necrosis factors (TNFs). Tumor necrosis factors are implicated in many pathological conditions. TNF-α, for example, is a cytokine that is presently thought to be produced initially as a 28 kD cell-associated molecule. It is released as an active, 17 kD form that can mediate a large number of deleterious effects in vitro and in vivo. TNF-α can cause and/or contribute to the effects of inflammation (e.g., rheumatoid arthritis), autoimmune disease, graft rejection, multiple sclerosis, fibrotic diseases, cancer, infectious diseases (e.g., malaria, mycobacterial infection, meningitis, etc.), fever, psoriasis, cardiovascular diseases (e.g., post-ischemic reperfusion injury and congestive heart failure), pulmonary diseases, hemorrhage, coagulation, hyperoxic alveolar injury, radiation damage, and acute phase responses like those seen with infections and sepsis and during shock (e.g., septic shock and hemodynamic shock). Chronic release of active TNF-α can cause cachexia and anorexia. TNF-α also can be lethal.

Inhibiting TNF (and related compounds) production and action is an important clinical disease treatment. Matrix metalloproteinase inhibition is one mechanism that can be used. MMP (e.g., collagenase, stromelysin, and gelatinase) inhibitors, for example, have been reported to inhibit TNF-α release. See, e.g., Gearing et al. Nature 370, 555-557 (1994). See also, McGeehan et al., “Regulation of Tumour Necrosis Factor-α Processing by a Metalloprotease Inhibitor,” Lett. Nature, 370, 558-561 (1994). MMP inhibitors also have been reported to inhibit TNF-α convertase, a metalloproteinase involved in forming active TNF-α. See, e.g., Crimmin et al., WIPO Int'l Pub. No. WO 94/24140 (filed Apr. 18, 1994 as PCT Appl. No. PCT/GB94/00808; published Oct. 27, 1994). See also, Kaltenbach et. al., WIPO Int'l Pub. No. WO 94/02466 (filed Jul. 22, 1993 as PCT Appl. No. PCT/US93/06771; published Feb. 3, 1994). See also, Zook et al., WIPO Int'l Pub. No. WO 97/20824 (filed Dec. 5, 1996 as PCT Appl. No. PCT/US96/19328; published Jun. 12, 1997).

Matrix metalloproteinases also are involved in other biochemical processes in mammals. These include, for example, control of ovulation, post-partum uterine involution, possibly implantation, cleavage of APP (β-amyloid precursor protein) to the ainyloid plaque, and inactivation of (α_{I}-protease inhibitor (α_{I}-PI). Inhibiting MMPs therefore may be a mechanism that may be used to control of fertility. In addition, increasing and maintaining the levels of an endogenous or administered serine protease inhibitor (e.g., α_{I}-PI) supports the treatment of pathological conditions such as emphysema, pulmonary diseases, inflammatory diseases, and diseases of aging (e.g., loss of skin or organ stretch and resiliency).

Numerous metalloproteinase inhibitors are known. See, generally, Brown, P. D., “Synthetic Inhibitors of Matrix Metalloproteinases,” in Matrix Metalloproteinases, pp. 243-61 (edited by Parks, W. C. & Mecham, R. P., Academic Press, San Diego, Calif. 1998).

Metalloproteinase inhibitors include, for example, natural biochemicals, such as tissue inhibitor of metalloproteinase (TIMP), α2-macroglobulin, and their analogs and derivatives. These are high-molecular-weight protein molecules that form inactive complexes with metalloproteinases.

A number of smaller peptide-like compounds also have been reported to inhibit metalloproteinases. Mercaptoamide peptidyl derivatives, for example, have been reported to inhibit angiotensin converting enzyme (also known as ACE) in vitro and in vivo. ACE aids in the production of angiotensin II, a potent pressor substance in mammals. Inhibiting ACE leads to lowering of blood pressure.

A wide variety of thiol compounds have been reported to inhibit MMPs. See, e.g., Montana et al., WIPO Int'l Publ. No. WO 95/13289 (filed Nov. 10, 1994 as PCT Appl. No. PCT/GB94/02471; published May 18, 1995). See also, Montana et al., WIPO Int'l Publ. No. WO 96/11209 (filed Oct. 5, 1995 as PCT Appl. No. PCT/GB95/02362; published Apr. 18, 1996). See also, Donald et al., U.S. Pat. No. 4,595,700 (filed Feb. 21, 1985 as U.S. application Ser. No. 703,973; issued Jun. 17, 1986). See also, Freskos et al., U.S. Pat. No. 6,013,649 (filed Jul. 22, 1997 as U.S. application Ser. No. 08/900,028; issued Jan. 11, 2000).

A wide variety of hydroxamic acid compounds also have been reported to inhibit MMPs. See e.g., Sandanayaka et al., U.S. patent Pre-Grant Publ. No. 2002/0099035 (filed Jan. 24, 2001 as U.S. application Ser. No. 09/769,107; published Jul. 25, 2002). Such compounds reportedly include hydroxamic acids having a carbon backbone. See, e.g., DeCicco et al., WIPO Int'l Pub. No. WO 95/29892 (filed Apr. 27, 1995 as PCT Appl. No. PCT/US95/05012; published Nov. 9, 1995). See also, Groneberg et al., WIPO Int'l Pub. No. WO 97/24117 (filed Jan. 2, 1997 as PCT Appl. No. PCT/US97/00264; published Jul. 10, 1997). See also, WIPO Int'l Pub. No. WO 97/49679 (filed Jun. 25, 1997 as PCT Appl. No. PCT/JP97/02200; published Dec. 31, 1997). See also, Lee et al., European Patent No. EP 0 780 386 (filed Dec. 10, 1996; published Jun. 25, 1997). Such compounds also reportedly include hydroxamic acids having peptidyl backbones or peptidomimetic backbones. See, e.g, Campion et al., WIPO Int'l Pub. No. WO 90/05719 (filed Nov. 23, 1989 as PCT Appl. No. PCT/GB89/01399; published May 31, 1990). See also, Crimmin et al., WIPO Int'l Pub. No. WO 93/20047 (filed Apr. 5, 1993 as PCT Appl. No. PCT/GB93/00706; published Oct. 14, 1993). See also, Crimmin et al., WIPO Int'l Pub. No. WO 95/09841 (filed Oct. 4, 1994 as PCT Appl. No. PCT/GB94/02145; published Apr. 13, 1995). See also, Beckett et al., WIPO Int'l Pub. No. WO 96/06074 (filed Aug. 18, 1995 as PCT Appl. No. PCT/GB95/01971; published Feb. 29, 1996). See also, Schwartz et al., Progr. Med. Chem., 29:271-334(1992). See also, Rasmussen et al., PharmacoL Ther., 75(1): 69-75 (1997). See also, Denis et al., Invest New Drugs, 15: 175-185 (1997). Various piperazinylsulfonyl α-substituted hydroxamic acids and piperidinylsulfonyl α-substituted hydroxamic acids also have been reported to inhibit MMPs. See, e.g., DeCrescenzo, et al., WIPO Int'l Publ. No. WO 00/46221 (filed Feb. 7, 2000 as PCT Application No. PCT/US00/03061; published Aug. 10, 2000); DeCrescenzo, et al., U.S. Pat. No. 6,372,758 (filed Jun. 19, 2991 as U.S. application Ser. No. 09/884,548; issued Apr. 16, 2002); DeCrescenzo, et al., U.S. Pat. No. 6,448,250 (filed Feb. 7, 2000 as U.S. application Ser. No. 09/499,276; issued Sep. 10, 2002); and DeCrescenzo, et al., U.S. Pat. No. 6,492,367 (filed Feb. 26, 2002 as U.S. application Ser. No. 10/084,713; issued Dec. 10, 2002). And various aromatic sulfone hydroxamic acids have been reported to inhibit MMPs. See, e.g., Barta et al., WIPO Int'l Pub. No. WO 99/25687 (filed Nov. 12, 1998 as PCT Appl. No. PCT/US98/23242; published May 27, 1999; Barta et al., U.S. Pat. No. 6,541,489 (filed May 12, 2000 as a U.S. national-phase application Ser. No. 09/554,082; issued Apr. 1, 2003). See also, Barta et al., WIPO Int'l Pub. No. WO 00/50396 (filed Feb. 22, 2000 as PCT Appl. No. PCT/US00/02518; Published Aug. 31, 2000); Barta et al., U.S. patent Pre-Grant Appl. Publ. No. 20020177588 (filed Sep. 17, 2001 as U.S. application Ser. No. 09/954,451; published Nov. 28, 2002); and Barta et al., U.S. patent Pre-Grant Publ. No. 20010039287 (filed Feb. 24, 1999; published Nov. 8, 2001). See also, Barta et al., WIPO Int'l Pub. No. WO 00/69821 (filed May 15, 2000 as PCT Appl. No. PCT/US00/06719; Published Nov. 23, 2000). See also, Barta et al., WIPO Int'l Pub. No. WO 02/092588 (file May 10, 2002 as PCT/US 02/15257; published Nov. 21, 2002). See also, Barta et al., U.S. patent Pre-Grant Publ. No. 20010014688 (filed Nov. 13, 1998; published Aug. 16, 2001). See also, Barta et al., PCT Appl. No. PCT/US02/37093 (filed Nov. 19, 2002).

It is often advantageous for an MMP-inhibitor drug to target a certain MMP(s) over another MMP(s). For example, it is typically preferred to inhibit MMP-2, MMP-3, MMP-9, and/or MMP-13 when treating cancer, inhibiting of metastasis, and inhibiting angiogenesis. It also is typically preferred to inhibit MMP-13 when treating osteoarthritis. See, e.g., Mitchell et al., J Clin. Invest., 97(3):761-768 (1996). See also, Reboul et al., J Clin. Invest., 97(9):2011-2019 (1996). Normally, however, it is preferred to use a drug that has little or no inhibitory effect on MMP-1 and MMP-14. This preference stems from the fact that both MMP-1 and MMP-14 are involved in several homeostatic processes, and inhibition of MMP-1 and/or MMP-14 consequently tends to interfere with such processes.

Another enzyme-implicated in pathological conditions associated with excessive degradation of connective tissue is aggrecanase, particularly aggrecanase-1 (also known as ADAMTS-4). Articular cartilage contains large amounts of the proteoglycan aggrecan. Proteoglycan aggrecan provides mechanical properties that help articular cartilage in withstanding compressive deformation during joint articulation. The loss of aggrecan fragments and their release into synovial fluid caused by proteolytic cleavages is a central pathophysiological event in osteoarthritis and rheumatoid arthritis. It has been reported that two major cleavage sites exist in the proteolytically sensitive interglobular domains at the N-terminal region of the aggrecan core protein. One of those sites has been reported to be cleaved by several matrix metalloproteases. The other site, however, has been reported to be cleaved by aggrecanase-1. Thus, inhibiting excessive aggrecanase activity provides an additional and/or alternative treatment method for inflammatory conditions. Such diseases reportedly include, for example, osteoarthritis, rheumatoid arthritis, joint injury, reactive arthritis, acute pyrophosphate arthritis, and psoriatic arthritis. See generally, WIPO Int'l Pub. No. WO 02/092588 (cited above). See also, Barta et al., WIPO Int'l Publ. No. WO 03/007930 (filed Jul. 19, 2002 as PCT Appl. No. PCT/US02/22867; published Jan. 30, 2003). See also, Tang, B. L., “ADAMTS: A Novel Family of Extracellular Matrix Proteases,” Int'l Journal of Biochemistry & Cell Biology, 33, pp. 33-44 (2001). See also, Noe, European Appl. Publ. No. EP 1 081 137 (filed Aug. 8, 2000; published Mar. 7, 2001).

In addition to inflammatory conditions, there also is evidence that inhibiting aggrecanase may be used for treating cancer. For example, excessive levels of aggrecanase-1 reportedly have been observed with a ghoma cell line. It also has been postulated that the enzymatic nature of aggrecanase and its similarities with the MMPs would support tumor invasion, metastasis, and angiogenesis. See Tang (cited above).

Various hydroxamic acid compounds have been reported to inhibit aggrecanase-1. Such compounds include, for example, those described in WIPO Int'l Pub. No. WO 02/092588 (cited above). Such compounds also include, for example, those described in WIPO Int'l Publ. No. WO 03/007930 (cited above). Such compounds also include, for example, those described in European Appl. Publ. No. EP 1 081 137 (cited above). Such compounds also include, for example, those described in Yao et al., WIPO PCT Int'l Publ. No. WO 00/09000 (filed Aug. 18, 1998 as PCT Appl. No. PCT/US98/17048; published Feb. 25, 1999). Such compounds further include, for example, those described in Duan, WIPO PCT Int'l Publ. No. WO 00/59874 (filed Mar. 30, 2000 as PCT Appl. No. PCT/US00/08362; published Oct. 12, 2000).

In view of the importance of hydroxamic acid compounds in the treatment of several pathological conditions, there continues to be a need for reliable and cost-effective processes that may be used for their preparation. The following disclosure describes such a process.

### SUMMARY OF THE INVENTION

This invention is directed to a process for making α-substituted hydroxamic acids, including α-substituted hydroxamic acid salts.

Briefly, therefore, this invention is directed to a process for making a hydroxamic acid compound corresponding in structure to Formula (I) or a salt thereof: 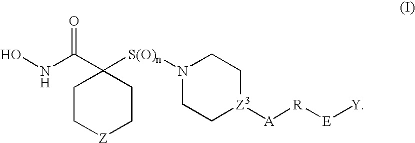
Here:

n is zero, 1, or 2.

Z is —O—, —S—, —S(O)—, —S(O)₂—, or —N(R^{x})—.

Z³ is nitrogen or carbon bonded to hydrogen.

R^{x} is hydrogen, alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, or heterocyclylsulfonyl. Any such substituent optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally substituted with one or more substituents independently selected from the group consisting of halogen, hydroxy, and alkyl.

A is —O—, —S—, —S(O)—, —S(O)₂—, —C(O)—, —NR^{b}—, —CO—N(R^{b}), —N(R_{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, or a bond.

R is alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, or heterocyclylthioalkyl. Any such substituent optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl. Any such optional substituent is, in turn, optionally is substituted with one or more substituents independently selected from the group consisting of halogen, hydroxy, cyano, carboxy, thiol, sulfo, nitro, nitroso, oxo, thioxo, and imino.

E is —O—, —C(O)—, —C(O)—O—, —O—C(O)—, —N(R^{b})—, —C(O)—N(R^{b}), —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of such a substituent optionally is substituted with one or more independently selected R^{c} substituents.

Y is hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

Each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Each R^{b} is independently selected aryl.

Each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{e} is independently selected from the group consisting of hydrogen alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{g} is independently selected from the group consisting of hydrogen, alkyl, —O—R^{h}, carbocyclylalkyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{h} is independently selected from the group consisting of hydrogen, alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

In some such embodiments, the process comprises reacting a 4-sulfonyloxy-heterocyclyl compound or a 4-halo-heterocyclyl compound with a metal thioester. Here:

The 4-sulfonyloxy-heterocyclyl compound corresponds in structure to the following formula:

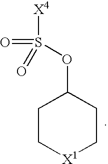

The 4-halo-heterocyclyl compound corresponds in structure to the following formula:

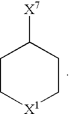

The metal thioester corresponds in structure to the following formula:

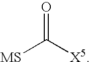

X¹ is —O—, —S—, —S(O)—, —S(O)₂—, or —N(R^{x1})—.

X⁴ is alkyl, haloalkyl, aryl, or haloaryl.

X⁵ is alkyl, aryl, or arylalkyl. The alkyl, aryl, and arylalkyl are, in turn, stituted with one or more independently selected halogen.

X⁷ is halogen.

M is a metal cation.

R^{x1} is a nitrogen-protecting group.

In other embodiments for making the hydroxamic acid of Formula (I) or a salt therof, the process comprises oxidatively halogenating a 4-thioester-heterocyclyl compound. Here:

The 4-thioester-heterocyclyl compound corresponds in structure to the following formula:

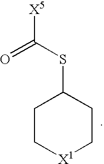

X¹ is —O—, —S—, —S(O)—, —S(O)₂—, or —N(R^{x1})—.

X⁵ is alkyl, aryl, or arylalkyl. The alkyl aryl, and arylalkyl are, in turn, optionally substituted with one or more independently selected halogen.

R^{x1} is a nitrogen-protecting group.

In other embodiments for making the hydroxamic acid of Formula (I) or a salt thereof, the process comprises reacting a 4-halosulfur-heterocyclyl compound with a cyclic amino compound. Here:

The 4-halosulfur-heterocyclyl compound corresponds in structure to the following formula:

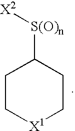

The cyclic amino compound corresponds in structure to the following formula:

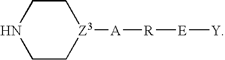

The n of the 4-halosulfur-heterocyclyl compound is zero, 1, or 2. This n may the same or different from the n in the hydroxamic acid compound of Formula (I), but preferably is the same.

X¹ is —O—, —S—, —S(O)—, —S(O)₂—, or —N(R^{x1})—.

X² is halogen.

R^{x1} is a nitrogen-protecting group.

In other embodiments for making the hydroxamic acid of Formula (I) or a salt thereof, the process comprises removing a nitrogen-protecting group (R^{x1}) from a piperidinyl nitrogen of a nitrogen-protected piperidinyl compound. Here:

Z is —N(R^{x})—.

The nitrogen-protected piperidinyl compound corresponds in structure to the following formula:

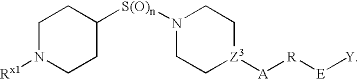

The n of the nitrogen-protected piperidinyl compound is zero, 1, or 2. This n may the same or different from the n in the hydroxamic acid compound of Formula (I), but preferably is the same.

In other embodiments for making the hydroxamic acid of Formula (I) or a salt thereof, the process comprises contacting an unprotected piperidinyl compound with an N-alkylating agent. Here:

The unprotected piperidinyl compound corresponds in structure to the following formula:

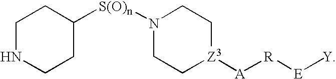

The n of the unprotected piperidinyl compound is zero, 1, or 2. This n may the same or different from the n in the hydroxamic acid compound of Formula (I), but preferably is the same.

Z is —N(R^{x})—.

R^{x} is alkyl optionally substituted with R^{a}-oxy, R^{a}R^{a}-amino (wherein each R^{a} is other than hydrogen), carbocyclyl, or heterocyclyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally substituted with one or more substituents independently selected from the group consisting of halogen, hydroxy, and alkyl.

Each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

In other embodiments for making the hydroxamic acid of Formula (I) or a salt thereof, the process comprises contacting a sulfuramine compound with a base to form an anion, and contacting the anion with a carbon dioxide source. Here:

The sulfuramine compound corresponds in structure to the following formula:

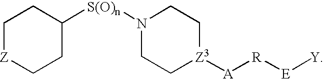

The n of the sulfuramine compound is zero, 1, or 2. This n may the same or different from the n in the hydroxamic acid compound of Formula (I), but preferably is the same.

This invention also is directed to processes for making compounds and salts that may, for example, be used as a starting material or intermediate in the above-described process for making the hydroxamic acid of Formula (I).

In some embodiments, the process is directed to making a 4-thioester-heterocyclyl compound. This process comprises reacting a 4-sulfonyloxy-heterocyclyl compound or a 4-halo-heterocyclyl compound with a metal thioester. Here:

The 4-thioester-heterocyclyl compound corresponds in structure to the following formula:

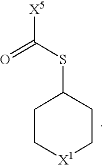

The 4-sulfonyloxy-heterocyclyl compound corresponds in structure to the following formula:

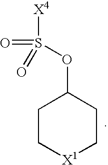

The 4-halo-heterocyclyl compound corresponds in structure to the following formula:

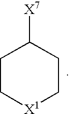

The metal thioester corresponds in structure to the following formula:

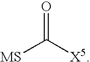

X¹ is —O—, —S—, —S(O)—, —S(O)₂—, or —N(R^{x1})—.

X⁴ is alkyl, haloalkyl, aryl, or haloaryl.

X⁵ is alkyl, aryl, or arylalkyl. The alkyl, aryl, and arylalkyl are, in turn, optionally substituted with one or more independently selected halogen.

X⁷ is halogen.

M is a metal cation.

R^{x1} is a nitrogen-protecting group.

In other embodiments, the process is directed to making a 4-halosulfonyl-heterocyclyl compound. This process comprises oxidatively halogenating a 4-thioester-heterocyclyl compound. Here:

The 4-halosulfonyl-heterocyclyl compound corresponds in structure to the following formula:

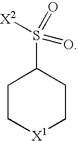

The 4-thioester-heterocyclyl compound corresponds in structure to the following formula:

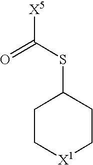

X¹ is —O—, —S—, —S(O)—, —S(O)₂—, or —N(R^{x1})—.

X² is halogen.

X⁵ is alkyl, aryl, or arylalkyl. The alkyl, aryl, and arylalkyl are, in turn, optionally substituted with one or more independently selected halogen.

R^{x1} is a nitrogen-protecting group.

In other embodiments, the process is directed to making an sulfuramine compound. This process comprises reacting a 4-halosulfur-heterocyclyl compound with a cyclic amino compound. Here:

The sulfuramine compound corresponds in structure to the following formula:

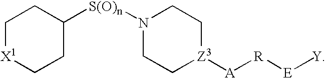

The 4-halosulfur-heterocyclyl compound corresponds in structure to the following formula:

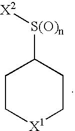

The cyclic amino compound corresponds in structure to the following formula:

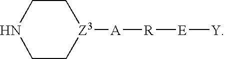

Each n is independently selected from the group consisting of zero, 1, and 2.

X¹ is —O—, —S—, —S(O)—, —S(O)₂—, or —N(R^{x1})—.

X² is halogen.

R^{x1} is a nitrogen-protecting group.

A, R, E, Y, and Z³ are as generally defined above for the hydroxamic acid of Formula (I).

In other embodiments, the process is directed to making an unprotected piperidinyl compound. This process comprises removing a nitrogen-protecting group (R^{x1}) from the piperidinyl nitrogen of a nitrogen-protected piperidinyl compound. Here:

The unprotected piperidinyl compound corresponds in structure to the following formula:

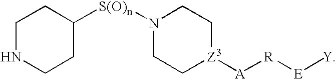

The nitrogen-protected piperidinyl compound corresponds in structure to the following formula:

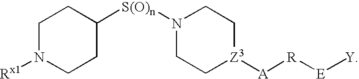

Each n is independently selected from the group consisting of zero, 1, and 2.

A, R, E, Y, and Z³ are as generally defined above for the hydroxamic acid of Formula (I).

In other embodiments, the process is directed to making an alkylated piperidinyl compound or a salt thereof. This process comprises contacting an unprotected piperidinyl compound with an N-alkylating agent.

The alkylated piperidinyl compound corresponds in structure to the following formula:

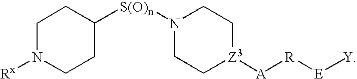

The unprotected piperidinyl compound corresponds in structure to the following formula:

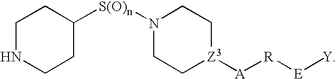

Each n is independently selected from the group consisting of zero, 1, and 2.

A, R, E, Y, and Z³ are as generally defined above for the hydroxamic acid of Formula (I).

R^{x} is alkyl optionally substituted with R^{a}-oxy, R^{a}R^{a}-amino (wherein each R^{a} is other than hydrogen), carbocyclyl, or heterocyclyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally substituted with one or more substituents independently selected from the group consisting of halogen, hydroxy, and alkyl.

Each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

In other embodiments, the process is directed to making a carboxylic acid compound. This process comprises contacting a sulfuramine compound with a base to form an anion, and contacting the anion with a carbon dioxide source. Here:

The carboxylic acid compound corresponds in structure to the following formula:

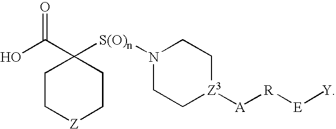

The sulfuramine compound corresponds in structure to the following formula:

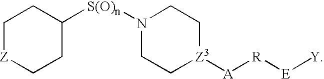

Each n is independently selected from the group consisting of zero, 1, and 2.

A, R, E, Y, Z, and Z³ are as generally defined above for the hydroxamic acid of Formula (I).

This invention also is directed to compounds and salts that may, for example, be used as a starting material or intermediate in the above-described process for making the hydroxamic acid of Formula (I).

In some such embodiments, the compound corresponds in structure to the following formula: 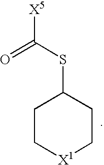
Here:

X¹ is —O—, —S—, —S(O)—, —S(O)₂—, or —N(R^{x1})—.

X⁵ is alkyl, aryl, or arylalkyl. The alkyl, aryl, and arylalkyl are, in turn, optionally substituted with one or more independently selected halogen.

R^{x1} is a nitrogen-protecting group.

In other embodiments, the compound corresponds in structure to the following formula: 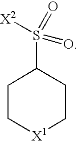
Here:

X¹ is —O—, —S—, —S(O)—, —S(O)₂—, or —N(R^{x1})—.

X² is halogen.

R^{x1} is a nitrogen-protecting group.

In other embodiments, the compound corresponds in structure to the following formula: 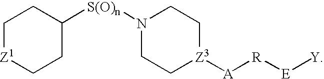
Here:

n is zero, 1, or 2.

Z¹ is —N(H)—, X¹, or —N(R^{x2})—.

X¹ is —O—, —S—, —S(O)—, —S(O)₂—, or —N(R^{x1})—.

R^{x1} is a nitrogen-protecting group.

R^{x2} is alkyl optionally substituted with R^{a}-oxy, R^{a}R^{a}-amino (wherein each R^{a} is other than hydrogen), carbocyclyl, or heterocyclyl. Any member of such group optionally is substituted with one or more substituents independently selected from the group consisting of halogen, cyano, carboxy, thiol, sulfo, nitro, nitroso, oxo, thioxo, imino, alkoxy, alkylthio, and alkoxyalkoxy. Any such optional substituent is, in turn, optionally substituted with one or more substituents independently selected from the group consisting of halogen, hydroxy, and alkyl.

Each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

A, R, E, Y, and Z³ are as generally defined above for the hydroxamic acid of Formula (I).

Further benefits of Applicants' invention will be apparent to one skilled in the art from reading this specification.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

### A. Compounds that may be Prepared by the Process of this Invention

Compounds that may be prepared using one or more steps of the process of this invention generally include those corresponding in structure to Formula (I): 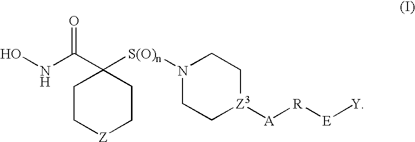
The following discussion describes preferred definitions for each of the variables in Formula (I).

### A-1. Preferred Values for n

The value of n is zero, 1, or 2.

In some preferred embodiments, n is 2. In such embodiments, the compound generally corresponds in structure to the following formula:

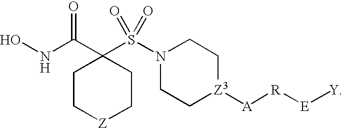

### A-2 Preferred Z³ Moieties

Z³ is (1) carbon bonded to hydrogen, or (2) nitrogen. Where Z³ is carbon bonded to hydrogen, the compound generally corresponds in structure to the following formula: 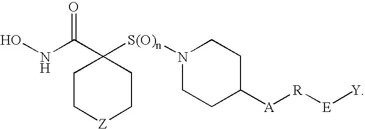
Where Z³ is instead nitrogen, the compound generally corresponds in structure to the following formula:

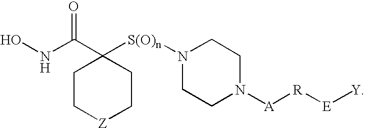

### A-3 Preferred Z Moieties

Z is —S—, —S(O)—, —S(O)₂—, —O—, or —N(R^{x})—.

In some preferred embodiments, Z is —O—. In such embodiments, the compound generally corresponds in structure to the following formula:

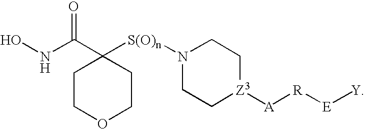

In some preferred embodiments, Z is —N(R^{x})—. In such embodiments, the compound generally corresponds in structure to the following formula:

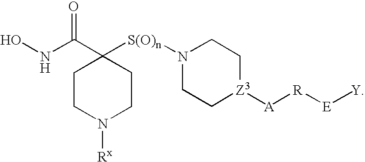

When Z is —N(R^{x})—, R^{x} is hydrogen, alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, or heterocyclylsulfonyl. Any such substituent optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally substituted with one or more substituents independently selected from the group consisting of halogen, hydroxy, and alkyl.

In some preferred embodiments, R^{x} is alkyl, alkenyl, alkynyl, R^{a}-oxyalkyl, R^{a}R^{a}-aminoalkyl, carbocyclylalkyl, or heterocyclylalkyl. Any such substituent optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally is substituted with one or more substituents independently selected from the group consisting of halogen, hydroxy, and alkyl.

In some preferred embodiments, R^{x} is alkyl optionally substituted with one or more substituents selected from the group consisting of R^{a}-oxy, R^{a}R^{a}-amino (wherein each R^{a} is other than hydrogen), carbocyclyl, heterocyclyl, halogen, cyano, carboxy, thiol, sulfo, nitro, nitroso, oxo, thioxo, imino, alkoxy, alkylthio, and alkoxyalkoxy. Any member of such group optionally is substituted with one or more substituents independently selected from the group consisting of halogen, hydroxy, and alkyl.

In some preferred embodiments, R^{x} is alkoxyalkyl.

In some preferred embodiments, R^{x} is methoxyethyl.

### A-4. Preferred A and R Moieties

A is —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C═C—, —N═N—, —C(S)—N(R^{b})—, —N(R^{b})—C(S)—, alkyl, alkoxy, oxyalkyl, alkylthio, thioalkyl, or a bond.

In some preferred embodiments, A is —O—, —S—, —S(O)₂—, —O—S(O)₂—, —S(O)₂—O—, —C(O)—, —C(O)—O—, —O—C(O)—, or a bond.

In some preferred embodiments, A is a bond.

In some preferred embodiments, A is —O—.

R is alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, or heterocyclylthioalkyl. Any such substituent optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino. It is generally preferred for R not to comprise any primary or secondary amine substituents.

In some preferred embodiments, R is heterocyclyl optionally substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R is heterocyclyl substituted on one or more atoms capable of such substitution with oxo.

In some preferred embodiments, R is heterocyclyl.

In some preferred embodiments, R is heteroaryl optionally substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R is heteroaryl.

In some preferred embodiments, R is aryl (preferably phenyl) optionally substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R is phenyl substituted with alkyl.

In some preferred embodiments, R is phenyl substituted with halogen.

In some preferred embodiments, R is phenyl substituted with fluoro.

In some preferred embodiments, R is phenyl.

In some preferred embodiments where Z³ is carbon bonded to hydrogen, A is a bond. In such embodiments, the compound generally corresponds in structure to the following formula:

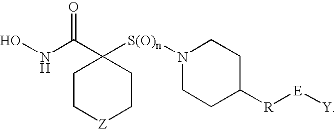

In some preferred embodiments where Z³ is carbon bonded to hydrogen, A is —O—. In such embodiments, the compound generally corresponds in structure to the following formula:

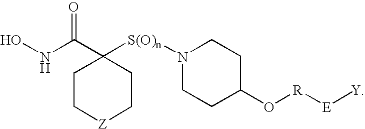

In some preferred embodiments where Z³ is nitrogen, A-R is R². In such embodiments, the compound generally corresponds in structure to the following formula:

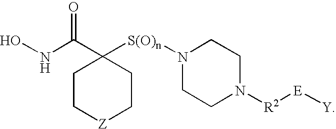

When A-R is R², R² is aryl or heteroaryl. The aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R² is heteroaryl optionally substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R² is heteroaryl.

In some preferred embodiments, R² is aryl (preferably phenyl) optionally substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl. Any such optional substituent is, in turn, optionally substituted with one or more substituents independently selected from the group consisting of halogen, hydroxy, cyano, carboxy, thiol, sulfo, nitro, nitroso, oxo, thioxo, and imino.

In some preferred embodiments, R² is phenyl substituted with alkyl.

In some preferred embodiments, R² is phenyl substituted with halogen.

In some preferred embodiments, R² is phenyl substituted with fluoro.

In some preferred embodiments, R² is phenyl.

### A-5. Preferred E and Y Moieties

E is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of any such substituent (to the extent there is an alkyl or alkenyl portion) optionally is substituted with one or more independently selected R^{c} substituents.

In some preferred embodiments, E is —C(O)—.

In some preferred embodiments, E is a bond.

In some preferred embodiments, E is —O—.

Y is hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents. It is generally preferred for Y not to comprise any primary or secondary amine substituents.

In some preferred embodiments, Y is alkyl optionally substituted with one or more independently selected R^{d} substituents.

In some preferred embodiments, Y is haloalkyl.

In some preferred embodiments, Y is fluoroalkyl.

In some preferred embodiments, Y is trifluoromethyl.

In some preferred embodiments, Y is alkyl.

In some preferred embodiments, Y is methyl.

In some preferred embodiments, -E-Y is hydrogen.

In some preferred embodiments, -E-Y is halogen.

In some preferred embodiments, -E-Y is fluoro.

In some preferred embodiments, -E-Y is alkyl optionally substituted with one or more independently selected R^{d} substituents.

In some preferred embodiments, -E-Y is haloalkyl.

In some preferred embodiments, -E-Y is fluoroalkyl.

In some preferred embodiments, -E-Y is trifluoromethyl.

In some preferred embodiments, -E-Y is alkyl.

In some preferred embodiments, -E-Y is methyl.

In some preferred embodiments, -E-Y is alkoxy optionally substituted with one or more independently selected R^{d} substituents.

In some preferred embodiments, -E-Y is haloalkoxy.

In some preferred embodiments, -E-Y is fluoroalkoxy.

In some preferred embodiments, -E-Y is trifluoromethoxy.

In some preferred embodiments, -E-Y is alkoxy.

In some preferred embodiments, -E-Y is methoxy.

### A-6. Preferred R^{a}, R^{b}, R^{c}, R^{d}, R^{e}, R^{g}, and R^{h} Moieties

Each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

In some preferred embodiments, each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Each R^{b} is independently selected aryl.

Each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{e} is independently selected from the group consisting of hydrogen alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{g} is independently selected from the group consisting of hydrogen, alkyl, —O—R^{h}, carbocyclylalkyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

Each R^{h} is independently selected from the group consisting of hydrogen, alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, and imino.

### A-7. Various Preferred Compounds

In some preferred embodiments, the compounds prepared by this invention include piperazinylsulfonyl α-substituted hydroxamic acids generally corresponding in structure to the following formula: 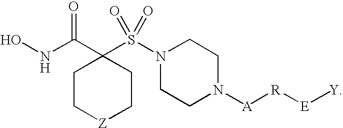
Such compounds include, for example, the following:

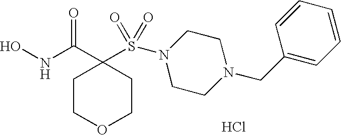

In some often more preferred embodiments, the compounds prepared by this invention include piperazinylsulfonyl α-substituted hydroxamic acids generally corresponding in structure to the following formula: 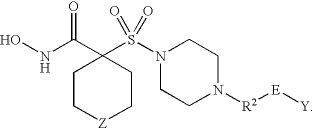
Such compounds include, for example, the following compounds wherein Z is —S—: 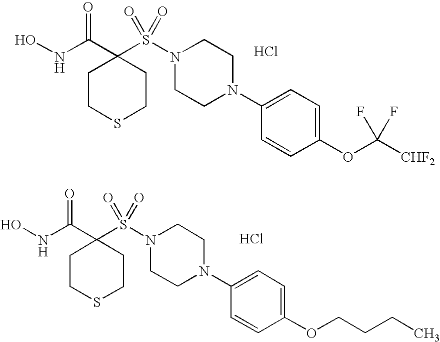
Such compounds also include, for example, the following compounds wherein Z is —S(O)₂—: 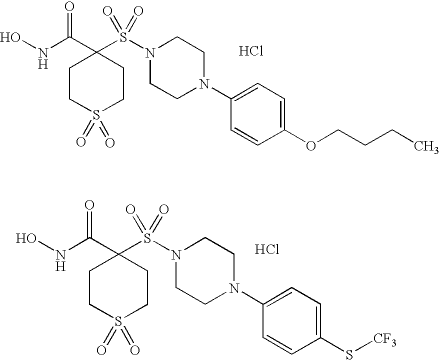
Such compounds also include, for example, the following compounds wherein Z is —O—:

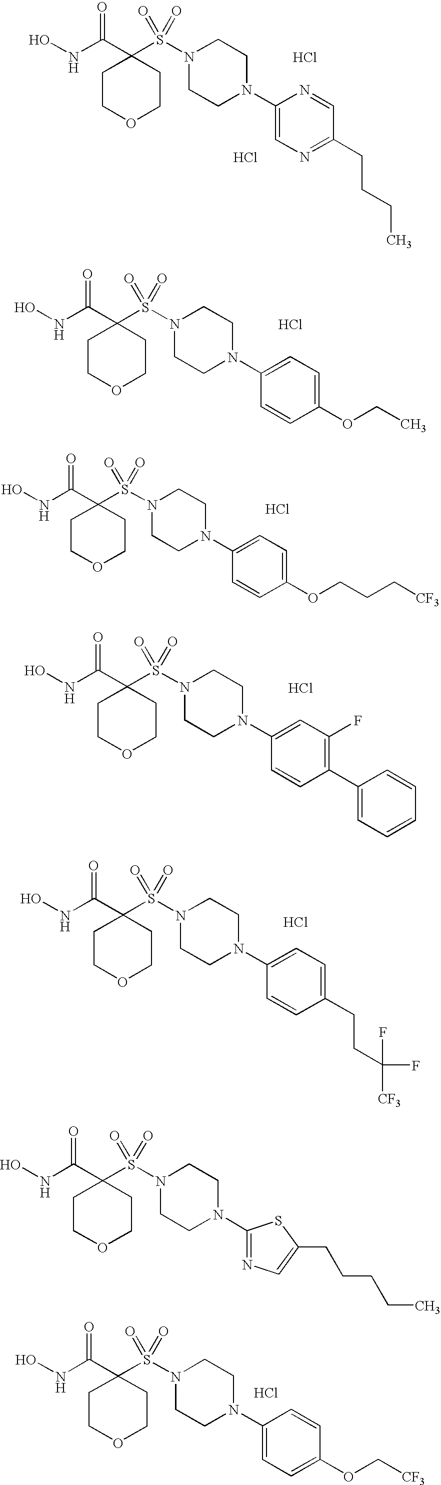

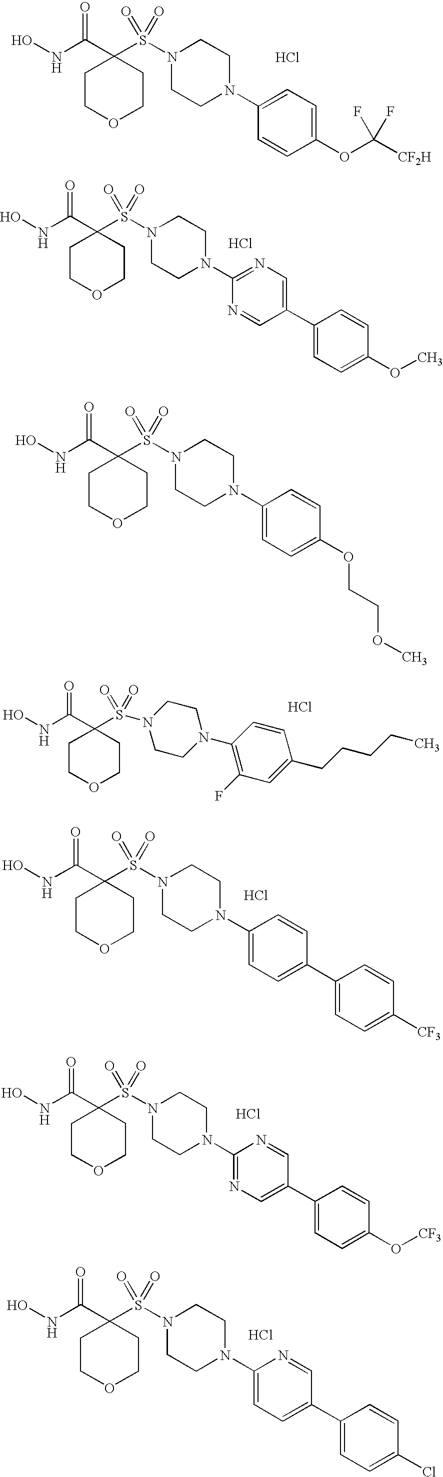

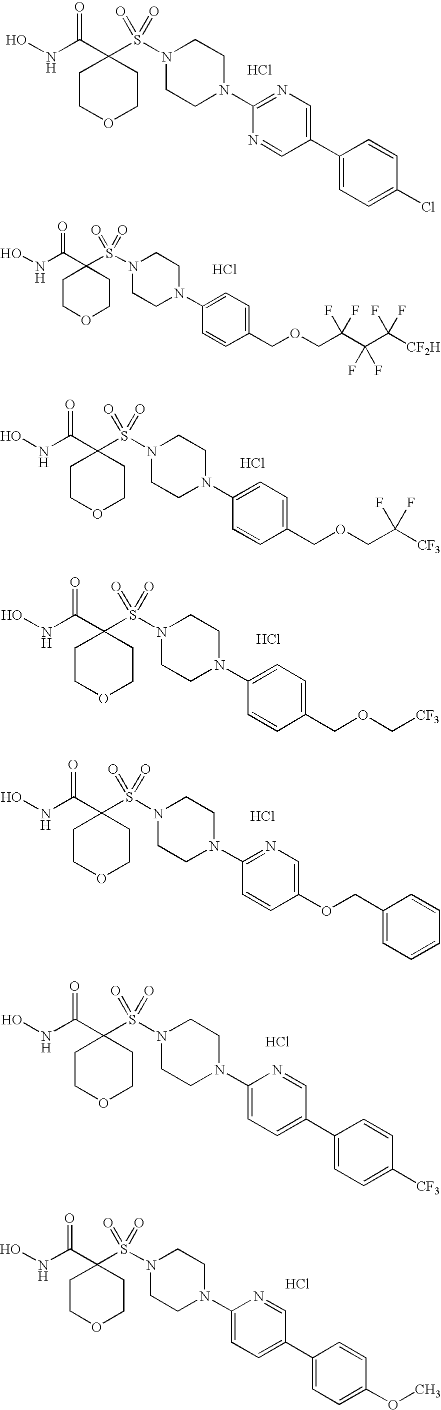

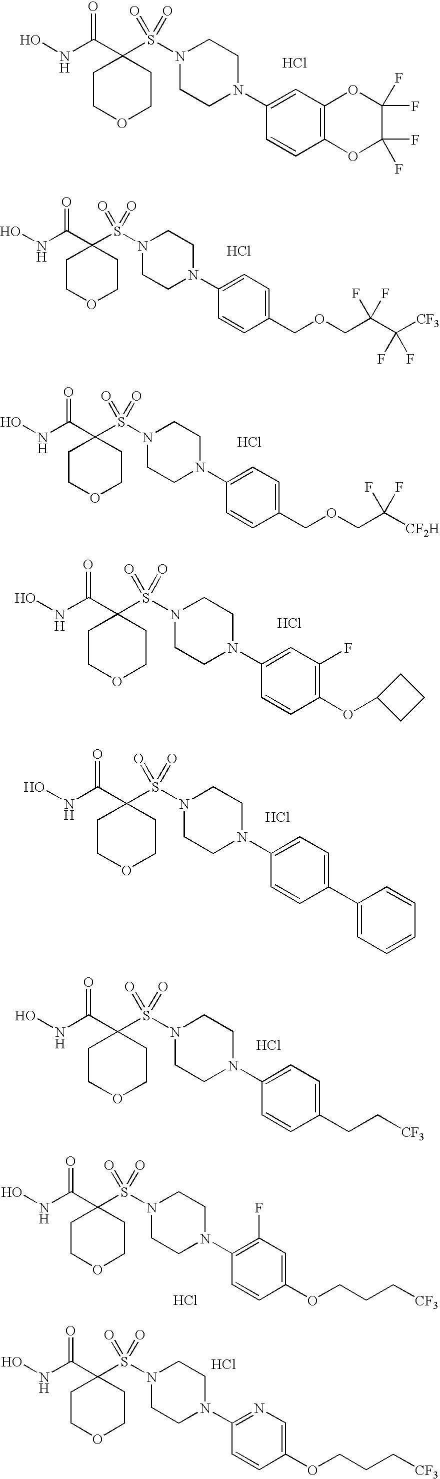

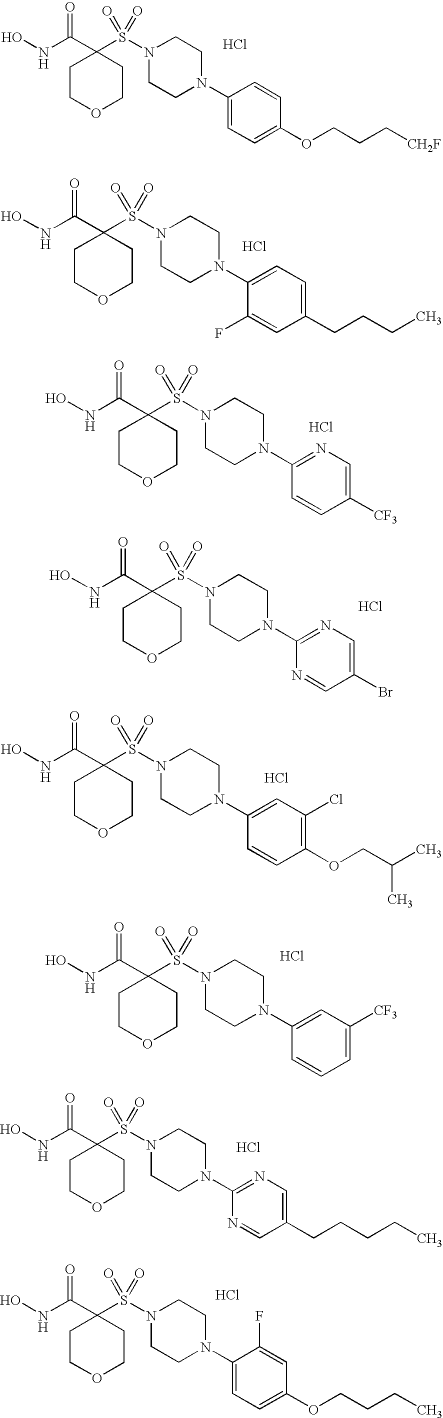

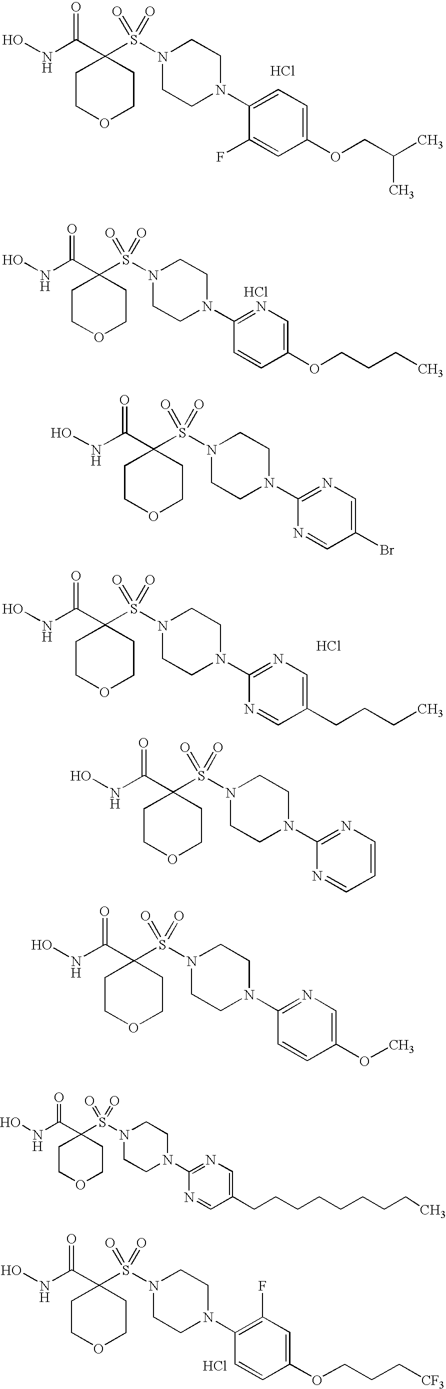

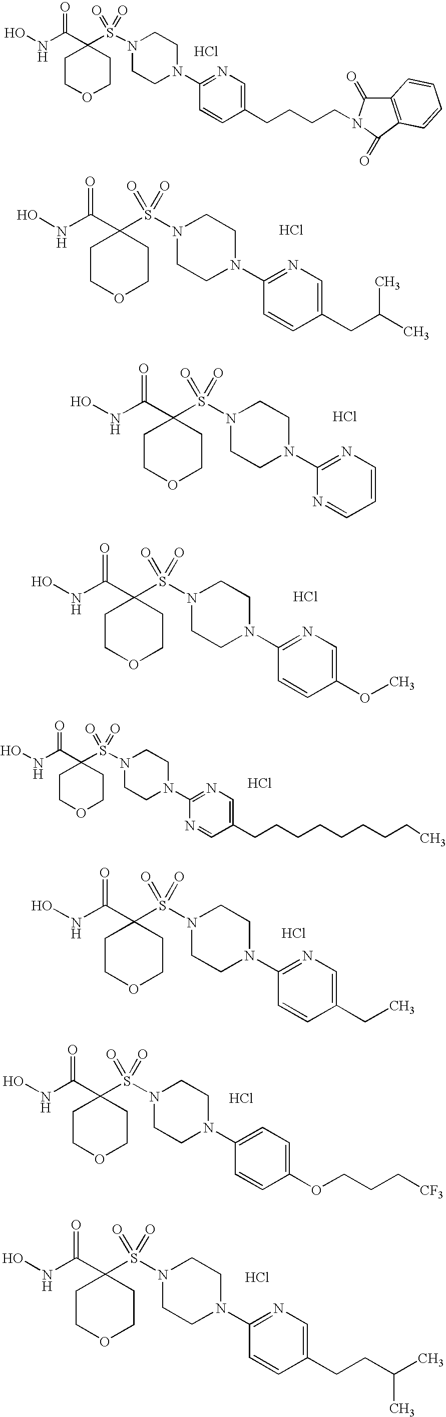

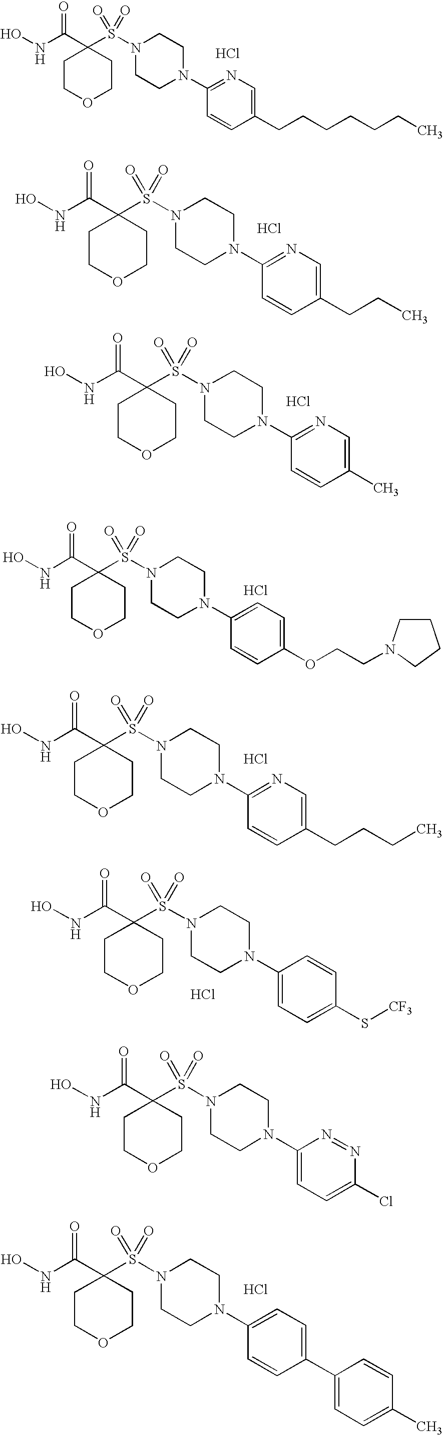

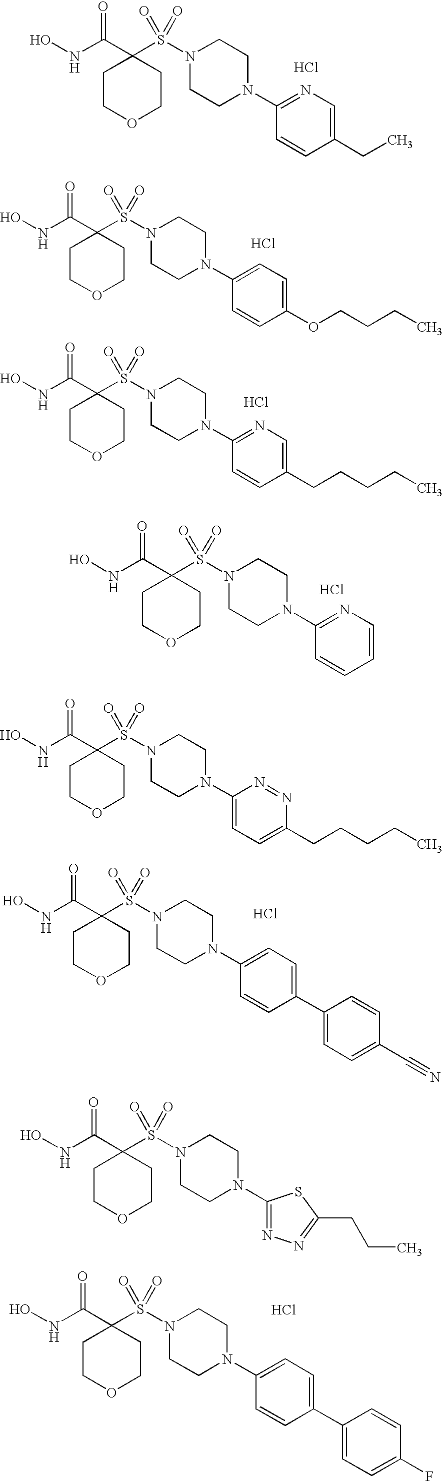

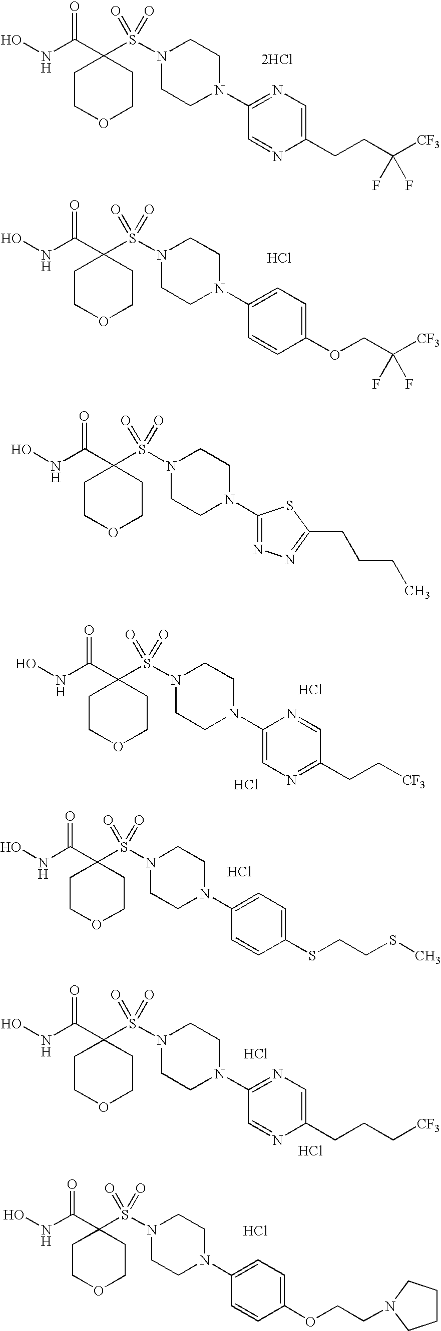

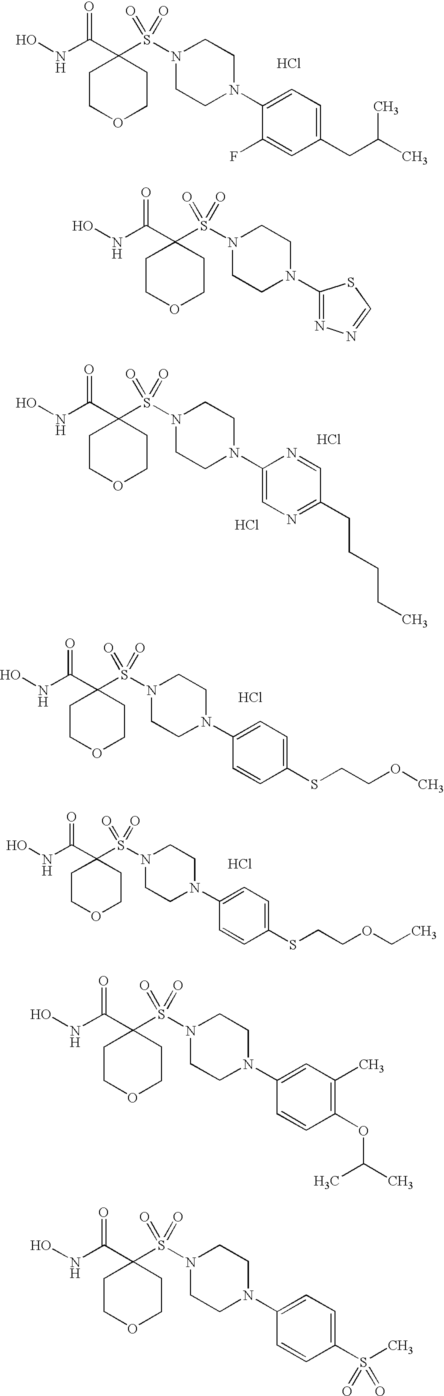

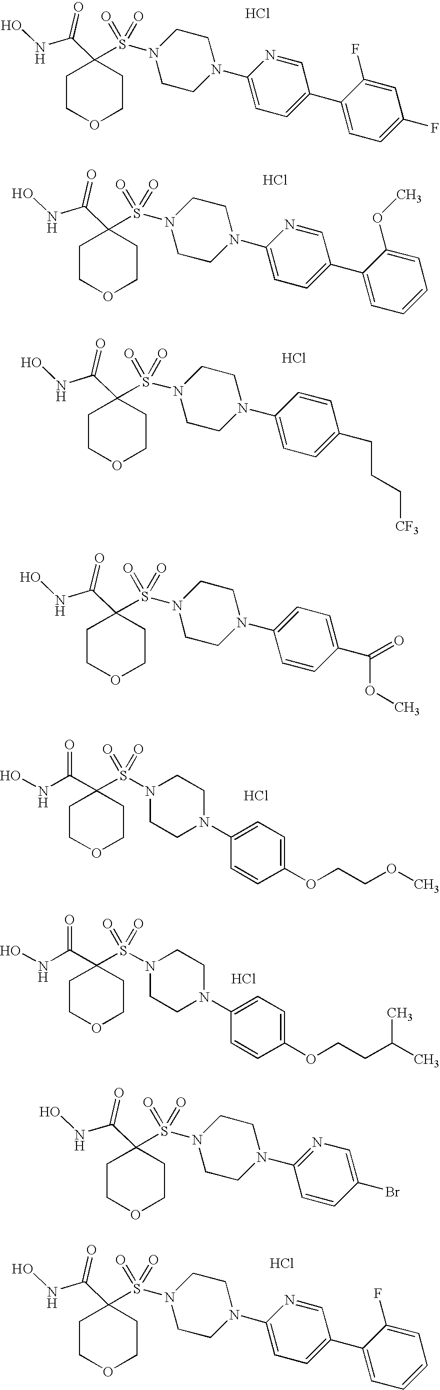

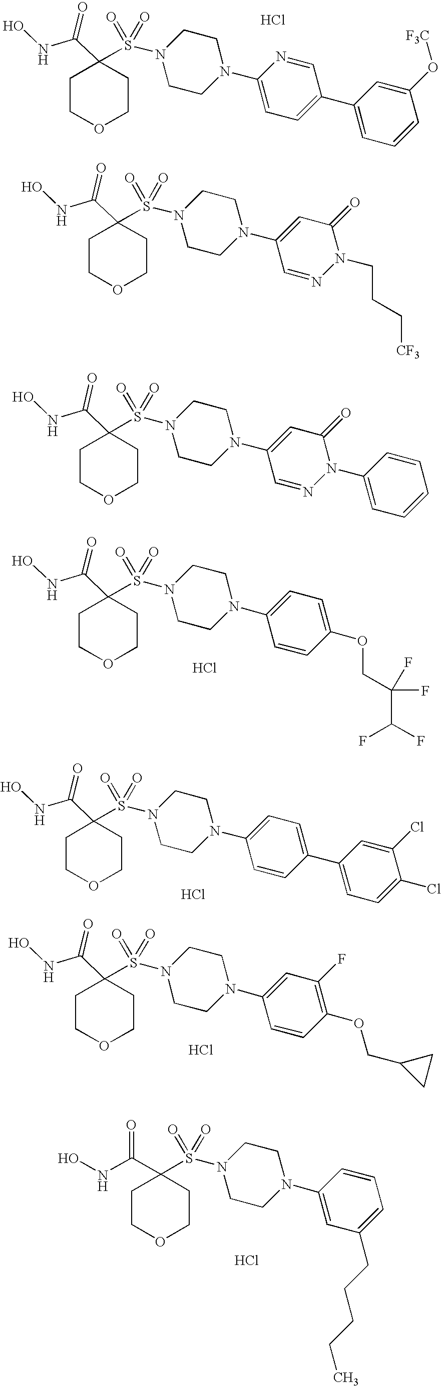

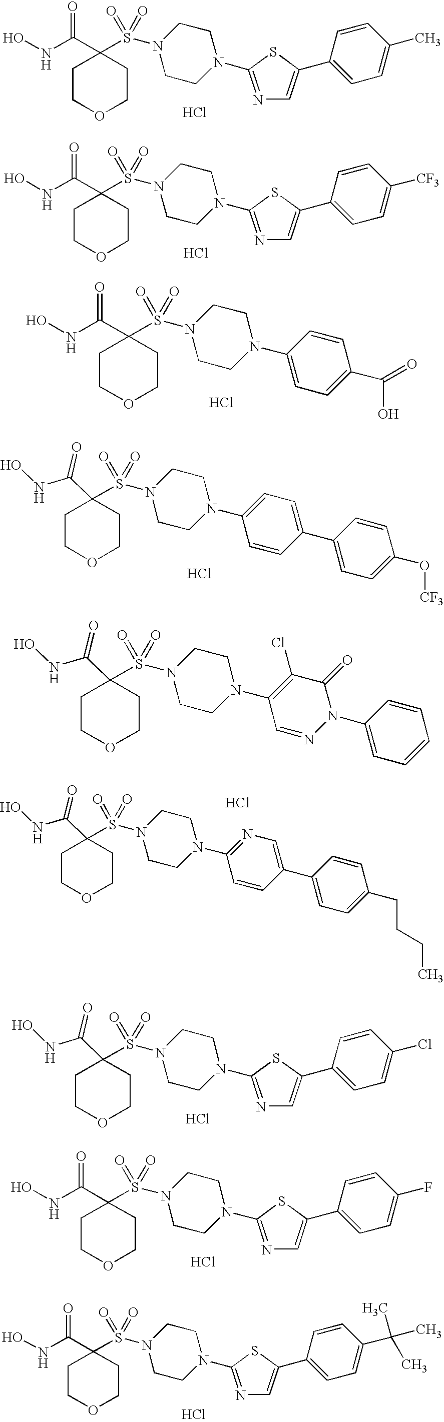

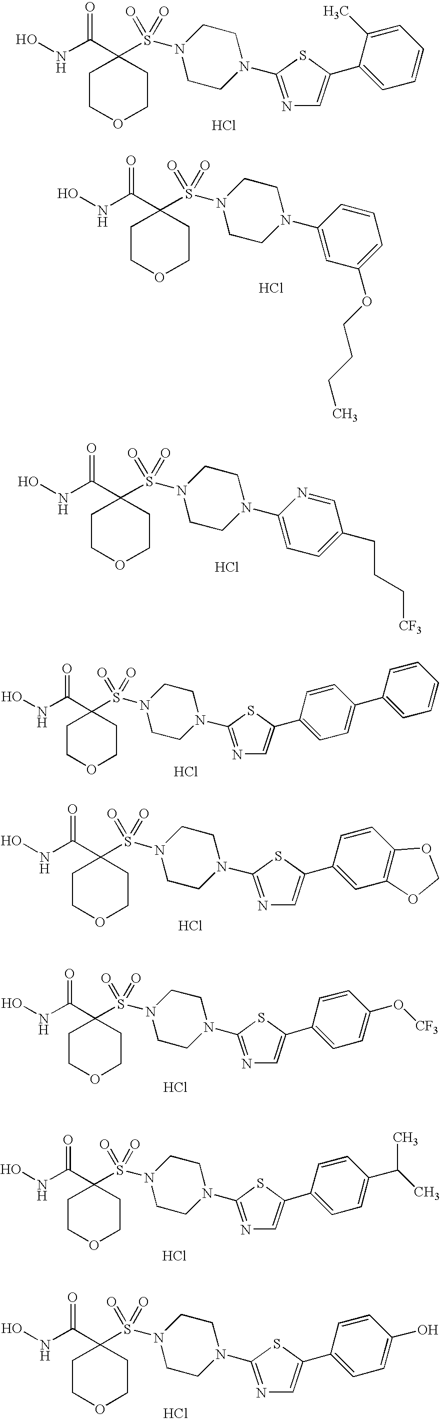

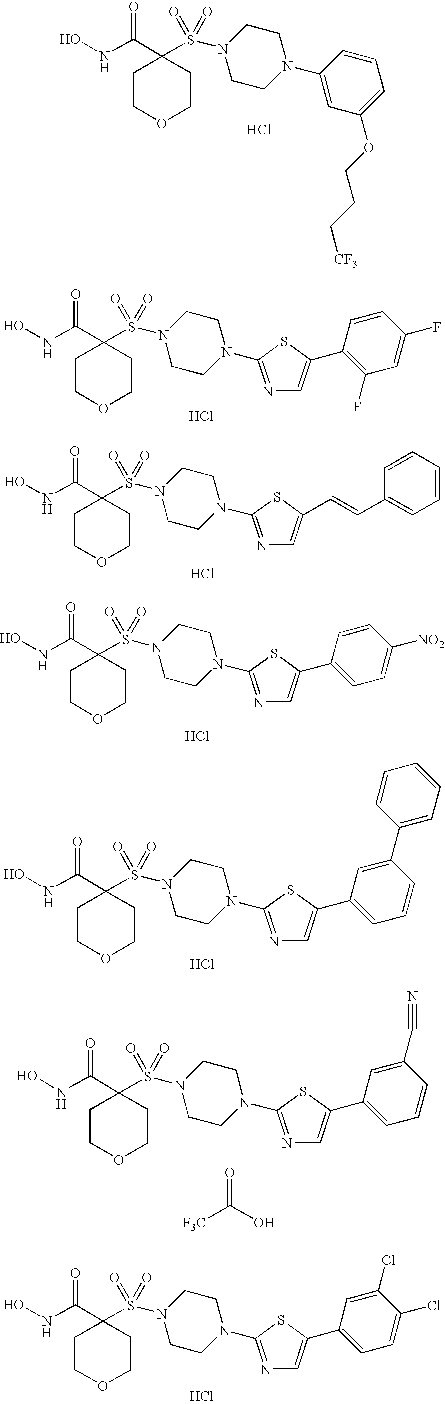

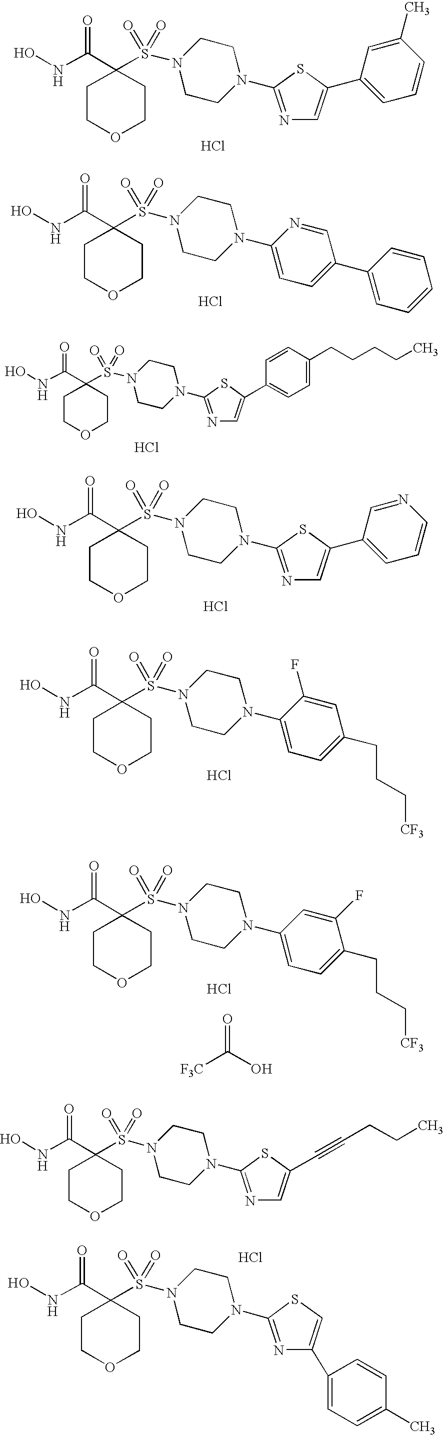

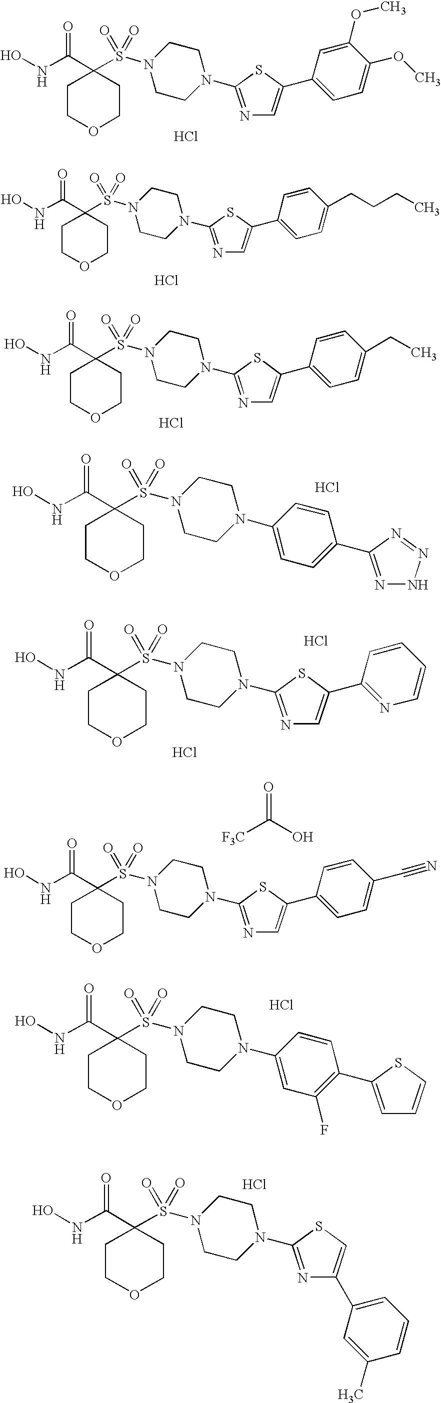

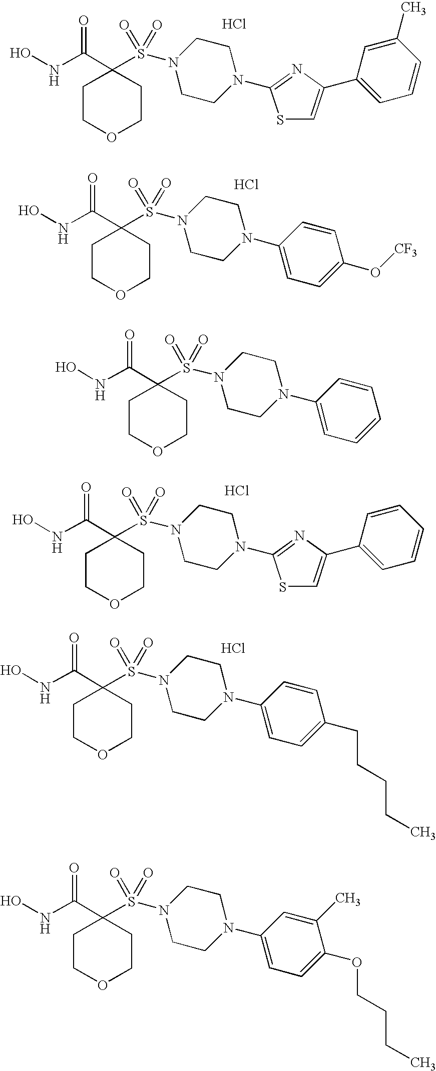

In some often particularly preferred embodiments, the compounds prepared by this invention include piperazinylsulfonyl α-substituted hydroxamic acids generally corresponding in structure to the following formula: 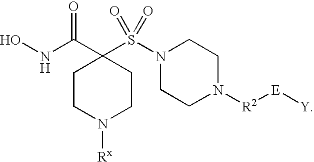
Such compounds include, for example, the following compounds wherein R^{x} is hydrogen: 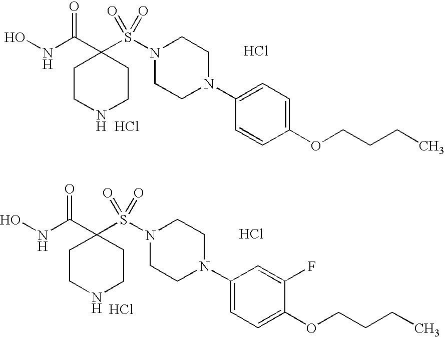
Such compounds also include, for example, compounds wherein R^{x} is other than hydrogen:

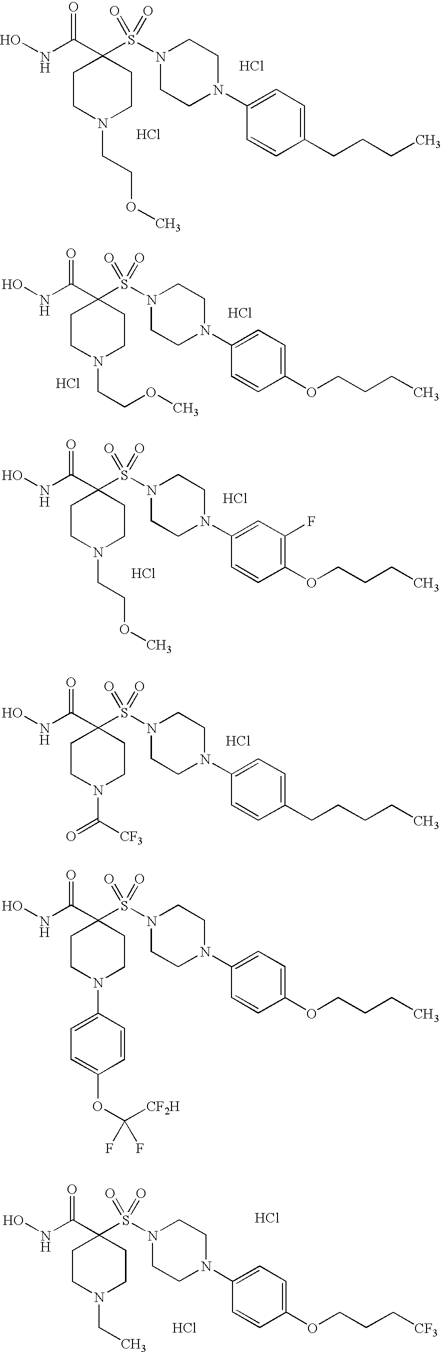

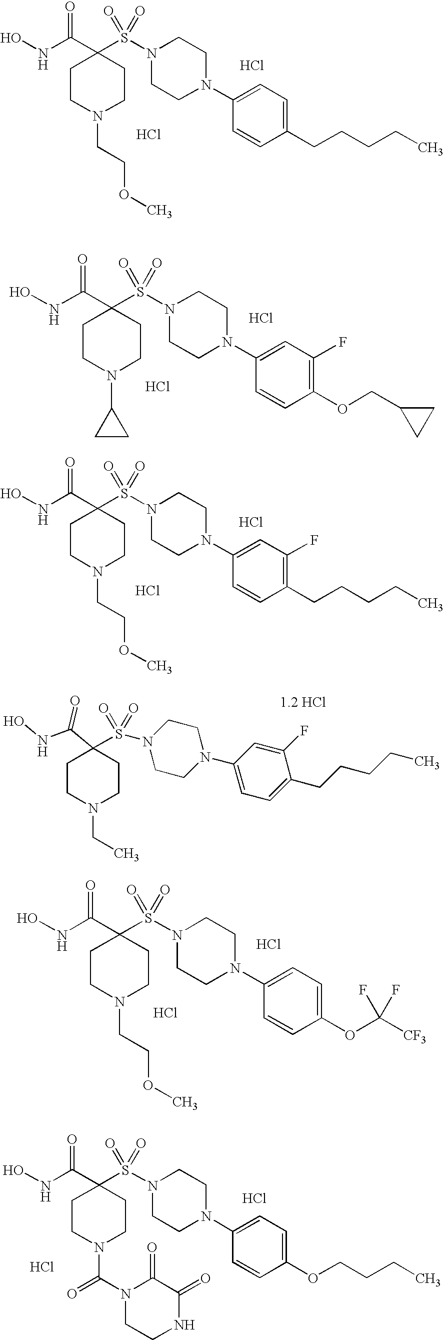

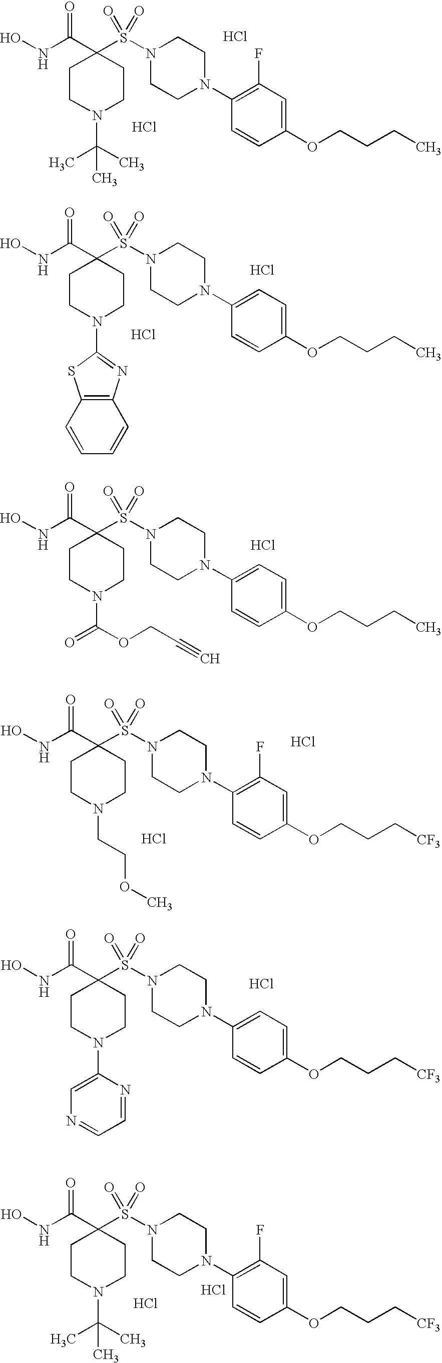

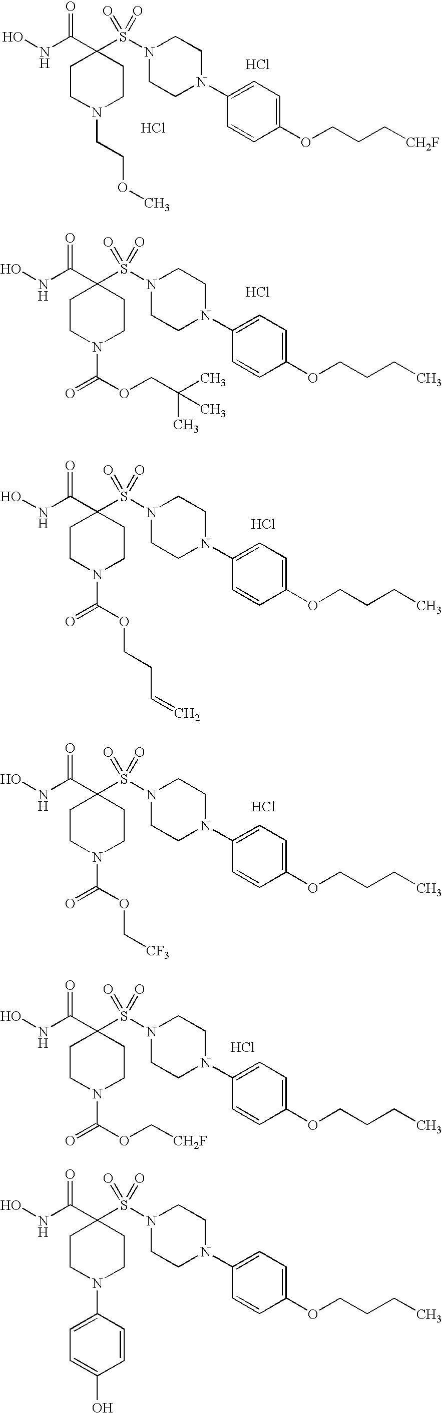

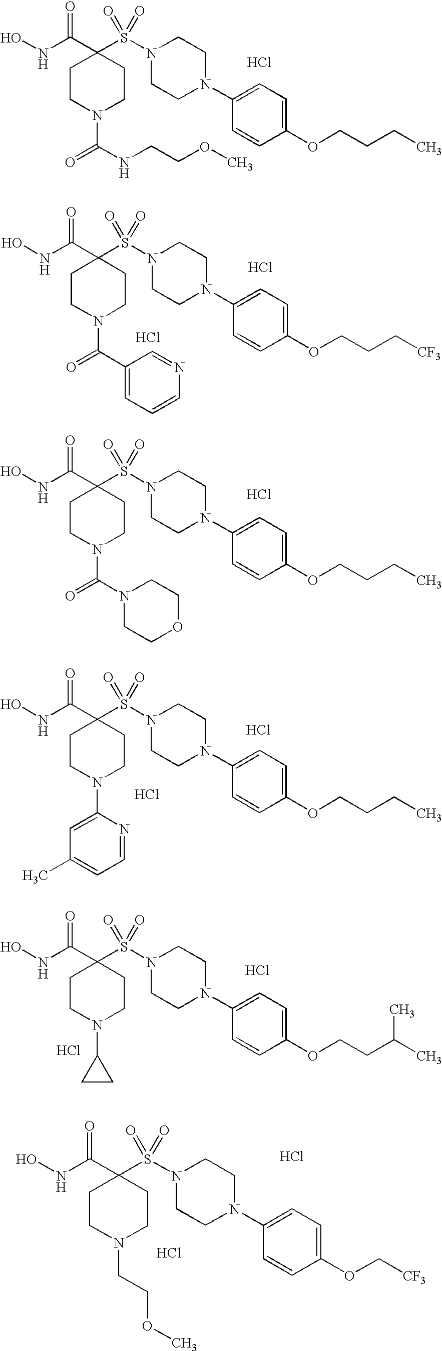

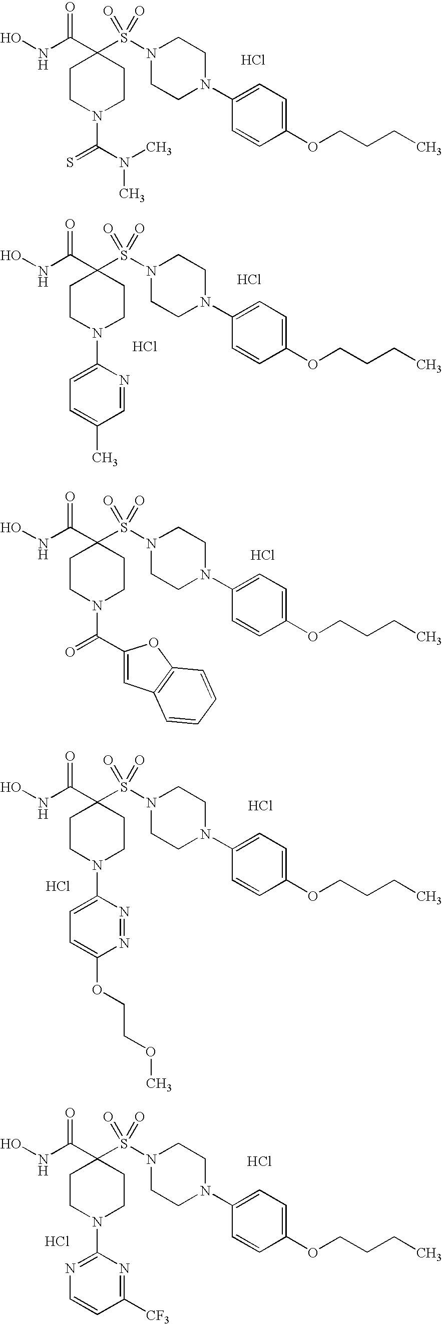

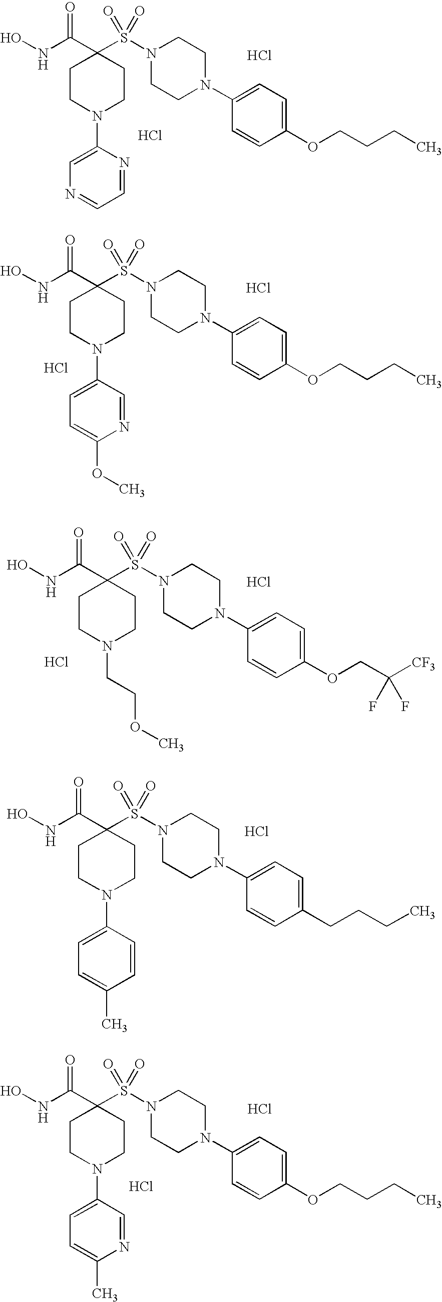

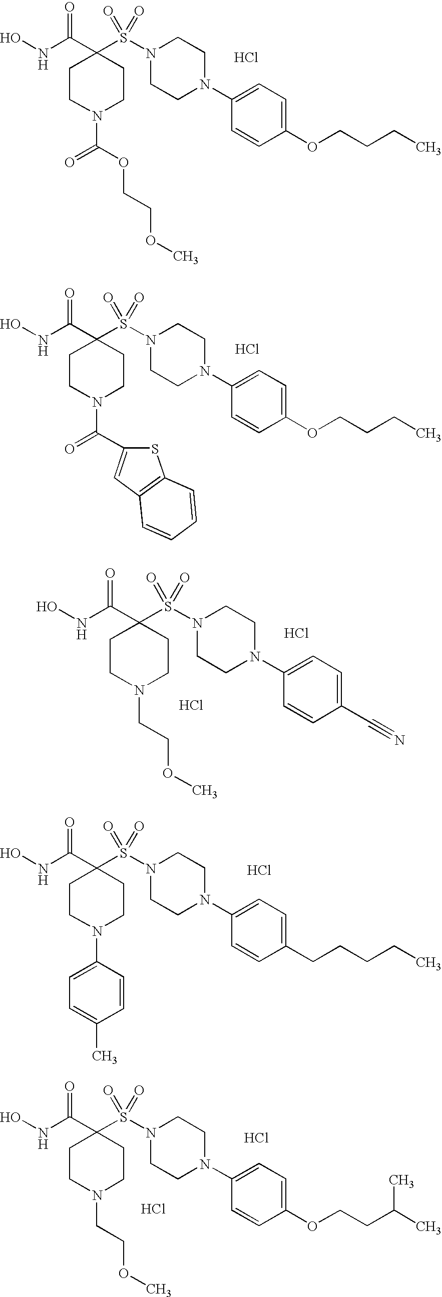

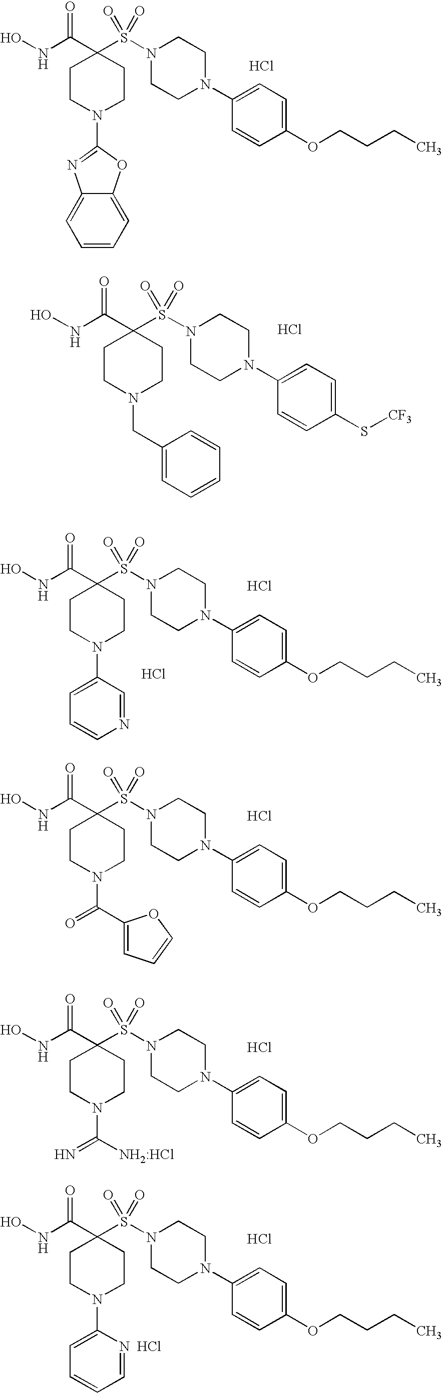

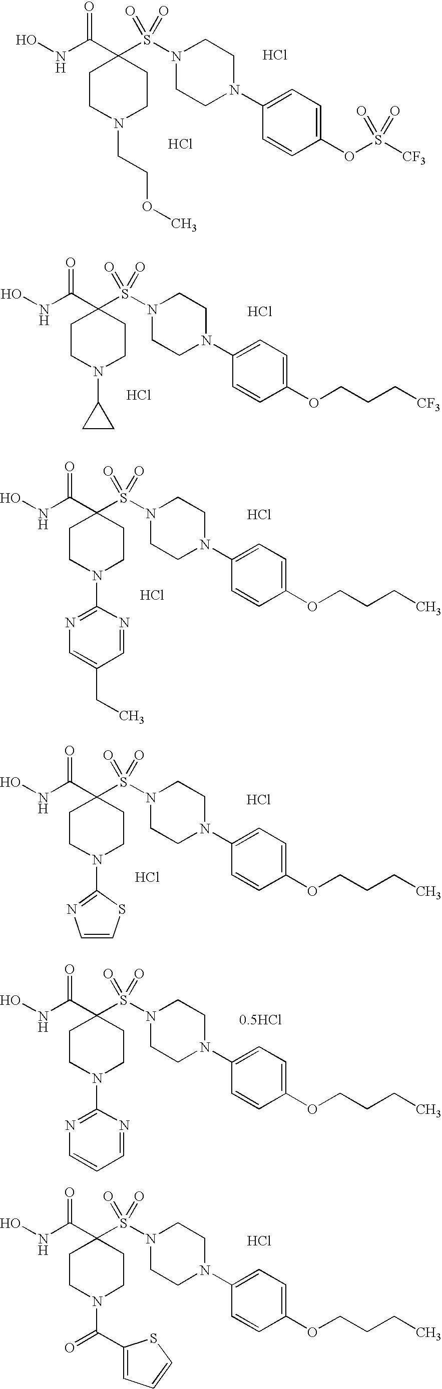

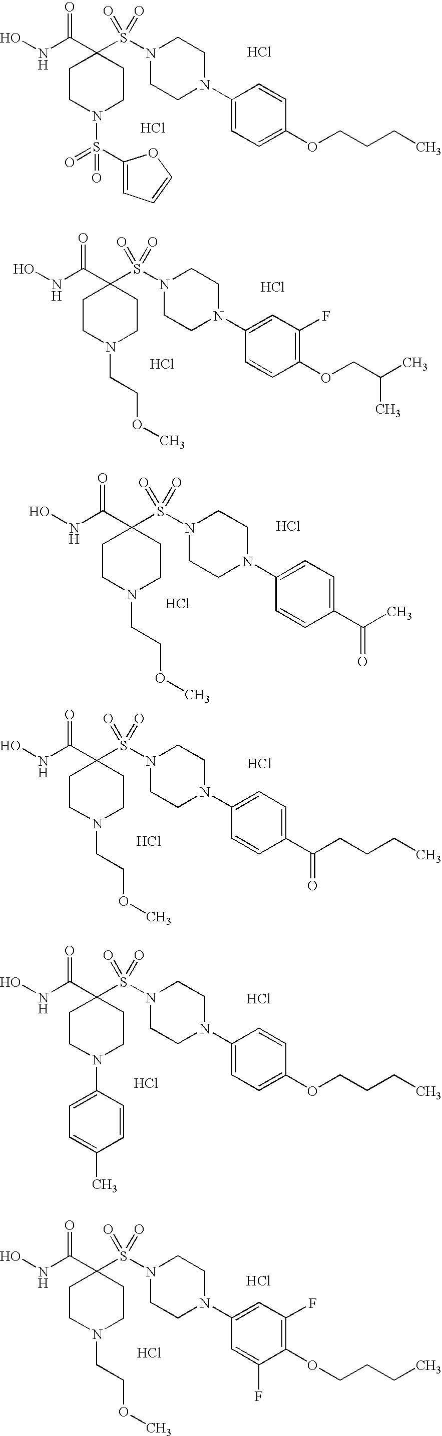

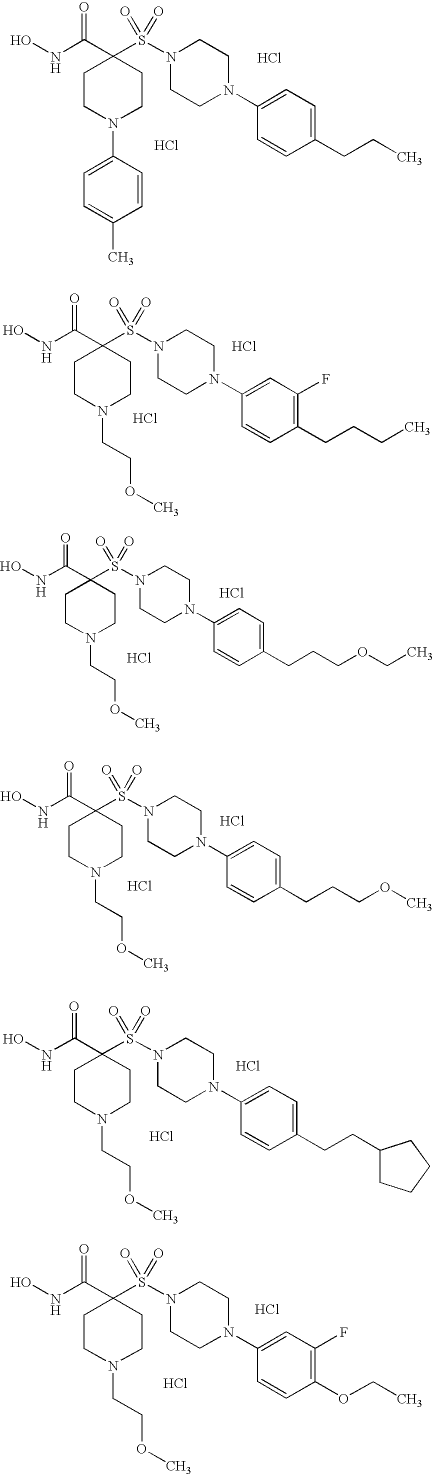

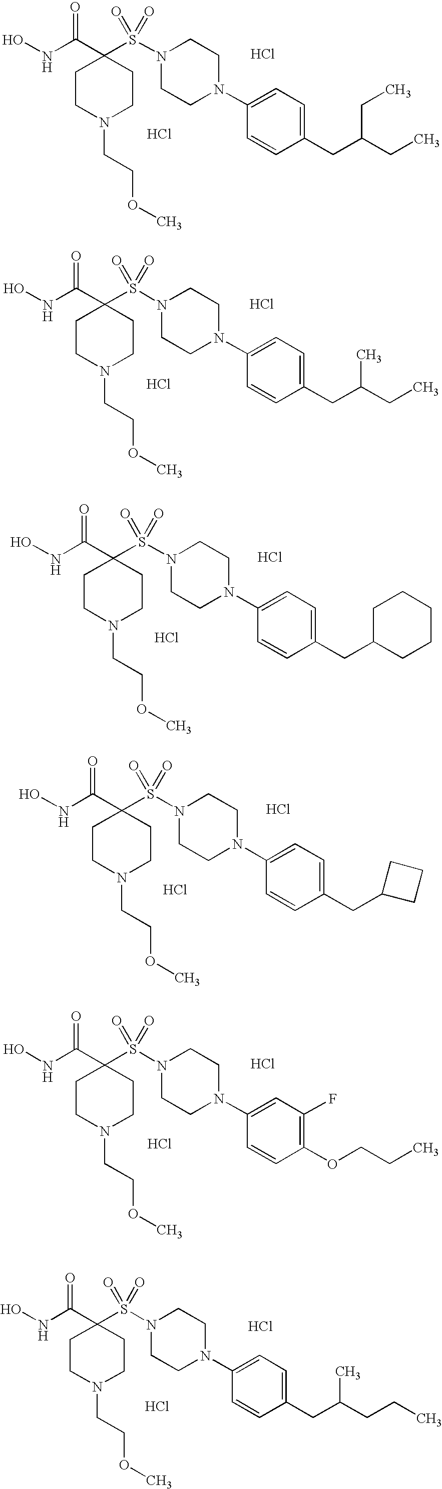

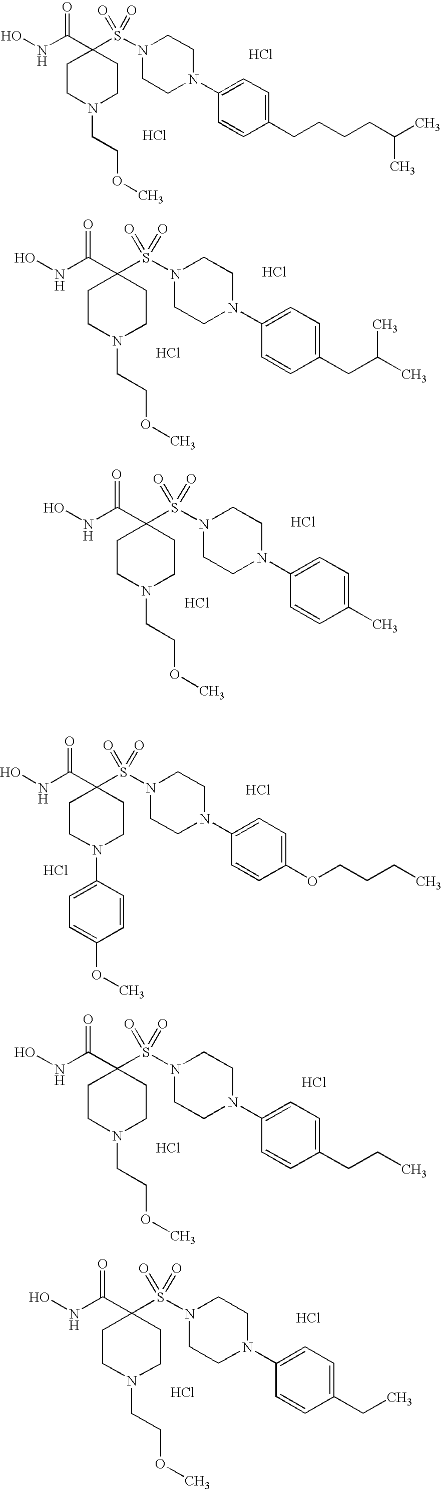

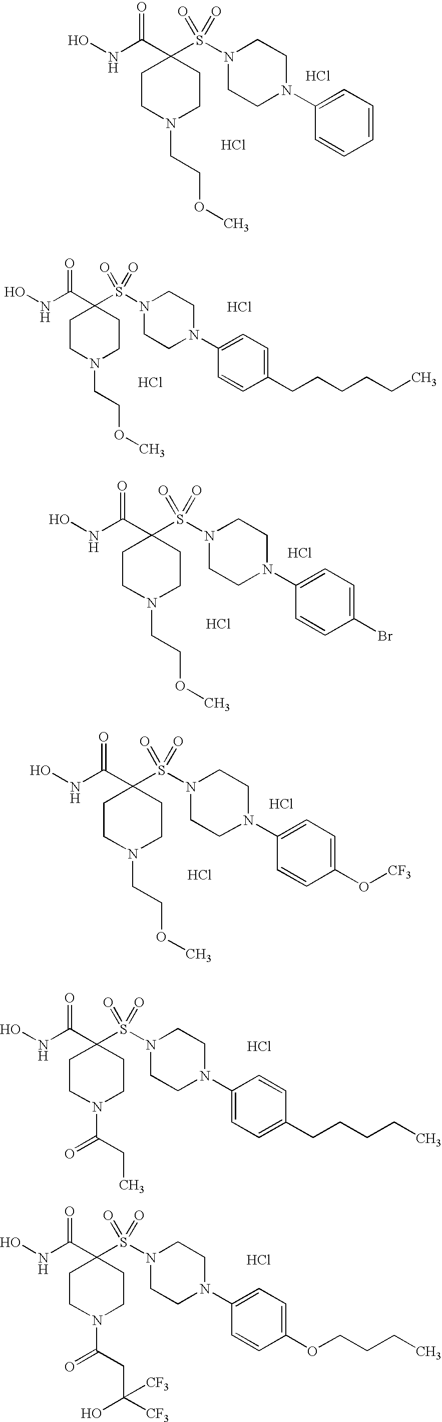

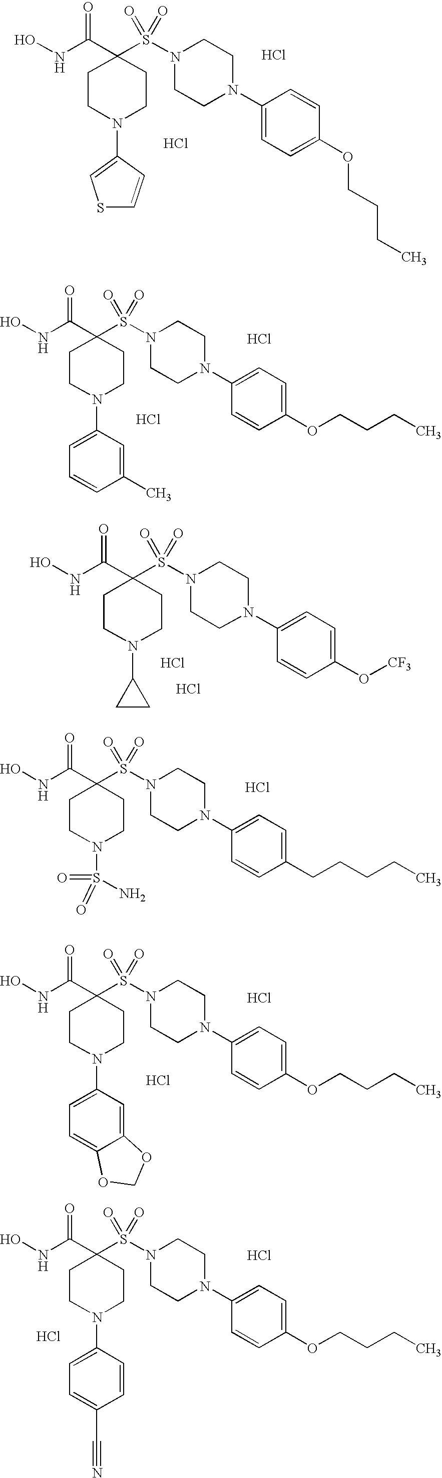

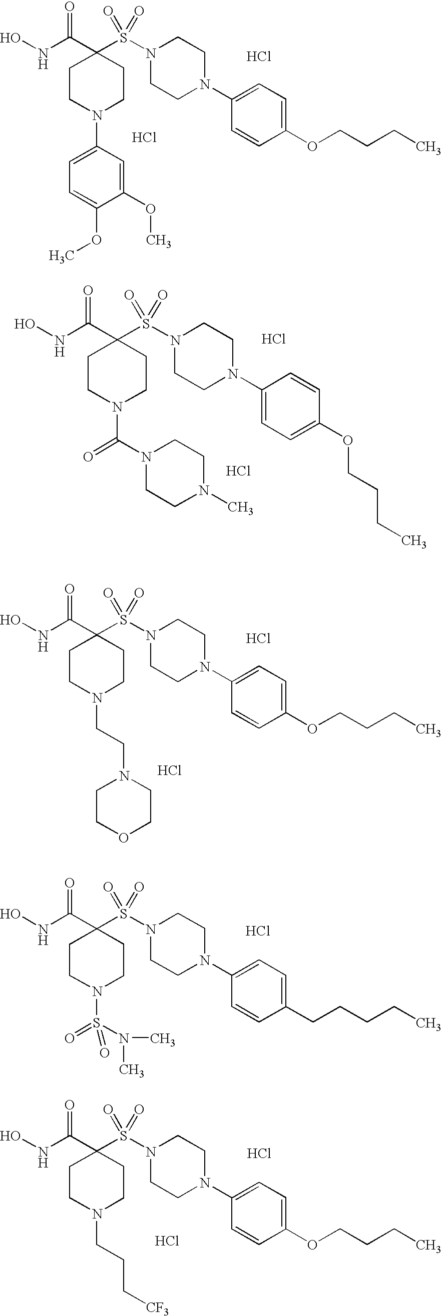

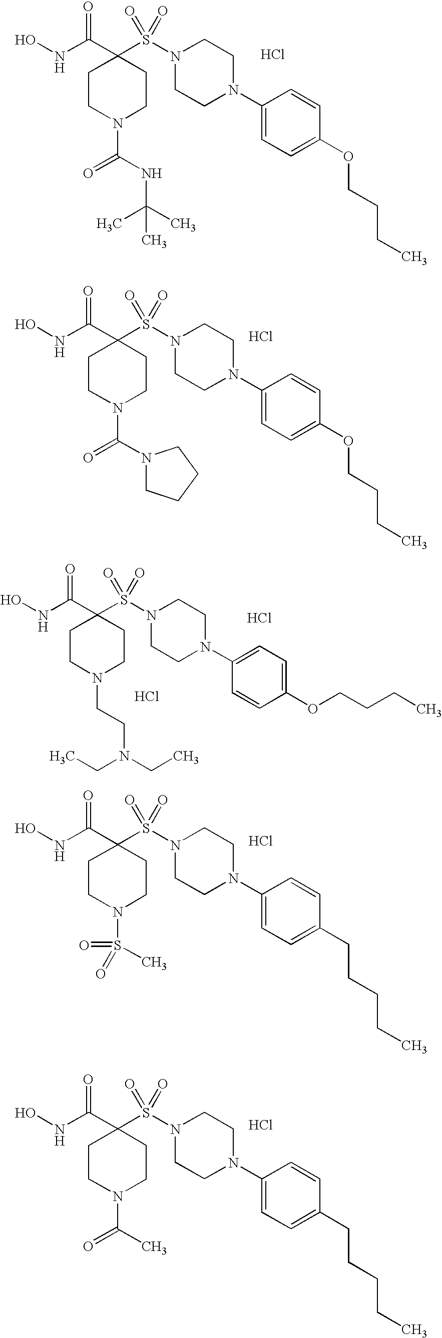

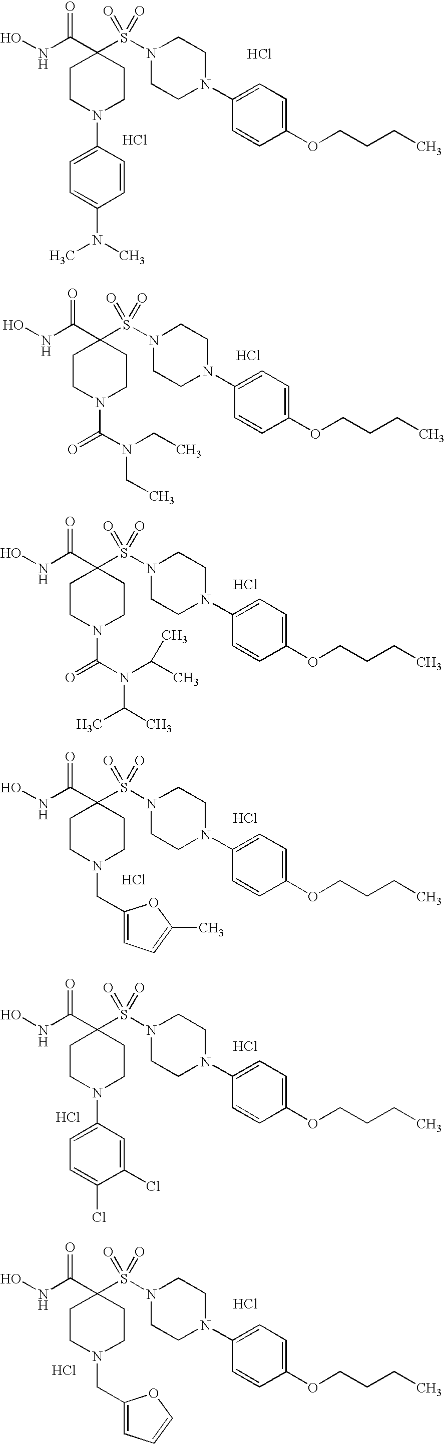

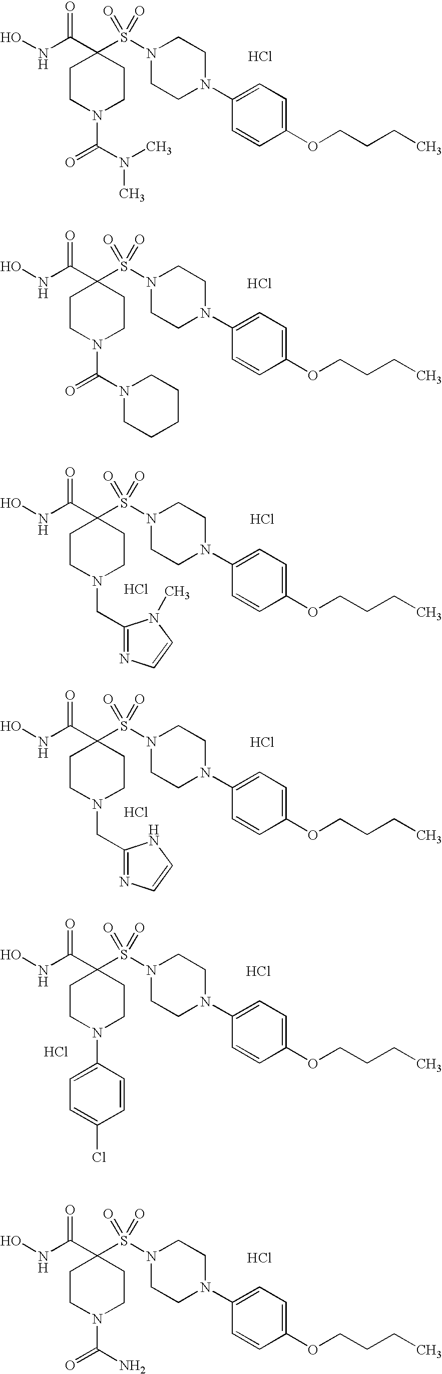

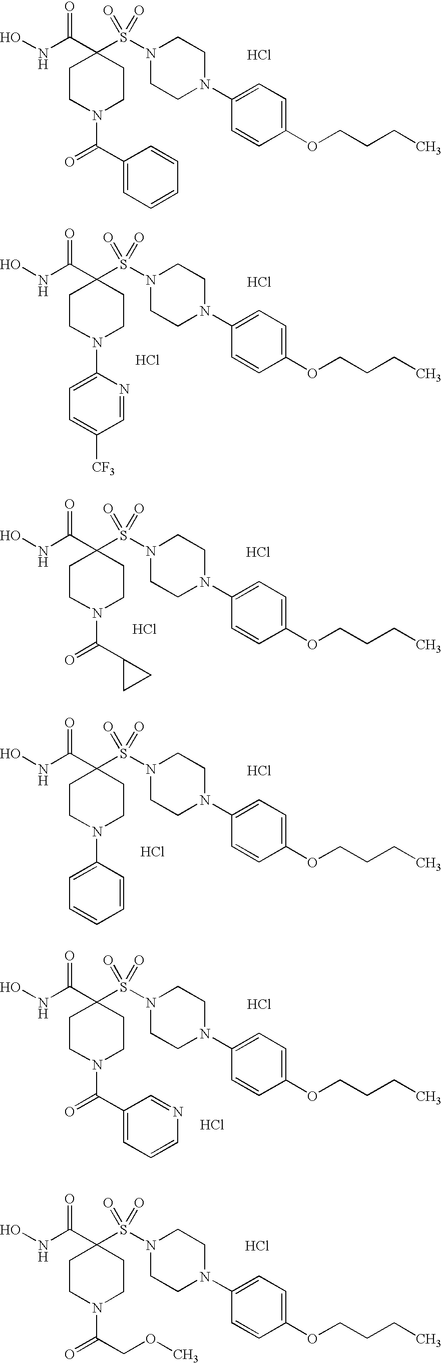

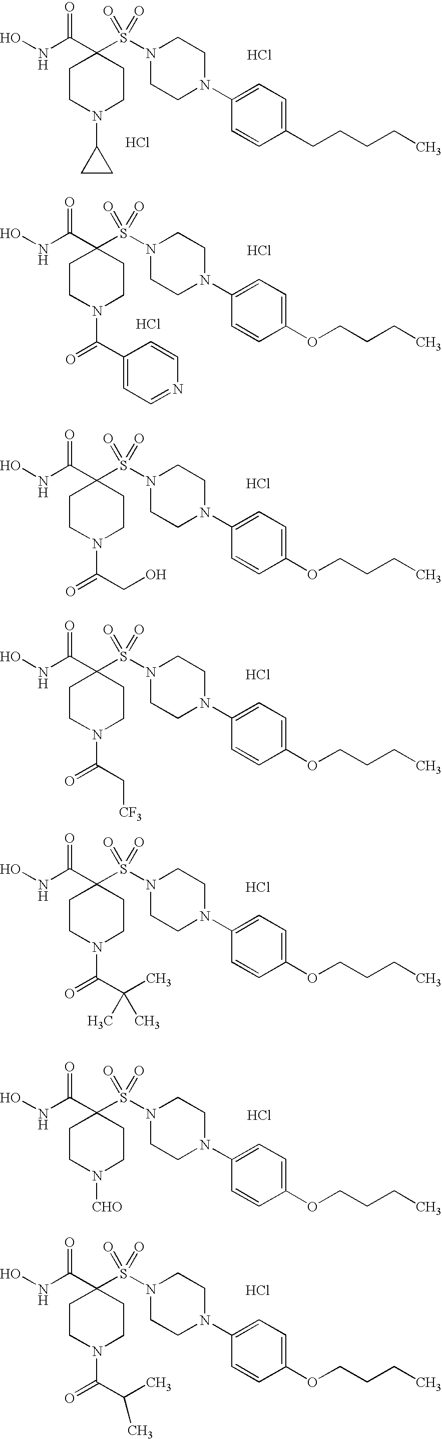

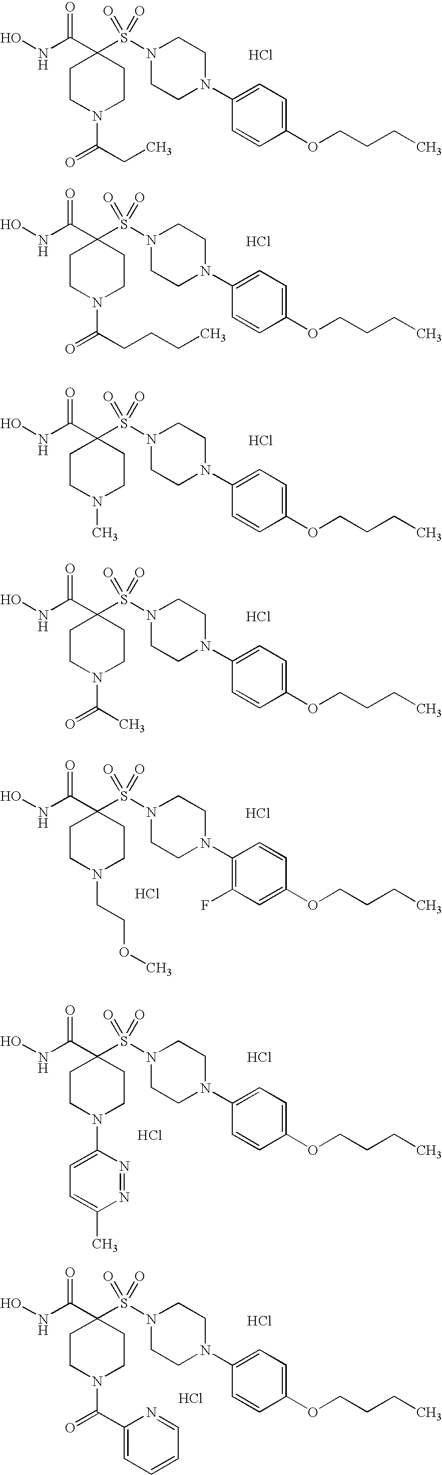

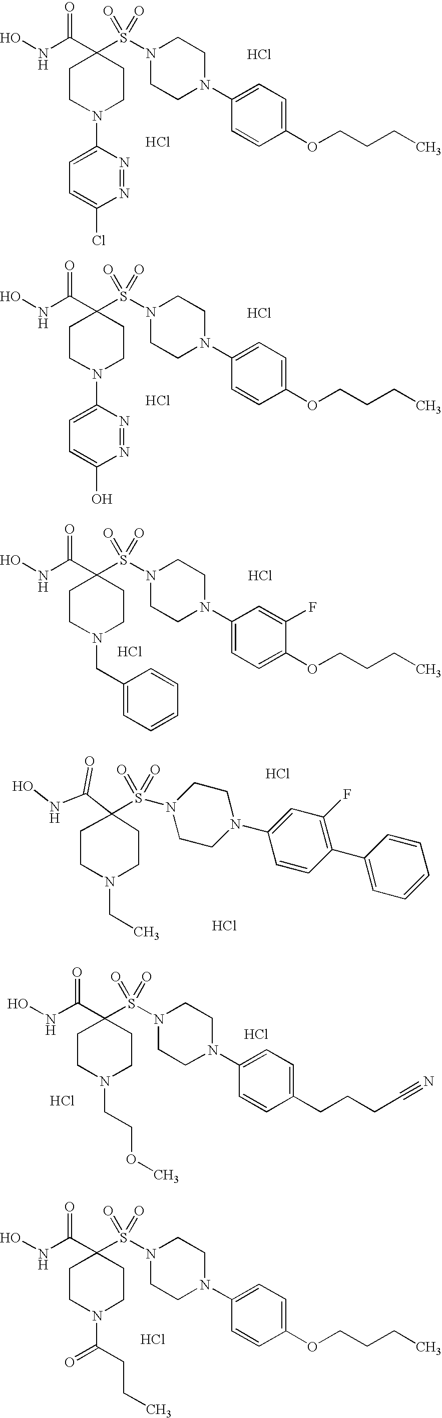

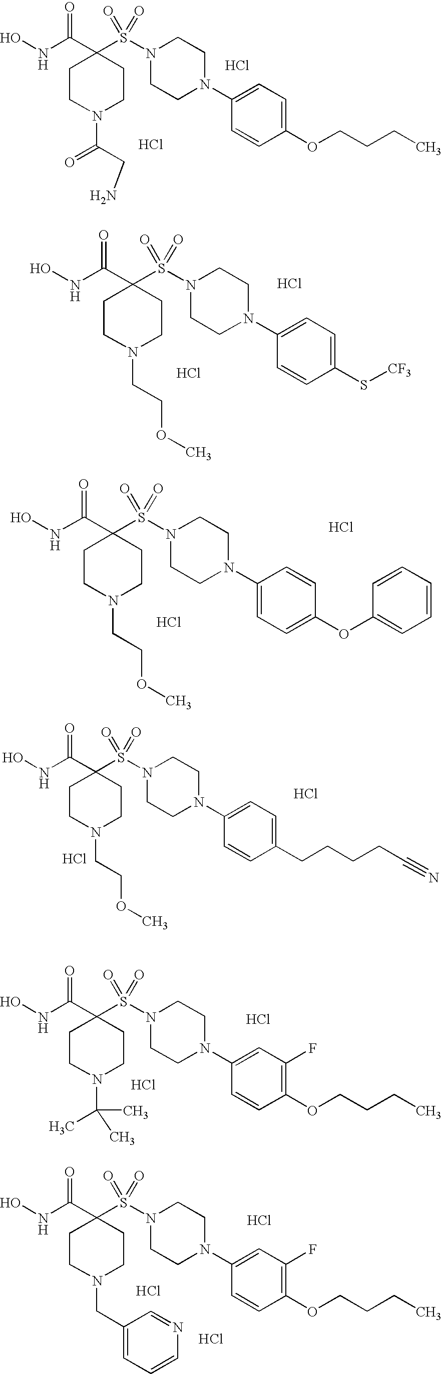

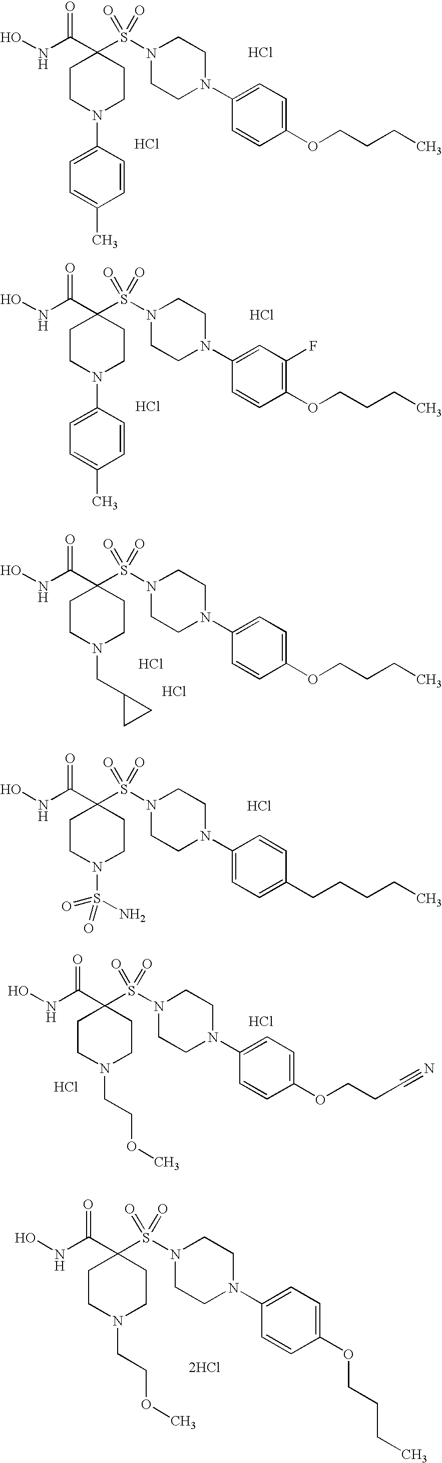

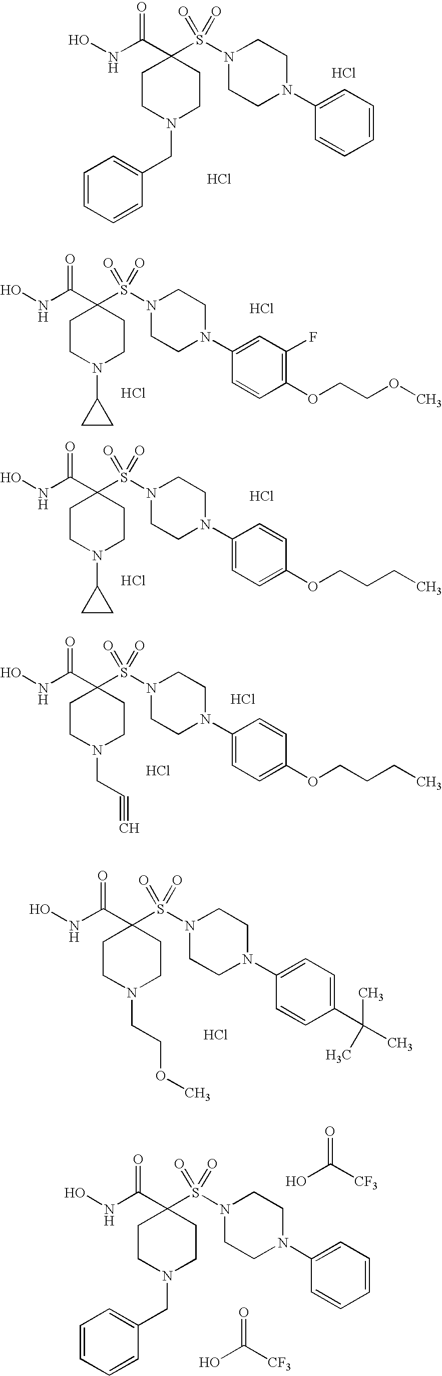

In some preferred embodiments, the compounds prepared by this invention include piperidinylsulfonyl α-substituted hydroxamic acids generally corresponding in structure to the following formula: 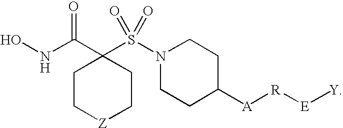
Such compounds include, for example, the following compounds wherein Z is —S—: 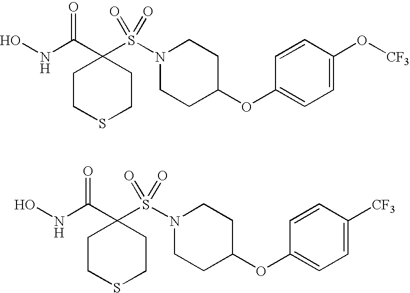
Such compounds also include, for example, the following compounds wherein Z is —S(O)₂—: 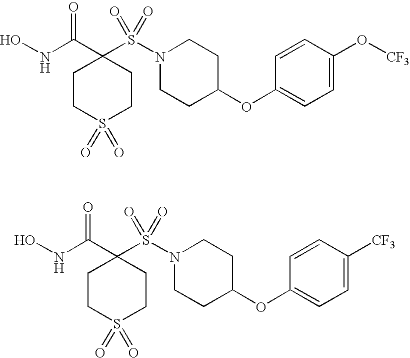
Such compounds also include, for example, the following compounds wherein Z is —O—:

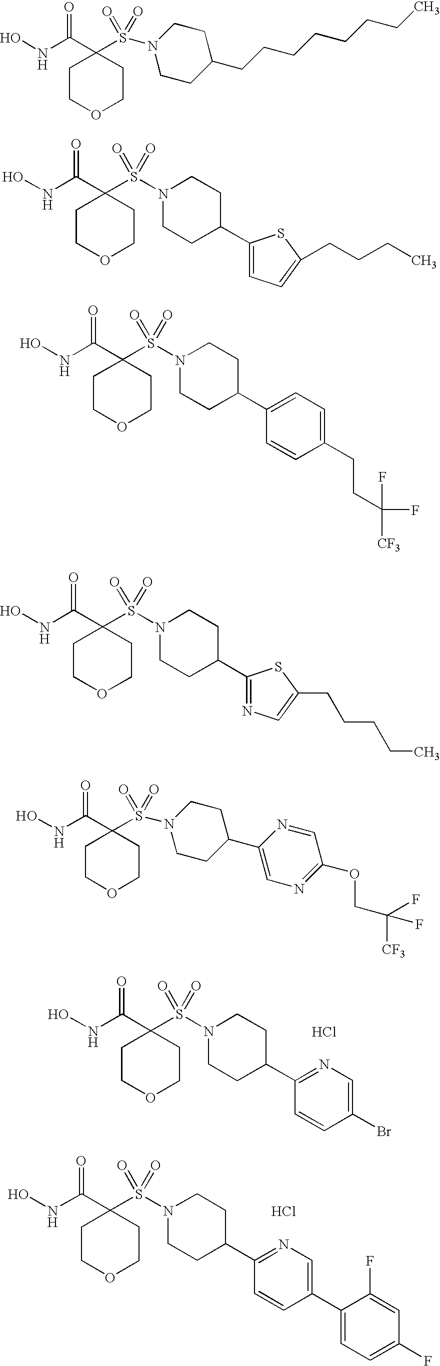

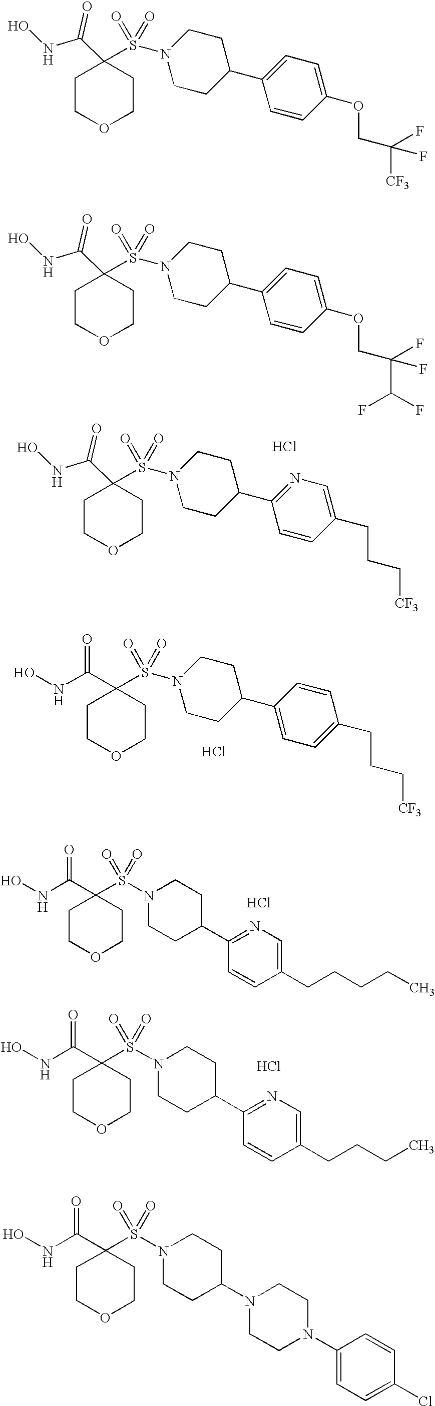

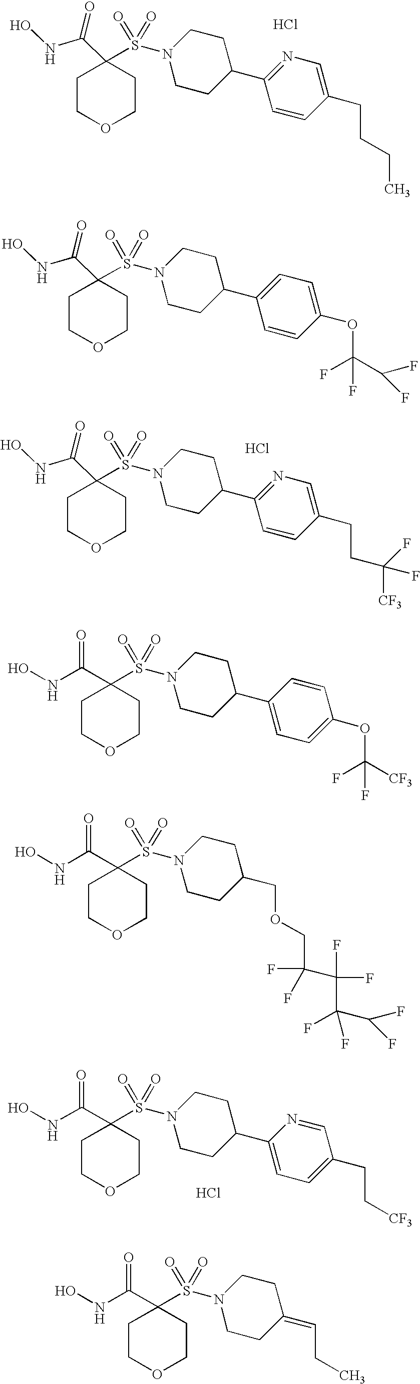

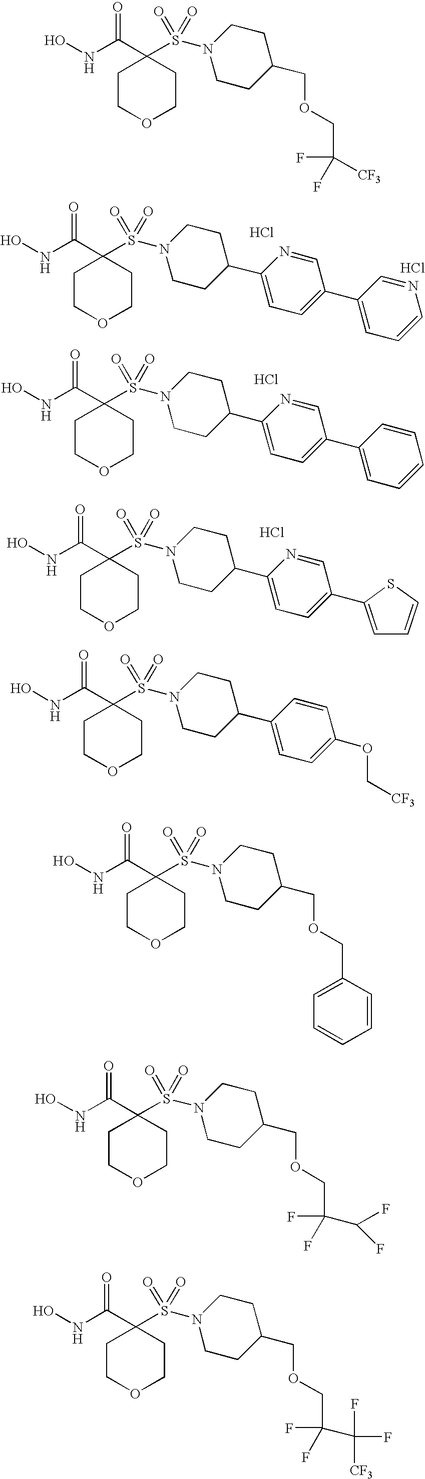

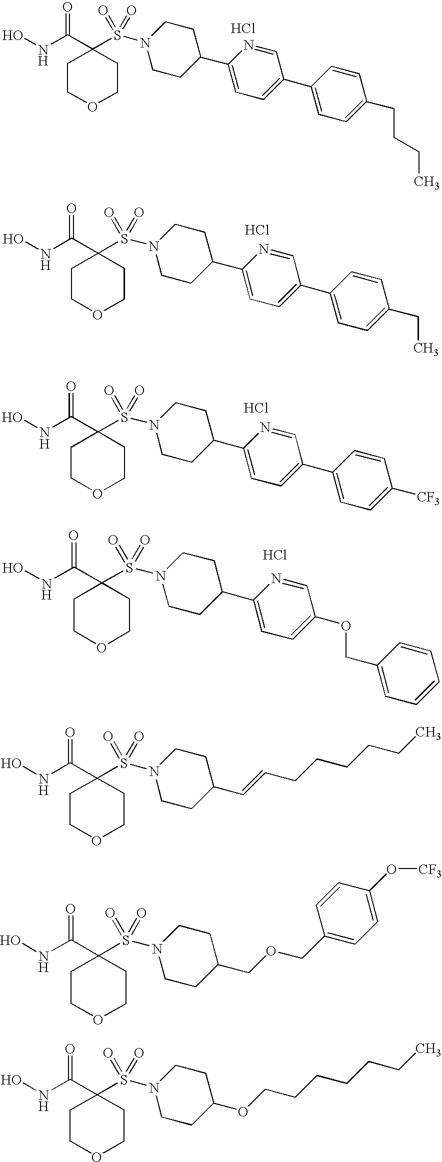

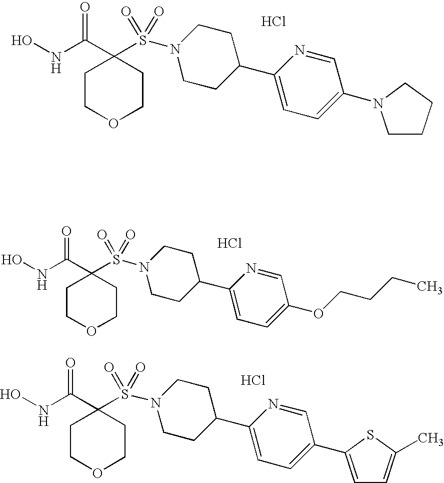

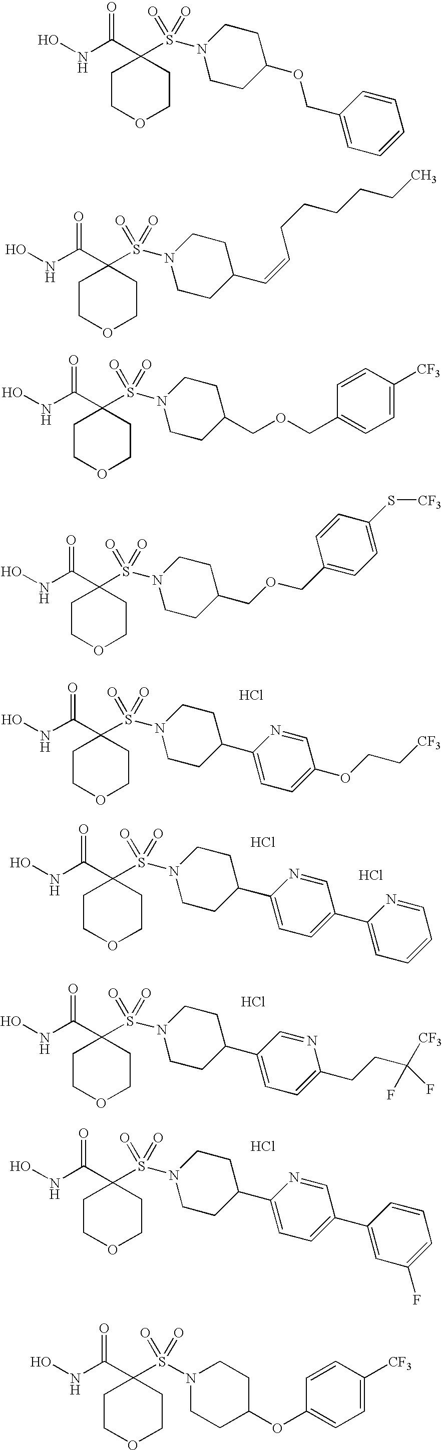

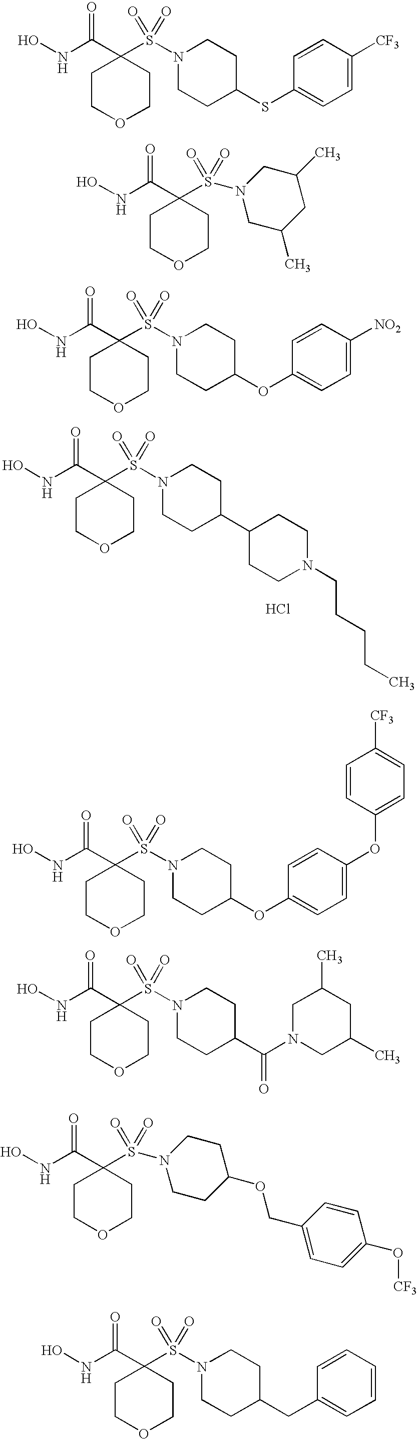

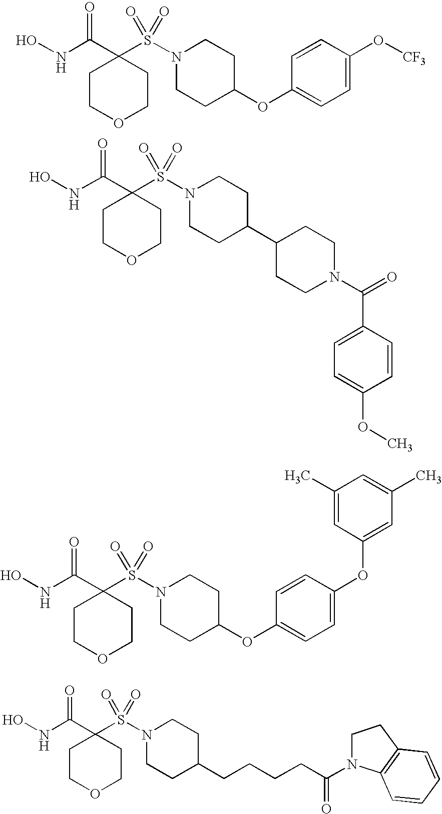

In some particularly preferred embodiments, the compounds prepared by this invention are piperidinylsulfonyl α-substituted hydroxamic acids generally corresponding in structure to the following formula: 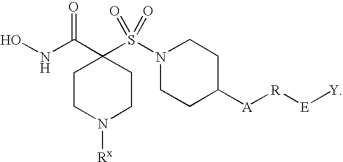
Such compounds include, for example, the following compounds wherein A is a bond and R is optionally-substituted heterocyclyl: 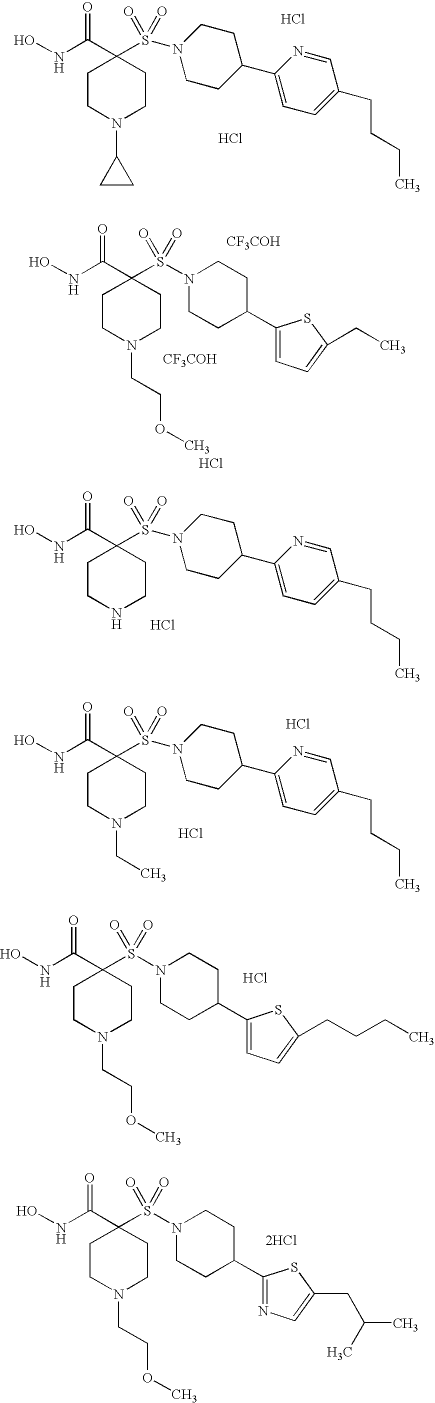 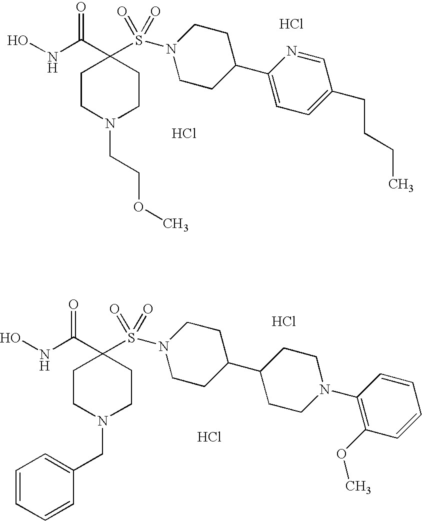
Such compounds also include, for example, the following compounds wherein A is a bond and R is optionally-substituted phenyl:

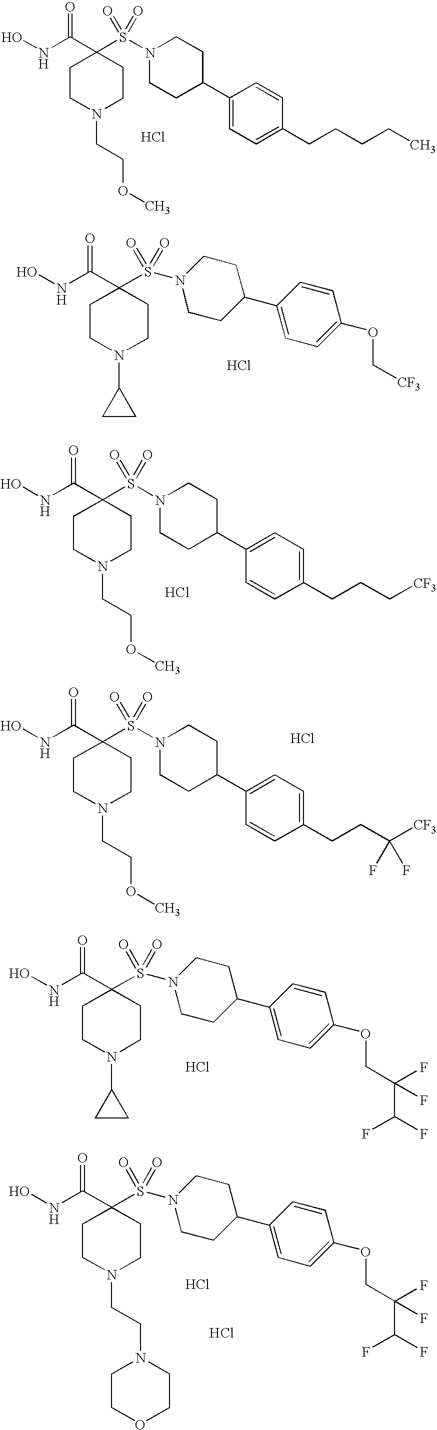

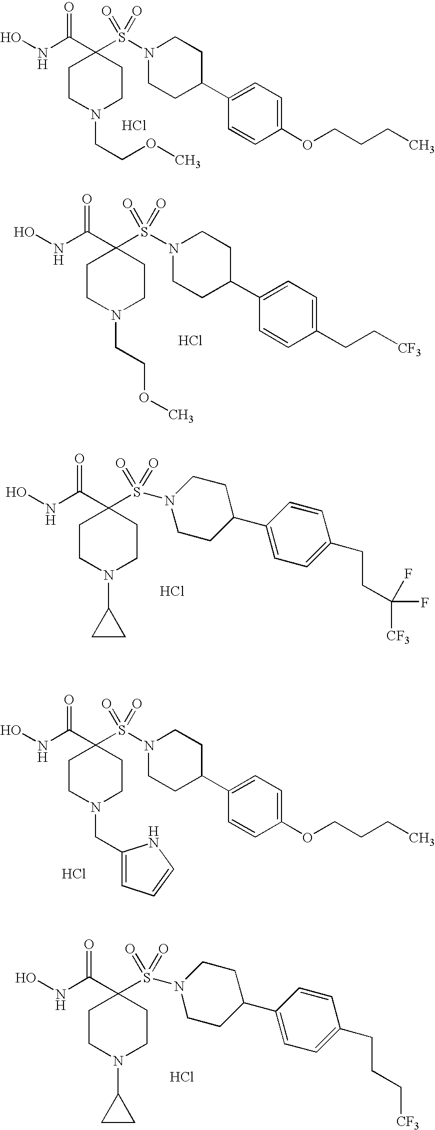

In some particularly preferred embodiments, the compounds prepared by this invention are piperidinylsulfonyl α-substituted hydroxamic acids generally corresponding in structure to the following formula: 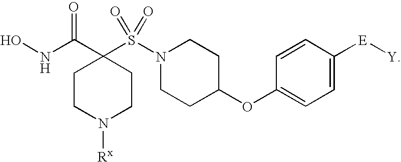
Such compounds include, for example, the following compounds wherein R^{x} is hydrogen, and -E-Y is trifluoromethoxy or trifluoromethyl: 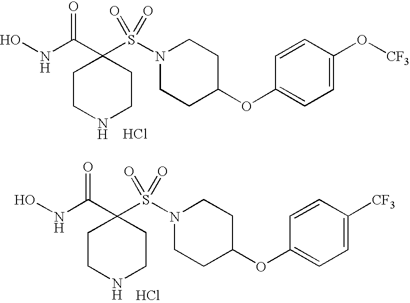
Such compounds also include, for example, the following compounds wherein R^{x} is other than hydrogen, and -E-Y is trifluoromethoxy or trifluoromethyl:

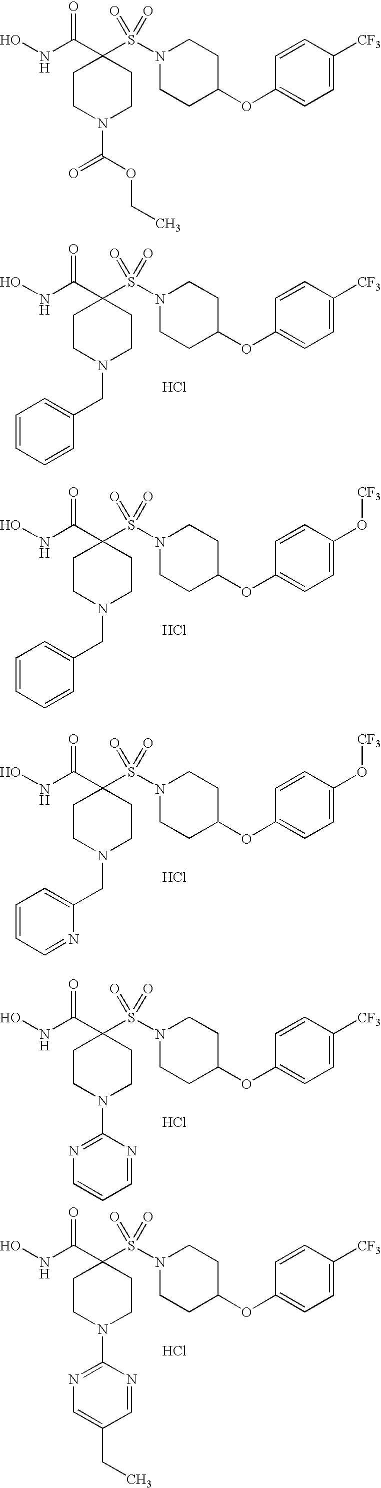

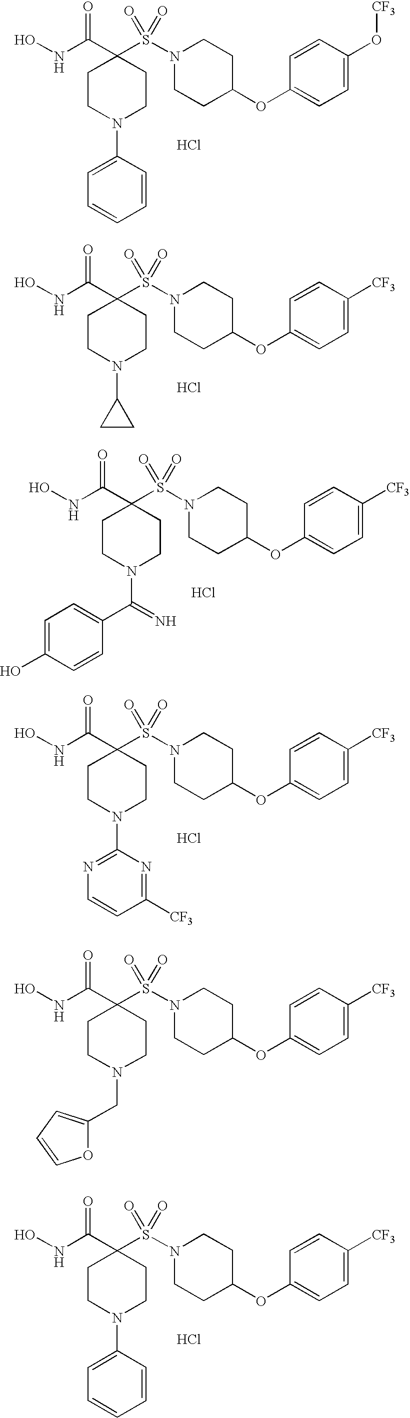

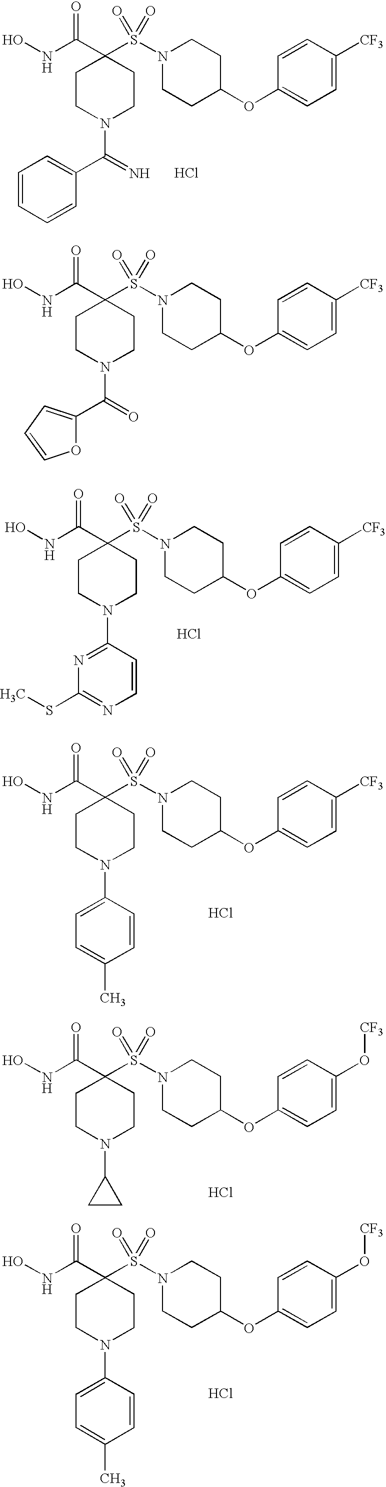

In some particularly preferred embodiments, the compounds prepared by this invention include piperidinylsulfonyl α-substituted hydroxamic acids generally corresponding in structure to the following formula: 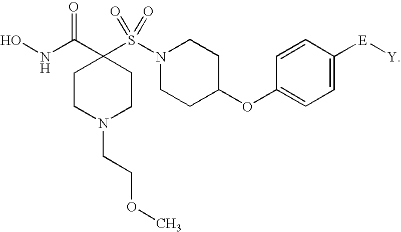
Such compounds include, for example, the following compound wherein -E-Y is trifluoromethyl: 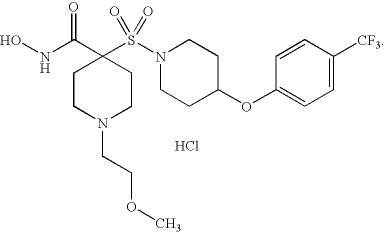
Such compounds also include, for example, the following compound wherein -E-Y is trifluoromethoxy:

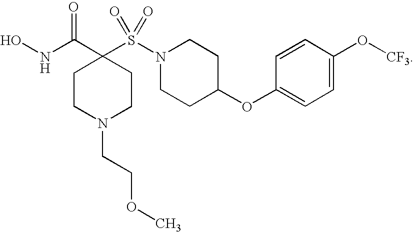

### B. Compound Preparation Process

The above-described hydroxamic acid compounds generally may be prepared by the process of this invention using materials that are commercially available and/or materials that may be readily prepared using methods well-known in the art. A preferred embodiment of the process of this invention is illustrated in Scheme (I): 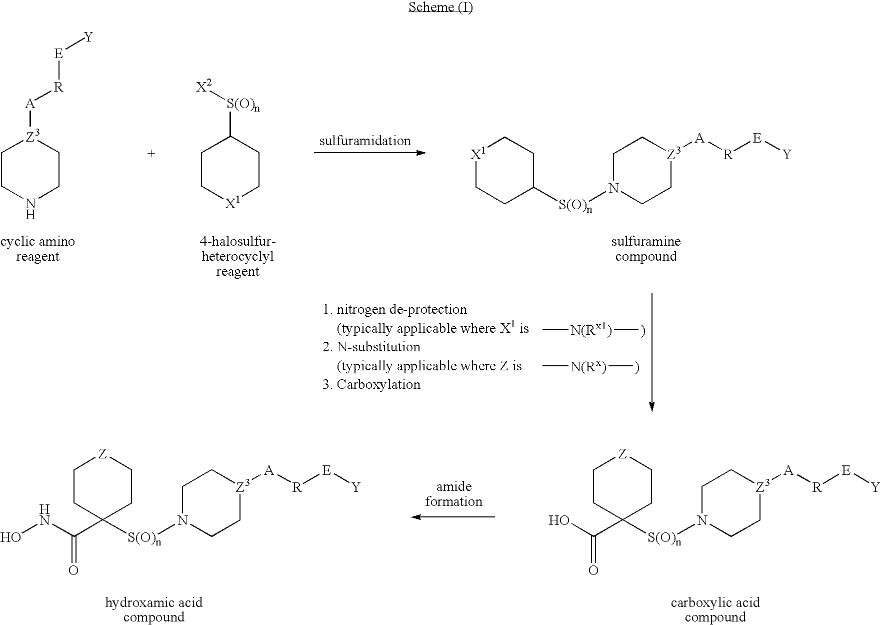
In the above scheme, Z and X¹ will generally be the same in instances where there is no nitrogen de-protection and N-substitution. The following discussion provides a more detailed description of the above scheme.

### B-1. Sulfuramidation

As indicated above, this invention contemplates reacting a cyclic amino reagent with a 4-halosulfur-heterocyclyl reagent to form a sulfuramine compound: 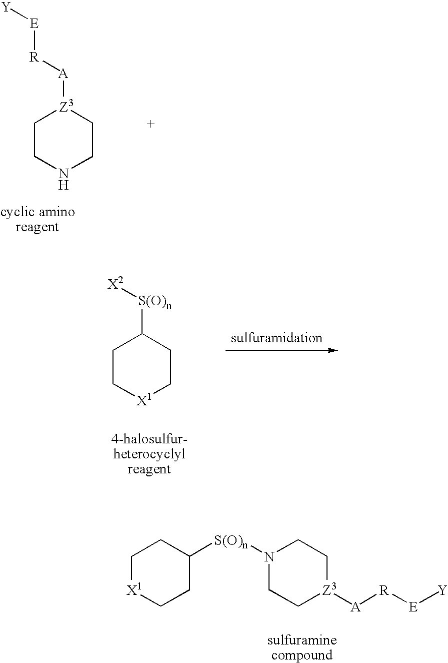
This reaction is preferably carried out as follows.

### B-1(a). The Cyclic Amino Reagent

The cyclic amino reagent generally corresponds in structure to the following formula: 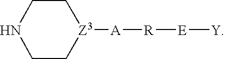
Here, A, R, E, Y, and Z³ are generally as defined above.

Where, for example, the desired hydroxamic acid compound corresponds in structure to the following formula: 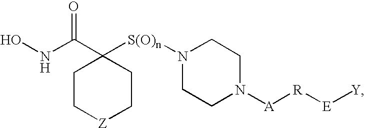
the cyclic amino reagent will typically correspond in structure to the following formula: 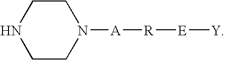
In some such instances, the desired hydroxamic acid compound corresponds in structure to the following formula: 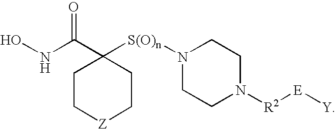
In those instances, the cyclic amino reagent will typically correspond in structure to the following formula:

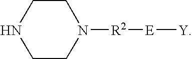

Where, for example, the desired hydroxamic acid compound corresponds in structure to the following formula: 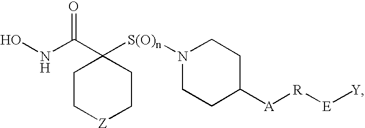
the cyclic amino reagent will typically correspond in structure to the following formula: 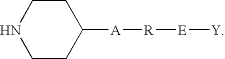
In some such instances, the desired hydroxamic acid compound corresponds in structure to the following formula: 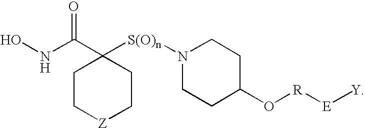
In those instances, the cyclic amino reagent will typically correspond in structure to the following formula: 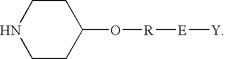
In other such instances, the desired hydroxamic acid compound corresponds in structure to the following formula: 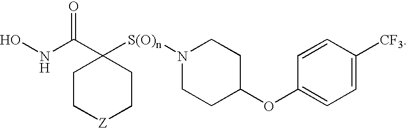
In those instances, the cyclic amino reagent will typically be: 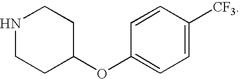
In yet other instances, the desired hydroxamic acid compound corresponds in structure to the following formula: 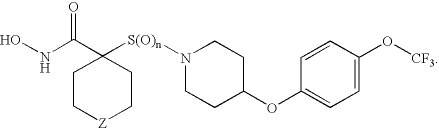
In those instances, the cyclic amino reagent will typically be:

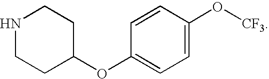

If not commercially available, the cyclic amino compound can generally be made from readily-available materials using methods well known in the art. Such methods include, for example, those disclosed in WIPO Intl. Publ. No. WO 00/46221; U.S. Pat. No. 6,448,250; U.S. Pat. No. 6,372,758; and U.S. Pat. No. 6,492,367 (all of which are cited above and incorporated by reference into this patent).

As indicated above, for example, the cyclic amino compound may generally correspond in structure to the following formula: 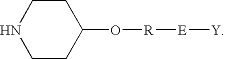
Such compounds can be prepared from, for example, 4-hydroxypiperidine (a commercially-available material). Scheme (II) illustrates an example of such a synthesis: 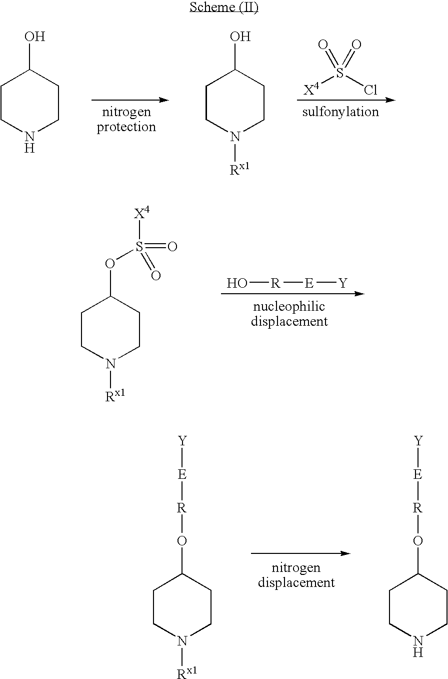
Here, the piperidine nitrogen is protected with a nitrogen protecting group, R^{x1} (e.g., alkoxycarbonyl or arylalkoxycarbonyl). Afterward, the hydroxy group is sulfonylated with a sulfonylchloride (—S(O)₂—X⁴, wherein X⁴ may be, for example, alkyl, haloalkyl, aryl, or haloaryl), to form a 4-sulfonyloxy-heterocyclyl compound, which preferably is isolated (typically as a solid) from at least a portion of the other components of sulfonylation product mixture. The sulfonate ester group of the 4-sulfonyloxy-heterocyclyl compound is, in turn, nucleophilically displaced with an alcohol group (HO—R-E-Y, wherein R, E, and Y are generally as defined above). Subsequently, the piperidine nitrogen is de-protected using, for example, acid hydrolysis. Example 1 (Parts A-C), Example 2 (Parts A-C), and Example 4 below illustrate examples of suitable methods for preparing a cyclic amino reagent.

### B-1(b). The 4-Halosulfur-Heterocyclyl Reagent

### The 4-halosulfur-heterocyclyl reagent generally corresponds in structure to the following formula:

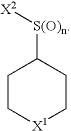 Here:

X² is halogen, and preferably chloro.

In many preferred embodiments, n is 2. In such embodiments, the 4-halosulfur-heterocyclyl compound is a 4-halosulfonyl-heterocyclyl compound, and generally corresponds in structure to the following formula: 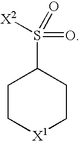
Such a 4-halosulfonyl-heterocyclyl reagent is particularly preferred where n is 2 in the desired hydroxamic acid compound, i.e., where the desired hydroxamic acid compound corresponds in structure to the following formula:

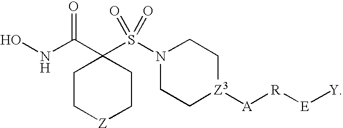

In some embodiments, X¹ is —O—. This may be particularly preferred when the desired hydroxamic acid compound has a tetrahydropyranyl group at the hydroxamic acid α-carbon, i.e., where Z is —O—.

In some embodiments, X¹ is —S—. This may be particularly preferred when the desired hydroxamic acid compound has a tetrahydrothiopyranyl group at the hydroxamic acid α-carbon, i.e., where Z is —S—. Given that the thio group of a tetrahydrothiopyranyl group may often be oxidized to form a sulfoxido or sulfonyl group using techniques well-known in the art, X¹ may also be —S— in some embodiments where the desired hydroxamic acid compound has an oxidized derivative of a tetrahydrothiopyranyl group at the hydroxamic acid α-carbon. Such an oxidized derivative may be, for example, 1-oxide-tetrahydrothiopyranyl (i.e., where Z is —S(O)—) or 1,1-dioxide-tetrahydrothiopyranyl (i.e., where Z is —S(O)₂—).

In some embodiments, X¹ is —S(O)—. This may be particularly preferred when the desired hydroxamic acid compound has a 1-oxide-tetrahydrothiopyranyl group at the hydroxamic acid α-carbon, i.e., where Z is —S(O)—. Given that the sulfoxido group of a 1-oxide-tetrahydrothiopyranyl group may often be oxidized to form a sulfonyl group using techniques well-known in the art, X¹ may also be —S(O)— in some embodiments where the desired hydroxamic acid compound has a 1,1-dioxide-tetrahydrothiopyranyl group at the hydroxamic acid α-carbon (i.e., where Z is —S(O)₂—).

In some embodiments, X¹ is —S(O)₂—. This may be particularly preferred when the desired hydroxamic acid compound has a 1,1-dioxide-tetrahydrothiopyranyl group at the hydroxamic acid α-carbon, i.e., where Z is —S(O)₂—.

In some embodiments, X¹ is —N(R^{x1})—. This may be particularly preferred when the desired hydroxamic acid compound has a piperidinyl group at the hydroxamic acid α-carbon, i.e., where Z is —N(R^{x})—.

Where X¹ is —N(R^{x1})—, R^{x1} is a nitrogen-protecting group.

In some embodiments, R^{x1} is alkoxyalkyl, e.g., methoxyethyl. In those instances, the 4-halosulfonyl-heterocyclyl compound may be, for example: 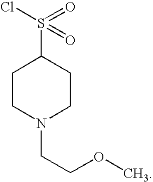
This reagent may be preferred where the desired hydroxamic acid compound corresponds in structure to the following formula:

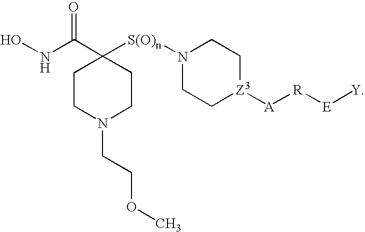

In other embodiments, R^{x1} is alkoxycarbonyl or arylalkoxycarbonyl. Such substituents are normally easily removable from the piperidinyl nitrogen. Thus, they are particularly preferred in embodiments where the R^{x1} is a temporary protecting group that is removed downstream in the process. In some such embodiments, R^{x1} is phenylmethoxycarbonyl. Here, the 4-halosulfonyl-heterocyclyl compound may be, for example: 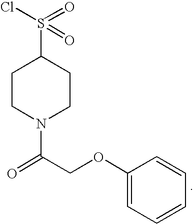
In other such embodiments, R^{x1} is t-butoxycarbonyl. Here, the 4-halosulfonyl-heterocyclyl compound may be, for example:

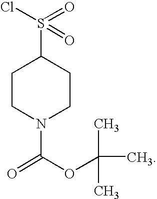

The 4-halosulfur-heterocyclyl compound may be prepared from commercially available materials using a variety of methods. Because 4-halosulfonyl-heterocyclyl compounds are generally preferred, the following discussion focuses on preparation of such compounds. Similar methods, however, may be used to prepare compounds wherein n is zero or 1 as well.

### B-1(b)(i). Example Embodiment for Preparing 4-Halosulfonyl-Heterocyclyl Compounds

Commercially-available starting materials for preparing the 4-halosulfonyl-heterocyclyl compounds include, for example, 4-halo-heterocyclyl compounds. Such compounds generally correspond in structure to the following formula: 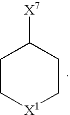
X⁷ may be, for example, bromo, iodo, or chloro, with bromo often being particularly preferred. In instances where the desired X¹ is —N(R^{x1})—, the 4-halo-heterocyclyl compound may be prepared from a commercially available 4-halo-piperidine (e.g., 4-bromo-piperidine) by protecting the piperidine nitrogen with the desired protecting group, R^{x1}. If, for example, the desired protecting group is benzyloxycarbonyl, the 4-halo-piperidine may be reacted with benzyloxycarbonylchloride (also known as benzylchloroformate) in the presence of a base (e.g., potassium carbonate) and a solvent (e.g., tetrahydrofuran) at, for example, ambient temperature and pressure: 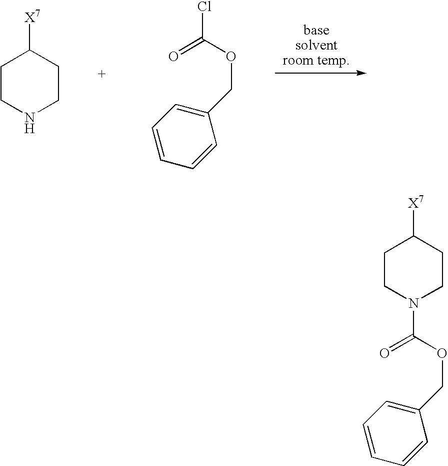
Example 1 (Part D) below illustrates such a preparation. Similar techniques may be used to protect the nitrogen with other protecting groups. Such protecting groups include, for example, t-butyloxycarbonyl. A 4-halo-piperidine protected with t-butyloxycarbonyl would generally correspond in structure to the following formula: 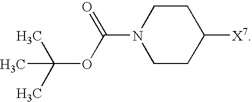
Other nitrogen protecting groups known in the art may also be used. Discussions relating to such protecting groups may be found in, for example, Greene, T. W.; Wuts, P. G. M.; Protective Groups in Organic Synthesis; 3rd Ed.; Wiley: New York, 1999 (incorporated by reference into this patent).

The 4-halosulfonyl-heterocyclyl compound may be prepared from a 4-halo-heterocyclyl compound by, for example, nucleophilically displacing the halogen (i.e., X⁷) of the 4-halo-heterocyclyl compound with a thioester group to form a 4-thioester-heterocyclyl compound (i.e., a “4-carbonylthio-heterocyclyl compound”), and oxidatively halogenating the 4-thioester-heterocyclyl compound: 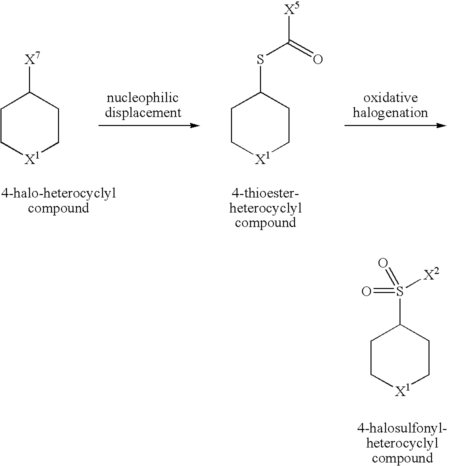
Here, X¹ and X⁷ are as generally defined above, and X² and X⁵ are as generally defined below.

The halogen (i.e., X⁷) of the 4-halo-heterocyclyl compound may be nucleophilically displaced with a thioester group to form the 4-thioester-heterocyclyl compound by, for example, reacting the 4-halo-heterocyclyl with a metal thioester: 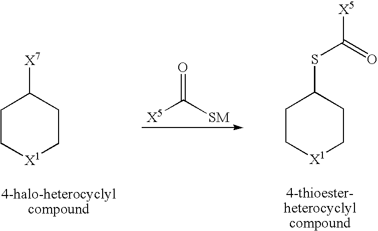
Here, M is a metal cation (preferably a potassium cation). X⁵ is preferably alkyl, aryl, or arylalkyl, with alkyl (particularly methyl) often being more preferred. Any such alkyl, aryl, or arylalkyl substituent optionally may be substituted with one or more independently selected halogen, although, in more preferred embodiments, the alkyl, aryl, and arylalkyl are typically unsubstituted.

In a particularly preferred embodiment, the metal thioester is potassium thioacetate (i.e., M is a potassium cation, and X⁵ is methyl): 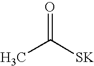
If the 4-halo-heterocyclyl reagent is a 4-halo-tetrahydropyranyl and the metal thioester is potassium thioacetate, the nucleophilic displacement product will be: 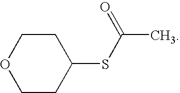
Illustrating further, if the 4-halo-heterocyclyl reagent is instead a 4-halo-piperidinyl compound having a benzyloxycarbonyl nitrogen-protecting group, the nucleophilic displacement product will be: 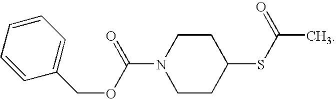
Similarly, if the 4-halo-heterocyclyl reagent is a 4-halo-piperidinyl compound having a t-butoxycarbonyl protecting group, the nucleophilic displacement product will be:

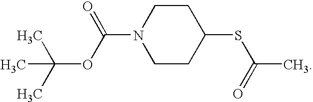

The nucleophilic displacement reaction may be conducted in a batch, semi-continuous, or continuous mode, with batch mode often being more preferred so that the reaction may be contained until the conversion of the 4-halo-heterocyclyl compound is at least essentially complete. Suitable reactor configurations include, for example, stirred-tank reactors. Other reactor configurations may be used, particularly when such configurations provide sufficient retention time and contact between the reagents for substantial conversion of the 4-halo-heterocyclyl compound. The reactor components in contact with the nucleophilic displacement reaction mixture preferably consist essentially of a material(s) that is non-reactive with the reaction reagents and products. Glass reactors are often preferred. Although the reaction may be conducted at a wide variety of pressures and temperatures, it preferably is conducted at ambient pressure, and at a temperature of greater than 25° C., more preferably greater than about 30° C., even more preferably from about 50 to about 70° C., and still even more preferably from about 55 to about 65° C. In general, the reaction is carried out under an inert gas (preferably N₂).

The nucleophilic displacement reaction preferably is conducted with a slight molar excess of the metal thioester relative to the 4-halo-heterocyclyl compound. For example, the amount of metal thioester compound charged to the reactor is preferably greater than 1 and no greater than about 1.2 moles per mole of the 4-halo-heterocyclyl compound, and, more preferably, from about 1.05 to about 1.1 moles per mole of 4-halo-heterocyclyl compound.

The nucleophilic displacement reaction is typically conducted in the presence of a solvent. The solvent preferably has the ability to solubilize the 4-halo-heterocyclyl compound and metal thioester, while not being reactive with any such compounds or the 4-thioester-heterocyclyl product. Suitable solvents include polar organic solvents, such as, for example, dimethylformamide, N-methyl-pyrrolidone, dimethylacetamide, acetonitrile, dimethylsulfoxide, hexamethylphosphorus triamide, nitromethane, and/or tetramethylurea. Dimethylformamide and N-methyl-pyrrolidone are often particularly preferred solvents. The amount of solvent charged to the reactor is preferably at least about 3 ml per gram of 4-halo-heterocyclyl compound, and more preferably from about 3 to about 6 ml per gram of 4-halo-heterocyclyl compound.

The nucleophilic displacement reaction preferably is carried out until at least about 98% of the 4-halo-heterocyclyl compound has been consumed (which may typically be determined using, for example, gas chromatography). When the reaction is carried out in a batch reactor, the reaction time is typically at least about 3.5 hours, and more typically from about 3.5 to about 10 hours.

Example 1 (Part E) illustrates the preparation of a 4-thioester-heterocyclyl compound using a nucleophilic displacement reaction.

As noted above, the 4-thioester-heterocyclyl nucleophilic displacement product preferably is, in turn, oxidatively halogenated to form the 4-halosulfonyl-hetercyclyl compound. In some particularly preferred embodiments, the 4-halosulfonyl-heterocyclyl product is a 4-chlorosulfonyl-heterocyclyl compound. In such embodiments, the 4-thioester-heterocyclyl may, for example, be reacted with a Cl₂ source, N-chlorosuccinimide, or 1,3-dichloro-5,5-dimethylhydantoin:

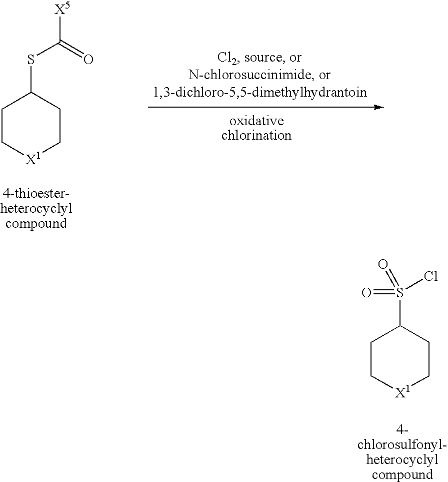

The above oxidative chlorination may be conducted in a batch, semi-continuous, or continuous mode, with batch mode often being more preferred so that the reaction may be contained until the conversion of the 4-thioester-heterocyclyl compound is at least essentially complete. Suitable reactor configurations include, for example, stirred-tank reactors. Other reactor configurations may be used, particularly where such configurations provide sufficient retention time and contact between the reagents for substantial conversion of the 4-thioester-heterocyclyl compound. The reactor components in contact with the oxidative chlorination reaction mixture preferably consist essentially of a material(s) that is non-reactive with the reaction reagents and products. Glass reactors are often preferred. Although the reaction may be conducted at a wide variety of pressures and temperatures, it preferably is conducted at ambient pressure, and at a temperature of less than about 100° C., more preferably at from about 0 to about 40° C., and still more preferably from about 5 to about 25° C. In some preferred embodiments, the temperature is about room temperature. In other preferred embodiments, the temperature is from about 0 to about to 10° C. In generally preferred embodiments, the oxidative chlorination reaction is conducted under an inert gas (preferably N₂).

As noted above, in some preferred embodiments, the 4-thioester-heterocyclyl compound is reacted with a source of Cl₂. The source of Cl₂ is typically a Cl₂-containing gas. A “Cl₂-containing gas” is any gaseous mixture comprising Cl₂ that optionally may also comprise one or more diluents which are non-reactive with the reactants and reaction products under the reaction conditions. Examples of such gases include argon, neon, and N₂. In many preferred embodiments, the Cl₂-containing gas consists essentially of Cl₂.

Where a Cl₂-containing gas is used, the 4-thioester-heterocyclyl compound preferably is contacted with the Cl₂-containing gas in the presence of a solvent and a source of oxygen. The solvent preferably has the ability to, for example, solubilize the 4-thioester-heterocyclyl compound, while not being reactive with the 4-thioester-carbonyl compound, the Cl₂, or the 4-chlorosulfonyl-heterocyclyl product. Often suitable solvents include, for example, chloroalkyl solvents (e.g., CCl₄, CHCl₃, or CH₂Cl₂). Glacial acetic acid is a particularly preferred solvent. In such an embodiment, the source of oxygen preferably is water. The amount of water charged to the reactor is preferably at least about 3 moles per mole of 4-thioester-heterocyclyl compound, more preferably from about 3 to about 10 moles per mole of 4-thioester-heterocyclyl compound, and still more preferably about 7 moles per mole of 4-thioester-heterocyclyl compound.

In some embodiments, the solvent used in the oxidative chlorination also acts as a source of oxygen. In such embodiments, the solvent and source of oxygen may be the same. Often suitable solvents in such embodiments include, for example, alcohols (particularly ethanol). The amount of solvent charged to the reactor is preferably at least about 5 ml per gram of 4-thioester-heterocyclyl compound, and more preferably from about 5 to about 10 ml per gram of 4-thioester-heterocyclyl compound.

The oxidative chlorination preferably is conducted with a molar excess of the Cl₂. The C₂-containing gas may be introduced by any convenient means into the reaction medium in a manner that offers controlled dissolution of Cl₂ into the reaction medium. In many preferred embodiments, the Cl₂-containing gas is introduced into the reaction medium in a manner that maximizes the contact of the gas with the reaction solution. Such contact may be obtained by, for example, dispersing the gas through a diffuser such as a porous glass frit, while shaking or stirring the reactor contents to improve liquid-gas contact and dissolution of the Cl₂. Less preferred, although suitable, alternative methods for introducing the Cl₂-containing gas include, for example, introducing the Cl₂-containing gas into the headspace of the reactor and then drawing it into the reaction mixture using a vortex created by an impeller (this method is sometimes described as a “back-mixed configuration”). It should be noted that when a Cl₂-containing gas is used for the oxidative chlorination, it may be desirable to conduct the reaction at a pressure greater than ambient pressure to increase the rate at which the Cl₂ dissolves into the reaction mixture.

In other preferred embodiments, the 4-thioester-heterocyclyl compound is reacted with N-chlorosuccinimide or 1,3-dichloro-5,5-dimethylhydantoin. The moles of N-chlorosuccinimide or 1,3-dichloro-5,5-dimethylhydantoin charged to the reactor preferably exceeds the moles of 4-thioester-heterocyclyl compound. In some preferred embodiments, the amount of N-chlorosuccinimide or 1,3-dichloro-5,5-dimethylhydantoin charged to the reactor is greater than 1 and no greater than about 10 moles per mole of the 4-thioester-heterocyclyl compound, more preferably from about 2 to about 7 moles per mole of the 4-thioester-heterocyclyl compound, and still more preferably about 5 moles per mole of the 4-thioester-heterocyclyl compound.

Oxidative chlorination with N-chlorosuccinimide or 1,3-dichloro-5,5-dimethylhydantoin preferably is conducted in the presence of a solvent and a source of oxygen. The source of oxygen may be, for example, an alcohol (particularly ethanol). The preferred amount of alcohol charged to the reactor is as described above for the embodiments using a Cl₂-containing gas. The solvent preferably has the ability to, for example, solubilize the 4-thioester-heterocyclyl compound and the N-chlorosuccinimide or 1,3-dichloro-5,5-dimethylhydantoin reagent, while not being reactive with the or the 4-chlorosulfonyl-heterocyclyl product. Often suitable solvents include, for example, heptane and/or cyclohexane. The amount of solvent charged to the reactor is preferably at least about 1 ml per gram of 4-thioester-heterocyclyl compound, more preferably from about 10 to about 20 ml per gram of 4-thioester-heterocyclyl compound, and still more preferably about 14 ml per gram of 4-thioester-heterocyclyl compound.

The oxidative chlorination reaction preferably is carried out until at least about 98% of the 4-thioester-heterocyclyl has been consumed. This typically may be determined using, for example, HPLC. When a Cl₂-containing gas is used, the reaction time (when a batch reactor is used) is typically at least about 2 hours, and more typically from about 2 to about 3 hours. When N-chlorosuccinimide or 1,3-dichloro-5,5-dimethylhydantoin is used, the reaction time (when a batch reactor is used) is typically at least about 3.5 hours, and more typically from about 3.5 to about 5 hours.

Example 1 (Part F) and Example 2 (Part E) illustrate the preparation of a 4-halosulfonyl-heterocyclyl compound using oxidative halogenation.

### B-1(b)(ii) Alternative Example Embodiment for Preparing the 4-Halosulfonyl-Heterocyclyl Compound

Other suitable commercially available starting materials for preparing the 4-halosulfonyl-heterocyclyl compound include, for example, 4-hydroxy-heterocyclyl compounds. Such compounds generally correspond in structure to the following formula: 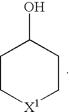
As with the 4-halo-heterocyclyl starting materials, X¹ may be —N(R^{x1})—, —O—, —S—, —S(O)—, or —S(O)₂—.

In instances where the desired X¹ is —N(R^{x1})—, the 4-hydroxy-heterocyclyl compound may be prepared from a commercially available 4-hydroxy-piperidine by protecting the piperidinyl nitrogen with the desired protecting group, R^{x1}: 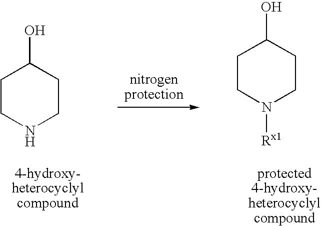
If, for example, the desired protecting group is t-butyloxycarbonyl, the 4-hydroxy-piperidine may be reacted with di-t-butylcarbonate in the presence of a solvent (e.g., toluene or tetrahydrofuran) at ambient temperature and pressure: 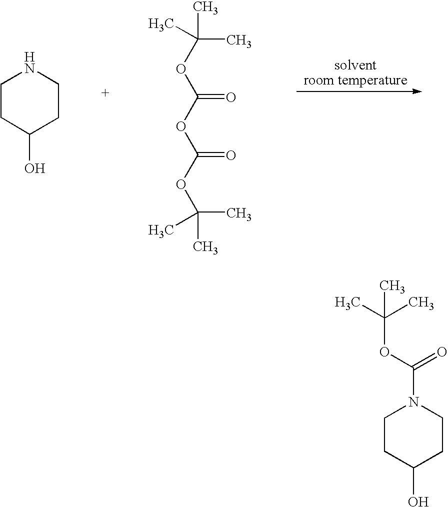
Example 2 (Part A) and Example 4 (Part A) illustrate such a nitrogen protection reaction. Similar techniques may be used to protect the nitrogen with other protecting groups. Such protecting groups include, for example, benzyloxycarbonyl. In that instance, the protected 4-hydroxy-piperidine would be: 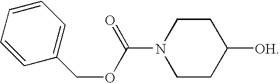
General techniques for such nitrogen protection include, for example, those disclosed in WIPO Intl. Publ. No. WO 00/46221; U.S. Pat. No. 6,448,250; U.S. Pat. No. 6,372,758; and U.S. Pat. No. 6,492,367 (all of which are cited above and incorporated by reference into this patent). Other discussions relating to nitrogen protecting groups may be found in, for example, Greene, T. W.; Wuts, P. G. M.; Protective Groups in Organic Synthesis; 3rd Ed.; Wiley: New York, 1999 (cited above and incorporated by reference into this patent).

The 4-halosulfonyl-heterocylcyl compound may be prepared from the 4-hydroxy-heterocyclyl compound by, for example, sulfonylating the hydroxy of the 4-hydroxy-heterocyclyl to form a 4-sulfonyloxy-heterocyclyl compound; nucleophilically displacing the sulfonyloxy group with a thioester group to form a 4-thioester-heterocyclyl compound; and oxidatively halogenating the 4-thioester-heterocyclyl compound to form the 4-halosulfonyl-heterocyclyl compound:

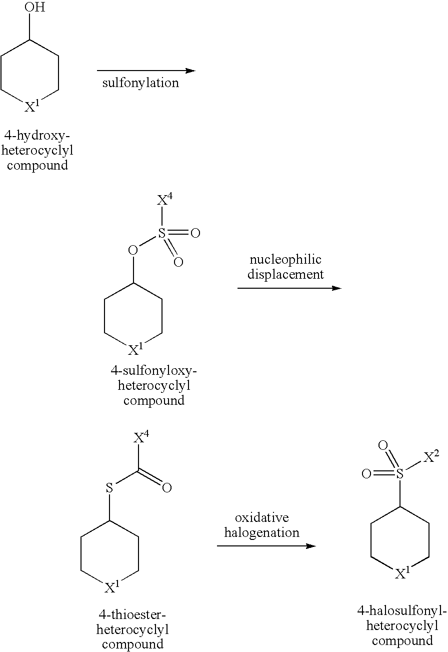

The hydroxy of the 4-hydroxy-heterocyclyl compound may be sulfonylated by, for example, reacting the 4-hydroxy-heterocyclyl compound with an alkyl- or aryl-sulfonylhalide (preferably an alkyl- or aryl-sulfonylchloride) reagent: 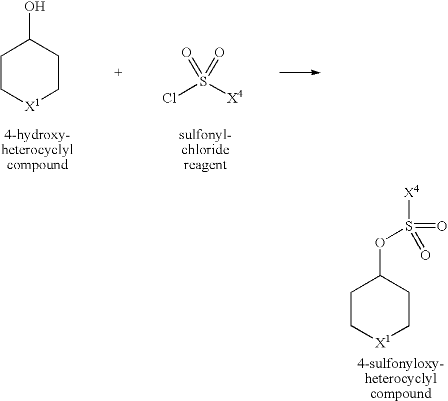
Here, X⁴ may be, for example, alkyl, haloalkyl, aryl, or haloaryl (preferably alkyl, and more preferably methyl). Preferably, the protected 4-hydroxy-heterocyclyl compound is reacted with the sulfonylchloride reagent in the presence of a base (e.g., triethylamine) and a solvent (e.g., toluene). Techniques for sulfonylation of 4-hydroxy-heterocyclyl compounds include, for example, those disclosed in WIPO Intl. Publ. No. WO 00/46221; U.S. Pat. No. 6,448,250; U.S. Pat. No. 6,372,758; and U.S. Pat. No. 6,492,367 (all of which are cited above and incorporated by reference into this patent). Following the sulfonylation reaction, the 4-sulfonyloxy-heterocyclyl compound preferably is isolated from at least a portion of the other components of the sulfonylation reaction.

Example 1 (Part A), Example 2 (Part A), and Example 4 (Part B) below illustrate the preparation of a 4-sulfonyloxy-heterocyclyl compound using a sulfonylation reaction.

The sulfonyloxy group of the 4-sulfonyloxy-heterocyclyl compound is preferably nucleophilically displaced with a thioester group by reacting the 4-sulfonyloxy-heterocyclyl compound with a metal thioester: 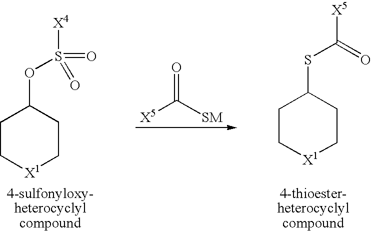
Here, M is a metal cation (preferably a potassium cation). X⁵ is preferably alkyl, aryl, or arylalkyl (preferably alkyl, and more preferably methyl). Any such alkyl, aryl, or arylalkyl substituent optionally may be substituted with one or more independently selected halogen, although, in more preferred embodiments, the alkyl, aryl, and arylalkyl are typically unsubstituted.

In some particularly preferred embodiments, the metal thioester is potassium thioacetate (i.e., M is a potassium cation and X⁵ is methyl): 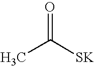
If, for example, the 4-hydroxy-heterocyclyl reagent is 4-hydroxy-tetrahydropyaranyl and the metal thioester is potassium thioacetate, the nucleophilic displacement product will be: 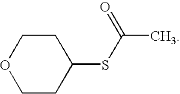
Illustrating further, if the 4-hydroxy-heterocyclyl reagent is instead a 4-hydroxy-piperidinyl compound having a benzyloxycarbonyl protecting group, the nucleophilic displacement product will be: 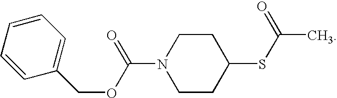
Similarly, if the 4-hydroxy-heterocyclyl reagent is a 4-hydroxy-piperidinyl compound having a t-butoxycarbonyl protecting group, the nucleophilic displacement product will be:

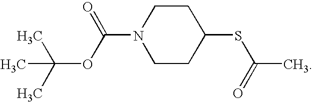

The nucleophilic displacement reaction may be conducted in a batch, semi-continuous, or continuous mode, with batch mode often being more preferred so that the reaction may be contained until the conversion of the 4-sulfonyloxy-heterocyclyl compound is at least essentially complete. Suitable reactor configurations include, for example, stirred-tank reactors. Other reactor configurations may be used, particularly when such configurations provide a sufficient retention time and contact between the reagents for substantial conversion of the 4-sulfonyloxy-heterocyclyl compound. The reactor components in contact with the nucleophilic displacement reaction mixture preferably consist essentially of a material(s) that is non-reactive with the reaction reagents and products. Glass reactors are often preferred. Although the reaction may be conducted at a wide variety of pressures and temperatures, it preferably is conducted at ambient pressure, and at a temperature of greater than 25° C., more preferably greater than about 30° C., even more preferably from about 50 to about 70° C., and still even more preferably from about 55 to about 65° C. In general, the reaction is carried out under an inert gas (preferably N₂).

The nucleophilic displacement preferably is conducted with a slight molar excess of the metal thioester relative to the 4-hydroxy-heterocyclyl compound. For example, the amount of metal thioester compound charged to the reactor is preferably greater than 1 and no greater than about 1.2 moles per mole of the 4-hydroxy-heterocyclyl compound, and, more preferably, from about 1.05 to about 1.1 moles per mole of 4-hydroxy-heterocyclyl compound.

The nucleophilic displacement is typically conducted in the presence of a solvent. The solvent preferably has the ability to solubilize the 4-sulfonyloxy-heterocyclyl compound and metal thioester, while not being reactive with any such compounds or the 4-thioester-heterocyclyl product. Suitable solvents include polar organic solvents, such as, for example, N-methyl-pyrrolidone, dimethylformnamide, dimethylacetamide, acetonitrile, dimethylsulfoxide, hexamethylphosphorus triamide, nitromethane, and/or tetramethylurea. Dimethylformamide and N-methyl-pyrrolidone are often particularly preferred solvents. The amount of solvent charged to the reactor is preferably at least about 7 ml per gram of 4-sulfonyloxy-heterocyclyl compound, and more preferably from about 7.5 to about 8.5 ml per gram of 4-sulfonyloxy-heterocyclyl compound.

The nucleophilic displacement reaction preferably is carried out until at least about 98% of the 4-sulfonyloxy-heterocyclyl compound has been consumed. This typically may be determined using, for example, gas chromatography. When the reaction is carried out in a batch reactor, the reaction time is typically at least about 8 hours, and more typically from about 15 to about 17 hours.

Example 2 (Part D) below illustrates preparation of a 4-thioester-heterocyclyl compound using a nucleophilic displacement reaction described above.

The 4-thioester-heterocyclyl preferably is, in turn, oxidatively halogenated to form the 4-halosulfonyl-heterocyclyl compound. This may be achieved using, for example, the oxidative halogenation protocols described above in Section B-1(b)(i).

### B-1(b)(iii). Particularly Preferred Embodiments for Making Hydroxamic Acids Where Z Is —N(R^{x})— and Z³ Is Carbon Bonded to Hydrogen

In some particularly preferred embodiments where Z is —N(R^{x})— and Z³ is carbon bonded to hydrogen, the cyclic amino and 4-halosulfur-heterocyclyl sulfuramidation reagents are prepared from the same material. Processes using such a starting material (or intermediate) for both these purposes are advantageous because, for example, they can generally be completed with fewer steps. In addition, the diversity of potential impurities from such processes tends to be narrower because fewer reagents are involved relative to processes using different starting materials.

Such embodiments are particularly suitable for preparing hydroxamic acid compounds generally corresponding in structure to the following formula: 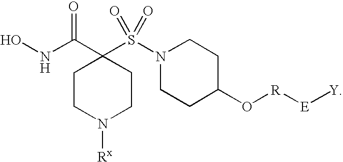
Particularly desirable hydroxamic acid compounds falling within this general formula include, for example:

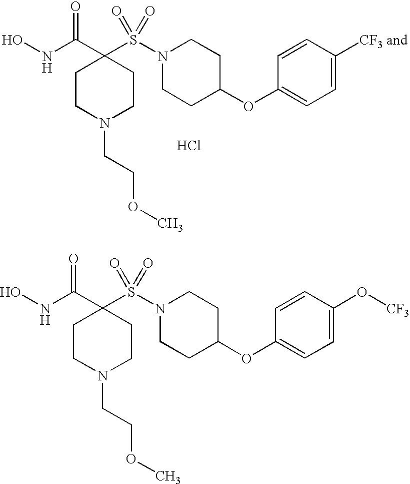

In the above embodiments, the starting material (or intermediate) may be, for example, a 4-sulfonyloxy-heterocyclyl compound generally corresponding in structure to the following formula: 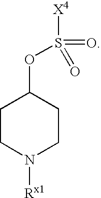
Scheme (III) illustrates how such a 4-sulfonyloxy-heterocyclyl compound may be used as a common material for preparing both the cyclic amino and 4-halosulfonyl-heterocyclyl reagents: 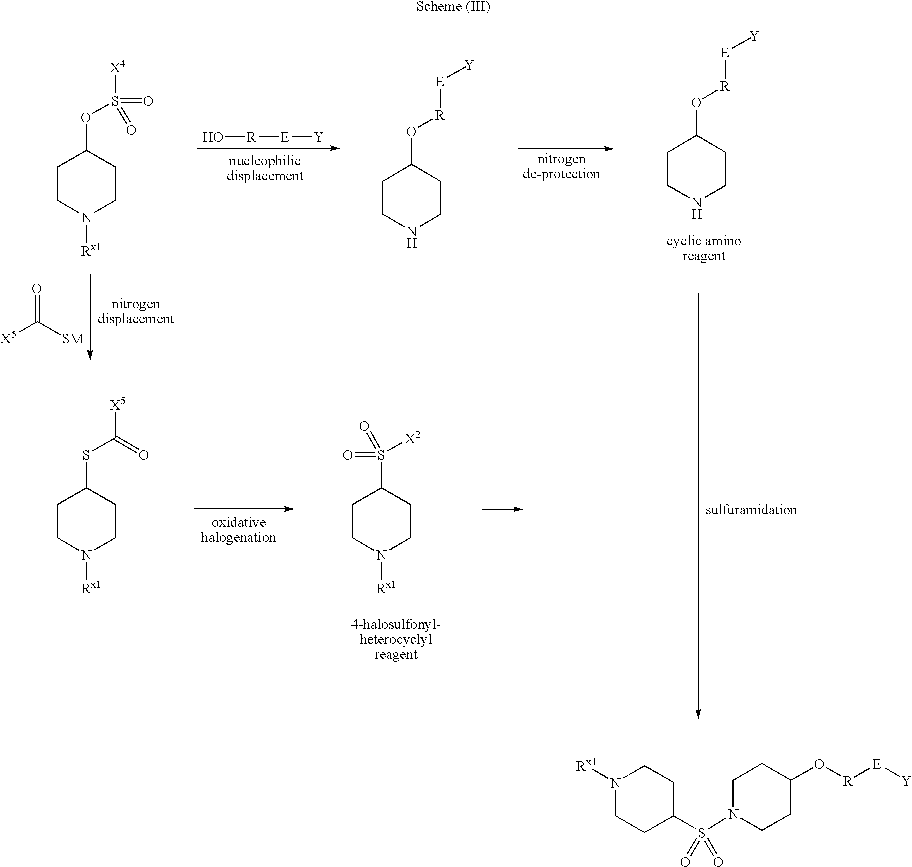
Section B-6 illustrates additional schemes using common 4-sulfonyloxy-heterocyclyl compounds to prepare the cyclic amino and 4-halosulfur-heterocyclyl sulfuramidation reagents. Example 2 below illustrates such a reaction scheme.

### B-1(c). Preferred Reaction Conditions for the Sulfuramidation Reaction

As noted above, the sulfuramine intermediate is preferably prepared by reacting the cyclic amino reagent with the 4-halosulfonyl-heterocyclyl reagent: 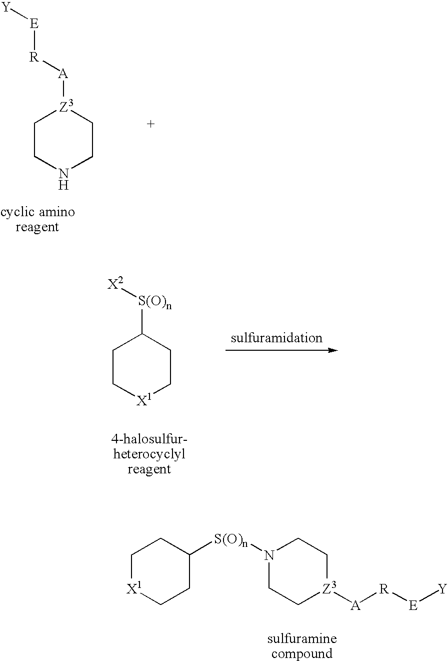
The preferred sulfuramine compound will vary, depending on the desired hydroxamic acid compound. Where, for example, the desired hydroxamic acid corresponds in structure to the following formula: 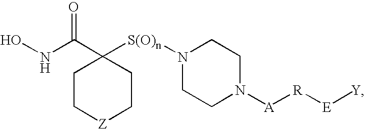
a suitable sulfuramine intermediate may be prepared via the following reaction: 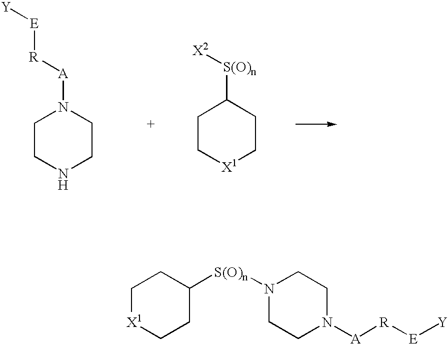
Where, for example, the desired hydroxamic acid corresponds in structure to the following formula: 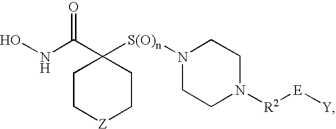
a suitable sulfuramine intermediate may be prepared via the following reaction: 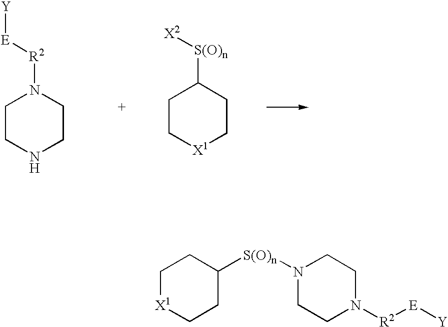
Where, for example, the desired hydroxamic acid corresponds in structure to the following formula: 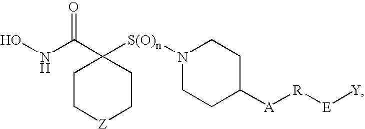
a suitable sulfuramine intermediate may be prepared via the following reaction: 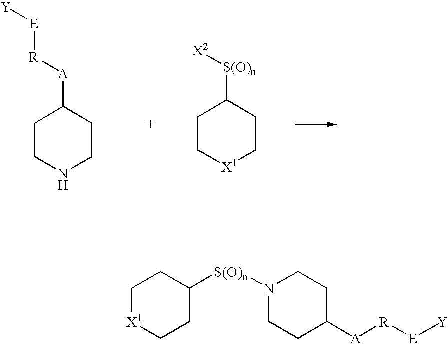
Where, for example, the desired hydroxamic acid corresponds in structure to the following formula: 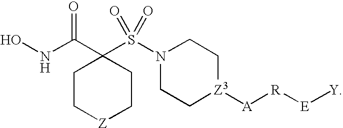
a suitable sulfuramine intermediate may be prepared via the following reaction: 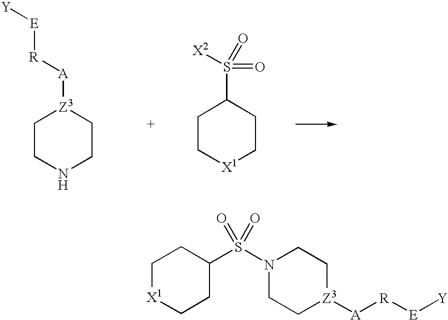
Where, for example, the desired hydroxamic acid corresponds in structure to the following formula: 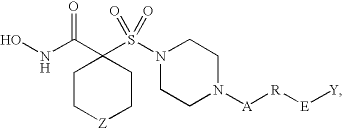
a suitable sulfuramine intermediate may be prepared via the following reaction: 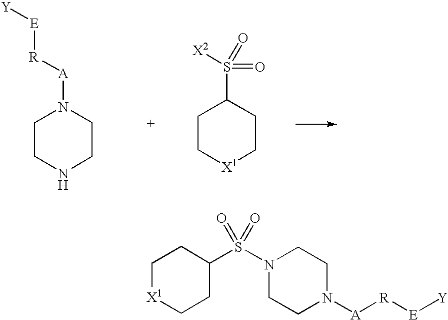
Where, for example, the desired hydroxamic acid corresponds in structure to the following formula: 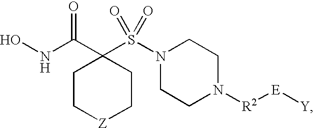
a suitable sulfuramine intermediate may be prepared via the following reaction: 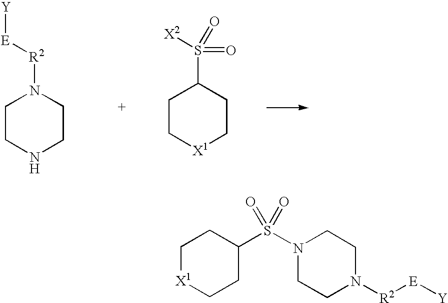
Where, for example, the desired hydroxamic acid corresponds in structure to the following formula: 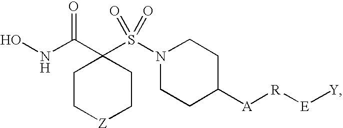
a suitable sulfuramine intermediate may be prepared via the following reaction: 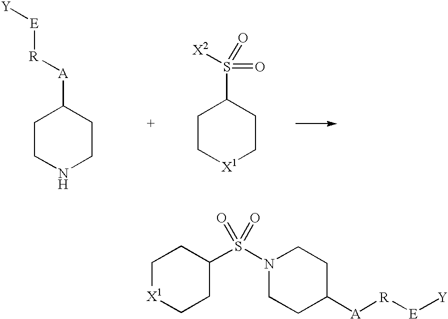
Where, for example, the desired hydroxamic acid corresponds in structure to the following formula: 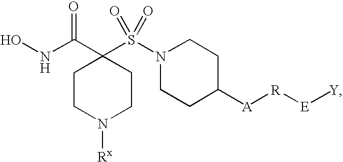
a suitable sulfuramine intermediate may be prepared via the following reaction: 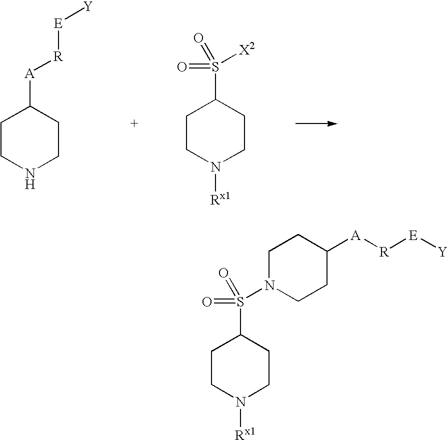
For example, R^{x1} may be t-butoxycarbonyl: 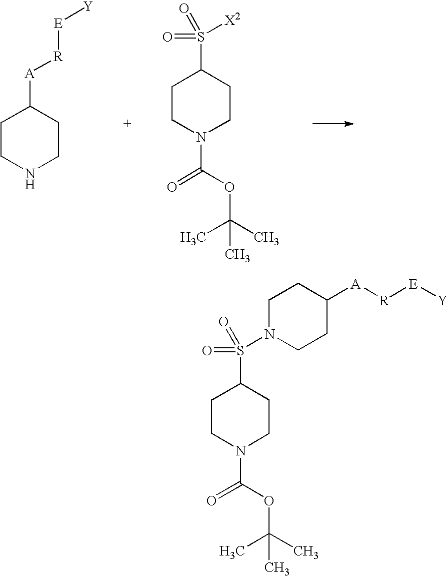
Or R^{x1} may, for example, be benzyloxycarbonyl: 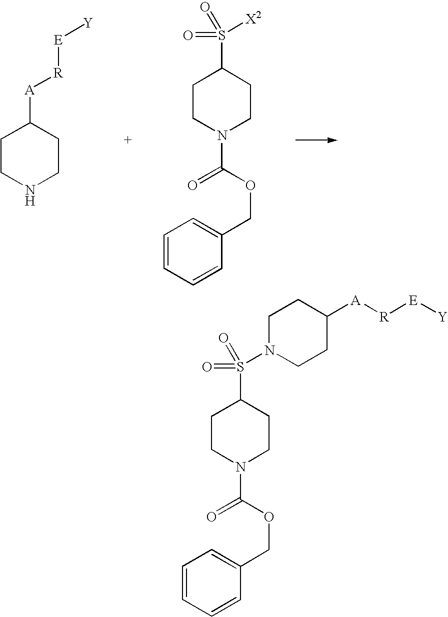
Where, for example, the desired hydroxamic acid corresponds in structure to the following formula: 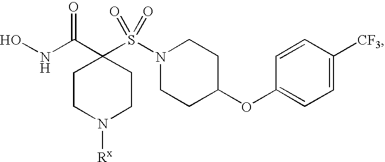
a suitable sulfuramine intermediate may be prepared via the following reaction: 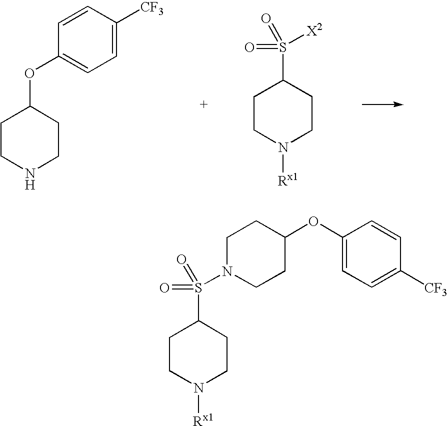
example, R^{x1} may be t-butoxycarbonyl: 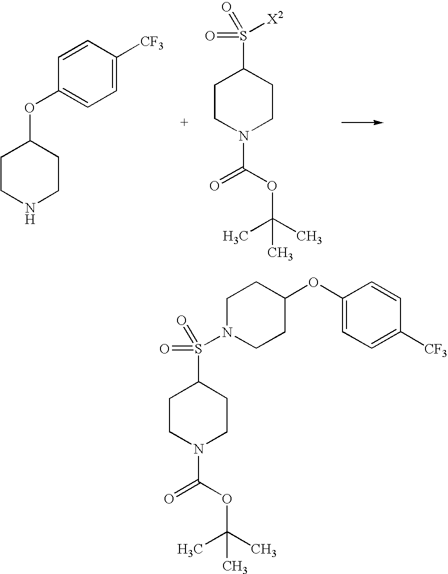
Or R^{x1} may, for example, be benzyloxycarbonyl: 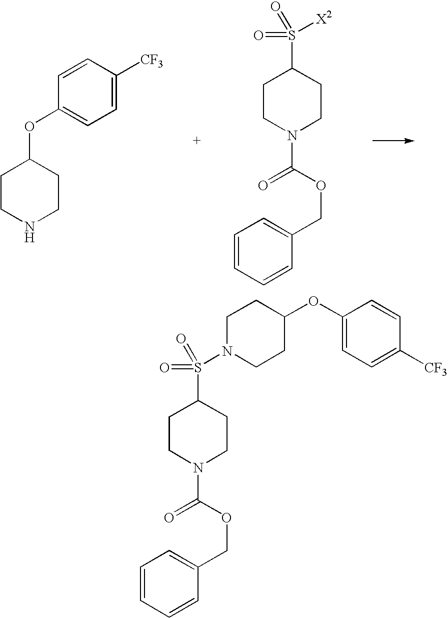
Where, for example, the desired hydroxamic acid corresponds in structure to the following formula: 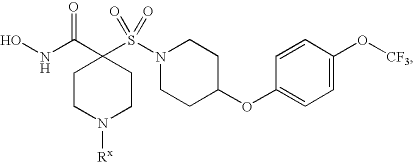
a suitable sulfuramine intermediate may be prepared via the following reaction: 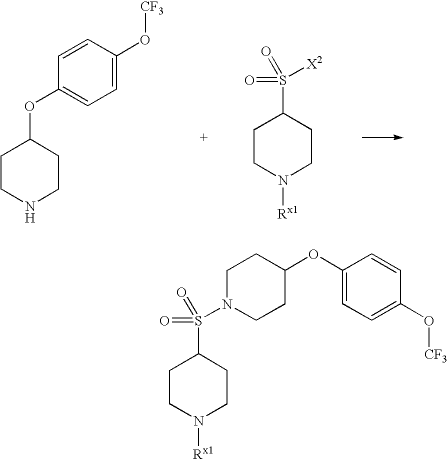
For example, R^{x1} may be t-butoxycarbonyl: 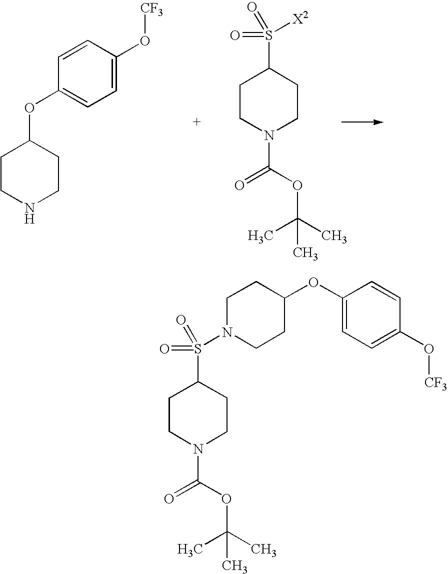
Or R^{x1} may, for example, be benzyloxycarbonyl:

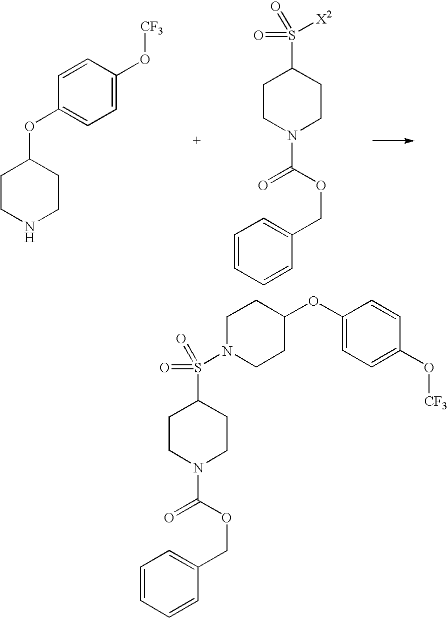

The sulfuramidation may be conducted in a batch, semi-continuous, or continuous mode, with batch mode often being more preferred so that the reaction may be contained until the conversion of the cyclic amino reagent is at least essentially complete. Suitable reactor configurations include, for example, stirred-tank reactors. Other reactor configurations may be used, particularly where such configurations provide a sufficient retention time and contact between the reagents for substantial conversion of the cyclic amino reagent. The reactor components in contact with the sulfuramidation reaction mixture preferably consist essentially of a material(s) that is non-reactive with the reaction reagents and products. Glass reactors are often preferred. Although the reaction may be conducted at a wide variety of pressures and temperatures, it preferably is conducted at ambient pressure, and at a temperature of from about 0 to about 30° C. In some preferred embodiments, the reaction is carried out at about room temperature. It is typically preferred to conduct the sulfuramidation reaction in anhydrous conditions due to the reactivity of water with the 4-halosulfonyl-heterocyclyl compound. In generally preferred embodiments, the reaction is carried out under a dry inert gas (preferably N₂).

The sulfuramidation preferably is conducted with a slight molar excess of the 4-halosulfur-heterocyclyl compound relative to the cyclic amino compound. For example, the amount of 4-halosulfur-heterocyclyl compound charged to the reactor is preferably greater than 1 and no greater than about 1.2 moles per mole of cyclic amino compound, and, more preferably, from about 1.05 to about 1.1 moles per mole of cyclic amino compound.

The sulfuramidation reaction typically produces acid (HCl, for example, is formed when the 4-halosulfur-heterocyclyl compound is a 4-chlorosulfonyl-heterocyclyl compound). The sulfuramidation reaction is therefore preferably conducted in the presence of a base to neutralize the acid produced. Triethylamine is often a preferred base due to, for example, its relatively low cost. Other often suitable bases include, for example, tertiary amines and inorganic bases (e.g., potassium carbonate).

The sulfuramidation reaction may be conducted without the presence of a solvent. In typically preferred embodiments, however, the reaction is conducted in the presence of a solvent. The solvent preferably has the ability to solubilize the cyclic amino compound, the 4-halosulfur-heterocyclyl compound, and the base (to the extent present), while not being reactive with any such compounds or the sulfuramine product. In many embodiments, an aprotic solvent is preferred. Toluene is a particularly preferred solvent. Methylene chloride also is a particularly preferred solvent. Other often suitable solvents include, for example, ethereal solvents, dioxane, N-methylpyrrolidone, aromatic hydrocarbon solvents, acetonitrile, and dimethylformamide. The amount of solvent charged to the reactor is preferably at least about 1 ml per gram of cyclic amino compound, more preferably from about 1 to about 100 ml per gram of cyclic amino compound, even more preferably from about 5 to about 20 ml per gram of cyclic amino compound, and still even more preferably about 10 ml per gram of cyclic amino compound.

The sulfuramidation reaction preferably is carried out until at least about 98% of the cyclic amino compound has been consumed. This typically may be determined using, for example, HPLC. When the reaction is carried out in a batch reactor, the reaction time is typically at least about 1 hour, and more typically from about 1 to about 5 hours.

The sulfuramidation product mixture preferably is washed with water. The amount of water used preferably is at least about 4 ml per gram of cyclic amino compound charged to the reactor. In some embodiments, the amount of water used is from about 4 to about 6 ml per gram of cyclic amino compound charged to the reactor, and still more preferably about 6 ml per gram of cyclic amino compound charged to the reactor. In an often more preferred embodiment, the product mixture is additionally washed with water and acid. The presence of the acid is generally beneficial for removing residual cyclic amino starting material.

In some embodiments, after washing, the sulfuramidation product mixture preferably is heated (via, for example, evaporation or distillation) to reduce the volume of solvent. Typically, it is preferable to reduce the solvent volume such that the ratio of solvent to sulfuramine product is from about 6:1 to about 1:1 (ml:g), and more preferably about 1:1 (ml:g). In some preferred embodiments, an anti-solvent is introduced. Often suitable anti-solvents include, for example, saturated aliphatic hydrocarbons comprising at least about 6 carbon atoms. Examples of often suitable anti-solvents include heptane, hexane, and isooctane, with heptane being particularly preferred. After the anti-solvent is charged, the mixture is preferably agitated (e.g., stirred) for from about 2 to about 3 hours (more preferably about 3 hours) while maintaining the temperature at from about 5 to about 25° C., and more preferably at about 20° C. The resulting precipitate preferably is isolated from the resulting mixture using, for example, filtration, centrifugation, settling, and/or another solid isolation technique(s). The isolated solid is then preferably washed with additional anti-solvent, and then further dried using, for example, vacuum drying.

Example 1 (Part G), Example 2 (Part F), and Example 3 illustrate the preparation of a sulfuramine compound using a sulfuramidation reaction.

### B-2. Nitrogen De-Protection

### Applicable to Embodiments where X¹ is —N(R^{x1})—

In some preferred embodiments, the Xi moiety of the sulfuramine compound will be the same as the Z moiety of the desired hydroxamic acid compound. In such instances, the X¹ moiety often will not participate in the remaining steps of the synthesis forming the hydroxamic acid compound. This is particularly true when X¹ and Z are both —O—, —S—, —S(O)—, or —S(O)₂—. It also may be true where X¹ is —N(R^{x1})—, Z is —N(R^{x})—, and R^{x1} and R^{x} are the same.

In some preferred embodiments, however, X¹ and Z are not the same. This, for example, occurs where X¹ is —N(R^{x1})—, Z is —N(R^{x})—, and R^{x1} and the desired R^{x} are not the same. In those instances, the piperidinyl nitrogen protecting group (R^{x1}) is typically removed after the sulfuramidation:

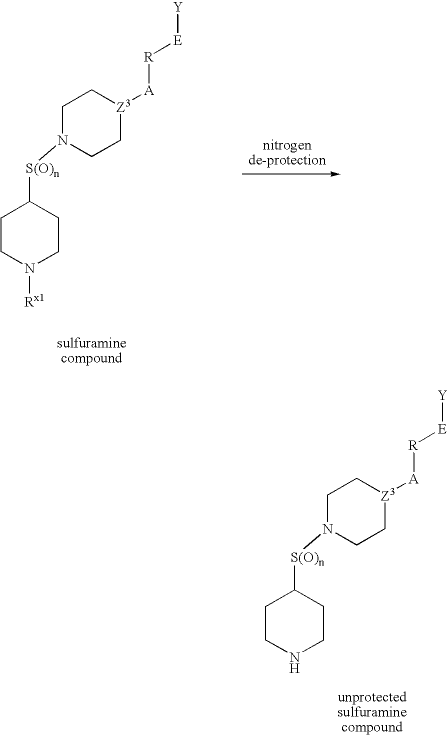

It is often preferred to use an R^{x1} substituent that is different from the desired R^{x}. In some particularly preferred embodiments, for example, R^{x} is methoxyethyl. A scheme using an identical substituent (i.e., methoxyethyl) as R^{x1} could potentially avoid the above de-protection step (and a subsequent N-alkylation step to attach the R^{x} methoxyethyl). Applicants have discovered, however, that a sulfuramine compound having a de-protected nitrogen is more easily purified than a sulfuramine compound having a methoxyethyl group bonded to the piperidinyl nitrogen. This benefit often justifies the extra steps of de-protecting the nitrogen and subsequent N-alkylation with the methoxyethyl group. This is particularly true if the R^{x1} substituent is easily removable. Such easily removable nitrogen protecting groups include, for example, alkoxycarbonyl (e.g., t-butoxycarbonyl), arylalkoxycarbonyl (e.g., phenylmethoxycarbonyl) groups, and the like. See, generally, Greene, T. W.; Wuts, P. G. M.; Protective Groups in Organic Synthesis; 3rd Ed.; Wiley: New York, 1999 (cited above and incorporated by reference into this patent).

It should be recognized that de-protection of the sulfuramine piperidinyl nitrogen may be desirable even in instances when R^{x1} and the desired R^{x} are the same. This is particularly preferable where, as discussed above, such de-protection improves purification of the sulfuramine compound.

In instances when an R^{x1} group is removed from the sulfuramine compound, the R^{x1} group may be removed using, for example, hydrogenolysis. In that instance, the sulfuramine compound is typically dissolved in a non-reactive solvent (e.g., ethanol or ethyl acetate), and then contacted with a source of H₂ (e.g., H₂ gas itself or ammonium formate) and a transition metal catalyst (e.g., cobalt, nickel, copper, zinc, and the like).

In generally more preferred embodiments, removal of the R^{x1} group is accomplished using hydrolysis. This preference for hydrolysis over hydrogenolysis stems from the fact that hydrolysis tends to be quicker and produce fewer impurities.

Although either base or acid hydrolysis may be used, acid hydrolysis is generally preferred because less-extreme conditions (e.g., temperatures) can typically be used. A wide variety of acids may be used. Typically, however, the acid is a strong acid, i.e., the acid preferably has a pKₐ of no greater than about −3. Suitable acids may include, for example, hydrochloride (HCl), hydrobromide (HBr), hydroiodide (HI), sulfuric acid (H₂SO₄), and trifluoracetic acid (CF₃COOH), with HCl, HBr, HI, and H₂SO₄ often being more preferred, and HCl often being even more preferred. In some particularly preferred embodiments, HCl is introduced into the reaction mixture in the form of HCl gas.

HCl gas may be charged to the reactor in any manner that achieves the desired dissolved HCl concentration in the reaction mixture. The concentration of HCl in the reaction mixture preferably is at least about 0.5% by weight, and more preferably from about 2 to about 5% by weight. In many preferred embodiments, the HCl is introduced into the reaction medium in a manner that maximizes the contact of the gas with the reaction solution. Such contact may be obtained, for example, by dispersing the gas through a diffuser such as a porous glass frit, while shaking or stirring the reactor contents to improve liquid-gas contact and dissolution of the HCl. Less preferred, although suitable, alternative methods for introducing the HCl include, for example, use of a back-mixed configuration. It should be noted that when HCl gas is used for the hydrolysis, it may be desirable to conduct the reaction at a pressure greater than ambient pressure to increase the rate at which the HCl dissolves into the reaction mixture.

The hydrolysis may be conducted in a batch, semi-continuous, or continuous mode, with batch mode often being more preferred so that the reaction may be contained until hydrolysis of the protected sulfuramine compound is at least essentially complete. Suitable reactor configurations include, for example, stirred-tank reactors. Other configurations may be used, particularly when such configurations provide a sufficient retention time and contact between the reagents for substantial conversion of the protected sulfuramine compound to the unprotected sulfuramine compound. The reactor components in contact with the hydrolysis reaction mixture preferably consist essentially of a material(s) that is non-reactive with the reaction reagents and products. Glass reactors are often preferred. Although the reaction may be conducted at a wide variety of pressures and temperatures, it preferably is conducted at ambient pressure, and at a temperature of greater than about 15° C. In some embodiments, the hydrolysis is conducted at a temperature of greater than about 30° C., more preferably from about 50° C. to about reflux, and still even more preferably at from about 60 to about 75° C. In other embodiments, the hydrolysis is carried out at from about 15 to about 30° C., and more preferably at about room temperature. Normally, it is preferred to carry out the reaction under an inert gas (preferably N₂).

Typically, the hydrolysis is conducted in the presence of a solvent. The solvent preferably has the ability to solubilize the sulfuramine compound and acid, while not being reactive with any such compounds or the de-protected product. Although water may be used, an alcohol solvent is often more preferred because, for example, the de-protected product tends to be less soluble in alcohol solvents than in, for example, water. Often suitable alcohols include, for example, hydroxyalkyls, particularly ethanol and isopropyl alcohol. The amount of solvent charged to the reactor is preferably from about 610 to about 1700 ml per mole of HCl, and more preferably about 1700 ml per mole of HCl.

The hydrolysis reaction preferably is carried out until at least about 98% of the sulfuramine compound has been consumed. This typically may be determined using, for example, HPLC. When the reaction is carried out in a batch reactor, the reaction time is typically from about 1 to about 50 hours, more typically from about 1 to about 5 hours, and still more typically from about 1.5 to about 2.5 hours.

In many embodiments, the unprotected sulfuramine product is isolated following the hydrolysis. This may be achieved using various techniques known in the art, such as, for example, filtration, centrifugation, settling, and/or another solid isolation technique(s).

Example 1 (Part H), Example 2 (Part G), and Example 3 illustrate the preparation of an unprotected sulfuramine using hydrolysis, as well as various methods for isolating the unprotected sulfuramine product following the hydrolysis.

### B-3. Nitrogen Substitution

### Applicable to Embodiments where Z is —N(R)—

In some embodiments, the desired hydroxamic acid will have an unprotected piperidine on the a-carbon (i.e., where R^{x} is hydrogen). In such embodiments, the hydroxamic acid compound will generally correspond in structure to the following formula: 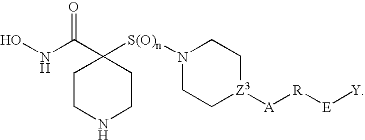
In other embodiments, however, the nitrogen of the a-carbon piperidine will be substituted (i.e., where R^{x} is other than hydrogen). In these embodiments, the hydroxamic acid compound will generally correspond in structure to the following formula: 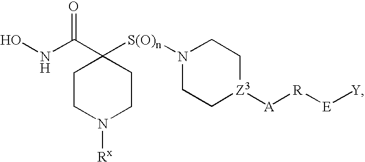
Here, R^{x} is alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, or heterocyclylsulfonyl. Any such substituent optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy. Any such optional substituent is, in turn, optionally substituted with one or more substituents independently selected from the group consisting of halogen, hydroxy, and alkyl.

In many such embodiments, the process will involve a sulfuramine intermediate corresponding in structure to the following formula: 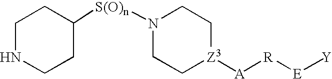
(such embodiments include, for example, those discussed above where the sulfuramine piperidinyl nitrogen has been de-protected for improved purification). In those instances, the desired non-hydrogen R^{x} substituent is substituted onto the unprotected piperidine nitrogen: 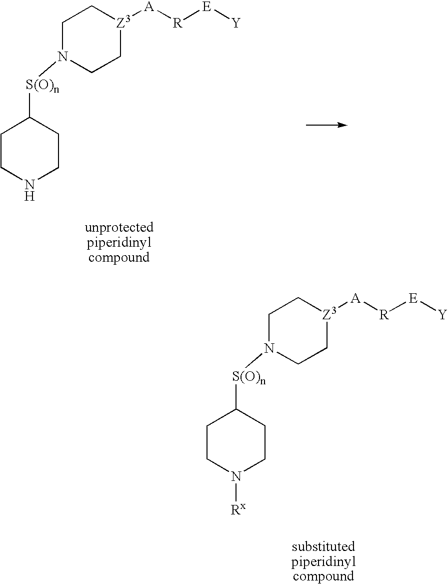
The preferred substituted piperidinyl compound will vary, depending on the desired hydroxamic acid compound. Where, for example, the desired hydroxamic acid corresponds in structure to the following formula: 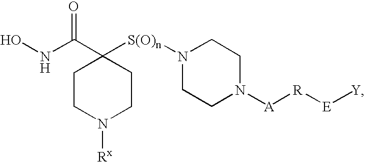
a suitable substituted piperidinyl compound may be prepared via the following conversion: 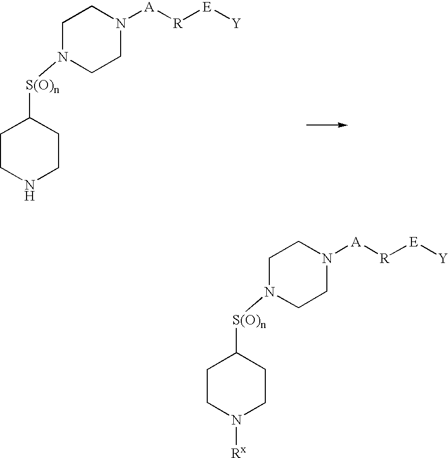
Where, for example, the desired hydroxamic acid corresponds in structure to the following formula: 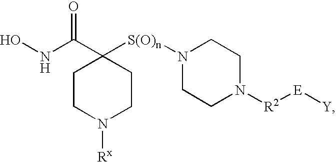
a suitable substituted piperidinyl compound may be prepared via the following conversion: 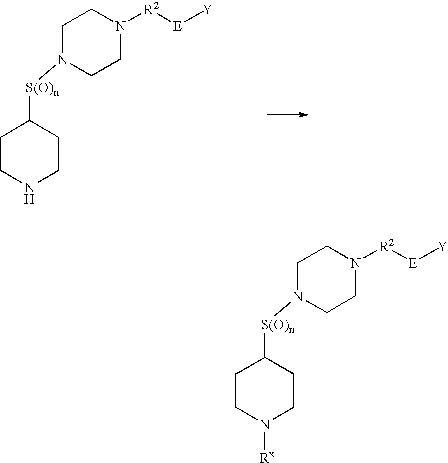
Where, for example, the desired hydroxamic acid corresponds in structure to the following formula: 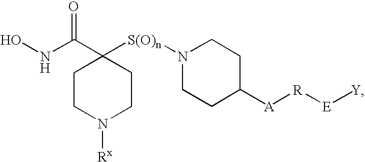
a suitable substituted piperidinyl compound may be prepared via the following conversion: 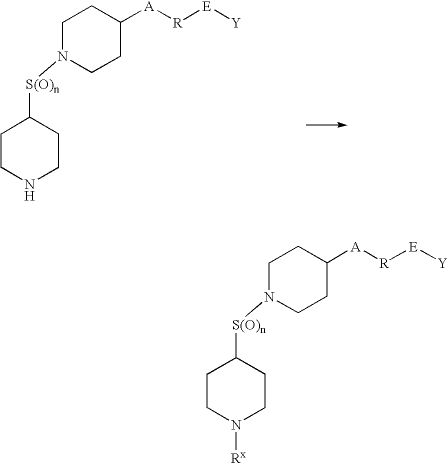
Where, for example, the desired hydroxamic acid corresponds in structure to the following formula: 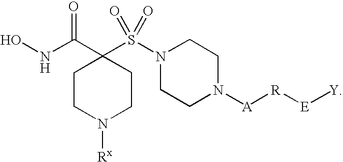
a suitable substituted piperidinyl compound may be prepared via the following conversion: 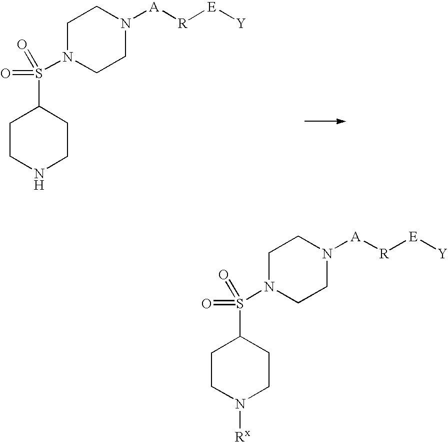
Where, for example, the desired hydroxamic acid corresponds in structure to the following formula: 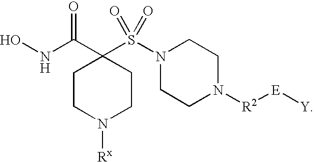
a suitable substituted piperidinyl compound may be prepared via the following conversion: 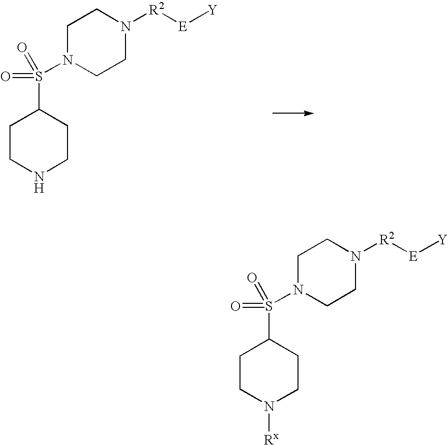
Where, for example, the desired hydroxamic acid corresponds in structure to the following formula: 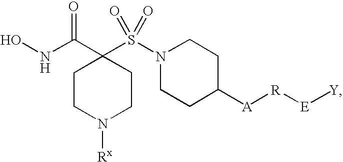
a suitable substituted piperidinyl compound may be prepared via the following conversion: 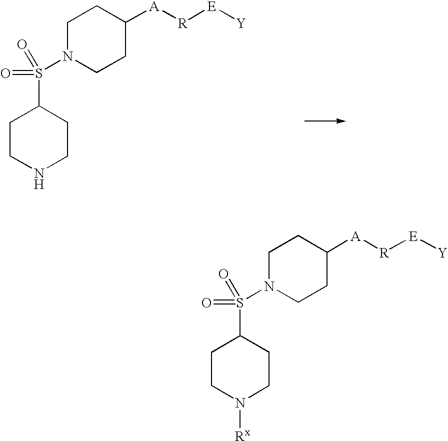
Where, for example, the desired hydroxamic acid corresponds in structure to the following formula: 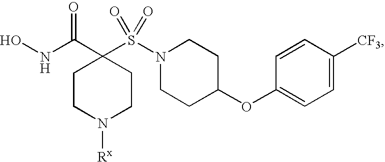
a suitable substituted piperidinyl compound may be prepared via the following conversion: 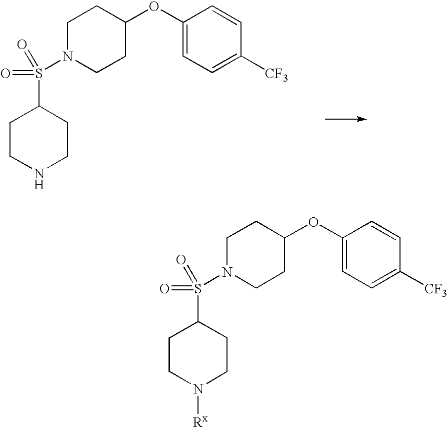
Where, for example, the desired hydroxamic acid corresponds in structure to the following formula: 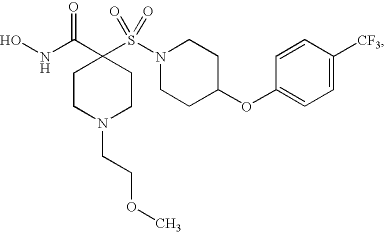
a suitable substituted piperidinyl compound may be prepared via the following conversion: 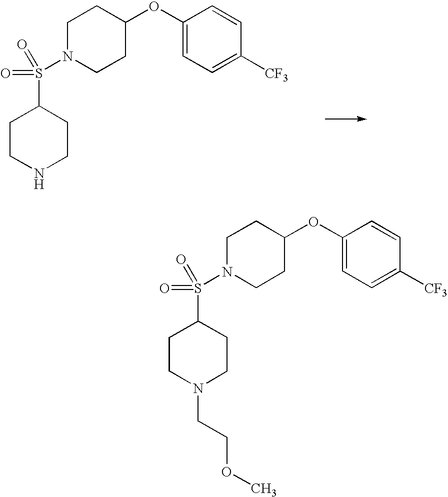
Where, for example, the desired hydroxamic acid corresponds in structure to the following formula: 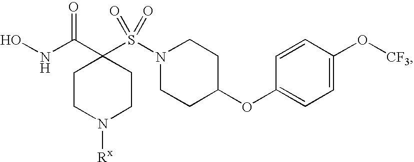
a suitable substituted piperidinyl compound may be prepared via the following conversion: 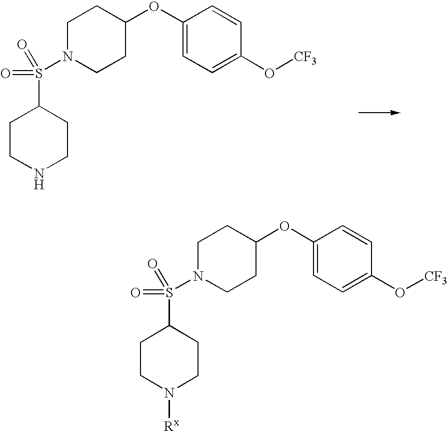
Where, for example, the desired hydroxamic acid corresponds in structure to the following formula: 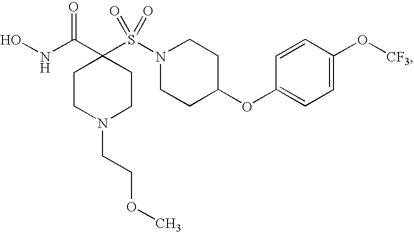
a suitable substituted piperidinyl compound may be prepared via the following conversion:

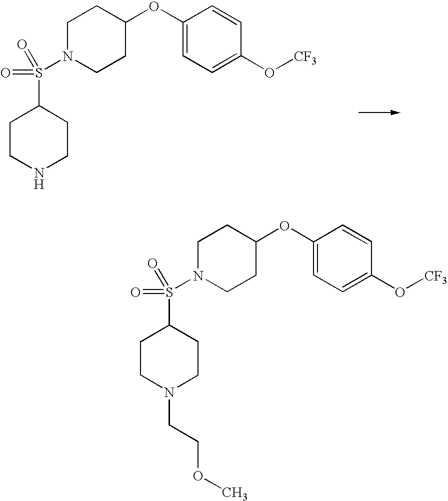

In many particularly preferred embodiments, the N-substitution of the R^{x} substituent is an N-alkylation reaction. Such a reaction may be used to substitute the nitrogen with alkyl (e.g., methyl, ethyl, t-butyl) or substituted alkyl. The alkyl may be substituted with, for example, halo (to form, for example, trifluoromethylpropyl)), di-substituted amino (to form, for example, N, N-diethylaminoethyl), optionally-substituted alkoxy (to form, for example, methoxyethyl), optionally-substituted aryl (to form, for example, phenylmethyl), optionally-substituted cycloalkyl (to form, for example, cyclopropylmethyl), and/or optionally-substituted heterocyclyl (to form, for example, morpholinylethyl, methylimidazolylmethyl, furanylmethyl, methylfuranylmethyl, pyridinylmethyl) onto the nitrogen. In some embodiments, the alkyl may be substituted with R^{a}-oxy, R^{a}R^{a}-amino (wherein each R^{a} is other than hydrogen), carbocyclyl, or heterocyclyl. Any member of such group optionally is substituted with one or more substituents independently selected from the group consisting of halogen, cyano, carboxy, thiol, sulfo, nitro, nitroso, oxo, thioxo, imino, alkoxy, alkylthio, and alkoxyalkoxy. Any such optional substituent is, in turn, optionally substituted with one or more substituents independently selected from the group consisting of halogen, hydroxy, and alkyl. Each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl. Any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

An N-alkylation may be achieved by, for example, reacting the unprotected piperidinyl compound with an N-alkylating agent. In some preferred embodiments, the N-alkylating agent is R^{x}—X³. Here, X³ is halogen (e.g., bromo, iodo, or chloro) or corresponds in structure to one of the following formulas: 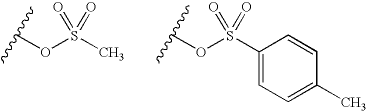
Thus, when, for example, the desired R^{x} is methoxyethyl, suitable N-alkylating agents include, for example, the following compounds: 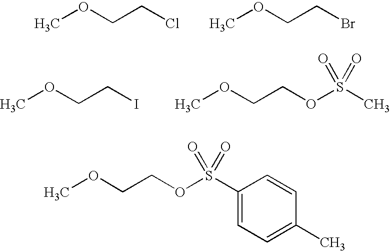
In many particularly preferred embodiments, X³ is chloro. This preference stems from, for example, Applicants' discovery that such an N-alkylating agent tends to advantageously minimize any impurities that may be formed.

The amount of N-alkylating agent charged to the reactor can vary, but preferably is at least 1 mole per mole of unprotected piperidinyl compound. In often more preferred embodiments, the amount of N-alkylating agent preferably is from about 1.1 to about 10 moles per mole of unprotected piperidinyl compound, and more preferably from about 1.1 to about 2.0 moles per mole of unprotected piperidinyl compound. Applicants have discovered that the conversion of the piperidinyl compound per unit of time tends to be optimized at these preferred ranges.

The N-alkylation reaction may be conducted in a batch, semi-continuous, or continuous mode, with batch mode often being more preferred so that the reaction may be contained until the conversion of the unprotected piperidinyl compound is at least essentially complete. Suitable reactor configurations include, for example, stirred-tank reactors. Other reactor configurations may be used, particularly when such configurations provide a sufficient retention time and contact between the reagents for substantial conversion of the unprotected piperidinyl compound to the substituted piperidinyl compound. The reactor components in contact with the N-alkylation reaction mixture preferably consist essentially of a material(s) that is non-reactive with the reaction reagents and products. Glass reactors are typically preferred. Although the reaction may be conducted at a wide variety of pressures and temperatures, it preferably is conducted at ambient pressure, and at a temperature of at least about 0° C., more preferably at least about 20° C., still more preferably at least about 30° C., and still yet even more preferably at least about 50° C. These temperatures are often particularly preferred when X³ is a halogen. For example, when the N-alkylating agent is R^{x}—Br (i.e., when X³ is bromo), the reaction temperature is often preferably from about 50 to about 60° C. When, on the other hand, the N-alkylating agent is R^{x}—Cl (i.e., when X³ is chloro), the reaction temperature is often preferably at least about 60° C., and more preferably from about 70 to about 90° C. Normally, the reaction is carried out under a dry, inert gas (preferably N₂).

Because the N-alkylation reaction generally produces an acid, it is preferably conducted in the presence of a base to neutralize the acid. The base may be, for example, an inorganic base such as NaH or sodium phosphate. In an often particularly preferred embodiment, the base is potassium carbonate. Potassium carbonate is often preferred because it is, for example, easy to handle. In addition, Applicants have discovered that use of potassium carbonate tends to advantageously minimize any impurities that may be formed during the reaction. The amount of base charged to the reactor is preferably at least 1 mole per mole of unprotected piperidinyl compound, more preferably from 1 to about 5 moles per mole of unprotected piperidinyl compound, and still more preferably about 2.1 moles per mole of unprotected piperidinyl compound.

In some preferred embodiments, KBr or KI also is charged to the N-alkylation reaction mixture. KI is particularly preferred. The amount of KBr or KI preferably is from about 1.1 to about 1.9 moles per mole of unprotected piperidinyl compound charged to the reactor, and more preferably about 1.5 moles per mole of unprotected piperidinyl compound charged to the reactor.

The N-alkylation reaction is typically conducted in the presence of a solvent. The solvent preferably has the ability to solubilize the unprotected piperidinyl compound, while not being reactive with any reagents or the protected piperidinyl product. The solvent preferably is polar and aprotic, although other solvents (e.g., toluene, ether, methylene chloride, etc.) may be used, particularly in the presence of a phase transfer reagent. Suitable solvents include, for example, N-methyl-pyrrolidone, dimethylacetamide, acetonitrile, dimethylsulfoxide, hexamethylphosphorus triamide, nitromethane, and/or tetramethylurea. In often more preferred embodiments, the solvent comprises dimethylformamide. The amount of solvent charged to the reactor preferably is from about 0.1 to about 1000 ml per gram of unprotected piperidinyl compound, and more preferably from about 1 to about 100 ml per gram of unprotected piperidinyl compound, with about 10 ml per gram of unprotected piperidinyl compound often being particularly preferred.

In some embodiments, water also is charged to the reactor. This water tends to be beneficial for minimizing impurity formation. The amount of water is preferably from about 2 to about 5% (by volume) of the reaction mixture.

The N-alkylation reaction is typically a heterogeneous reaction. In such instances, the reaction time typically will vary, depending on, for example, the scale of the reaction (i.e., reaction times for smaller scale reactions will typically be less than larger scale reactions). The N-alkylation reaction preferably is carried out until at least about 98% of the unprotected piperidinyl compound is consumed. This typically may be determined using, for example, HPLC. When the reaction is carried out in a batch reactor, the reaction time is typically from about 1 to about 50 hours, and more typically from about 2 to about 20 hours. The reaction time for a lab scale batch reaction, for example, is typically from about 2 to about 3 hours. The reaction time for a pilotor commercial scale batch production often is at least about 18 hours.

In many embodiments, the N-alkylation product is isolated. This may be achieved using, for example, various methods known in the art. In some embodiments, the reaction mixture is diluted with toluene, and then washed with water to remove inorganic salts, and, in some instances, solvent (e.g., dimethylformamide). In those embodiments, the toluene extract is preferably concentrated to afford an oil product. In some particularly preferred embodiments, toluene is added to the toluene extract, and the resulting mixture is concentrated again. In such embodiments, these steps are typically repeated from 2 to 3 times.

Example 1 (Part I) and Example 2 (Part H) illustrate the preparation of a substituted piperidinyl compound using an N-alkylation reaction.

### B-4. Carboxylation

The sulfuramidation product (or the N-substitution product in embodiments where there is an N-substitution following the sulfuramidation) preferably is carboxylated:

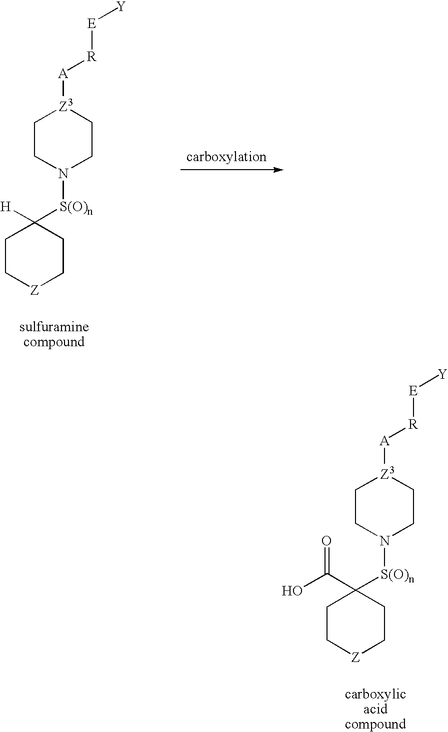

In some preferred embodiments, the sulfuramine compound is carboxylated by contacting the sulfuramine compound with a base to form an anion, and contacting the anion with a carbon dioxide source: 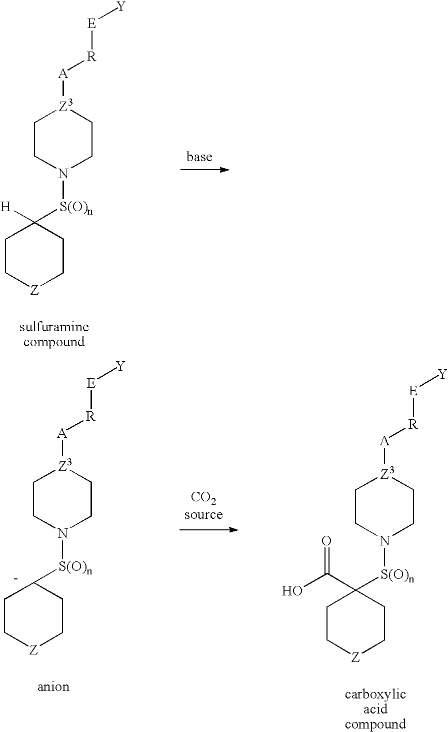
To avoid reaction of the base with the carbon dioxide, these steps are typically conducted sequentially. In addition, this carboxylation method is preferably used with sulfuramine compounds that tend to form an anion only at the desired location (i.e., the heterocyclyl carbon adjacent to the sulfur) when exposed to the base at the preferred conditions.

Both steps of the N-carboxylation reaction may be conducted in a batch, semi-continuous, or continuous mode, with batch mode often being more preferred so that the reaction may be contained until the conversions of the sulfuramine and anion are at least essentially complete. Suitable reactor configurations include, for example, stirred-tank reactors. Other reactor configurations may be used, particularly when such configurations provide sufficient retention time and contact between the reagents for substantial conversions of the sulfuramine and anion. The reactor components in contact with the carboxylation reaction mixture preferably consist essentially of a material(s) that is non-reactive with the reaction reagents, intermediates, and products. Glass reactors are often preferred. Although both steps of the carboxylation may be conducted at a wide variety of pressures and temperatures, they are preferably conducted at ambient pressure, and at a temperature of less than about 50° C., more preferably less than about 20° C., still more preferably from about −20 to about 15° C., and still yet even more preferably from about −15 to about 0° C. Such temperatures tend to advantageously minimize the risk of impurity formation. Because the carboxylation is exothermic, the reactor is typically equipped with a cooling source to maintain the desired temperature.

The carboxylation is normally conducted in the presence of a solvent. The solvent preferably has the ability to solubilize the sulfuramine compound, while not being reactive with any reagents, intermediates, or the carboxylic acid product. The solvent typically comprises an ethereal or aromatic solvent. Suitable solvents include, for example, benzene, tetrahydrofuran, dioxane, diethyl ether, and tert-butylmethyl ether. In some particularly preferred embodiments, the solvent comprises toluene. The amount of solvent charged to the reactor preferably is from about 5 to about 30 ml per gram of sulfuramine compound, and more preferably from about 13 to about 15 ml per gram of sulfuramine compound.

A strong, non-aqueous base is typically preferred for the anion formation reaction. Often suitable bases include, for example, alkyl lithium bases, such as butyl lithium. In some particularly preferred embodiments, the base comprises lithium diisopropylamide. The amount of base charged to the reactor is preferably at least 1 mole per mole of sulfuramine compound, and more preferably from 1 to about 1.5 moles per mole of sulfuramine compound. Normally, the anion formation reaction is conducted under a dry, inert gas (preferably N₂).

When the reaction is carried out in a batch reactor, the anion formation reaction typically is carried out for from about 0.25 to about 10 hours, more typically from about 0.5 to about 2 hours, even more typically from about 0.75 to 1.5 hours, and still even more typically about 1 hour.

The anion may be contacted with a wide variety of carbon dioxide sources. Such sources include, for example, methylchloroformate. In many particularly preferred embodiments, however, the source is a CO₂-containing gas. As used herein, a “CO₂-containing gas” is any gaseous mixture comprising CO₂ that optionally may also comprise one or more diluents which are non-reactive with the reactants and reaction products under the reaction conditions. Examples of such gases include argon, neon, and N₂. Preferably, at least about 95% of the CO₂-containing gas is CO₂. Such a gas preferably is dry and contains essentially no molecular oxygen.

The CO₂-containing gas may be introduced by any convenient means into the reaction medium in a manner that achieves the desired dissolved carbon dioxide concentration in the reaction mixture (normally saturation). In many preferred embodiments, the CO₂-containing gas is introduced into the reaction medium in a manner that maximizes the contact of the gas with the reaction solution. Such contact may be obtained, for example, by dispersing the gas through a diffuser such as a porous glass frit, while shaking or stirring the reactor contents to improve liquid-gas contact and dissolution of the carbon dioxide. Less preferred, although suitable, alternative methods for introducing the carbon dioxide include, for example, use of a back-mixed configuration. It should be noted that when a CO₂-containing gas is used for the carboxylation, it may be desirable to conduct the reaction at a pressure greater than ambient pressure to increase the rate at which the CO₂ dissolves into the reaction mixture.

The CO₂-containing gas preferably is charged to the reactor until the exotherm dissipates. When the reaction is carried out in a batch reactor, the reaction time is typically from about 5 minutes to about 10 hours, more typically from about 10 minutes to about 1 hour, and even more typically from about 15 to about 30 minutes.

To isolate the carboxylic acid product, an NaCl solution is preferably charged to the carboxylation product mixture. The NaCl concentration in the NaCl solution is typically from about 0.5 to about 1% (grams/ml). In some embodiments, the NaCl concentration is about 0.88% (grams/mL). The amount of NaCl solution charged is preferably from about 10 to about 20 mL per gram of the sulfuramine compound, and more preferably from about 17 to about 18 mL per gram of the sulfuramine compound.

The organic layer then preferably is removed, leaving the carboxylic acid product in the aqueous phase. The aqueous phase then preferably is washed with an organic solvent (such as methyl tertiary-butyl ether or toluene, with toluene being particularly preferred) at least one time (and more preferably two times). The amount of organic solvent used for this washing preferably is at least about 7 ml per gram sulfuramine compound, and more preferably from about 12 to about 15 ml per sulfuramine compound. An acid (preferably a strong acid, such as, for example, HCl) is preferably added to the washed aqueous layer in an amount that is sufficient to decrease the pH of the aqueous layer to a pH of from about 7 to about 8.5, and more preferably from about 7.2 to about 8.2. In some embodiments, the temperature of the mixture during the pH adjustment is maintained at from about 50 to about 70° C. In other embodiments, the temperature of the mixture during the pH adjustment is maintained at less than 30° C., and more preferably at from about 20 to about 25° C. The carboxylic acid product will typically precipitate under these conditions.

The carboxylic acid precipitate may be obtained directly from the pH-adjusted mixture using various well-known techniques, including, for example, filtration, settling, and/or centrifugation, with filtration generally being particularly preferred. Applicants have found that product recovery via filtration may be improved by first adding 2-propanol to the mixture, and then allowing the resulting mixture to stand idle for at least about 5 minutes, more preferably from about 0.5 to about 15 hours, even more preferably from about 5 to about 10 hours, and still even more preferably about 8 hours. In such embodiments, the amount of 2-propanol added preferably is at least about 3.5 ml per gram of sulfuramine compound, more preferably from about 3.8 to about 8 ml per gram of sulfuramine compound, and even more preferably from about 4.1 to about 4.3 ml per gram of sulfuramine compound. The improvement in filtration stems, at least in part, from the fact that the 2-propanol addition tends to make the consistency of the carboxylic acid precipitate less pasty.

In some preferred embodiments, the precipitate is additionally or alternatively washed with 2-propanol. In some particularly preferred embodiments, for example, the precipitate is washed with a mixture of water and 2-propanol. The amount of 2-propanol in such a mixture preferably is at least about 0.2 ml 2-propanol per ml water, and more preferably from about 0.25 to about 0.3 ml 2-propanol per ml water. Preferably, the precipitate is washed with at least about 12 ml of this mixture per gram of precipitate, and more preferably at least about 19 ml of this mixture per gram of precipitate.

After the carboxylic acid precipitate has been washed, the precipitate preferably is dried. A wide variety of drying methods may be used. In some preferred embodiments, for example, the precipitate is dried under vacuum (e.g., 25 torr) at a temperature of greater than about 25° C., more preferably from about 30 to about 110° C., even more preferably from about 50 to about 110° C., and still even more preferably from about 75 to about 105° C.

Example 1 (Part J) and Example 2 (Part I) below illustrate the preparation of a carboxylic acid compound using examples of suitable carboxylation protocols.

### B-5. Conversion of the Carboxylic Acid Compound to the Hydroxamic Acid Compound

The carboxylic acid compound can be converted into the hydroxamic acid compound by various methods known in the art. Various techniques for converting carboxylic acids into hydroxamic acids are described in, for example, WIPO Int'l Publ. No. WO 99/25687; and U.S. Pat. No. 6,541,489 (cited above and incorporated by reference into this patent). Such techniques also are described in, for example, WIPO Int'l Publ. No. WO 00/50396; U.S. Patent Pre-Grant Publ. No. 20020177588; and U.S. Patent Pre-Grant Publ. No. 20010039287 (all of which are cited above and incorporated by reference into this patent). Such techniques also are disclosed by in, for example, U.S. Patent Pre-Grant Publ. No. 20010014688 (cited above and incorporated by reference into this patent). Such techniques also are disclosed in WIPO Intl. Publ. No. WO 00/46221; U.S. Pat. No. 6,448,250; U.S. Pat. No. 6,372,758; and U.S. Pat. No. 6,492,367 (all of which are cited above and incorporated by reference into this patent).

In some preferred embodiments, the hydroxamic acid compound is prepared directly from the carboxylic acid compound by reacting the carboxylic acid with N-hydroxylamine (i.e., NH₂OH) in the presence of a coupling reagent (e.g., a carbodiimide, such as 1-(3-dimethylaminopropyl)-3-ethylcarbodiimide hydrochloride)):

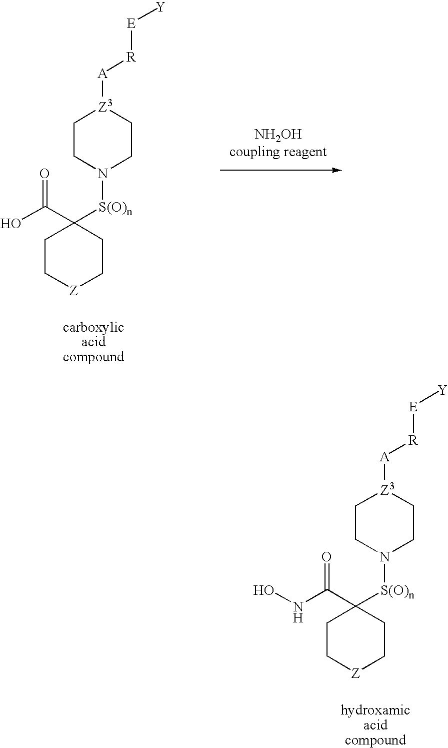

In other embodiments, the carboxylic acid is converted into a compound that is more reactive with N-hydroxyamine. This more reactive compound, in turn, is reacted with the N-hydroxylamine without the presence of a coupling reagent. This avoids any risk of impurities forming due to the N-hydroxylamine reacting with a coupling reagent. In some such embodiments, the carboxylic acid compound is converted into a carboxylic acid chloride compound, which, in turn, is reacted with the N-hydroxylamine.

Such a carboxylic acid chloride may be formed by, for example, reacting the carboxylic acid compound with oxalyl chloride (i.e., ClC(O)C(O)Cl).

In some preferred embodiments, the carboxylic acid is first converted into a hydroxamic acid ester, which, in turn, is cleaved (typically via hydrolysis) to form the hydroxamic acid: 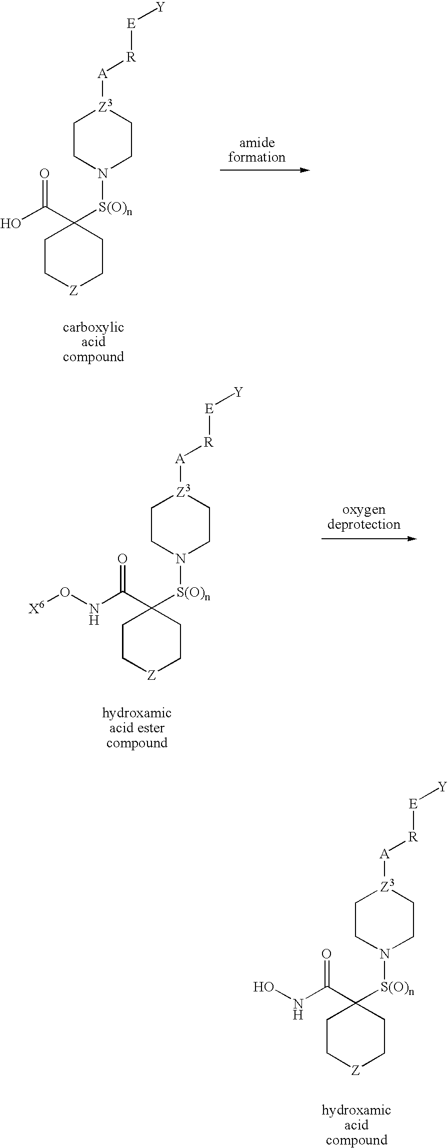
Here, X⁶ is preferably a selectively-removable oxygen-protecting group. Such oxygen-protecting groups include, for example, 2-tetrahydropyranyl, benzyl, p-methoxybenzyl, C₁-C₆-alkoxy-carbonyl, o-nitrophenyl, a trisubstituted silyl group (e.g., those discussed in Greene, T. W.; Wuts, P. G. M.; Protective Groups in Organic Synthesis; 3rd Ed.; Wiley: New York, 1999 (cited above and incorporated by reference into this patent)), and a peptide synthesis resin (e.g., those discussed in, for example, WIPO Int'l Publ. No. WO 00/50396 (cited above and incorporated by reference into this patent); and Floyd et al., Tetrahedron Let., 37(44), 8048 (1996) (incorporated by reference into this patent)).

In some particularly preferred embodiments, X⁶ is 2-tetrahydropyranyl. This hydroxamic acid ester (or “THP amide”) may be prepared from the carboxylic acid compound by, for example, reacting the carboxylic acid compound with O-(tetrahydro-2H-pyran-2-yl)hydroxylamine:

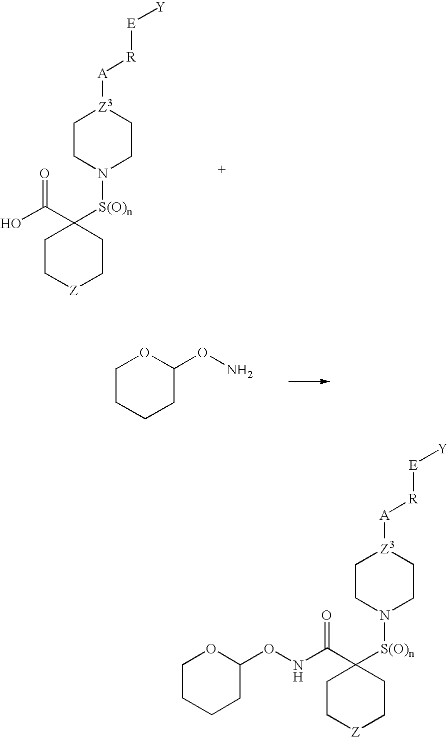

Typically, this reaction is conducted in the presence of a coupling reagent. The coupling reagent is preferably a carbodiimide, such as 1-(3-dimethylaminopropyl)-3-ethylcarbodiimide hydrochloride. The amount of coupling reagent is preferably from about 1.2 to about 2 moles per mole of the carboxylic acid compound, and more preferably from about 1.5 to about 1.8 moles per mole of carboxylic acid compound. In some embodiments, the reaction also is conducted in the presence of a base. A particularly preferred base is triethylamine. Other often suitable bases include, for example, N-methylmorpholine, Hunig's base (generally a trialkylamine), and pyridine.

Formation of the THP amide preferably is conducted in the presence of a solvent. Suitable solvents include, for example, polar aprotic solvents. Examples of various suitable solvents include dimethylformamide, dimethylacetamide, acetonitrile, ethyl acetate, dimethyl sulfoxide, hexamethylphosphorus triamide, nitromethane, tetramethylurea, N-methylpyrrolidone, or a combination thereof. In some particularly preferred embodiments, the solvent comprises dimethylformamide. In other particularly preferred embodiments, the solvent comprises both dimethylformamide and triethylamine. In still other particularly preferred embodiments, the solvent comprises both dimethylformamide and ethyl acetate, with the preferred volumetric ratio of dimethylformamide to ethyl acetate being from about 1:4 to about 1:20, and more preferably from about 1:8 to about 1:10. The amount of solvent is preferably from about 4 to about 8 ml per grams of the carboxylic acid compound, and more preferably from about 7 to about 8 ml per grams of carboxylic acid compound.

Although the THP amide formation may be conducted at a wide variety of pressures and temperature, it preferably is conducted at ambient pressure, and at a temperature of from about 0 to about 100° C., more preferably from about 15 to about 60° C., even more preferably from about 20 to about 75° C., and still even more preferably from about room temperature to about 60° C. These temperature ranges are particularly suitable when the protecting group is 2-tetrahydropyranyl. Normally, the amide formation is conducted under an inert gas (preferably N₂).

The amide formation reaction preferably is carried out until at least about 98% of the carboxylic acid has been consumed. This typically may determined using, for example, HPLC. When, for example, the reaction is carried out in a batch reactor at about 60° C., the reaction time is typically at least about 2 hours, and more typically from about 2 to about 3 hours. Illustrating further, when the reaction is carried out in a batch reactor at about 30° C., the reaction time is typically at least about 12 hours, and more typically from about 12 to about 18 hours.

As noted above, after the hydroxamic acid ester is formed, it is preferably cleaved to remove the selectively-removable oxygen-protecting group to form the hydroxamic acid. This cleavage may be achieved using a wide variety of methods known in the art.

When, for example, the protecting group is a benzyl group, the ester is preferably cleaved using reductive removal of the benzyl group with hydrogen and a metal catalyst (e.g., palladium, platinum, palladium on carbon, or palladium nickel).

When, on the other hand, the protecting group is 2-tetrahydropyranyl, the ester is preferably cleaved using hydrolysis. Acid hydrolysis is a particularly preferred mechanism. Here, the hydroxamic acid ester compound is combined with a strong acid, preferably HCl. An excess of acid is preferably used in this reaction. In some embodiments, the amount of acid charged to the reactor is from about 1.5 to about 4 moles per mole of the hydroxamic acid ester compound, and, more preferably, from about 1.7 to about 2.4 moles per mole of hydroxamic acid ester compound.

The hydrolysis is preferably conducted in the presence of a solvent. Suitable solvents often include water and C₁-C₆-alcohols (e.g., methanol or isopropyl alcohol). Alcohol solvents are typically more preferred, with isopropyl alcohol being particularly preferred. In some embodiments, the solvent may additionally comprise a second solvent that enhances the solubility of the acid. Such a second solvent may be, for example, 1,4-dioxane (particularly when the acid is HCl). The amount of solvent preferably is from about 3 to about 6 ml per grams of hydroxamic acid ester compound, and more preferably from about 5 to about 6 ml per grams of hydroxamic acid ester compound.

Although the hydrolysis may be conducted at a wide variety of pressures and temperatures, it preferably is conducted at ambient pressure, and at a temperature of from about room temperature to about 75° C., and more preferably from about 65 to about 75° C. Normally, the reaction is conducted under an inert gas (preferably N₂).

The hydrolysis reaction preferably is carried out until at least about 98% hydroxamic acid ester has been consumed. This typically may be determined using, for example HPLC. When the reaction is carried out in a batch reactor, the reaction time is typically at least about 4.5 hours, and more typically from about 4.5 to about 5.5 hours.

Example 1 (Parts K and L), Example 2 (Part J), and Example 5 illustrate suitable protocols for converting a carboxylic acid compound to a hydroxamic acid compound.

### B-6. Schemes Illustrating Various Preferred Embodiments

The following schemes illustrate various embodiments for preparing various preferred hydroxamic acid compounds. 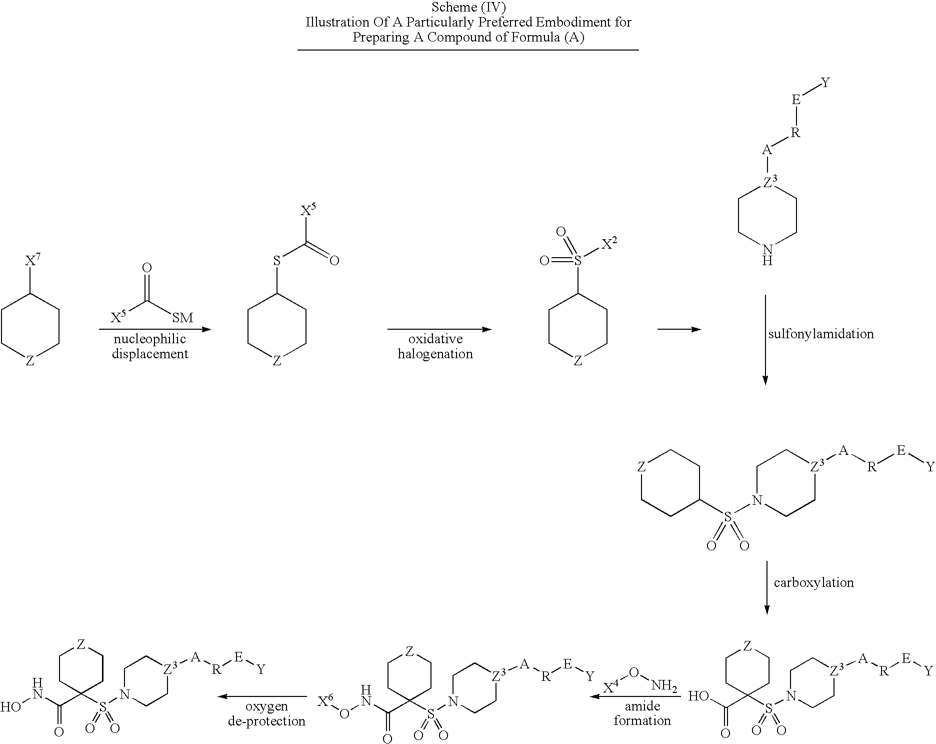
The above scheme is particularly suitable wherein Z is —O—, —S—, —S(O)—, or —S(O)₂—. Although such a scheme is also suitable where Z is —N(R^{x})—, R^{x} preferably is not hydrogen such an instance. 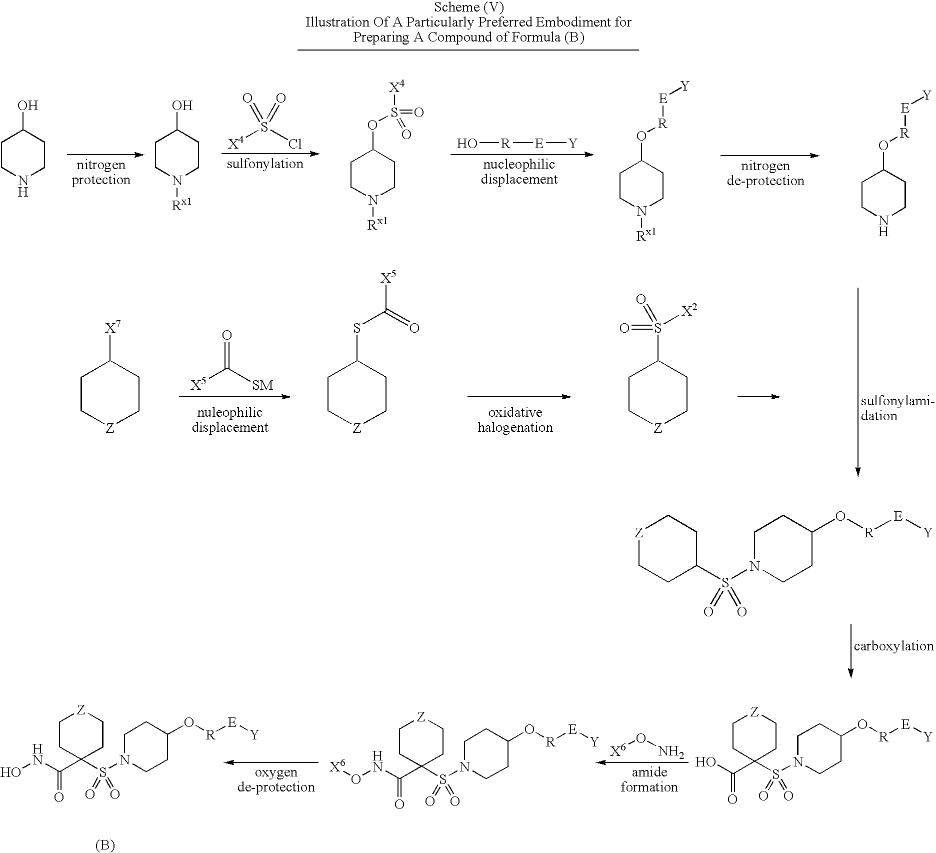
The above scheme is particularly suitable wherein Z is —O—, —S—, —S(O)—, or —S(O)₂—. Although such a scheme is also suitable where Z is —N(R^{x})—, R^{x} preferably is not hydrogen such an instance. 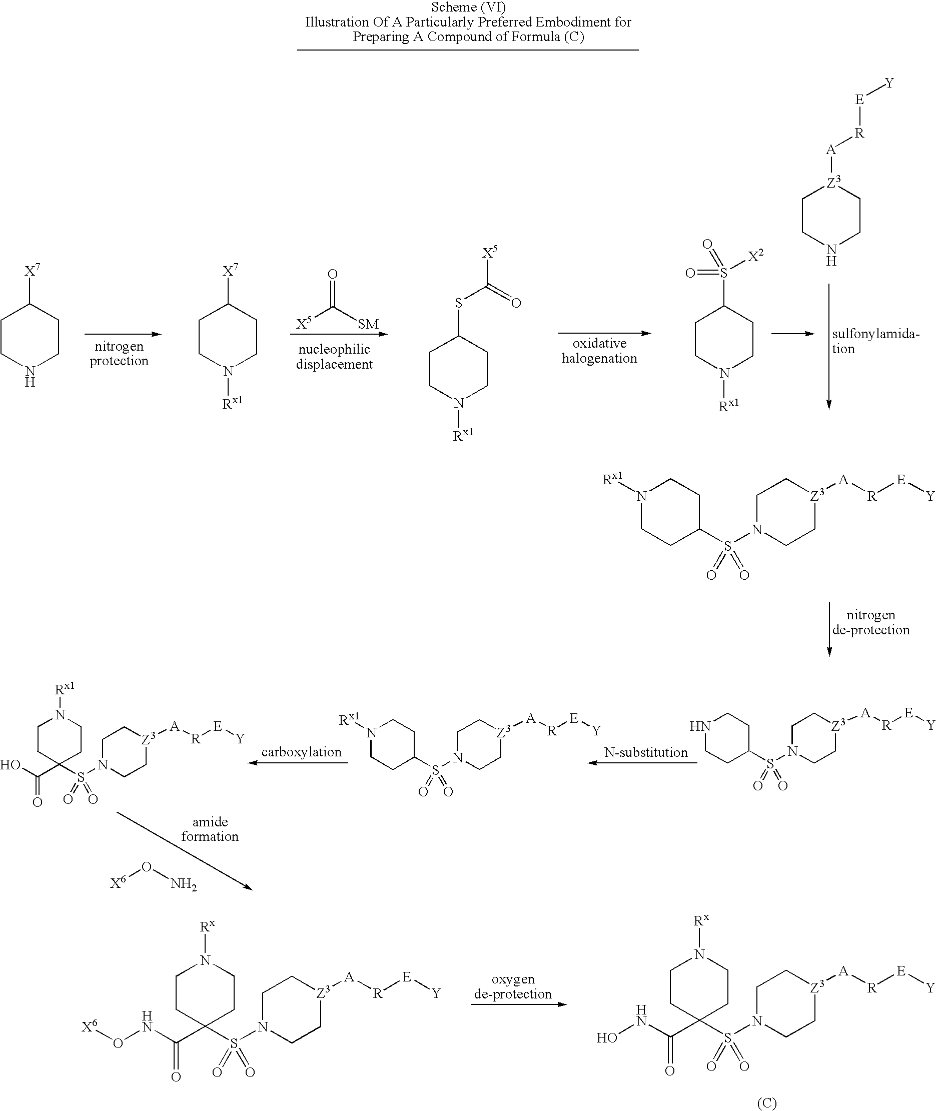 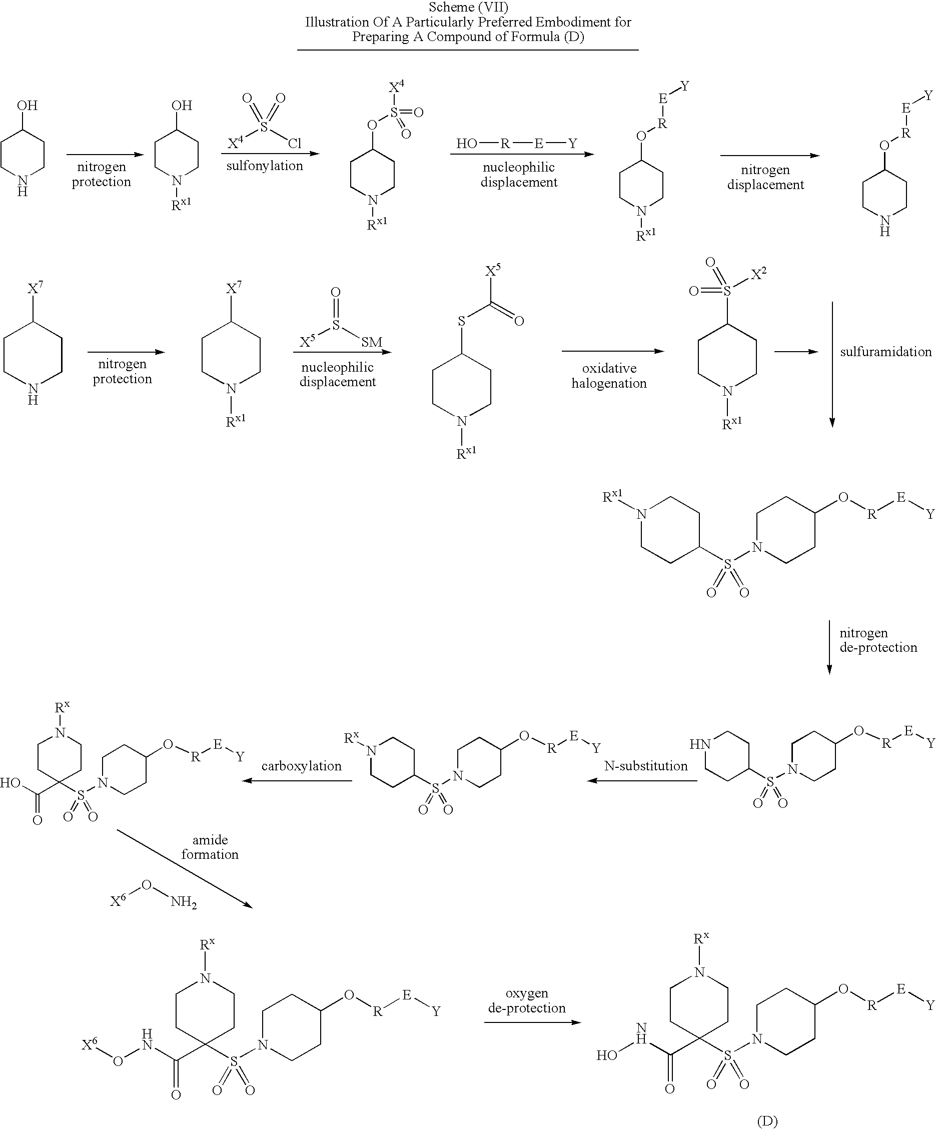
In the above scheme, the nitrogen protecting group (designated as R^{x1}) in the 4-halosulfonyl-piperidinyl compound may be the same as or different than the nitrogen protecting group (also designated as R^{x1}) of the protected 4-hydroxy-piperidinyl compound used to prepare the cyclic amino sulfonylamidation reagent. 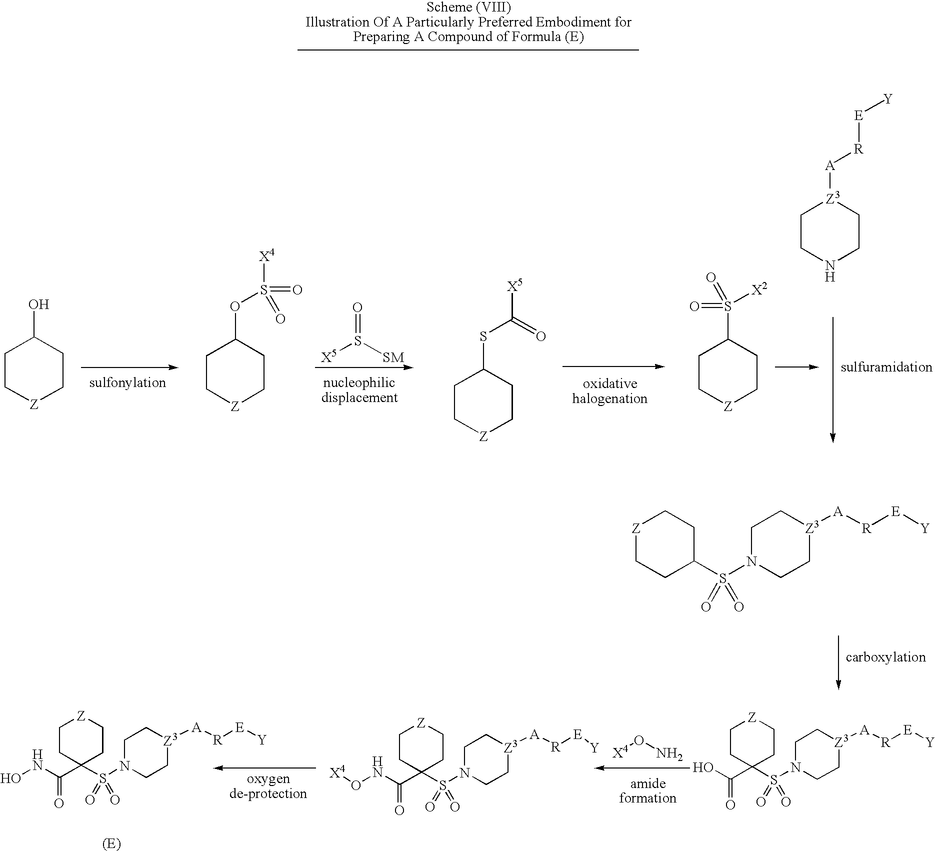
The above scheme is particularly suitable wherein Z is —O—, —S—, —S(O)—, or —S(O)₂—. Although such a scheme is also suitable where Z is —N(R^{x})—, R^{x} preferably is not hydrogen such an instance. 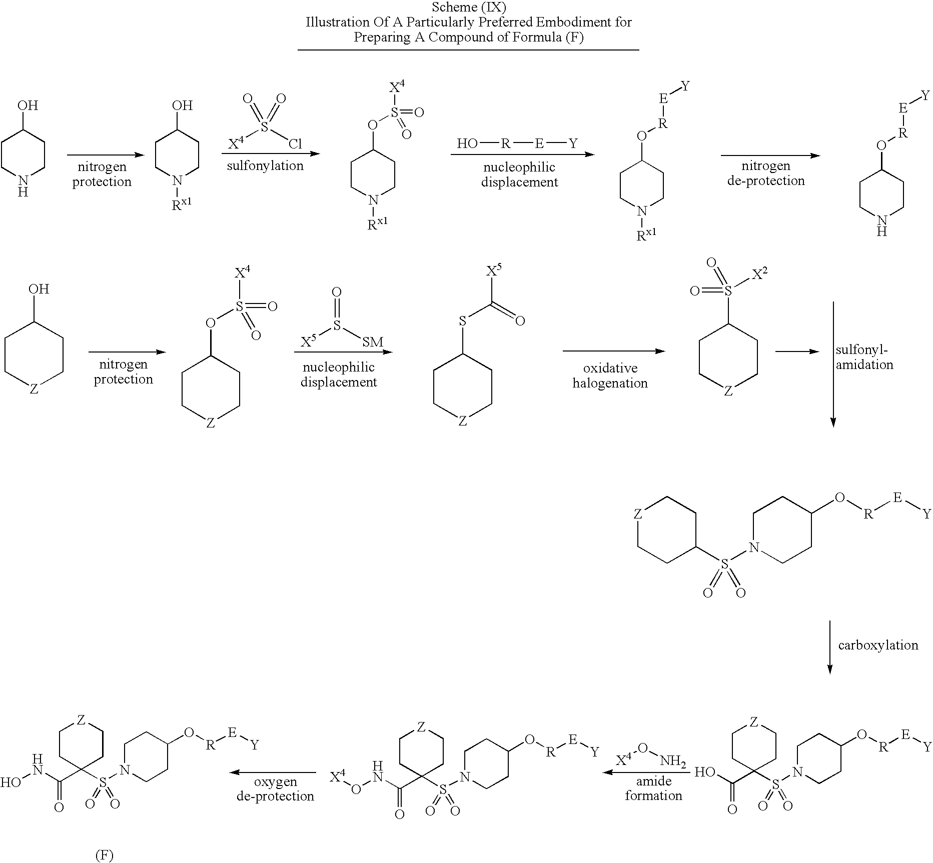
The above scheme is particularly suitable wherein Z is —O—, —S—, —S(O)—, or —S(O)2—. Although such a scheme is also suitable where Z is —N(R^{x})—, R^{x} preferably is not hydrogen such an instance.

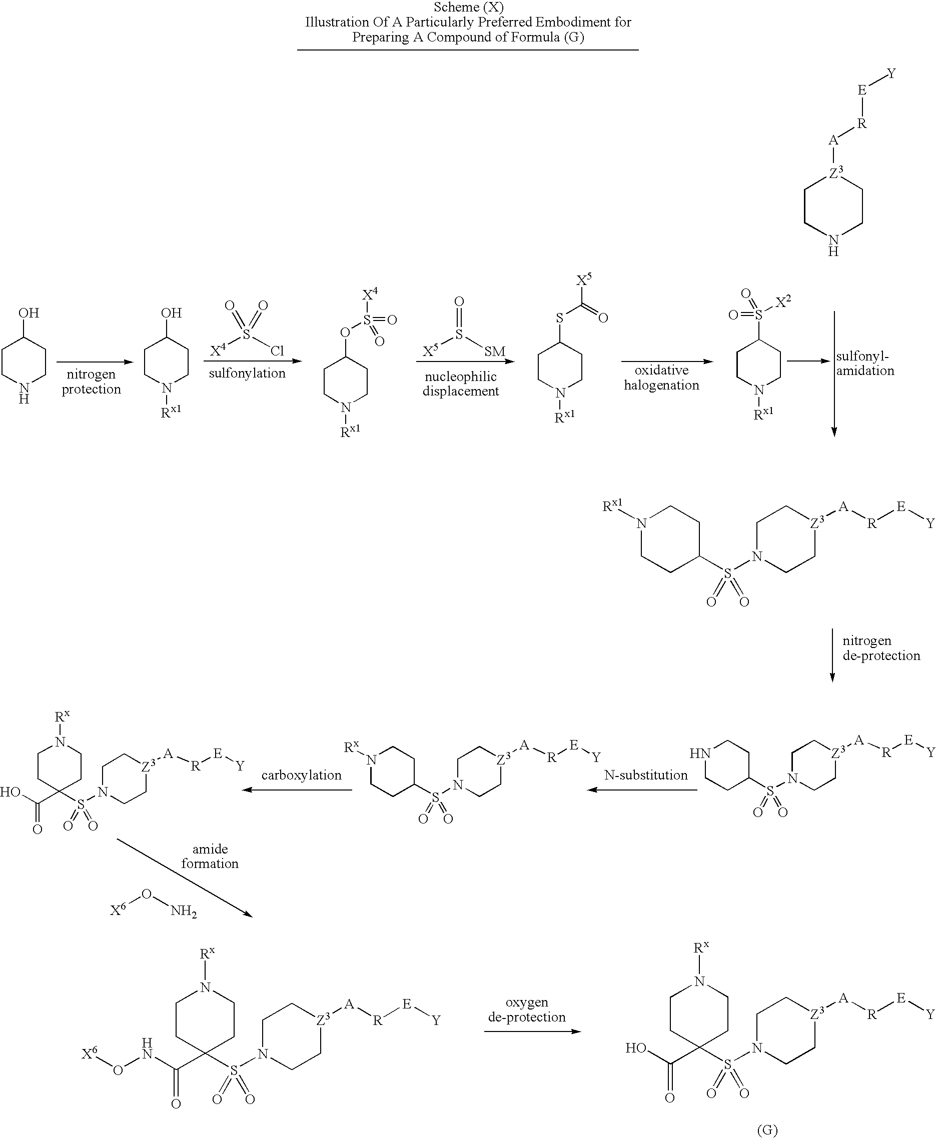

The following schemes illustrate embodiments that use a common compound to form both a cyclic amino intermediate and a 4-halosulfonyl-heterocyclyl intermediate.

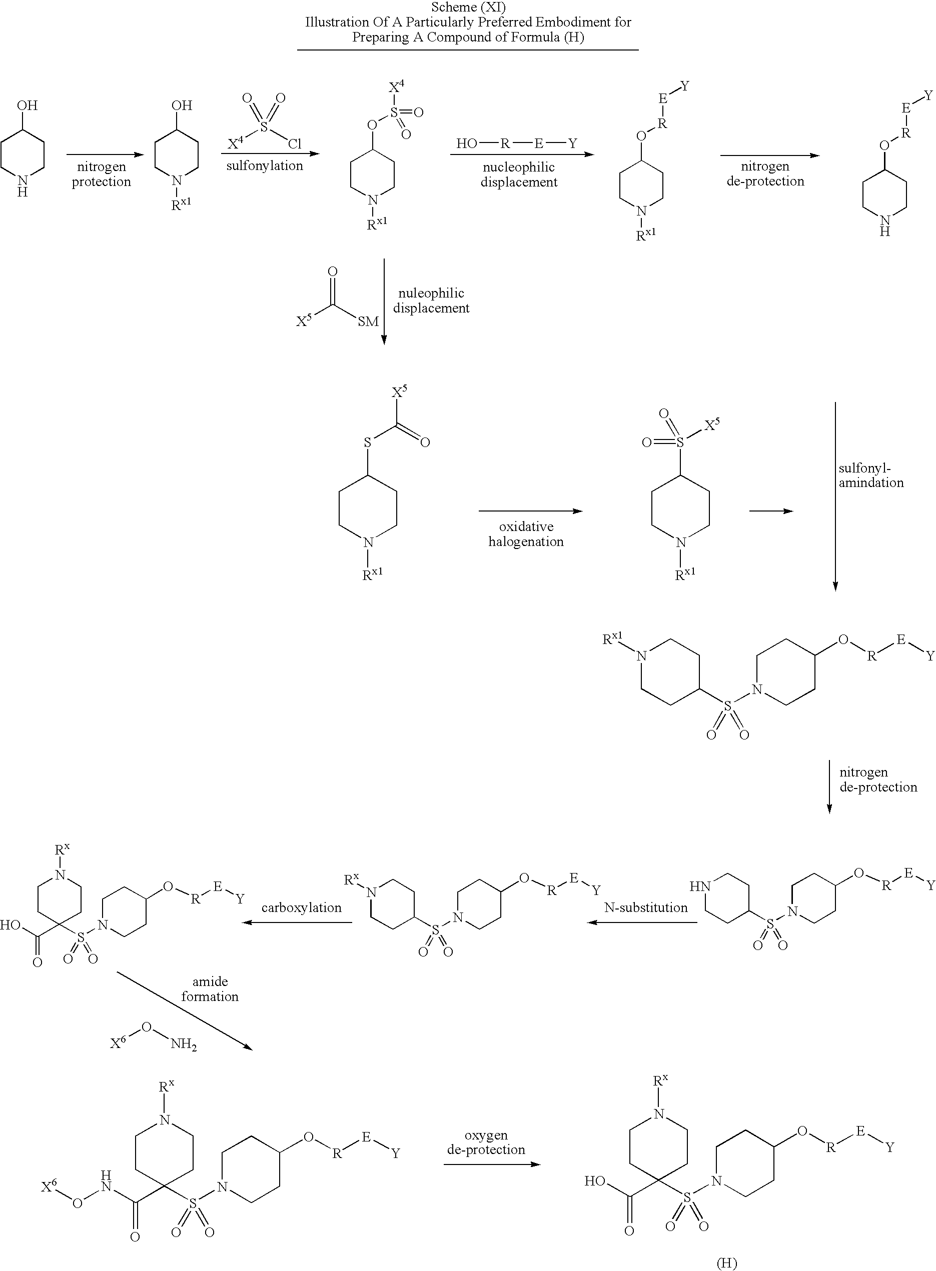

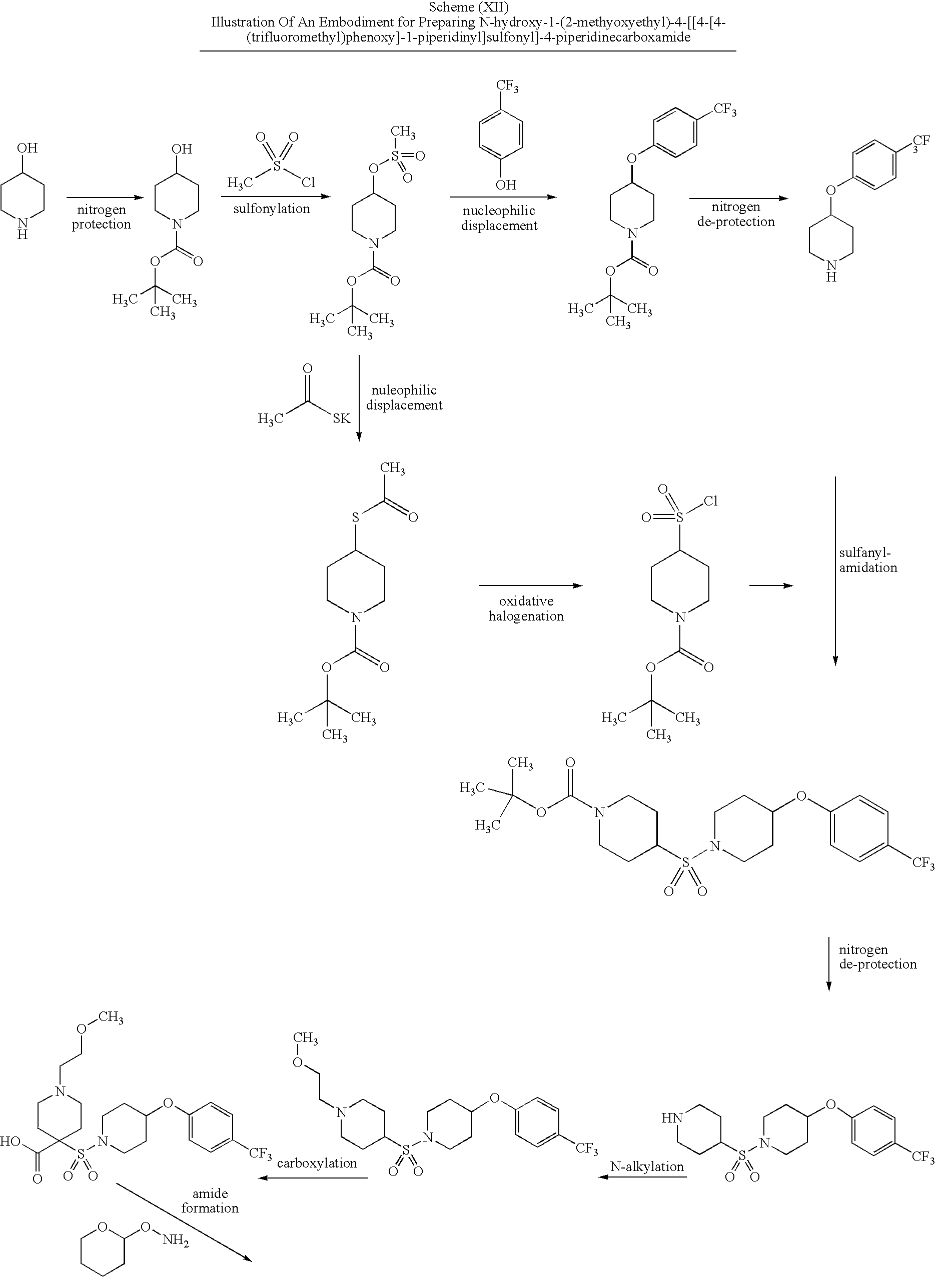

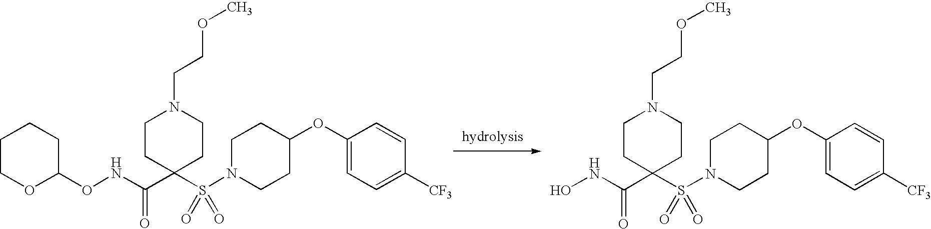

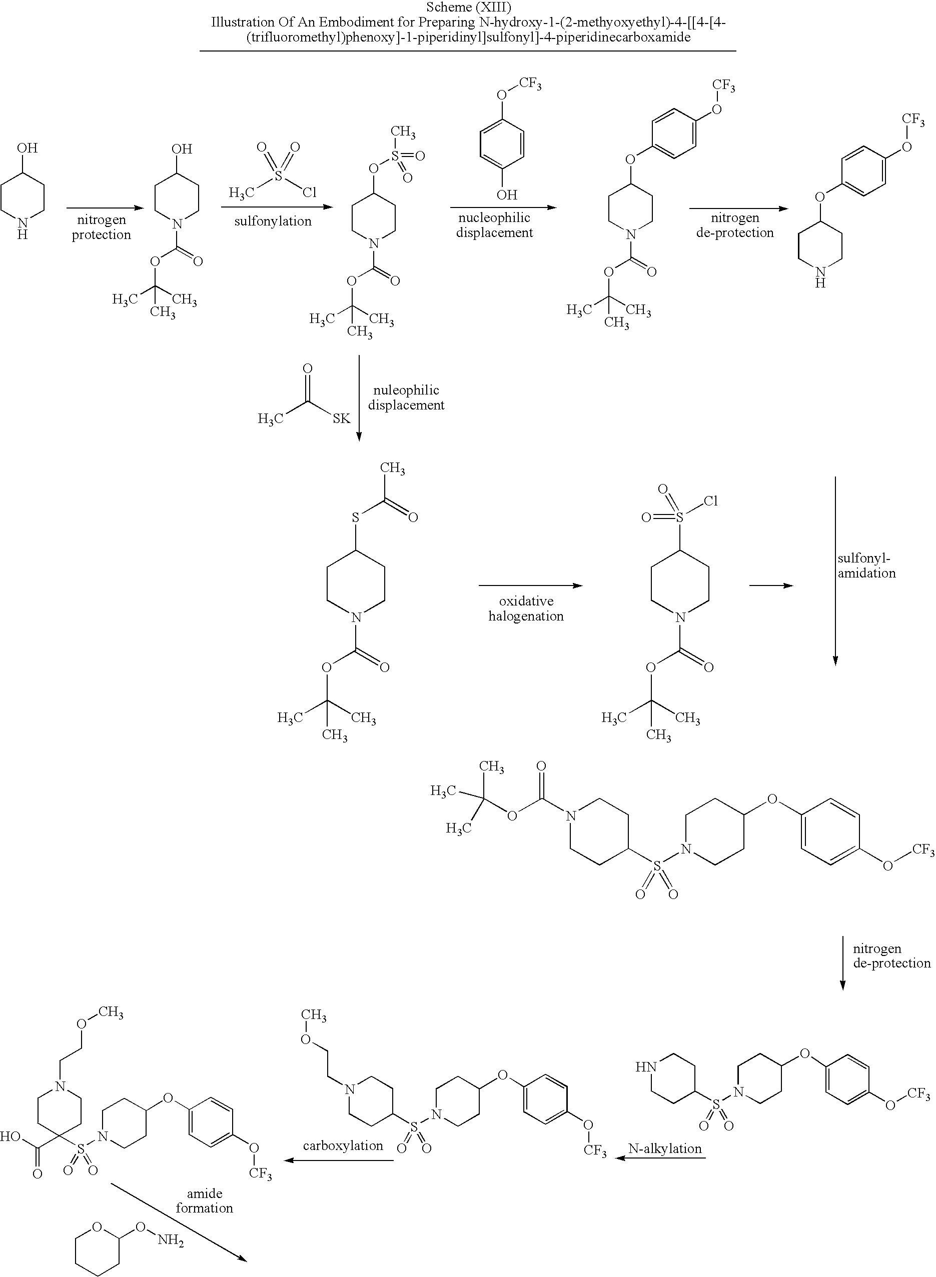

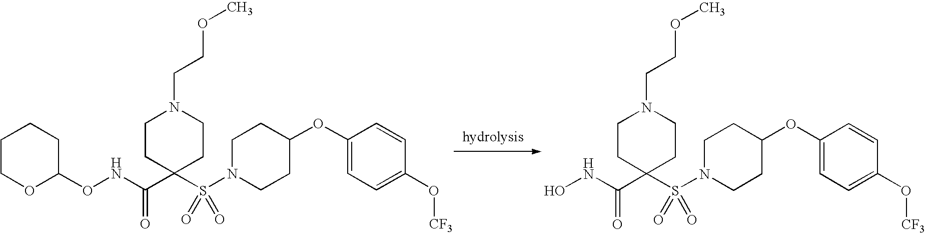

### C. Salts of the Compounds of this Invention

The compounds made in accordance with this invention can be used in the form of salts. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient, the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically-acceptable base addition salts of the compounds of this invention include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, diethanolamine, ethylenediamine, meglumine (N-methylglucamine), and procaine. Basic nitrogen-containing groups can be quaternized with agents such as lower alkyl (C₁ -C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), aralkyl halides (e.g., benzyl and phenethyl bromides), and others.

Particularly preferred salts of the compounds of this invention include hydrochloric acid (HCl) salts and trifluoroacetate (CF₃COOH or “TFA”) salts.

### D. Pharmaceutical Compositions Containing the Compounds and Salts Made in Accordance with this Invention

The hydroxamic acid compounds (including the hydroxamic acid salts) prepared in accordance with this invention may be used in pharmaceutical compositions (or medicaments).

The preferred composition depends on the method of administration, and typically comprises one or more conventional pharmaceutically acceptable carriers, adjuvants, and/or vehicles. Formulation of drugs is generally discussed in, for example, Hoover, John E., Remington's Pharmaceutical Sciences (Mack Publishing Co., Easton, Pa.: 1975). See also, Liberman, H. A. See also, Lachman, L., eds., Pharmaceutical Dosage Forms (Marcel Decker, New York, N.Y., 1980).

Solid dosage forms for oral administration include, for example, capsules, tablets, pills, powders, and granules. In such solid dosage forms, the hydroxamic acids or salts thereof are ordinarily combined with one or more adjuvants. If administered per os, the hydroxamic acids or salts thereof can be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the hydroxamic acid or salt thereof in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the carriers or diluents mentioned for use in the formulations for oral administration. The hydroxamic acids or salts thereof can be dissolved in water, polyethylene glycol, propylene glycol, ethanol, corn oil, cottonseed oil, peanut oil, sesame oil, benzyl alcohol, sodium chloride, and/or various buffers.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols

“Topical administration” includes the use of transdermal administration, such as transdermal patches or iontophoresis devices.

Other adjuvants and modes of administration well-known in the pharmaceutical art may also be used.

### E. Definitions

The term “alkyl” (alone or in combination with another term(s)) means a straight-or branched-Chain saturated hydrocarbyl typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, octyl, and the like.

The term “alkenyl” (alone or in combination with another term(s)) means a straight- or branched-Chain hydrocarbyl containing one or more double bonds and typically from 1 to about 20 carbon atoms, more typically from 2 to about 20 carbon atoms, still more typically from about 2 to about 8 carbon atoms, and even more typically from about 2 to about 6 carbon atoms. Examples of such substituents include ═CH₂; ethenyl (vinyl); 2-propenyl; 3-propenyl; 1,4-pentadienyl; 1,4-butadienyl; 1-butenyl; 2-butenyl; 3-butenyl; decenyl; and the like.

The term “alkynyl” (alone or in combination with another term(s)) means a straight- or branched-Chain hydrocarbyl containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, 3-butynyl, and the like.

The term “carbocyclyl” (alone or in combination with another term(s)) means a saturated cyclic (i.e., “cycloalkyl”), partially saturated cyclic, or aryl hydrocarbyl containing from 3 to 14 carbon ring atoms (“ring atoms” are the atoms bound together to form the ring or rings of a cyclic group). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated cyclic hydrocarbyl containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be 2 or 3 carbon rings fused together, such as, decalinyl or norpinanyl.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl (e.g., alkyl, alkenyl, alkynyl, or cycloalkyl) is indicated by the prefix “Cₓ-C_{y}-” wherein x is the minimum and y is the maximum number of carbon atoms in the substituent. Thus, for example, “C₁-C₆-alkyl” refers to an alkyl containing from 1 to 6 carbon atoms. Illustrating further, C₃-C₆-Cycloalkyl means a saturated hydrocarbyl ring containing from 3 to 6 carbon ring atoms.

The term “hydrogen” (alone or in combination with another term(s)) means a hydrogen radical, and may be depicted as —H.

The term “hydroxy” (alone or in combination with another term(s)) means OH.

The term “nitro” (alone or in combination with another term(s)) means —NO₂.

The term “cyano” (alone or in combination with another term(s)) means —CN, which also may be depicted as:

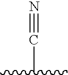

The term “keto” (alone or in combination with another term(s)) means an oxo radical, and may be depicted as ═O.

The term “carboxy” (alone or in combination with another term(s)) means —C(O)—OH, which also may be depicted as:

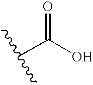

The term “amino” (alone or in combination with another term(s)) means —NH₂. The term “monosubstituted amino” (alone or in combination with another term(s)) means an amino wherein one of the hydrogen radicals is replaced by a non-hydrogen substituent. The term “disubstituted amino” (alone or in combination with another term(s)) means an amino wherein both of the hydrogen atoms are replaced by non-hydrogen substituents, which may be identical or different.

The term “cyclic amino” (alone or in combination with another term(s)) means a heterocyclyl moiety comprising at least one nitrogen ring atom, with the remaining ring atoms being carbon and optionally nitrogen. Examples of such moieties include piperidinyl and piperazinyl groups.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I). Typically, a fluorine radical or chlorine radical is preferred, with a fluorine radical often being particularly preferred.

If a substituent is described as being “substituted”, a non-hydrogen radical is in the place of a hydrogen radical on, for example, a carbon or nitrogen of the substituent. Thus, for example, a substituted alkyl substituent is an alkyl substituent wherein at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitutions on a substituent, each non-hydrogen radical may be identical or different (unless otherwise stated).

If a substituent is described as being “optionally substituted”, the substituent is either (1) substituted, or (2) not substituted. Where the members of a group of substituents are described generally as being optionally substituted, any atom capable of substitution in each member of such group may be (1) substituted, or (2) not substituted. Such a characterization contemplates that some members of the group are not substitutable. Atoms capable of substitution include, for example, carbon bonded to at least one hydrogen, oxygen bonded to at least one hydrogen, sulfur bonded to at least one hydrogen, or nitrogen bonded to at least one hydrogen. On the other hand, hydrogen alone, halogen, oxo, and cyano do not fall within the definition of being capable of substitution.

This specification uses the terms “substituent” and “radical” interchangeably.

The prefix “halo” indicates that the substituent to which the prefix is attached is substituted with one or more independently selected halogen radicals. For example, haloalkyl means an alkyl wherein at least one hydrogen radical is replaced with a halogen radical. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyoxy”), 1,1,1,-trifluoroethoxy, and the like. It should be recognized that if a substituent is substituted by more than one halogen radical, those halogen radicals may be identical or different (unless stated otherwise).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl wherein a fluorine radical is in the place of each hydrogen radical. Examples of perfluoroalkyl substituents include trifluoromethyl (—CF₃), perfluorobutyl, perfluoroisopropyl, perfluorododecyl, perfluorodecyl, and the like. To illustrate further, the term “perfluoroalkoxy” means an alkoxy wherein each hydrogen radical is replaced with a fluorine radical. Examples of perfluoroalkoxy substituents include trifluoromethoxy (—O—CF₃), perfluorobutoxy, perfluoroisopropoxy, perfluorododecoxy, perfluorodecoxy, and the like.

The term “carbonyl” (alone or in combination with another term(s)) means —C(O)—, which also may be depicted as: 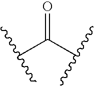
This term also is intended to encompass a hydrated carbonyl substituent, i.e., —C(OH)₂—.

The term “aminocarbonyl” (alone or in combination with another term(s)) means —C(O)—NH₂, which also may be depicted as:

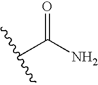

The term “oxy” (alone or in combination with another term(s)) means an ether substituent, and may be depicted as —O—.

The term “alkoxy” (alone or in combination with another term(s)) means an alkylether, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

The term “alkylcarbonyl” (alone or in combination with another term(s)) means —C(O)-alkyl. For example, “ethylcarbonyl” may be depicted as:

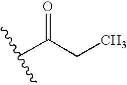

The term “alkoxycarbonyl” (alone or in combination with another term(s)) means —C(O)—O—alkyl. For example, “ethoxycarbonyl” may be depicted as:

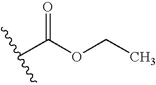

The term “carbocyclylcarbonyl” (alone or in combination with another term(s)) means —C(O)-carbocyclyl. For example, “phenylcarbonyl” may be depicted as: 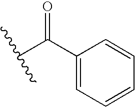
Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl. For example, “phenylethylcarbonyl” may be depicted as: 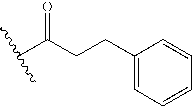
Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl. For example, “phenyloxycarbonyl” may be depicted as:

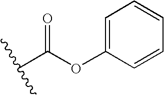

The term “carbocyclylalkoxycarbonyl” (alone or in combination with another term(s)) means —C(O)—O-alkyl-carbocyclyl. For example, “phenylethoxycarbonyl” may be depicted as:

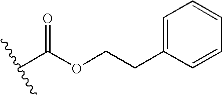

The term “thio” or “thia” (alone or in combination with another term(s)) means a thiaether, i.e., an ether substituent wherein a divalent sulfur atom is in the place of the ether oxygen atom. Such a substituent may be depicted as —S—. This, for example, “alkyl-thio-alkyl” means alkyl-S-alkyl.

The term “thiol” or “sulffiydryl” (alone or in combination with another term(s)) means a sulffiydryl, and may be depicted as —SH.

The term “sulfonyl” (alone or in combination with another term(s)) means —S(O)₂—, which also may be depicted as: 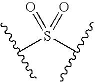
Thus, for example, “alkyl-sulfonyl-alkyl” means alkyl—S(O)₂-alkyl.

The term “aminosulfonyl” (alone or in combination with another term(s)) means —S(O)₂—NH₂, which also may be depicted as:

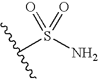

The term “sulfoxido” (alone or in combination with another term(s)) means —S(O)—, which also may be depicted as: 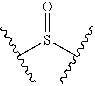
Thus, for example, “alkylsulfoxidoalkyl” means alkyl—S(O)-alkyl.

The term “heterocyclyl” (alone or in combination with another term(s)) means a saturated (i.e., “heterocycloalkyl”), partially saturated, or heteroaryl ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetrahydrofuranyl, thiophenyl (also known as “thiofuranyl” or “thienyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl and 1,2,6-oxathiazinyl), oxadiazinyl (including 1,4,2-oxadiazinyl and 1,3,5,2-oxadiazinyl), morpholinyl, azepinyl, oxepinyl, thiepinyl, and diazepinyl.

A heterocyclyl alternatively may be 2 or 3 rings fused together, such as, for example, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, pyrido[4,3-b]-pyridinyl, and naphthyridinyl), and pteridinyl. Other examples of fused-ring heterocyclyls include benzo-fused heterocyclyls, such as indolyl, isoindolyl, indoleninyl (also known as “pseudoindolyl”), isoindazolyl (also known as “benzpyrazolyl”), benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including chromenyl and isochromenyl), benzothiopyranyl (also known as thiochromenyl), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, or “benzothiofuranyl”), isobenzothienyl (also known as “isobenzothiophenyl”, “isothionaphthenyl”, or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), tetrahydroisoquinolinyl, carbazolyl, xanthenyl, and acridinyl.

The term “2-fused-ring” heterocyclyl (alone or in combination with another term(s)) means a saturated, partially saturated, or heteroaryl containing 2 fused rings. Examples of 2-fused-ring heterocyclyls include indolizinyl, pyrindinyl, pyranopyrrolyl, 4H-quinolizinyl, purinyl, pyridopyridinyl, pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridinyl, pyrazinyl, pyrimidinyl, pyridazinyl, and 1,3,5-, 1,2,4-, and 1,2,3-triazinyl; 5-membered ring substituents such as imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, and quinazolinyl.

A carbocyclyl or heterocyclyl can optionally be substituted with, for example, one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, hydroxy, carboxy, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) optionally may further be substituted with, for example, one or more halogen. The aryls or cycloalkyls are typically single-ring substituents containing from 3 to 6 ring atoms, and more typically from 5 to 6 ring atoms.

An aryl or heteroaryl can optionally be substituted with, for example, one or more substituents independently selected from the group consisting of halogen, hydroxy, cyano, amino, thiol, carboxy, amino, aminocarbonyl, aminoalkyl, alkyl, alkylthio, carboxyalkylthio, alkylcarbonyl, alkylcarbonyloxy, alkoxy, alkoxyalkyl, alkoxycarbonyl, alkoxycarbonylalkoxy, alkoxyalkylthio, alkoxycarbonylalkylthio, carboxyalkoxy, alkoxycarbonylalkoxy, carbocyclyl, carbocyclylalkyl, carbocyclyloxy, carbocyclylthio, carbocyclylalkylthio, carbocyclylamino, carbocyclylalkylamino, carbocyclylcarbonylamino, carbocyclylcarbonyl, carbocyclylalkyl, carbocyclylcarbonyloxy, carbocyclyloxycarbonyl, carbocyclylalkoxycarbonyl, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyl, heterocyclylalkylcarbonyl, heterocyclyloxycarbonyl, heterocyclylcarbonyloxy, heterocyclylalkoxycarbonyl, heterocyclyloxyalkoxyheterocyclyl, heterocyclylthioalkylthioheterocyclyl, heterocyclylthioalkoxyheterocyclyl, and heterocyclyloxyalkylthioheterocyclyl. More typically, an aryl or heteroaryl may, for example, optionally be substituted with one or more substituents independently selected from the group consisting of halogen, hydroxy, cyano, amino, thiol, carboxy, amino, aminocarbonyl, amino—C₁-C₆-alkyl, C₁-C₆-alkyl, C₁-C₆-alkylthio, carboxy-C₁-C₆-alkylthio, C₁-C₆-alkylcarbonyl, C₁-C₆-alkylcarbonyloxy, C₁-C₆-alkoxy, C₁-C₆-alkoxy-C₁-C₆-alkyl, C₁-C₆-alkoxycarbonyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, aryl-C₁-C₆-alkyl, aryloxy, arylthio, aryl-C₁-C₆-alkylthio, arylamino, aryl-C₁-C₆-alkylamino, arylcarbonylamino, arylcarbonyl, aryl-C₁-C₆-alkylcarbonyl, arylcarbonyloxy, aryloxycarbonyl, aryl-C₁-C₆-alkoxycarbonyl, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyl, cycloalkyl-C₁-C₆-alkylcarbonyl, cycloalkylcarbonyloxy, cycloalkyloxycarbonyl, cycloalkyl-C₁-C₆-alkoxycarbonyl, heteroaryl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, heteroarylcarbonyl, heteroaryl-C₁-C₆-alkylcarbonyl, heteroaryloxycarbonyl, heteroarylcarbonyloxy, and heteroaryl-C₁-C₆-alkoxycarbonyl. Here, one or more hydrogen bound to a carbon in any such substituent may, for example, optionally be replaced with halogen. In addition, the cycloalkyl, aryl, and heteroaryl are typically single-ring substituents containing 3 to 6 ring atoms, and more typically 5 or 6 ring atoms.

A prefix attached to a multi-Component substituent only applies to the first component. To illustrate, the term “alkylcycloalkyl” contains two components: alkyl and cycloalkyl. Thus, the C₁-C₆-prefix on “C₁-C₆-alkylcycloalkyl” means that the alkyl component of the alkylcycloalkyl contains from 1 to 6 carbon atoms; the C₁-C₆-prefix does not describe the cycloalkyl component. To illustrate further, the prefix “halo” on haloalkoxyalkyl indicates that only the alkoxy component of the alkoxyalkyl substituent is substituted with one or more halogen radicals. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other substituent(s).

When words are used to describe a substituent, the rightmost-described component of the substituent is the component that has the free valence. To illustrate, benzene substituted with methoxyethyl has the following structure: 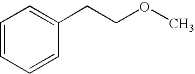
As can be seen, the ethyl is bound to the benzene, and the methoxy is the component of the substituent that is the component furthest from the benzene. As further illustration, benzene substituted with cyclohexanylthiobutoxy has the following structure:

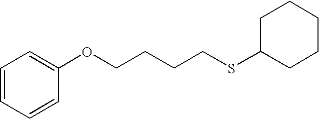

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-Cyclohexanyl-methyl-Y.

When a chemical formula is used to describe a substituent, a hanging dash in the formula indicates a free valence. To illustrate, benzene substituted with —C(O)—OH has the following structure:

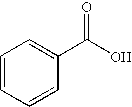

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

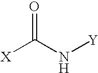

The term “pharmaceutically acceptable” is used adjectivally in this specification to mean that the modified noun is appropriate for use as a pharmaceutical product or as a part of a pharmaceutical product.

The term “ambient pressure” means about 1 atmosphere.

The terms “room temperature” and “ambient temperature” mean a temperature of from about 20 to about 25° C.

The abbreviation “DMF” means dimethylformamide (also called “N,N-dimethylformamide”).

The abbreviation “NMP” means N-methyl-pyrrolidone.

The abbreviation “LDA” means lithium diisopropylamide.

The abbreviation “THP” means 2-tetrahydropyranyl.

The abbreviation “EDC” means 1-(3-dimethylaminopropyl)-3-ethylcarbodiimide hydrochloride.

The abbreviation “NMM” means N-methylmorpholine.

The abbreviation “DMAc” means dimethylacetamide.

The abbreviation “DMSO” means dimethyl sulfoxide.

The abbreviation “HMPA” means hexamethylphosphorus triamide.

The abbreviation “TBME” or “MTBE” means tert-butylmethyl ether or methyl tertiary-butyl ether.

The abbreviation “THF” means tetrahydrofuran.

The abbreviation “BOC” means t-butyloxycarbonyl.

With reference to the use of the words “comprise” or “comprises” or “comprising” in this specification, Applicants note that unless the context requires otherwise, those words are to be interpreted inclusively, rather than exclusively, and that Applicants intend each of those words to be so interpreted in construing this specification.

### F. EXAMPLES OF COMPOUND PREPARATION

The following examples are merely illustrative of the process of this invention, and not limiting to the remainder of this disclosure in any way. Other compounds and salts of this invention may be prepared using the process illustrated in these examples (either alone or in combination with techniques generally known in the art). Such known techniques include, for example, those disclosed in WIPO Intl. Publ. No. WO 00/46221; U.S. Pat. No. 6,448,250; U.S. Pat. No. 6,372,758; and U.S. Pat. No. 6,492,367 (all of which are cited above and incorporated by reference into this patent).

### Example 1

### Preparation of N-hydroxy-1-(2-methoxyethyl)-4-[[4-[4-(trifuoromethoxy)phenoxy]-1-piperidinyl]sulfonyl]-4-piperidinecarboxamide, monohydrochloride

### Part A: Preparation of 4-(methylsulfonyl)hydroxy-1-piperidinecarboxylic acid, 1,1-dimethylethyl ester

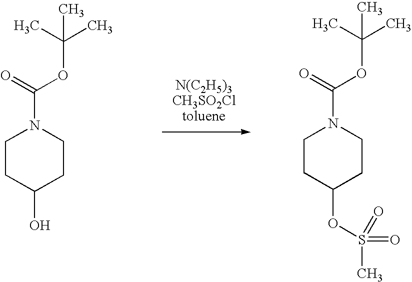 The following reaction was carried out in a 2.5 L 4-Neck jacketed cylindrical glass reactor with an overhead stirrer and a nitrogen atmosphere. To a cold (10° C.) solution of tert-butyl 4-hydroxy-1-piperidinecarboxylate (174 g, 0.866 mol, Sigma-Aldrich (St. Louis, Mo.)) in toluene (805 mL) was added triethylamine (135 mL, 0.969 mol), followed by methanesulfonyl chloride (111 g, 0.970 mol) at a rate that allowed the temperature to be maintained at less than 20° C. The resulting heterogeneous mixture was stirred at 20° C. for at least 1 hr. Water (310 mL) was then added to dissolve the solids, and a biphasic mixture was obtained. The upper organic layer, which contained about 242 g (or 0.866 mol, assuming 100% yield) of the 4-(methylsulfonyl)hydroxy-1-piperidinecarboxylic acid, 1,1-dimethylethyl ester in toluene, was separated and used as is in the next step.

### Part B. Preparation of 4-[4-(trifluoromethoxy)-phenoxyl-1-piperidinecarboxylic acid, 1,1-dimethylethyl ester

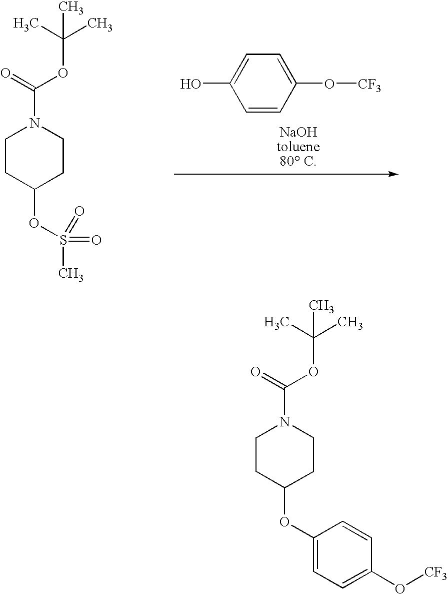 The following reaction was carried out in a 2.5 L 4-Neck jacketed cylindrical glass reactor with an overhead stirrer and a nitrogen atmosphere. To the solution of 4-(methylsulfonyl)hydroxy-1-piperidinecarboxylic acid, 1,1-dimethylethyl ester in toluene from Part A (242 g, 0.866 mol) was added 4-(trifluoromethoxy)phenol (119 mL, 0.919 mol). Additional toluene (70 mL) was used to rinse the transfer line and charge port. Aqueous NaOH (30 weight %, 350 g, 2.62 mol) was added, and the resulting mixture was heated at 80° C. for no greater than 2.5 hr. After cooling to ambient temperature, water (525 mL) was added. The lower aqueous layer was removed, and the upper organic layer was washed with 1 N NaOH solution (350 mL) and water (200 mL). The organic layer was concentrated under reduced pressure to about one-third of the original weight. The concentrate contained a mixture of 4-[4-(trifluoromethoxy)-phenoxy]-1-piperidinecarboxylic acid, 1,1-dimethylethyl ester (188 g, 0.520 mol), and tert-butyl 3,6-dihydropyridine-1(2H)-carboxylate (64 g, 0.35 mol).

### Part C: Preparation of 4-[4-(trifluoromethoxy)-phenoxy]piperidine

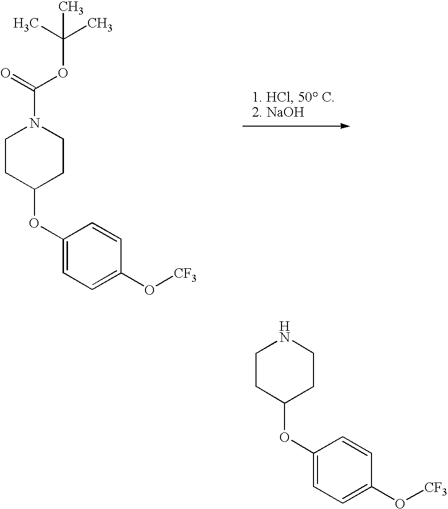 The following reaction was carried out in a 2.5 L, 4-Neck, jacketed cylindrical glass reactor equipped with an overhead stirrer and a nitrogen atmosphere. The concentrate containing a mixture of 4-[4-(trifluoromethoxy)-phenoxy]-1-piperidinecarboxylic acid, 1,1-dimethylethyl ester (188 g, 0.520 mol), and tert-butyl 3,6-dihydropyridine-1(2H)-carboxylate (64 g, 0.35 mol) from Part B was heated to 50° C. Concentrated HCl solution (37 weight %, 225 mL, 2.80 mol) was added at a rate that allowed the temperature to be maintained at about 50° C. Carbon dioxide gas evolution was observed during the addition. The reaction mixture was stirred at 50° C. for 1 hr. Cessation of gas evolution indicated that the reaction was complete. The reaction mixture was cooled to 5-10° C., and then aqueous NaOH solution (30 weight %, 350 g, 2.62 mol) was added at a rate which allowed the temperature to be maintained at less than 20° C. The resulting mixture was warmed to 20° C., and then toluene (700 mL) and water (350 mL) was added. The organic layer was separated and washed with water (350 mL). The organic layer was then concentrated under reduced pressure to remove toluene and 1,2,3,6-tetrahydropyridine (In some runs, if 1,2,3,6-tetrahydropyridine was still present in the concentrate (as determined by GC analysis), additional toluene was added and a second concentration was performed). Heptane (500 mL) was added to the concentrate at 60° C., and the mixture was heated at 60° C. until a clear solution was obtained. The solution was then slowly cooled to −10° C. in 10° C. increments with short hold times. Crystallization occurred at just above 30° C. Good agitation was required to minimize crusting of material on the reactor walls. The mixture was filtered, and the cake was washed with cold (−10° C.) heptane rinses (200 mL, 150 mL) of the reactor walls. The cake was air dried on a filter for 1 hr, and then dried in a vacuum oven (house vacuum, ambient temperature) for 2 hr to give 94.8 g of 4-[4-(trifluoromethoxy)-phenoxy]piperidine as a white to off-white solid. Analysis by GC showed 97.4 area % purity. The overall yield of 4-[4-(trifluoromethoxy)-phenoxy]piperidine from tert-butyl 4-hydroxy-1-piperidinecarboxylate was 42%. ¹H NMR (CDCl₃, 400 MHz): 1.55-1.70 (m, 5H), 1.95-2.05 (m, 2H), 2.72 (ddd, 2H), 3.13 (dt, 2H), 4.43 (septet, 1H), 6.89 (d, 2H), 7.13 (d, 2H).

### Part D. Preparation of benzyl 4-bromopiperidine-1-carboxylate

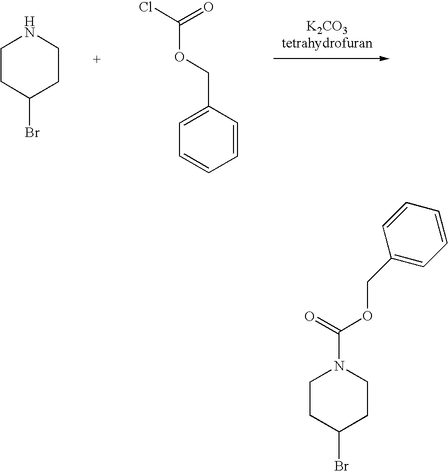 The following reaction was carried out in a 5 L, three-Neck, round-bottomed flask with an overhead stirrer and a nitrogen atmosphere. To a solution of 4-bromopiperidine hydrobromide (400 g, 1.63 mol) in THF (650 mL) was added a solution of powdered (−325 mesh) potassium carbonate (226 g, 1.63 mol) in water (650 mL). The mixture was cooled to 5° C., and then benzyl chloroformate (95 weight %, 279 g, 1.55 mol) was added at a rate that allowed the temperature to be maintained at less than 10° C. After the addition was complete, the reaction mixture was allowed to warm to ambient temperature and stirred for 1 hr. Ethyl acetate (1.6 L) was added, and the mixture was stirred for 10 min. The organic layer was separated and concentrated under reduced pressure to give 473 g (102% crude yield) of product. Analysis by GC showed 94 area % purity. ¹H NMR (CDCl₃, 400 MHz): 1.95-2.02 (m, 2H), 2.02-2.18 (m, 2H), 3.40-3.50 (m, 2H), 3.70-3.80 (m, 2H), 4.19 (septet, 1H), 5.16 (s, 2H), 7.30-7.40 (m, 5H).

### Part E. Preparation of benzyl 4-(acetylthio)piperidine-1-carboxylate

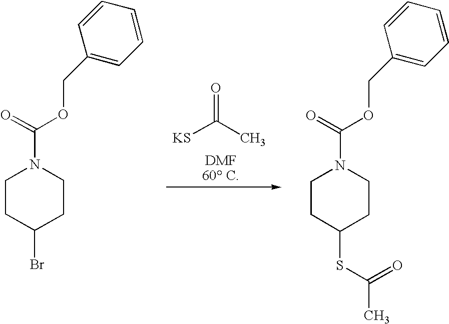 The following reaction was carried out in a 5 L three-Neck round-bottomed flask with an overhead stirrer and a nitrogen atmosphere. A mixture of benzyl 4-bromopiperidine-1-carboxylate from Part D (442 g, 1.48 mol) and potassium thioacetate (220 g, 1.93 mol) in DMF (1.3 L) was heated at 60° C. for 3.5 hr. The mixture was then cooled to ambient temperature, and water (2.6 L) was added. An exotherm to 41° C. was initially observed during the first half of the water addition, but the temperature decreased during the second half of the water addition. Ethyl acetate (700 mL) was added, and the mixture was stirred for 10 min. The organic layer was separated, and the aqueous layer was back-extracted with ethyl acetate (700 mL). The organic extracts were combined, washed twice with water (1 L, 0.5 L), and concentrated under reduced pressure to give 426 g (98% crude yield) of product. Analysis by GC showed 82 area % purity; impurities consisted of 2-3% benzyl thioacetate and 10-11% benzyl 3,6-dihydropyridine-1(2H)-carboxylate. ¹H NMR (CDCl₃, 400 MHz): 1.50-1.62 (m, 2H), 1.89-1.98 (m, 2H), 2.32 (s, 3H), 3.17 (broad) triplet of triplets, 2H), 3.64 (triplet of triplets, 2H), 3.90-4.00 (m, 1H), 5.12 (s, 2H), 7.30-7.40 (m, 5H).

### Part F. Preparation of benzyl 4-(chlorosulfonyl)piperidine-1-carboxylate

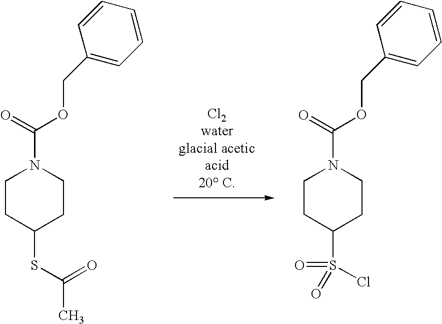 The following reaction was carried out in a 5 L 4-Neck jacketed cylindrical glass reactor with an overhead stirrer and a nitrogen atmosphere. A solution of benzyl 4-(acetylthio)piperidine-1-carboxylate from Part E (82% purity, 312 g, 1.06 mol) and water (134 mL, 7.44 mol) in glacial acetic acid (2.58 L) was stirred at 20° C. Chlorine gas (241 g, 3.40 mol) was added slowly over 140 min. A maximum temperature of 25.8° C. was reached after 32 min of chlorine addition. The color of the solution turned from a very dark amber color before the addition to a copper color at the end of the addition. The reactor was purged with N₂ to flush out excess chlorine gas out of the reactor. The following workup procedure was carried out in two portions. Ethyl acetate (3.0 L) and 7% aqueous NaCl solution (3.0 L) was charged to the reactor, and the mixture was vigorously stirred for 5 min. The upper organic layer was separated and washed with water (3.0 L) and 7% aqueous NaCl solution (3.0 L). The organic layer was diluted with heptanes (1.3 L), and the resulting mixture was concentrated under reduced pressure at 40° C. to 50% of the initial volume. Additional heptanes (1.3 L) was added, and the resulting mixture was again concentrated under reduced pressure at 40° C. to 50% of the initial volume. Heptanes (1.0 L) was added, and the resulting mixture was cooled to 0° C. over 10 h. A white voluminous precipitate appeared at around 35° C., and an even greater amount of precipitate was observed flowing freely within the reactor at 0° C. The slurry was filtered through a coarse frit, and the solid cake was washed with a cold (5° C.) heptanes reactor rinse (1.5 L). The solids were dried in a vacuum oven at ambient temperature overnight to give 171 g (51% yield; 62% yield when purity of starting material is considered) of product as a white solid. Analysis by HPLC showed >95% purity. ¹H NMR (CDCl₃, 400 MHz): 1.95 (br doublet of quartets, 2H), 2.36 (br doublet, 2H), 2.90 (br triplet, 2H), 3.68 (triplet of triplets, 2H), 4.42 (br s, 1H), 5.17 (s, 2H), 7.30-7.42 (m, 5H). It was later discovered on a smaller scale run that the addition of sodium acetate (5.5 equivalents) and a greater amount of water (43 equivalents, to solubilize the sodium acetate) to the reaction mixture improved the yield of benzyl 4-(chlorosulfonyl)piperidine-1-carboxylate to about 80%. It is believed that sodium acetate acts as a buffer in the highly acidic environment to minimize deprotection of the benzyl carbamate group of the product.

### Part G. Preparation of benzyl 4-({4-[4-(trifluoromethoxy)phenoxy]piperidin-1-yl}sulfonyl)piperidine-1-carboxylate

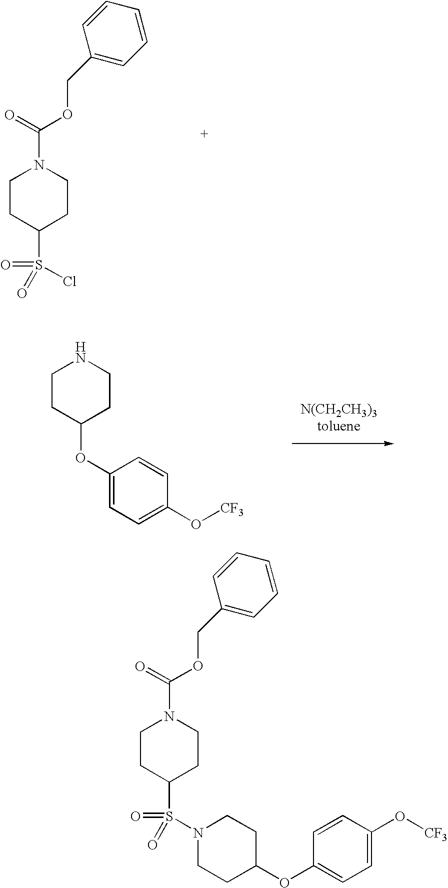 The 4-[4-(trifluoromethoxy)-phenoxy]piperidine from Part C (14.1 kg, 54.0 mol) was dissolved in toluene (63 L) in a 100 gallon glass-lined reactor. Triethylamine (9.1 kg, 90 mol) was charged, and the solution was cooled to 6° C. The benzyl 4-(chlorosulfonyl)piperidine-1-carboxylate from Part F (19.7 kg, 62.0 mol) was dissolved in toluene (83 L) in a 50 gallon glass-lined reactor. The toluene solution of benzyl 4-(chlorosulfonyl)piperidine-1-carboxylate was then charged through a 10 micron polypropylene filter bag to the cold toluene solution of 4-[4-(trifluoromethoxy)-phenoxy]piperidine and triethylamine. Because the reaction was exothermic, the charge was dose controlled to maintain a temperature at less than 30° C. (the maximum temperature reached was 14° C., and the addition time was 55 min). The reaction mixture was held for 4 hr, after which an in-process sample was taken which showed 1.37% of starting 4-[4-(trifluoromethoxy)-phenoxy]piperidine (relative to benzyl 4-({4-[4-(trifluoromethoxy)phenoxy]piperidin-1-yl}sulfonyl)piperidine-1-carboxylate) left. Water (90 L) was added to the reaction mixture. The mixture was stirred for 35 min, and then agitation was stopped to allow the layers to separate (1 hr). The aqueous layer was removed, and the organic layer was washed with water (90 L). The mixture was again stirred for 35 min, and agitation was stopped to allow the layers to separate (1 hr). The aqueous layer was removed, and the organic layer was distilled at 50 mm Hg with a maximum temperature of 46° C. for 3 hr to remove 117 L of toluene. Product precipitation was initiated by charging heptane (40 L) to the concentrate over 30 min at ambient temperature. After a 3 hr hold time, another charge of heptane (226 L) was added to precipitate out more product. The solids were collected by centrifugation in three batches and washed with heptane (13 L per batch). The solids were then dried at 60 mm Hg at 23° C. to give a total of 26.7 kg (91% yield) of product. Analysis by HPLC showed 97.8 area % purity. ¹H NMR (CDCl₃, 400 MHz): 1.65-1.80 (m, 2H), 1.85-2.03 (m, 4H), 2.03-2.10 (m, 2H), 2.30 (br s, 2H), 3.08 (triplet of triplets, 1H), 3.38-3.57 (m, 4H), 4.35 (br, s 2H), 4.52 (septet, 1H), 5.13 (s, 2H), 6.88 (d, 2H), 7.15 (d, 2H), 7.30-7.50 (m, 5H).

### Part H. Preparation of 1-(piperidin-4-ylsulfonyl)-4-[4-(trifluoromethoxy)phenoxy]piperidine hydrochloride

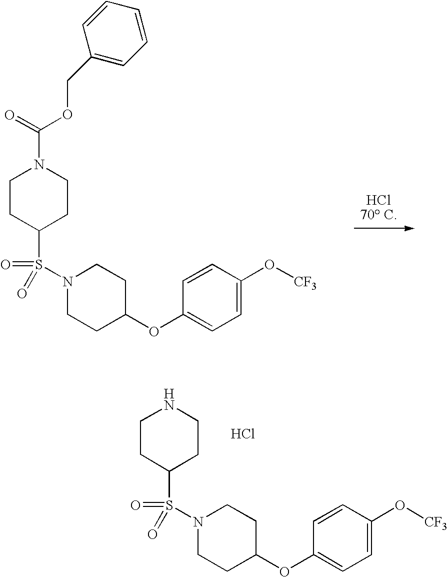 The benzyl 4-({4-[4-(trifluoromethoxy)phenoxy]piperidin-1-yl}sulfonyl)piperidine-1-carboxylate from Part G (52.0 kg, 95.8 mol) and concentrated HCl solution (32 weight %, 229 kg, 2010 mol) was charged to a 300 gallon glass-lined reactor. The resulting suspension was heated to 70° C. During heating, foaming was observed to occur at 40° C., which increased the total reaction mixture volume 2- to 3-fold. When the reaction mixture reached 70° C., the foaming stabilized. An in-process sample taken after 130 min at 70° C. showed no benzyl 4-({4-[4-(trifluoromethoxy)phenoxy]piperidin-1-yl}sulfonyl)piperidine-1-carboxylate remaining. Toluene (312 L) was charged to perform an azeotropic distillation (with a Dean—Stark apparatus) to remove water from the reaction mixture. Initially, foaming was an issue again as the vacuum and heat were applied to the system. The foaming, however, was controlled by careful adjustment of the vacuum (around 200 mm Hg). Applicants have observed that this foaming typically lasts for 1 hr, after which the mixture becomes stable, at which point no more foaming is observed. The final distillation temperature was 38° C. at 56 mm Hg, while the maximum temperature reached during distillation was 42° C. Distillation took 48 hr to remove 220 kg of water. Upon completion of the distillation, ethanol (41.3 kg) was charged, and the resulting slurry was heated to 62° C. to dissolve most of the solids. The slurry was then held at this temperature for 135 min. Afterward, the slurry was cooled from 62° C. to 50° C. over 34 min, held at 50° C. for 35 min, and finally cooled from 50° C. to 20° C. over 85 min. The solids were isolated by an Estrella filter. Several toluene washes and a re—Slurry of the wet cake were performed to remove any residual benzyl chloride to a level of less than 0.005 weight %. The wet cake was dried in a vacuum dryer at 80° C. and 31 mm Hg to give 39.3 kg (92% yield) of product as a white solid. Analysis by HPLC showed 99.7 area % purity. ¹H NMR (DMSO-d₆, 400 MHz): 1.62-1.78 (m, 2H), 1.80-1.92 (m, 2H), 1.93-2.04 (m, 2H), 2.10 (br d, 2H), 2.83-3.00 (br q, 2H), 3.20-3.42 (m, 4H), 3.42-3.58 (m, 3H), 4.60-4.64 (m, 1H), 7.10 (d, 2H), 7.30 (d, 2H), 8.80 (br d, 1H), 9.10 (br d, 1H).

### Part I. Preparation of 1-(2-methoxyethyl)-4-({4-[4-(trifluoromethoxy)phenoxylpiperidin-1-yl}sulfonyl)piperidine

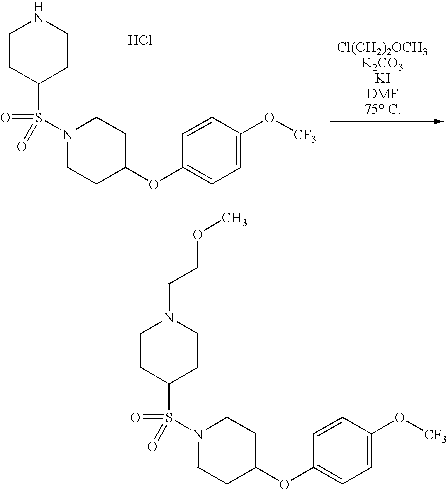 The 1-(piperidin-4-ylsulfonyl)-4-[4-(trifluoromethoxy)phenoxy]piperidine hydrochloride from Part H (19.6 kg, 44.1 mol), potassium iodide (11.0 kg, 66.3 mol), potassium carbonate (12.8 kg, 92.6 mol), and N,N-dimethylformamide (225 L) were charged to a 300 gallon glass-lined reactor, followed by 2-chloroethyl methyl ether (5.1 kg, 53.9 mol). The mixture was heated to 75° C. over 37 min. An in-process sample taken after 21 hr at 70° C. showed 1.6% of starting 1-(piperidin-4-ylsulfonyl)-4-[4-(trifluoromethoxy)phenoxy]piperidine hydrochloride (relative to 1-(2-methoxyethyl)-4-({4-[4-(trifluoromethoxy)phenoxy]piperidin-1-yl}sulfonyl)piperidine). The mixture was cooled to ambient temperature, and toluene (392 L) and water (195 L) were charged to the reactor. The contents were stirred for 50 min, and then agitation was stopped to allow the layers to separate (83 min). The aqueous layer was removed, and the organic layer was washed with 15% NaCl solution (196 kg). The contents were stirred for 60 min, and then agitation was stopped to allow the layers to separate (105 min). The aqueous layer was removed, and the organic layer was distilled under vacuum with a maximum temperature of 22° C. for 2 hr to remove 298 kg of toluene. Additional toluene (298 kg) was charged to the concentrate, and then the solution was distilled under vacuum with a maximum temperature of 20° C. for 2 hr to remove 296 kg of toluene. Both of these toluene distillations served to remove residual water down to below 0.02%. Analysis of the concentrate (47.5 kg) showed 37.6 weight % of 1-(2-methoxyethyl)-4-({4-[4-(trifluoromethoxy)phenoxy]piperidin-1-yl}sulfonyl)piperidine, which corresponds to 17.9 kg or 87% yield. The concentrate was used as is in the next step. ¹H NMR (CDCl₃, 400 MHz): 1.83-1.93 (m, 4H), 1.93-2.10 (m, 7H), 2.59 (t, 2H), 2.92 (triplet of triplets, 1H), 3.08-3.15 (m, 2H), 3.38 (s, 3H), 3.40-3.50 (m, 2H), 3.50-3.60 (m, 3H), 4.50-4.58 (m, 1H), 6.90 (d, 2H), 7.18 (d, 2H).

### Part J. Preparation of 1-(2-methoxyethyl)-4-[[4-[4-(trifluoromethoxy)phenoxy]-1-piperidinyl]-sulfonyl1-4-piperidinecarboxylic acid

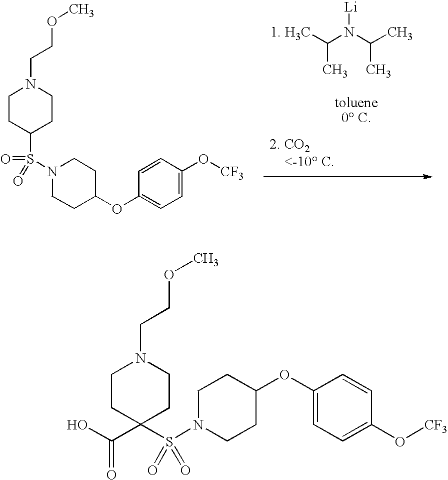 A concentrate containing 28.2 weight % of 1-(2-methoxyethyl)-4-({4-[4-(trifluoromethoxy)phenoxy]piperidin-1-yl}sulfonyl)piperidine in toluene from Part I (61.3 kg of solution which corresponds to 17.3 kg or 37.0 mol of 1-(2-methoxyethyl)-4-({4-[4-(trifluoromethoxy)phenoxy]piperidin-1-yl}sulfonyl)piperidine) and less than 0.02% water was charged to a 300 gallon glass-lined reactor. Toluene (153 kg) was added, and the solution was cooled to −15° C. A 2.0 M solution of lithium diisopropylamide in tetrahydrofuran/heptane/ethylbenzene (25.1 L, 50.2 mol) was added at a rate that allowed the temperature to be maintained at less than −10° C. This was followed by a toluene rinse (7.0 kg). Carbon dioxide (3.70 kg, 77.1 mol) was then charged through a subsurface sparge tube at a rate that allowed the temperature to be maintained at less than −10° C. During the carbon dioxide charge, the reactor vent was kept closed, but vented if the pressure increased to greater than 17.7 psia. The pressure in the reactor typically did not increase until after the first equivalent of carbon dioxide was added. Also, the pressure in the vessel was maintained at approximately 17.7 psia for 30 min after the charge to ensure complete consumption of starting material. Nitrogen was sparged into the reactor for 30 min, and then the reaction mixture was warmed to 25° C. An in-process sample showed 1.6% starting 1-(2-methoxyethyl)-4-({4-[4-(trifluoromethoxy)phenoxy]piperidin-1-yl}sulfonyl)piperidine (relative to 1-(2-methoxyethyl)-4-[[4-[4-(trifluoromethoxy)phenoxy]-1-piperidinyl]-sulfonyl]-4-piperidinecarboxylic acid). Toluene (7.0 kg) and 0.88% NaCl solution (313 kg) were added, and the contents were stirred at no greater than 50 rpm for 60 min. Agitation was stopped to allow the layers to separate (65 min). The aqueous layer was then separated and washed with toluene (208 kg). The contents were stirred at no greater than 50 rpm for 40 min. Agitation was stopped to allow the layers to separate (70 min). The aqueous layer was separated, and isopropanol (81.3 kg) was added. The solution was neutralized to a pH of 8.10 with 6 N HCl solution (7.89 kg, 40.0 mol), followed by a water rinse (5.0 kg). During the neutralization, some foaming was observed while solids came out of solution. Filtration of the slurry was performed in a 40″ glass-lined Estrella filter for 30 min. The solid cake was washed twice with a mixture of isopropanol/water (23 kg/142 kg each wash), which took 60 min for each wash. The solid cake was pulled dry for 4 days, and then dried under 28″ Hg vacuum for 17 hr at 75° C. in a vacuum shelf dryer to give 18.0 kg (95% yield) of 1-(2-methoxyethyl)-4-[[4-[4-(trifluoromethoxy)phenoxy]-1-piperidinyl]-sulfonyl]-4-piperidinecarboxylic acid. Analysis by HPLC showed 97.7 weight % purity, while Karl-Fischer analysis showed 0.41% water. ¹H NMR (D₂O+drop of NaOD, 400 MHz): 1.70-1.82 (m, 2H), 1.90-2.12 (m, 4H), 2.19 (d, 2H), 2.59 (t, 2H), 3.01 (d, 2H), 3.30-3.40 (m, 4H), 3.38 (s, 3H), 3.62 (t, 2H), 3.60-3.70 (m, 2H), 4.60-4.70 (m, 1H), 7.12 (d, 2H), 7.37 (d, 2H).

### Part K Preparation of 1-(2-methoxyethyl)-N-[(tetrahydro-2H-pyran-2-yl)oxyl-4-[[4-[4-(trifluoromethoxy)phenoxyl-1-piperidinyl]-sulfonyl]-4-piperidinecarboxylate

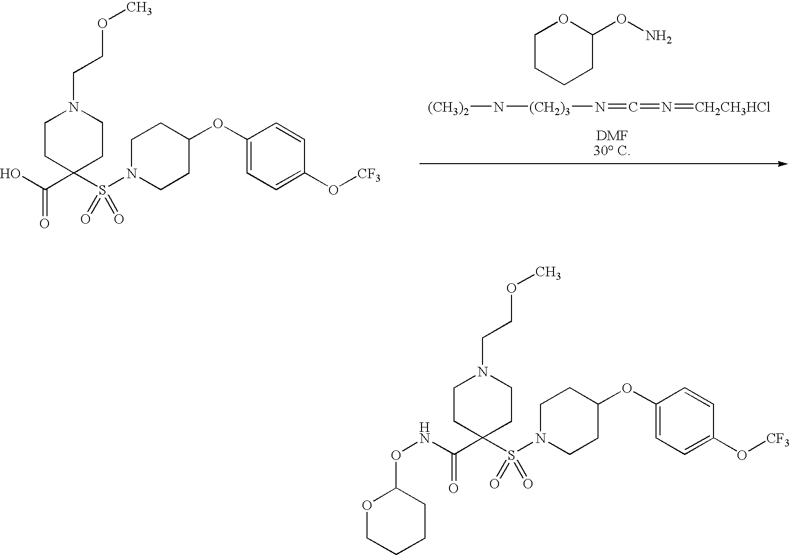 The 1-(2-methoxyethyl)-4-[[4-[4-(trifluoromethoxy)phenoxy]-l-piperidinyl]-sulfonyl]-4-piperidinecarboxylic acid from Part J (24.4 kg, 47.8 mol), O-tetrahydro-2H-pyran-2-ylhydroxylamine (7.40 kg, 63.2 mol), 1-ethyl-3-(-3-dimethylaminopropyl)-carbodiimide hydrochloride (16.5 kg, 86.1 mol), and N,N-dimethylformamide (185 kg) were charged to a 300 gallon glass-lined reactor. The reaction mixture was heated to 30° C., and stirred at this temperature for 12 h. An in-process sample showed 0.73% starting 1-(2-methoxyethyl)-4-[[4-[4-(trifluoromethoxy)phenoxy]-1-piperidinyl]-sulfonyl]-4-piperidinecarboxylic acid relative to (1-(2-methoxyethyl)-N-[(tetrahydro-2H-pyran-2-yl)oxy]-4-[[4-[4-(trifluoromethoxy)phenoxy]-1-piperidinyl]-sulfonyl]-4-piperidinecarboxylate). A 2:1 (w/w) mixture of ethyl acetate/heptane (158 kg) and water (390 kg) were added to the reactor, and the contents were stirred for 15 min. Agitation was stopped to allow the layers were allowed to separate (100 min). The organic layer was separated and set aside while the aqueous layer was back-extracted with a 2:1 (w/w) mixture of ethyl acetate/heptane (155 kg). The contents were stirred for 40 min. Agitation was then stopped to allow the layers to separate (130 min). The organic extracts were combined and distilled to the minimum stir volume of the reactor (ca. 80 L). Isopropanol (65 kg) was added, and the mixture was distilled to the minimum stir volume again. Another portion of isopropanol (65 kg) was added, and the mixture was again distilled to the minimum stir volume. Each distillation took 2-6 hr under approximately 1.0 psia with a maximum temperature of approximately 53° C. A larger portion of isopropanol (130 kg) was added to the concentrate, resulting in formation of a slurry. The slurry was heated to 45° C. to dissolve the solids (14 min) and then cooled to 10° C. over 89 min, resulting in re-formation of the solids. The slurry was held at 10° C. for 140 min and then filtered using a 40″ glass-lined Estrella filter for 30 min. The solid cake was washed with isopropanol (26 kg) and dried on the filter at 25° C. for 24 hr under 28″ Hg vacuum to give 27.8 kg (93% yield) of 1-(2-methoxyethyl)-N-[(tetrahydro-2H-pyran-2-yl)oxy]-4-[[4-[4-(trifluoromethoxy)phenoxy]-1-piperidinyl]-sulfonyl]-4-piperidinecarboxylate. Analysis by HPLC showed 93.0 weight % (the remaining 7% is primarily isopropanol). ¹H NMR (CDCl₃, 400 MHz, complex due to the presence of diastereomers, integration not indicated): 1.11 (d), 1.55-1.75 (m), 1.75-2.10 (m), 2.20-2.32 (m), 2.80-3.10 (m), 3.38 (s), 3.30-3.50 (m), 3.50-3.70 (m), 3.70-3.90 (m), 3.92-4.10 (m), 4.40-4.60 (m), 5.12 (s), 6.90 (d), 6.95 (d), 7.15 (d).

### Part L. Preparation of N-hydroxy-1-(2-methoxyethyl)-4-[[4-[4-(trifluoromethoxy)phenoxyl-1-piperidinyllsulfonyl]4-piperidinecarboxamide, monohydrochloride

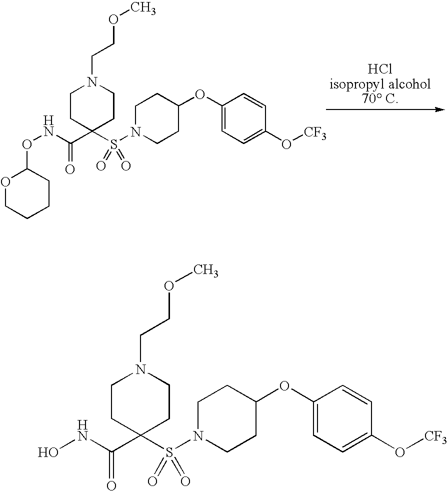 The 1-(2-methoxyethyl)-N-[(tetrahydro-2H-pyran-2-yl)oxyl-4-[[4-[4-(trifluoromethoxy)phenoxy]-1-piperidinyl]-sulfonyl]-4-piperidinecarboxylate from Part K (27.8 kg, 45.6 mol), isopropanol (109 kg), and concentrated HCl solution (32 weight %, 8.80 kg, 77 mol) were charged to a 100 gallon glass-lined reactor. The contents were heated to 70° C. over 2 hr, and held at this temperature for 4.5 hr. The resulting clear solution was cooled to 25° C. over 10 hr, during which solids precipitate out of solution. The resulting slurry was held at this temperature for 7 hr, and then filtered using a 40″ glass-lined Estrella filter in 30 min. The solid cake was washed with isopropanol (99 kg), and the solids were pulled dry for 13 hr. The solids were then dried at 45° C. under 1 psia vacuum for 48 hr to give 22.6 kg (89% yield) of N-hydroxy-l-(2-methoxyethyl)-4-[[4-[4-(trifluoromethoxy)phenoxy]-1-piperidinyl]sulfonyl]-4-piperidinecarboxamide, monohydrochloride. Analysis by HPLC showed 100 weight %. ¹H NMR (DMSO-d₆, 500 MHz): 1.68 (br s, 2H), 1.97 (br s, 2H), 2.40 (triplet of doublets, 2H), 2.64 (d, 2H), 2.84 (app q, 2H), 3.27 (q, 2H), 3.28 (s, 3H), 3.53 (br s, 2H), 3.53 (br s, 2H), 3.57 (d, 2H), 3.73 (t, 2H), 4.60 (br s, 1H), 7.11 (distorted d, 2H), 7.29 (d, 2H), 9.3 (very br s, 1H), 11.07 (br s, 1H), 11.18 (brs, 1H), 11.30(brs, 1H).

### Example 2

### Second Preparation of N-hydroxy-1-(2-methoxyethyl)-4-[[4-[4-(trifluoromethoxy)phenoxy]-1-piperidiny]sulfonyl]-4-piperidinecarboxamide, monohydrochloride

### Part A. Preparation of tert-butyl 4-hydroxy-1-piperidinecarboxylate

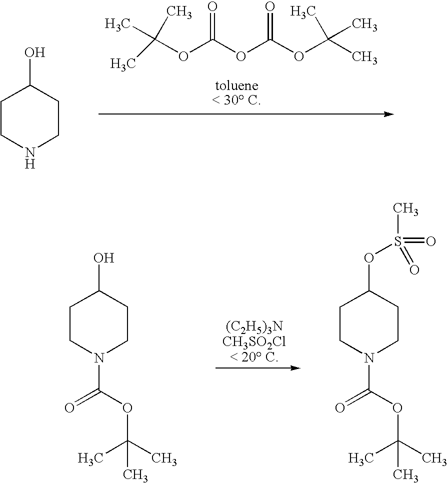 This reaction was carried out in a 3 L jacketed reactor with a mechanical stirrer, heater/chiller, thermocouple/J-KEM temperature controller, water condenser, and nitrogen. 4-hydroxypiperidine (173 g) was charged to the reactor. Toluene (900 mL) was then charged, and the resulting mixture was stirred to form a hazy solution. A solution of di-t-butyl dicarbonate (“Boc₂O”) (380.95 g) and 800 mL toluene was prepared and charged dropwise to the reaction mixture while maintaining temperature at <30° C. The addition took 38 min, during which time CO₂ evolution occurred. The reaction was monitored by following the disappearance of 4-hydroxypiperidine by GC. The reaction was complete at the end of the Boc₂O addition. Triethylamine (262 mL) was next charged to the reaction mixture. The resulting mixture was cooled to less than 5° C.

Methanesulfonyl chloride (148 mL) was added dropwise to the reaction mixture while maintaining the temperature less than 20° C. The addition took 46 min, during which time a yellow slurry formed. The consumption of tert-butyl 4-hydroxypiperidine-1-carboxylate was monitored by GC. The reaction was complete at the end of the methanesulfonyl chloride. The mixture was then heated to 25° C. and transferred to a 5 L, 3-Neck Morton flask with a mechanical stirrer. Water (1.7 L) and ethyl acetate (400 mL) were charged. The resulting layers were mixed and phase separated. The organic phase was washed with 1.7 L water, and phase separated to provide a hazy aqueous phase and hazy organic phase. The organic phase was filtered through a sintered glass filter to provide a clear filtrate. Solvent was removed by distillation under reduced pressure (15 mbar) on a rotary evaporator at <55° C. The 1-(tert-Butoxycarbonyl)-4-piperidinyl methanesulfonate product (464.97 g, 97% yield) was isolated as a beige solid.

### Part B. Preparation of tert-butyl 4-[4-(trifluoromethoxy)phenoxy]piperidine-1-carboxylate

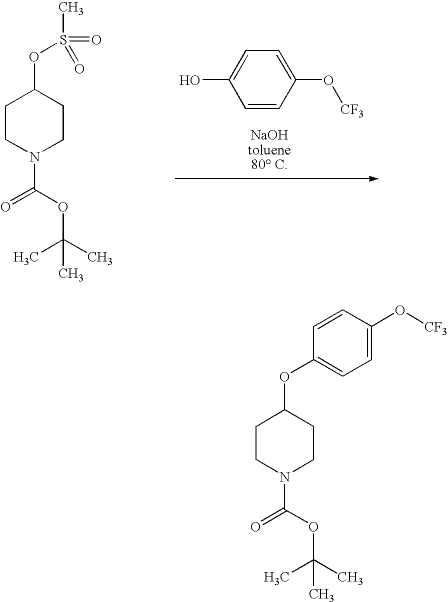

This reaction was carried out in a 5 L, 3-Neck Morton flask equipped with a mechanical stirrer, thermocouple/J-KEM temperature controller/heating mantle, water condenser, and nitrogen. 1-(tert-butoxycarbonyl)-4-piperidinyl methanesulfonate from Part A (460.07 g) and 2.3 L toluene were charged to the reactor. The resulting mixture was stirred to form a hazy solution. Trifluoromethoxy phenol (213 mL) and 662 mL 30% NaOH were then charged to the reactor. The resulting mixture was heated to 80° C. and stirred for 1 hr. Afterward, the reaction mixture was cooled to 25° C. using an ice bath and 1.15 L water was charged. The layers were phase separated, and the organic phase was washed with 1.15 L water. The layers were phase separated, and the top organic layer was distilled under reduced pressure (15 mbar) at <55° C. using a rotary evaporator. Crude tert-butyl 4-[4-(trifluoromethoxy)phenoxy]piperidine-1-carboxylate was isolated (394.67 g, 66% yield) as a hazy yellow oil.

### Part C. Preparation of 4-[4-(trifluoromethoxy)phenoxy]-piperidine

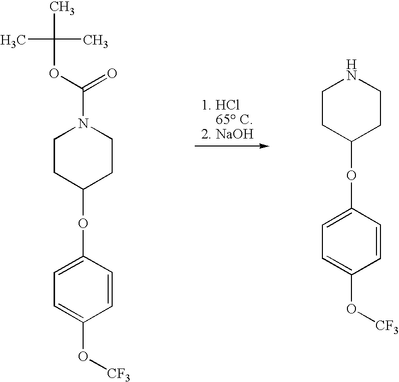 This reaction was carried out in a 5 L, 3-Neck Morton flask equipped with a mechanical stirrer, thermocouple/J-KEM temperature controller/heating mantle, water condenser, and nitrogen. Crude tert-butyl 4-[4-(trifluoromethoxy)phenoxy]piperidine-1-carboxylate from Part B (394.67 g) was transferred to the reactor. Water (1.15 L) water was then charged, and the mixture was stirred. Next, 37% HCl (540 mL was charged to the reactor). Some gas evolution and foaming was observed. The mixture was heated to 65° C. and stirred for 1 hr. The reaction mixture was then cooled to 20° C. using an ice bath, and the pH was adjusted to 12.5 with 30% NaOH (1317.0 g). An exotherm of approximately 37° C. was observed. The reaction mixture was cooled to 20° C. using an ice bath, and 1.15 L toluene was charged. The resulting layers were phase separated. The top organic phase was washed twice with 1.15 L water. The organic layer was then distilled under reduced pressure (15 mbar) <55° C. using a rotary evaporator to provide 4-[4-(trifluoromethoxy)phenoxy]-piperidine (174.75 g, 61% yield) as an off-white solid.

### Part D. Preparation of tert-butyl 4-(acetylthio)piperidine-1-carboxylate

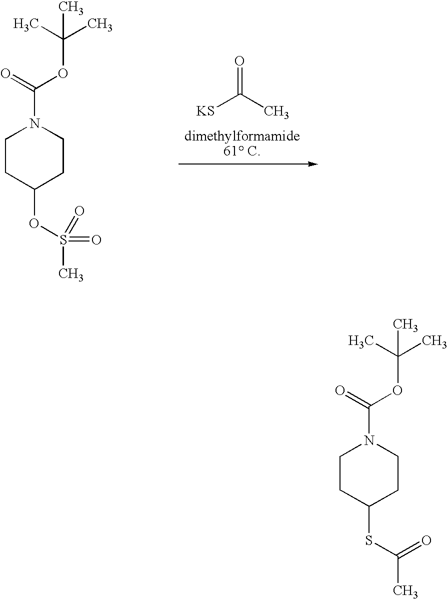

1-(tert-butoxycarbonyl)-4-piperidinyl methanesulfonate from Part A (200.31 g, 0.717 mol) was charged to a 2 L jacketed reactor under nitrogen. N,N-dimethylformamide (1.6 L) was then added, and the slurry was stirred until a solution was obtained. Afterward, potassium thioacetate (110.47 g, 0.967 mol) was added, and the solution was heated to 61° C. The solution was held at 61° C. for 16.5 h, and then cooled to ambient temperature. The reaction was divided into two equal portions. Each portion was then treated as follows. Water (0.7 L) and methyl t-butyl ether (0.6 L) were charged. The resulting layers were mixed by stirring. The stirrer was then turned off, and the layers were separated. Both layers were drained from the reactor, and the aqueous bottom layer was returned to the reactor. Methyl t-butyl ether (0.6 L) was charged. The resulting layers were mixed by stirring. The stirrer was then turned off, and the layers were separated. Both layers were drained from the reactor, and the combined organic layers were returned to the reactor. Water (0.5 L) was charged to the reactor. The resulting layers were mixed by stirring. The stirrer was then turned off, and the layers were separated. The work up procedure was then repeated on the second half of the crude reaction mixture. Afterward, the combined organic layers were dried over MgSO₄, which was filtered off using a coarse glass frit. The solvents were removed using rotary evaporation to afford tert-butyl 4-(acetylthio)piperidine-1-carboxylate as a brown oil (182.11 g, 98% crude yield, 80 area % by GC) that was used in the next step.

### Part E. Preparation of tert-butyl 4-(chlorosulfonyl)piperidine-1-carboxylate

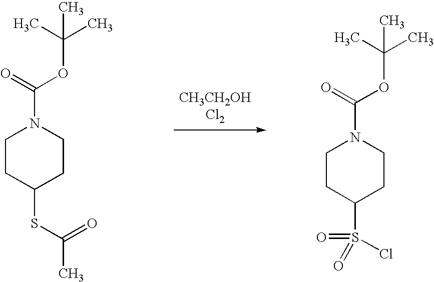

Crude tert-butyl 4-(acetylthio)piperidine-1-carboxylate from Part D (182.11 g, 0.702 mol) was charged to a 2 L jacketed reactor under nitrogen. Absolute ethanol (1.0 L) was added, and the solution was cooled to −8° C. Chlorine gas (189 g, 2.67 mol) was bubbled into the solution over 1.5 hr. The maximum temperature observed was 7° C. The solution was warmed to room temperature, and worked up in two equal portions using the following procedure. Toluene (1.0 L) and 10 wt % NaCl (aq) (0.65 L) were charged. The resulting layers were mixed by stirring. The stirrer was then turned off, and the layers were separated. The lower aqueous layer was drained from the reactor, and 10 wt % NaCl(aq) (0.65 L) was charged. The layers were mixed by stirring. The stirrer was then turned off, and the layers were separated. The lower aqueous layer was drained from the reactor, and water (0.65 L) was charged. The resulting layers were mixed by stirring. The stirrer was then turned off, and the layers separated. This work up procedure was repeated on the second half of the crude reaction mixture. Afterward, the combined organic layers were concentrated using a rotary evaporator and then high vacuum to afford an off white solid (162 g, 81% crude yield). The crude product was recrystallized by charging heptanes (0.4 L) and heating to 60° C. to dissolve all the solids. The mixture was cooled to ambient temperature slowly with stirring, and then cooled to 0° C. in an ice-water bath. The slurry was filtered through a coarse glass frit and dried in a vacuum oven to obtain tert-butyl 4-(chlorosulfonyl)piperidine-1-carboxylate as a white solid (112 g, 69% yield). HRMS Calculated for (M+Na) C₁₀H₁₈NO₄SNa: 306.0543; Found (M+Na): 306.0566.

### Part F. Preparation of tert-butyl 4-({4-[4-(trifluoromethoxy)phenoxy]piperidin-1-yl}sulfonyl)piperidine-1-carboxylate

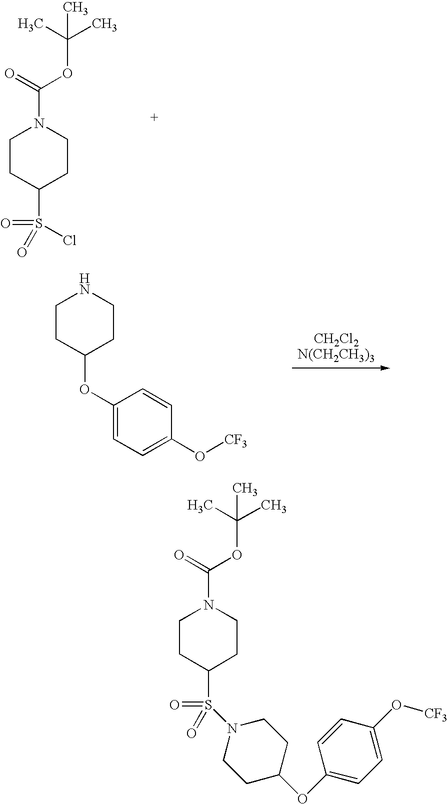

This reaction was carried out in a 1000 mL jacketed reactor with a bottom valve, equipped with mechanical stirrer, cold water condenser, J-KEM thermocouple, and N₂ or vacuum inlet. To the reactor was charged 4-[4-(trifluoromethoxy)phenoxy]-piperidine from Part C (41.0 g) and CH₂Cl₂ (78 mL). The mixture was stirred, and triethylamine (36.8 mL) was then charged. A solution of tert-butyl 4-(chlorosulfonyl)piperidine-1-carboxylate from Part E (50.0 g) and CH₂Cl₂ (114 mL) was prepared, and added via addition funnel to the reactor over 18 min. The addition funnel was rinsed with CH₂Cl₂ (6.2 mL), and the rinse was added to the reactor. After 1 hr, HPLC indicated reaction completion. Water (190 mL) was then added to the reactor, and the contents were mixed and allowed to settle. The lower organic layer was washed with 1N HCl (190 mL), followed by saturated NaHCO₃ solution (190 mL). The organic layer was dried over Na₂SO₄ and filtered. The solvent was removed by rotary evaporation to provide 4-({4-[4-(trifluoromethoxy)phenoxy]piperidin-1-yl}sulfonyl)piperidine-1-carboxylate as an off-white solid. HRMS Calculated for (M+Na) C₂₂H₃₁F₃N₂O₆S Na: 531.1753; Found (M+Na): 531.1771.

### Part G. Preparation of 1-(piperidin-4-ylsulfonyl)-4-[4-(trifluoromethoxy)phenoxy]piperidine hydrochloride

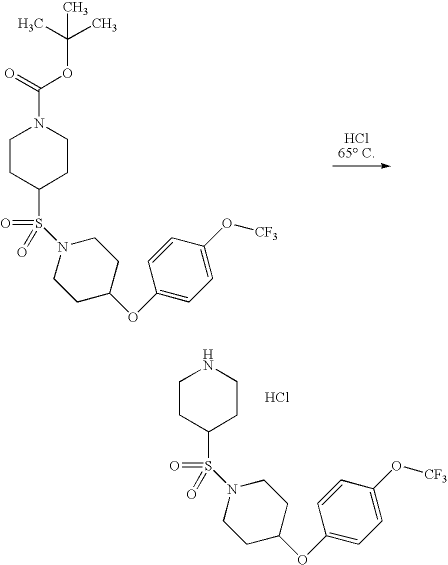

This reaction was carried out in a 1000 mL jacketed reactor equipped with a bottom valve, a mechanical stirrer; cold water condenser; J-KEM thermocouple; and N₂, HCl, or vacuum inlet. The reactor was charged with 4-({4-[4-(trifluoromethoxy)phenoxy]piperidin-1-yl}sulfonyl)piperidine-1-carboxylate from Part F (50.0 g), 2-propanol (50 mL), and toluene (450 mL). The contents were stirred, and HCl gas (10.75 g) was bubbled into the reactor over 30 min. Upon completion of the HCl addition completion, the jacket temperature was set to 65° C. The reactor contents were heated for 90 min, at which time HPLC indicated reaction completion. The reactor contents were cooled from 65° C. to 5° C. at 0.5° C./min, and then stirred at 5° C. overnight. The mixture was filtered at 5° C. through No 1. Whatman filter paper using a Buchner funnel and the solid was dried in a vacuum oven at 50° C. with N₂ purge. The product 1-(piperidin-4-ylsulfonyl)-4-[4-(trifluoromethoxy)phenoxy]piperidine hydrochloride (41.5 g, 95% yield) was obtained as a white solid.

### Part H. Preparation of 1-(2-methoxyethyl)-4-({4-[4-(trifluoromethoxy)phenoxy]piperidin-1-yl}sulfonyl)piperidine

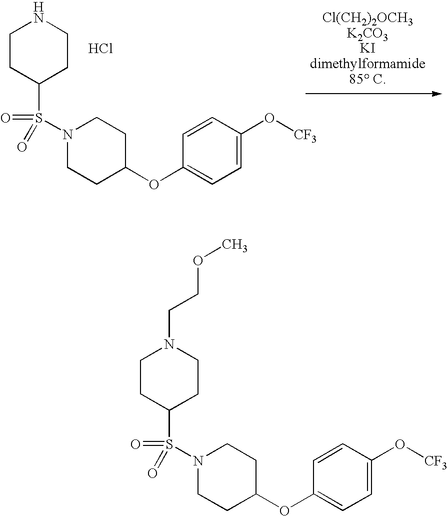

This reaction was carried out in a 3 L jacketed reactor equipped with a mechanical stirrer (double turbine blades), distillation/reflux head, J-KEM thermocouple, heater/chiller, and N₂ or vacuum inlet. To the reactor was charged 1-(piperidin-4-ylsulfonyl)-4-[4-(trifluoromethoxy)phenoxy]piperidine hydrochloride from Part G (170.07 g), potassium carbonate (100.42 g), potassium iodide (97.72 g), DMF (835 mL), and water (17 mL). The resulting mixture was then stirred. To the beige slurry was charged 2-chloroethylmethyl ether (50 mL), and the mixture was heated to 85° C. The reaction was monitored by HPLC until less than 1% starting material remained (5 hr). The mixture was then cooled to 25° C. While cooling, toluene (850 mL) and water (850 mL) were charged to the reactor. The phases were separated, and the organic layer was washed with 10% NaCl (850 mL). The organic layer was then distilled under reduced pressure (60 torr) until 415 mL of solvent had been removed. The reactor contents were cooled to 25° C., and toluene (415 mL) was charged. Some solids formed during the distillation and solvent addition. The mixture was filtered using a pressure filter to provide 839.76 g clear orange solution. HPLC assay of 1-(2-methoxyethyl)-4-({4-[4-(trifluoromethoxy)phenoxy]piperidin-1-yl) sulfonyl)piperidine in solution was 19.1 wt % (90% yield).

### Part I. Preparation of 1-(2-methoxyethyl)-4-[[4-[4-(trifluoromethoxy)phenoxy]-1-piperidinyl]sulfonyl]-4-piperidinecarboxylic acid

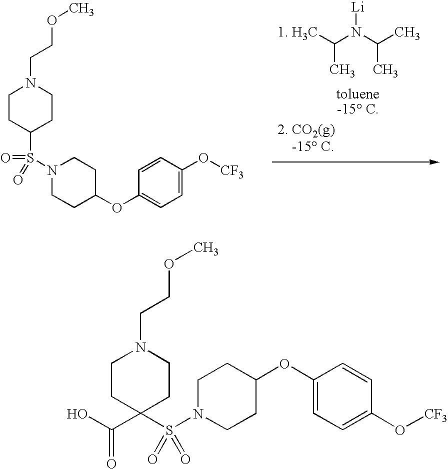

This reaction was carried out in a 500 mL jacketed reactor equipped with an overhead stirrer, nitrogen inlet, carbon dioxide inlet (316 SS regulator (Aldrich, Z14,850-4), FEP tubing, 0.062×⅛″ (Upchurch Scientific, inc.), J-KEM thermometer probe, and pH meter and probe. To the reactor was charged of a toluene solution of 1-(2-methoxyethyl)-4-({4-[4-(trifluoromethoxy)phenoxy]piperidin-1-yl}sulfonyl)piperidine (10.0 g contained) from Part H (52.4 g). The contents were cooled to −15° C. with a circulating chiller, and 15.5 mL LDA solution in THF/ethylbenzene/Heptane (1.8 M) was added to the reactor over ˜32 seconds. The reaction mixture was stirred for 30 min, then 2.0 g CO₂ gas was bubbled into the reaction mixture. The mixture was stirred for 30 min at −15° C. The jacket temperature was set to 25° C., and the reaction mixture was sparged with nitrogen gas during the warm-up time (45-60 min). To the reactor was charged 120 mL water and 8 mL NaCl solution (15% wt/wt). The contents were mixed for 5 min, and then the phases were allowed to separate. The aqueous layer was washed twice with 75 mL toluene. Afterward, 2-propanol (41 mL) was added to the aqueous phase in the reactor and heated to 61° C. The pH of the mixture was then adjusted to 7.4 through addition of 6 N HCl. Solids precipitated during the pH adjustment. The mixture was cooled to 21° C. at a rate of 0.5° C./min, and then filtered. The reactor and product cake was subsequently rinsed twice with 25:75 (v/v) 2-propanol:water (57 mL). The solid was dried in a vacuum oven at 105° C. for 22 hr. The desired product, 1-(2-methoxyethyl)-4-[[4-[4-(trifluoromethoxy)phenoxy]-1-piperidinyl]sulfonyl]-4-piperidinecarboxylic acid, was obtained as a white solid (9.60 g 91% yield).

### Part J. Preparation of 1-(2-methoxyethyl)-N-[(tetrahydro-2H-pyran-2-yl)oxy]-4-[[4-[4-(trifluoromethoxy)phenoxy]-1-piperidinyl]-sulfonyl]-4-piperidinecarboxylate

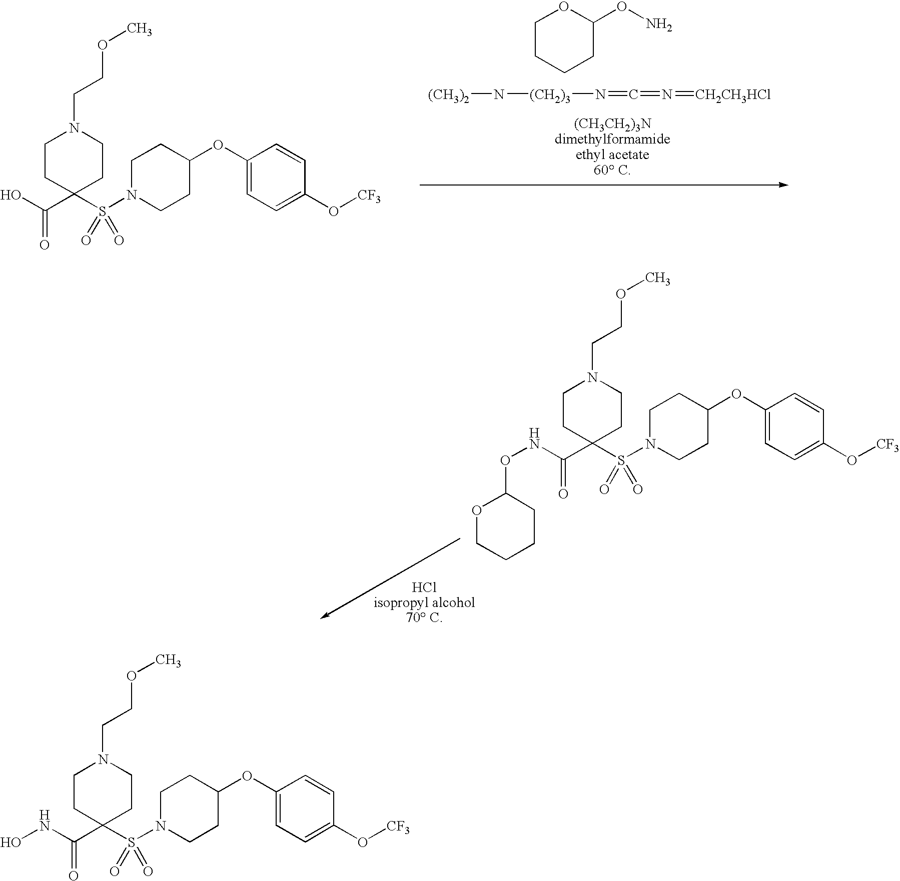

This reaction was carried out in a 500 mL jacketed flask equipped with a mechanical stirrer, cold water condenser, J-KEM thermocouple, and N₂ or vacuum inlet. To the reactor was charged 20.00 g 1-(2-methoxyethyl)-4-[[4-[4-(trifluoromethoxy)phenoxy]-1-piperidinyl]sulfonyl]-4-piperidinecarboxylic acid from Part I (20.00 g), EDC (9.77 g), and 2-(aminooxy)tetrahydro-2H-pyran (5.52 g). Triethylamine (6.55 mL), DMF (9.6 mL), and ethyl acetate (100.4 mL) were then charged to the reactor with stirring. The reactor jacket temperature was then set to 60° C., and the mixture was stirred for 2.0 hr at 60° C. The reaction mixture was then cooled to 30° C., and water (120 mL) and ethyl acetate (40 mL) were added. The resulting mixture was then stirred for 15 min. The layers were separated, and the organic layer was distilled using 100 torr vacuum. After approximately 120 mL had been removed from the reactor, the vacuum was released, and 2-propanol (160 mL) and conc. HCl (3.25 mL) were added to the reactor. The vacuum distillation was resumed at 100 torr. After approximately 80 mL had been removed from the reactor, the vacuum was released, and 2-propanol (80 mL) was added to the reactor. Vacuum distillation was then resumed at 100 torr. After approximately 80 mL had been removed from the reactor, the vacuum was released and the jacket temperature was set to 70° C. When the jacket temperature reached 70° C., conc. HCl (3.25 mL) was added to the reactor. The reaction mixture was held at 70° C. for a total of 4 hr, during which time solids began to crystallize. The reaction mixture was cooled from 70° C. to 10° C. at 0.2° C./min, and then stirred overnight at 10° C. Afterward, the reaction mixture was filtered through No 1. Whatman filter paper using a 8.5 cm Buchner funnel. The reactor was rinsed twice with 2-propanol (40 mL), which was transferred to the filter to wash the cake. The solid was dried in a vacuum oven at 50° C. with N₂ purge overnight to provide 19.41 g (88% yield) of N-hydroxy-1-(2-methoxyethyl)-4-[[4-[4-(trifluoromethoxy)phenoxy]-1-piperidinyl]sulfonyl]-4-piperidinecarboxamide hydrochloride as fine white crystals.

### Example 3

### Alternative preparation of 1-(piperidin-4-ylsulfonyl)-4-[4-(trifluoromethoxy)phenoxy]piperidine hydrochloride

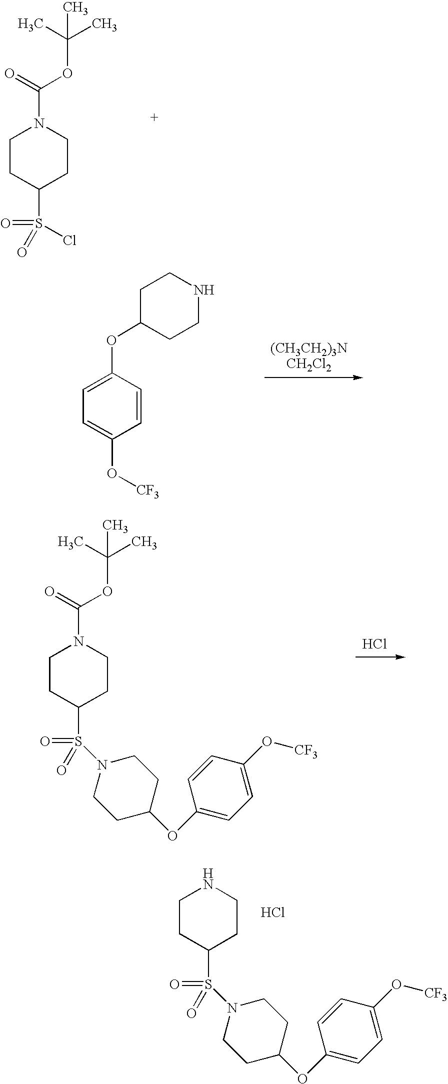

This reaction was carried out in a 4000 mL jacketed reactor equipped with a bottom valve, mechanical stirrer, cold water condenser, J-KEM thermocouple, and N₂. To the reactor was charged 148 g 4-[4-(trifluoromethoxy)phenoxy]-piperidine from Part C of Example 2 (148 g), CH₂Cl₂, (740 mL), and triethylamine (133 mL). The jacket temperature was set to 20° C., and the contents were stirred. A solution of tert-butyl 4-(chlorosulfonyl)piperidine-1-carboxylate from Part E of Example 2 (180 g) and CH₂Cl₂ (1080 mL) was charged to the reactor over a period of 24 min via addition funnel. The funnel was rinsed with CH₂Cl₂ (60 mL) and added to the reactor. After 1 hr, HPLC indicated 2% 4-[4-(trifluoromethoxy)phenoxy]-piperidine remaining relative to product. An additional charge of tert-butyl 4-(chlorosulfonyl)piperidine-l-carboxylate (3.85 g) in CH₂Cl₂ (40 mL) was added and the reaction mixture stirred for an additional 30 min. Water (1800 mL) was added to the reactor and the layers mixed, then allowed to separate. The CH₂Cl₂ layer was washed with 1 N HCl (1800 mL), followed by saturated NaHCO₃ (1800 mL). The CH₂Cl₂ layer was dried over Na₂SO₄, then filtered and charged to a 3000 mL jacketed reactor with bottom valve, equipped with mechanical stirrer, cold water condenser, J-KEM thermocouple, and N₂, HCl, or vacuum inlet. The jacket temperature was set to 20° C. and HCl gas (70.7 g) was bubbled through the stirred solution over ˜30 min. Nitrogen gas was passed through the delivery system and the reaction mixture for 5 min, then CH₂Cl₂ (1000 mL) was distilled. Toluene (600 mL) was added to the reactor and mixed, then the contents were filtered using a pressure filter. The reactor and cake were washed with 50:50 toluene:CH₂Cl₂ (200 mL) and nitrogen passed through the filter overnight. The solid was dried in a vacuum oven at 50° C. with N₂ purge overnight. The product 1-(piperidin-4-ylsulfonyl)-4-[4-(trifluoromethoxy)phenoxy]piperidine hydrochloride (233.05 g, 92% yield) was obtained as an off-white solid.

### Example 4

### Alternative preparation of 4-[4-(trifluoromethoxy)-phenoxy]piperidine

### Part A: Preparation of 1,1-dimethylethyl 4-hydroxy-1-piperidinecarboxylate

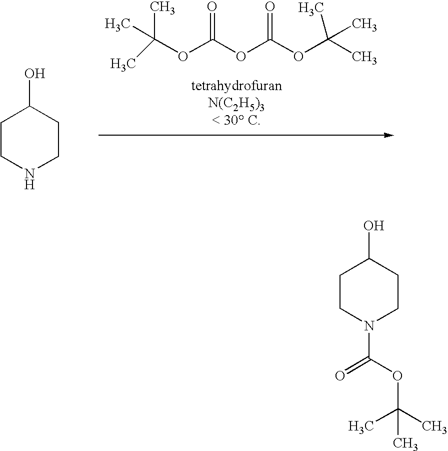

In dry equipment under N₂, 4-hydroxypiperidine (20.2 g, 0.2 mol) was dissolved in tetrahydrofuran (200 mL) and triethylamine (29 mL, 0.21 mol). A solution of di-t-butyldicarbonate (43.65 g, 0.2 mol) was added at such a rate that the temperature remained at less than 30° C. After stirring at ambient temperature for 4 hr, the reaction was concentrated in vacuo. The residue was dissolved in ethyl acetate, washed with water, washed with 5% KHSO₄, washed with saturated NaHCO₃, washed with saturated NaCl solution, dried over Na₂SO₄, filtered, and concentrated in vacuo to afford the t-butyloxycarbonyl piperidine as a white solid (37.7 g, 94%).

### Part B: Preparation of 4-(methylsulfonyl)hydroxy-1-piperidinecarboxylic acid, 1,1-dimethylethyl ester

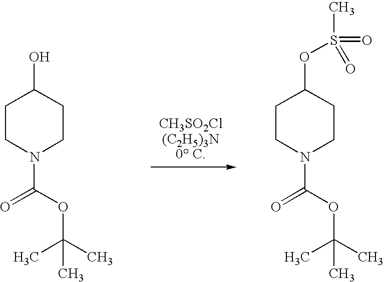

To a solution of the BOC piperidine of Part A (5.00 g, 24.84 mmol) in dichloromethane (50 mL) at 0° C. was added triethylamine (3.81 mL, 27.32 mmol), followed by methane sulfonyl chloride (2.02 mL, 26.08 mmol). When the addition was complete, the cooling bath was removed. After stirring for 2 hr, the reaction mixture was concentrated in vacuo. The residue was taken up in ethyl acetate, washed with water twice, saturated NaCl solution, dried over Na₂SO₄, filtered, and concentrated in vacuo to afford the mesylate as an off-white solid (7.34 g, >100%).

### Part C: Preparation of 4-[4-(trifluoromethoxy)-phenoxy]-1-piperidinecarboxylic acid, 1,1-dimethylethyl ester

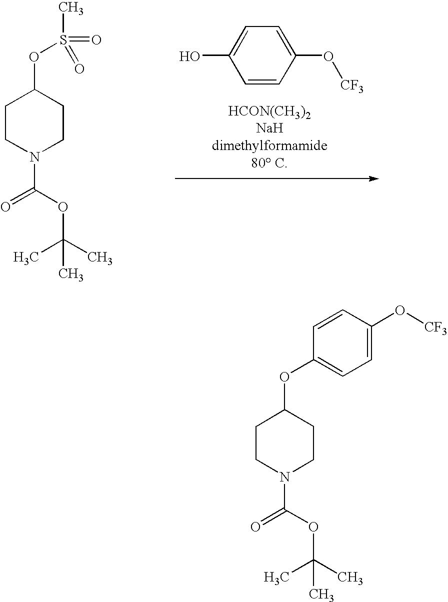

In dry equipment under N₂, 4-trifluoromethoxyphenol (10.15 g, 57 mmol) was dissolved in dry dimethylformamide (125 mL). Sodium hydride (2.74 g, 68.4 mmol of the 60% oil dispersion) was then added at −5° C. The ice bath was subsequently removed. After 1 hr at ambient temperature, the mesylate from Part B (15.9 g, 57 mmol) was added. The resulting mixture was then stirred at 80° C. After stirring at 80° C. for 4 hr, the mixture was concentrated in vacuo. The resulting residue was dissolved in diethyl ether, washed with water, washed with saturated NaCl solution, dried over Na₂SO₄, filtered, and concentrated in vacuo to afford the substituted BOC-piperidine as a beige solid (20.6 g, 100%).

### Part D: Preparation of 4-[4-(trifluoromethoxy)-phenoxylpiperidine

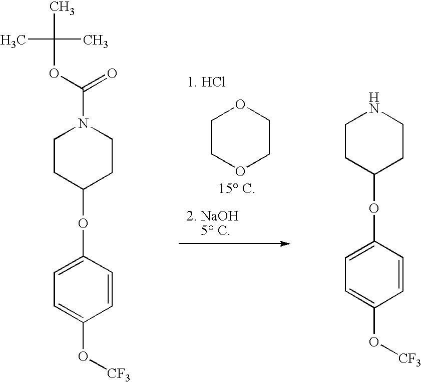

At 15° C., 4 N HCl in dioxane (125 mL) was slowly added to the substituted BOC-piperidine from Part C (20.6 g, 57 mmol) and stirred for 90 min. The mixture was then concentrated in vacuo. The resulting residue was dissolved in water (150 mL), and then washed twice with ethyl acetate. The aqueous solution was cooled to 5° C., and the pH was adjusted to 11 with 5 N NaOH solution. Extraction was performed using ethyl acetate. The ethyl acetate was then dried over Na₂SO₄, filtered, and concentrated in vacuo to provide the substituted piperidine as a beige solid (11.9 g, 80%).

### Example 5

### Alternative Preparation of N-hydroxy-1-(2-methoxyethyl)-4-[[4-[4-(trifluoromethoxy)phenoxy]-1-piperidinyl]sulfonyl]-4-piperidinecarboxamide, monohydrochloride

### Part A. Preparation of 1-(2-methoxyethyl)-N-[(tetrahydro-2H-pyran-2-yl)oxy]-4-[[4-[4-(trifluoromethoxy)phenoxy]-1-piperidinyl]-sulfonyl]-4-piperidinecarboxylate

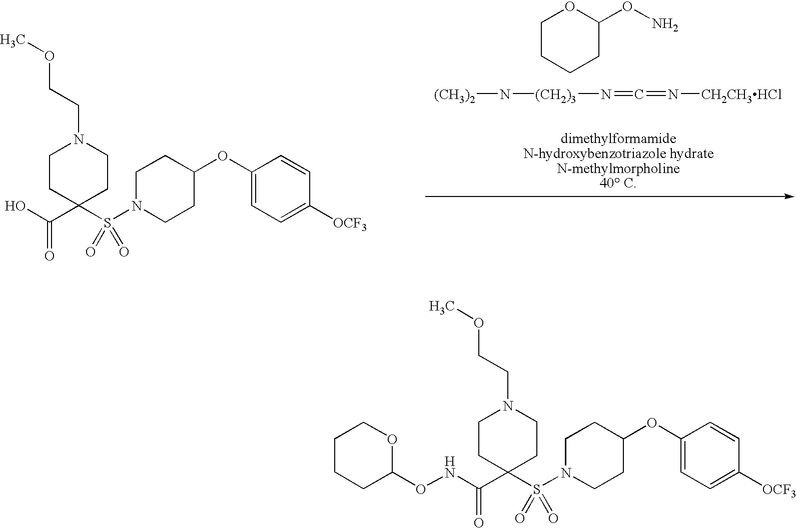

In dry equipment under N₂, 1-(2-methoxyethyl)-4-[[4-[4-(trifluoromethoxy)phenoxy]-1-piperidinyl]-sulfonyl]-4-piperidinecarboxylic acid (1.36 g, 2.67 mmol, prepared in accordance with Part J of Example 15 of U.S. Pat. No. 6,372,758 (cited above and incorporated herein by reference) was dissolved in dry dimethylformamide (9 mL). The following reagents were then added to the solution in the following order: N-hydroxybenzotriazole hydrate (0.43 g, 3.2 mmol), N-methylmorpholine (0.88 mL, 8.0 mmol), O-(tetrahydro-2H-pyran-2-yl)hydroxylamine (0.97 g, 8.0 mmol), and 1-(3-dimethylaminopropyl)-3-ethylcarbodiimide hydrochloride (0.72 g, 3.7 mmol). The resulting mixture was then stirred at 40° C. After 20 hr, the mixture was concentrated in vacuo. The resulting residue was dissolved in ethyl acetate, washed with water, washed with 5% KHSO₄, washed with saturated NaHCO₃, washed with saturated NaCl solution, dried over Na₂SO₄, filtered, and concentrated in vacuo. Chromatography (on silica, ethyl acetate/hexanes) provided the tetrahydropyranyl hydroxamate as a white solid (1.42 g, 90%).

### Part B. Preparation of N-hydroxy-1-(2-methoxyethyl)-4-[[4-[4-(trifluoromethoxy)phenoxy]-1-piperidinyl]sulfonyl]-4-piperidinecarboxamide, monohydrochloride

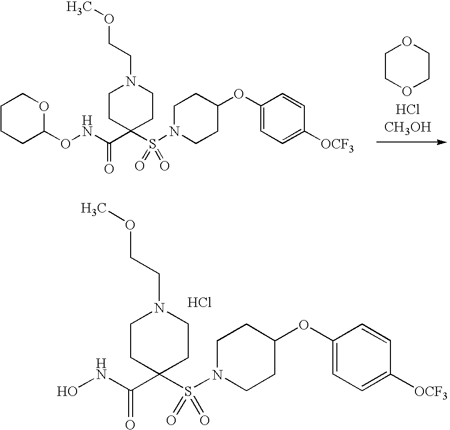

To a solution of the THP hydroxamate from Part A (1.3 g, 2.13 mmol) in 1,4-dioxanes (2 mL) was added 4 N HCl dioxane solution (5.3 mL) and methanol (0.5 mL). After 10 min, the mixture was diluted with diethyl ether. The solids were then filtered under N₂ and dried in vacuo to give the title compound as a white solid (1.15 g, 96%). HRMS (ES+) M+H⁺ calculated for C₂₁H₃₀F₃N₃O₇S₁: 526.1835, found 526.1805.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A process for making a hydroxamic acid compound or a salt thereof, wherein:
the process comprises reacting a 4-sulfonyloxy-heterocyclyl compound or a 4-halo-heterocyclyl compound with a metal thioester; and
the hydroxamic acid compound corresponds in structure to Formula (1-1): 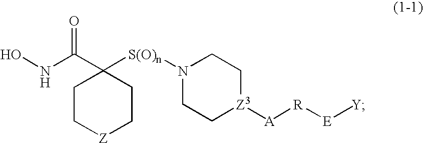
the 4-sulfonyloxy-heterocyclyl compound corresponds in structure to Formula (1-2): 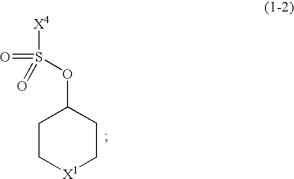
the 4-halo-heterocyclyl compound corresponds in structure to Formula (1-3): 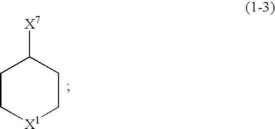
the metal thioester corresponds in structure to Formula (1-4): 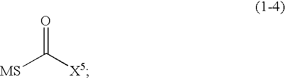
n is selected from the group consisting of zero, 1, and 2; and
X¹ is selected from the group consisting of —O—, —S—, —S(O)—, —S(O)₂—, and —N(R^{x1})—; and
X⁴ is selected from the group consisting of alkyl, haloalkyl, aryl, and haloaryl; and
X⁵ is selected from the group consisting of alkyl, aryl, and arylalkyl, wherein:
the alkyl, aryl, and arylalkyl are optionally substituted with one or more
independently selected halogen; and
X⁷ is halogen; and
M is a metal cation; and
Z is selected from the group consisting of —O—, —S—, —S(O)—, —S(O)₂—, and —N(R^{x})—; and
Z³ is selected from the group consisting of nitrogen and carbon bonded to hydrogen; and
R^{x} is selected from the group consisting of hydrogen, alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
R^{x1} is a nitrogen-protecting group; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

2. A process according to claim 1, wherein:
the hydroxamic acid compound corresponds in structure to a formula selected from the group consisting of: 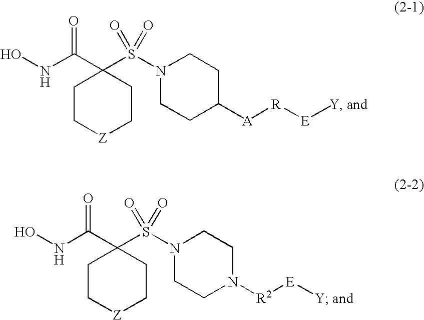
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

3. A process according to claim 2, wherein Z is —N(R^{x})—:

4. A process according to claim 2, wherein the metal thioester comprises potassium thioacetate.

5. A process according to claim 2, wherein the 4-sulfonyloxy-heterocyclyl compound corresponds in structure to Formula (5-1):

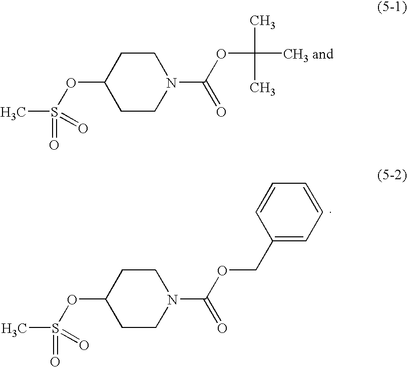

6. A process according to claim 2, wherein the hydroxamic acid compound corresponds in structure to Formula (6-1):

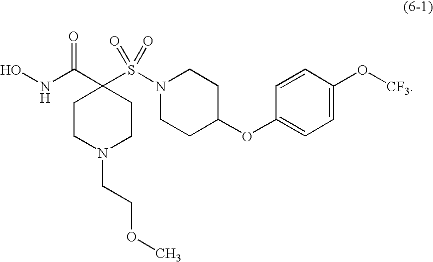

7. A process according to claim 2, wherein the process comprises reacting the 4-halo-heterocyclyl compound with the metal thioester.

8. A process according to claim 2, wherein the process comprises reacting the 4-sulfonyloxy-heterocyclyl compound with the metal thioester.

9. A process according to claim 8, wherein:
the process comprises:
reacting the 4-sulfonyloxy-heterocyclyl compound with the metal thioester to form a 4-thioester-heterocyclyl compound, and
oxidatively halogenating the 4-thioester-heterocyclyl compound to form a 4-halosulfonyl-heterocyclyl compound; and
the 4-thioester-heterocyclyl compound corresponds in structure to Formula (9-1): 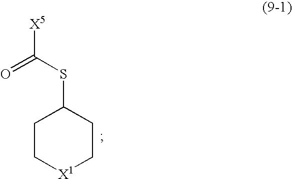
and
the 4-halosulfonyl-heterocyclyl compound corresponds in structure to Formula (9-2): 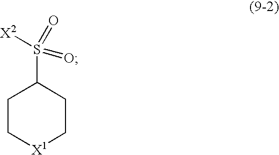
and
X² is halogen.

10. A process according to claim 9, wherein:
X² is chloro; and
the oxidative halogenation comprises combining the 4-thioester-heterocyclyl compound with a source of Cl₂, N-chlorosuccinimide, or 1,3-dichloro-5,5-dimethylhydantoin.

11. A process according to claim 9, wherein X¹ is —N(R^{x1})—.

12. A process according to claim 11, wherein:
the process further comprises:
reacting a second amount of the 4-sulfonyloxy-heterocyclyl compound with an alcohol to form a protected cyclic amino compound having a nitrogen protecting group (R^{x1}), and
removing the nitrogen protecting group from the cyclic amino compound to form an unprotected cyclic amino compound, and
reacting the 4-halosulfonyl-heterocyclcyl compound with the unprotected cyclic amino compound; and
the alcohol is HO—R-E-Y; and
the protected cyclic amino compound corresponds in structure to Formula (12-1): 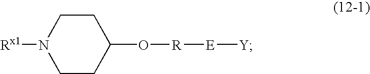
the unprotected cyclic amino compound corresponds in structure to Formula (12-2):

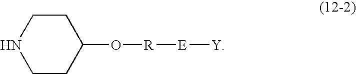

13. A process according to claim 12, wherein:
R is phenyl, and
E-Y is selected from the group consisting of fluoroalkyl and fluoroalkoxy.

14. A process according to claim 13, wherein the hydroxamic acid compound corresponds in structure to Formula (14-1):

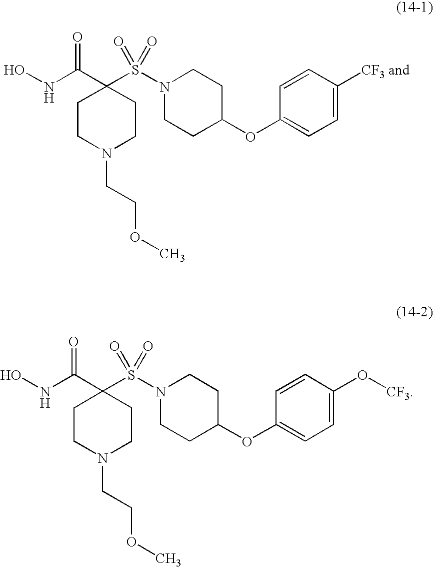

15. A process for making a 4-thioester-heterocyclyl compound, wherein:
the process comprises reacting a 4-sulfonyloxy-heterocyclyl compound or a 4-halo-heterocyclyl compound with a metal thioester; and
the 4-thioester-heterocyclyl compound corresponds in structure to Formula (15-1): 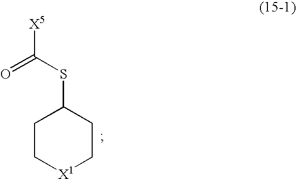
the 4-sulfonyloxy-heterocyclyl compound corresponds in structure to Formula (15-2): 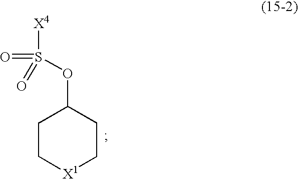
the 4-halo-heterocyclyl compound corresponds in structure to Formula (15-3): 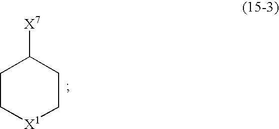
and
the metal thioester corresponds in structure to Formula (15-4): 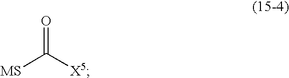
X¹ is selected from the group consisting of —O—, —S—, —S(O)—, —S(O)₂—, and —N(R^{x1})—; and
X⁴ is selected from the group consisting of alkyl, haloalkyl, aryl, and haloaryl; and
X⁵ is selected from the group consisting of alkyl, aryl, and arylalkyl, wherein:
the alkyl, aryl, and arylalkyl are optionally substituted with one or more independently selected halogen; and
X⁷ is halogen; and
M is a metal cation; and
R^{x1} is a nitrogen-protecting group.

16. A process according to claim 15, wherein the 4-thioester-heterocyclyl compound corresponds in structure to Formula (16-1):

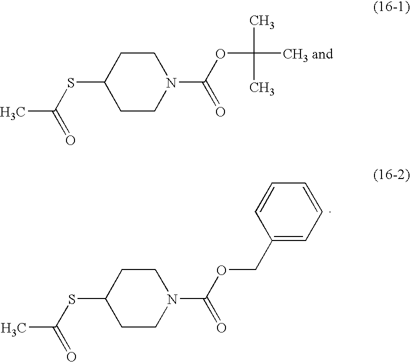

17. A compound or a salt thereof, wherein:
the compound corresponds in structure to Formula (17-1): 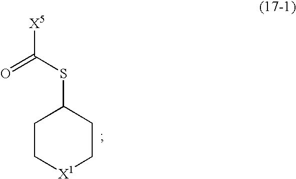
X¹ is selected from the group consisting of —O—, —S—, —S(O)—, —S(O)₂—, and —N(R^{x1})—; and
X⁵ is selected from the group consisting of alkyl, aryl, and arylalkyl, wherein:
the alkyl, aryl, and arylalkyl are optionally substituted with one or more independently selected halogen; and
R^{x1} is a nitrogen-protecting group.

18. A compound or salt thereof according to claim 17, wherein the compound corresponds in structure to Formula (18-1):

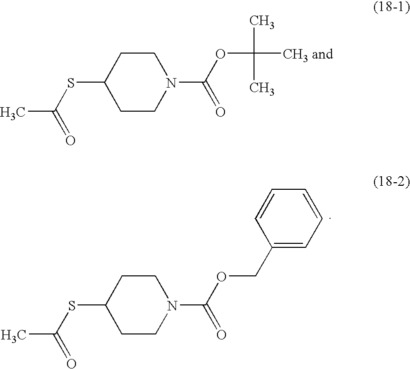

19. A process for making a hydroxamic acid compound or a salt thereof, wherein:
the process comprises oxidatively halogenating a 4-thioester-heterocyclyl compound; and
the hydroxamic acid compound corresponds in structure to Formula (19-1): 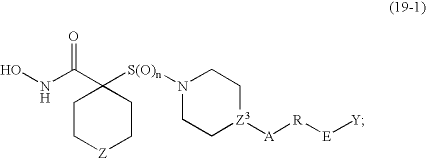
the 4-thioester-heterocyclyl compound corresponds in structure to Formula (19-2): 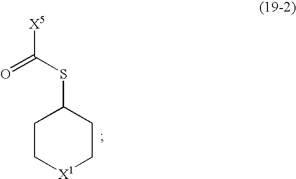
and
n is selected from the group consisting of zero, 1, and 2; and
X¹ is selected from the group consisting of —O—, —S—, —S(O)—, —S(O)₂—, and —N(R^{x1})—; and
X⁵ is selected from the group consisting of alkyl, aryl, and arylalkyl, wherein:
the alkyl, aryl, and arylalkyl are optionally substituted with one or more independently selected halogen; and
Z is selected from the group consisting of —O—, —S—, —S(O)—, —S(O)₂—, and —N(R^{x})—; and
Z³ is selected from the group consisting of nitrogen and carbon bonded to hydrogen; and
R^{x} is selected from the group consisting of hydrogen, alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
R^{x1} is a nitrogen-protecting group; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(k^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

20. A process according to claim 19, wherein R^{x1} is selected from the group consisting of alkoxyalkyl, alkoxycarbonyl, and arylalkoxycarbonyl.

21. A process according to claim 19, wherein:
the hydroxamic acid compound corresponds in structure to a formula selected from the group consisting of: 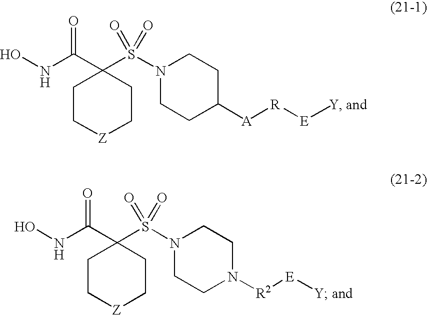
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

22. A process according to claim 21, wherein Z is —N(R^{x})—.

23. A process according to claim 21, wherein the oxidative halogenation comprises combining the 4-thioester-heterocyclyl compound with a source of Cl₂, N-chlorosuccinimide, or 1,3-dichloro-5,5-dimethylhydantoin.

24. A process according to claim 23, wherein the combination of the 4-thioester-heterocyclyl compound with the source of Cl₂, N-chlorosuccinimide, or 1,3-dichloro-5,5-dimethylhydantoin occurs in the presence of an alcohol, acetic acid, or a chloroalkyl solvent.

25. A process according to claim 24, wherein the 4-thioester-heterocyclyl compound is combined with a source of Cl₂.

26. A process according to claim 25, wherein the combination of the 4-thioester-heterocyclyl compound with the source of Cl₂ occurs in the presence of ethanol.

27. A process according to claim 25, wherein the combination of the 4-thioester-heterocyclyl compound with the source of Cl₂ occurs in the presence of glacial acetic acid.

28. A process according to claim 25, wherein the combination of the 4-thioester-heterocyclyl compound with the source of Cl₂ occurs in the presence of a chloroalkyl solvent selected from the group consisting of CCl₄, CHCl₃, and CCl₂.

29. A process according to claim 24, wherein:
the 4-thioester-heterocyclyl compound corresponds in structure to Formula (29-1): 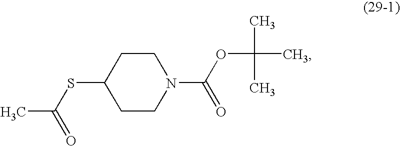
the 4-thioester-heterocyclyl compound is combined with a source of Cl₂ in the presence of ethanol.

30. A process according to claim 24, wherein:
the 4-thioester-heterocyclyl compound corresponds in structure to Formula (30-1): 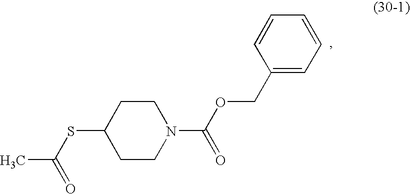
the 4-thioester-heterocyclyl compound is combined with a source of Cl₂ in the presence of glacial acetic acid.

31. A process according to claim 24, wherein the hydroxamic acid compound corresponds in structure to Formula (31-1):

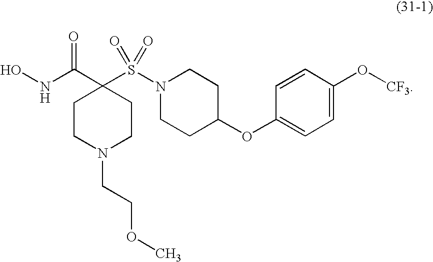

32. A process for making a 4-halosulfonyl-heterocyclyl compound, wherein:
the process comprises oxidatively halogenating a 4-thioester-heterocyclyl compound; and
the 4-halosulfonyl-heterocyclyl compound corresponds in structure to Formula (32-1): 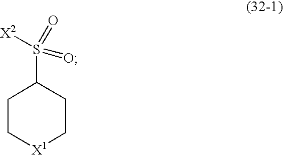
the 4-thioester-heterocyclyl compound corresponds in structure to Formula (32-2): 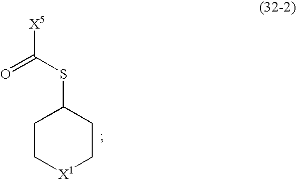
and
X¹ is selected from the group consisting of —O—, —S—, —S(O)—, —S(O)₂—, and —N(R^{x1})—; and
X² is halogen; and
X⁵ is selected from the group consisting of alkyl, aryl, and arylalkyl, wherein:
the alkyl, aryl, and arylalkyl are optionally substituted with one or more independently selected halogen; and
R^{x1} is a nitrogen-protecting group.

33. A compound or a salt thereof, wherein:
the compound corresponds in structure to Formula (33-1): 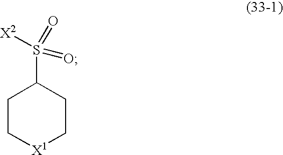
and
X¹ is selected from the group consisting of —O—, —S—, —S(O)—, —S(O)₂—, and —N(R^{x1})—; and
x² is halogen; and
R^{x1} is a nitrogen-protecting group.

34. A compound or salt thereof according to claim 33, wherein the compound corresponds in structure to Formula (34-1):

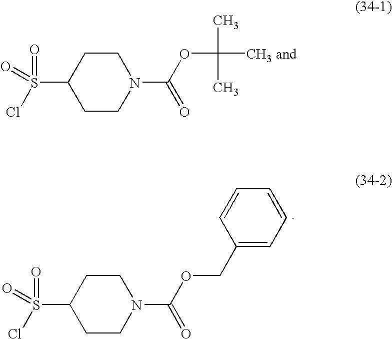

35. A process for making a hydroxamic acid compound or a salt thereof, wherein:
the process comprises reacting a 4-halosulfur-heterocyclyl compound with a cyclic amino compound; and
the hydroxamic acid compound corresponds in structure to Formula (35-1): 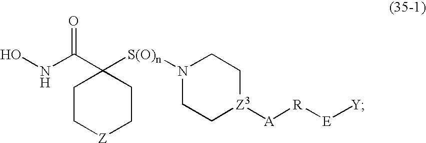
the 4-halosulfur-heterocyclyl compound corresponds in structure to Formula (35-2): 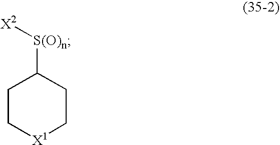
the cyclic amino compound corresponds in structure to Formula (35-3): 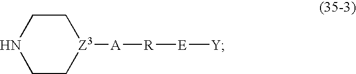
and
each n is independently selected from the group consisting of zero, 1, and 2; and
X¹ is selected from the group consisting of —O—, —S—, —S(O)—, —S(O)₂—, and —N(R^{x1})—; and
X² is halogen; and
Z is selected from the group consisting of —O—, —S—, —S(O)—, —S(O)₂—, and —N(R^{x})—; and
Z³ is selected from the group consisting of nitrogen and carbon bonded to hydrogen; and
R^{x} is selected from the group consisting of hydrogen, alkyl, alkenyl, alkynyl, R^{a}-oxyalkyl, aminosulfonyl, alkylsulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
R^{x1} is a nitrogen-protecting group; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

36. A process according to claim 35, wherein R^{x1} is selected from the group consisting of alkoxyalkyl, alkoxycarbonyl, and arylalkoxycarbonyl.

37. A process according to claim 35, wherein:
the hydroxamic acid compound corresponds in structure to a formula selected from the group consisting of: 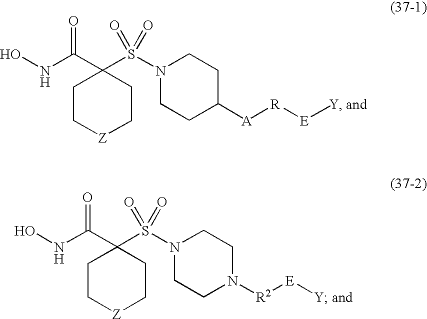
the cyclic amino compound corresponds in structure to a formula selected from the group consisting of: 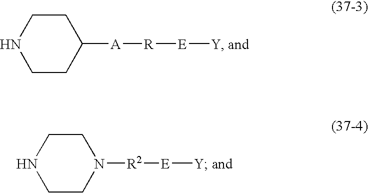
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

38. A process according to claim 37, wherein:
R and R² are optionally-substituted aryl; and
A is selected from the group consisting of —O—, —S—, —S(O)₂—, —O—S(O)₂—, —S(O)₂—O—, —C(O)—, —C(O)—O—, —O—C(O)—, and a bond; and
E is selected from the group consisting of —O—, —C(O)—, and a bond.

39. A process according to claim 38, wherein:
X¹ is —N(R^{x1})—; and
R^{x1} is selected from the group consisting of alkoxyalkyl, alkoxycarbonyl, and arylalkoxycarbonyl.

40. A process according to claim 39, wherein the 4-halosulfur-heterocyclyl compound corresponds in structure to Formula (40-1):

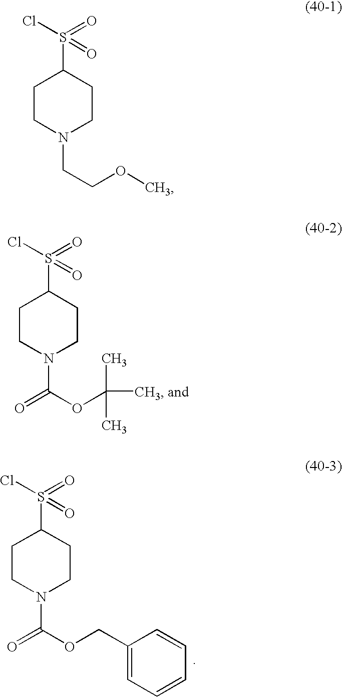

41. A process according to claim 37, wherein the hydroxamic acid compound corresponds in structure to Formula (41-1):

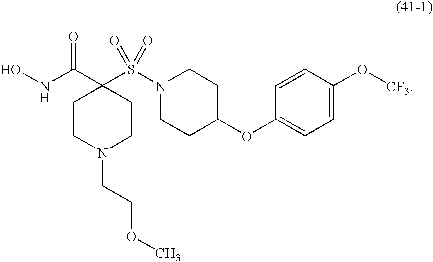

42. A process according to claim 38, wherein:
the cyclic amino compound is prepared by a process comprising a reaction wherein a reagent is 4-hydroxy-piperidine, and
the 4-halosulfur-heterocyclyl compound is prepared by a process comprising a reaction wherein a reagent is 4-hydroxy-piperidine.

43. A process according to claim 38, wherein:
the cyclic amino compound is prepared by a process comprising a reaction wherein a reagent is a first amount of a protected 4-hydroxy-piperidinyl compound; and
the 4-halosulfur-heterocyclyl compound is prepared by a process comprising a reaction wherein a reagent is a second amount of the protected 4-hydroxy-piperidinyl compound; and
the protected 4-hydroxy-piperidinyl compound corresponds in structure to Formula (43-1): 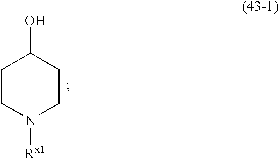
R^{x1} is selected from the group consisting of alkoxycarbonyl and arylalkoxycarbonyl.

44. A process according to claim 38, wherein:
the cyclic amino compound is prepared by a process comprising a reaction wherein a reagent is a first amount of a 4-sulfonyloxy-piperidinyl compound; and
the 4-halosulfur-heterocyclyl compound is prepared by a process comprising a reaction wherein a reagent is a second amount of the 4-sulfonyloxy-piperidinyl compound; and
the 4-sulfonyloxy-piperidinyl compound corresponds in structure to Formula (44-1): 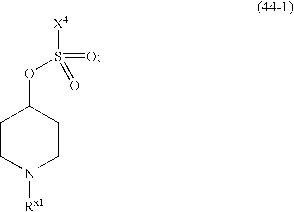
and
R^{x1} is selected from the group consisting of alkoxycarbonyl and arylalkoxycarbonyl; and
X⁴ is selected from the group consisting of alkyl, haloalkyl, aryl, and haloaryl.

45. A process according to claim 44, wherein:
the cyclic amino compound is prepared by a process comprising:
reacting the 4-sulfonyloxy-heterocyclyl compound with an alcohol to form a protected cyclic amino compound having a nitrogen protecting group (R^{x1}), and
removing the nitrogen protecting group from the protected cyclic amino compound; and
the alcohol is HO—R-E-Y; and
the protected cyclic amino compound corresponds in structure to Formula (45-1):

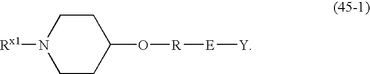

46. A process according to claim 44, wherein:
the 4-halosulfur-heterocyclyl compound is prepared by a process comprising:
reacting the 4-sulfonyloxy-heterocyclyl compound with a metal thioester to form a 4-thioester-heterocyclyl compound, and
oxidatively halogenating the 4-thioester-heterocyclyl compound; and
the metal thioester corresponds in structure to Formula (46-1): 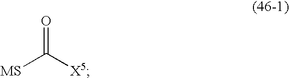
the 4-thioester-heterocyclyl compound corresponds in structure to Formula (46-2): 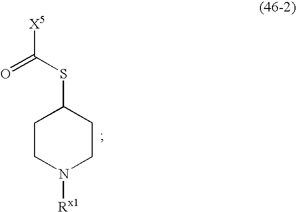
and
M is a metal cation; and
X⁵ is selected from the group consisting of alkyl, aryl, and arylalkyl, wherein:
the alkyl, aryl, and arylalkyl are optionally substituted with one or more independently selected halogen.

47. A process according to claim 46, wherein the oxidative halogenation comprises combining the 4-thioester-heterocyclyl compound with a source of Cl₂, N-chlorosuccinimide, or 1,3-dichloro-5,5-dimethylhydantoin.

48. A process for making an sulfuramine compound, wherein:
the process comprises reacting a 4-halosulfur-heterocyclyl compound with a cyclic amino compound; and
the sulfuramine compound corresponds in structure to Formula (48-1): 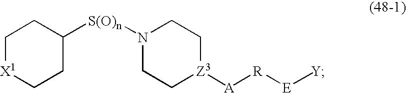
the 4-halosulfur-heterocyclyl compound corresponds in structure to Formula (48-2): 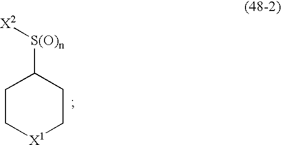
and
the cyclic amino compound corresponds in structure to Formula (48-3): 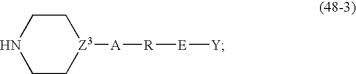
and
each n is independently selected from the group consisting of zero, 1, and 2; and
Z³ is selected from the group consisting of nitrogen and carbon bonded to hydrogen; and
X¹ is selected from the group consisting of —O—, —S—, —S(O)—, —S(O)₂—, and —N(R^{x1})—; and
X² is halogen; and
R^{x1} is a nitrogen-protecting group; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

49. A process according to claim 48, wherein the sulfuramine compound corresponds in structure to Formula (49-1):

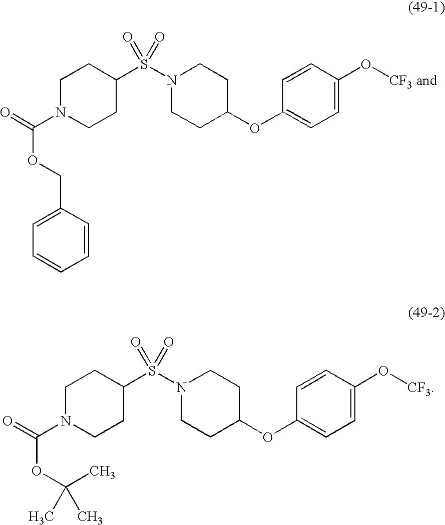

50. A process for making a hydroxamic acid compound or a salt thereof, wherein:
the process comprises removing a nitrogen-protecting group (R^{x1})) from a piperidinyl nitrogen of a nitrogen-protected piperidinyl compound; and
the hydroxamic acid compound corresponds in structure to Formula (50-1): 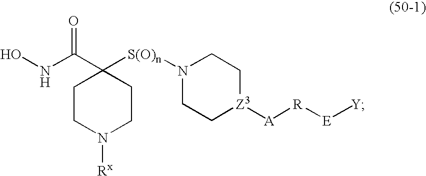
and
the nitrogen-protected piperidinyl compound corresponds in structure to Formula (50-2): 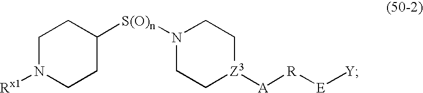
Z³ is selected from the group consisting of nitrogen and carbon bonded to hydrogen; and
each n is independently selected from the group consisting of zero, 1, and 2; and
R^{x} is selected from the group consisting of hydrogen, alkyl, alkenyl, alkynyl, R^{a}-oxyalkyl, alkylsulfonyl, aminosulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
A is selected from the group consisting of —O—, —S—, —S(O)—, —S()₂—, —C(O)—, —NR^{b}—, —CO—N(k^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

51. A process according to claim 50, wherein R^{x1} is selected from the group consisting of alkoxycarbonyl and arylalkoxycarbonyl.

52. A process according to claim 50, wherein:
the hydroxamic acid compound corresponds in structure to a formula selected from the group consisting of: 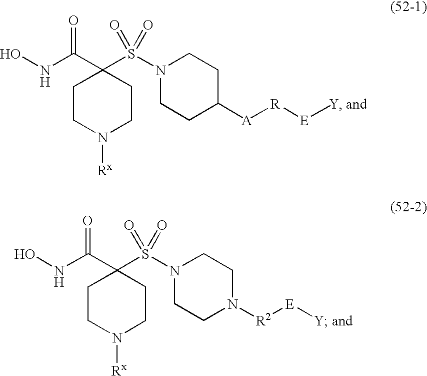
the nitrogen-protected piperidinyl compound corresponds in structure to a formula selected from the group consisting of: 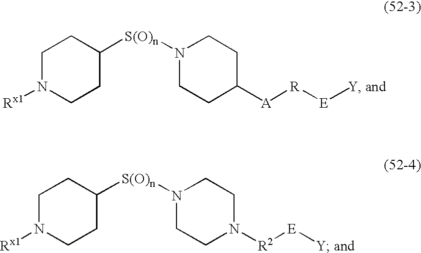
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

53. A process according to claim 52, wherein the process comprises contacting the nitrogen-protected piperidinyl compound with a hydrogen source and a transition metal catalyst.

54. A process according to claim 53, wherein the hydrogen source comprises ammonium formate.

55. A process according to claim 52, wherein the process comprises contacting the nitrogen-protected piperidinyl compound with an acid.

56. A process according to claim 55, wherein the acid has a pKa of no greater than about −3.

57. A process according to claim 55, wherein contacting of the nitrogen-protected piperidinyl compound with the acid occurs at a temperature of greater than 30° C.

58. A process according to claim 56, wherein the acid comprises HCl gas.

59. A process according to claim 56, wherein the acid comprises HCl in an alcohol.

60. A process according to claim 55, wherein the nitrogen-protected piperidinyl compound corresponds in structure selected from the group consisting of:

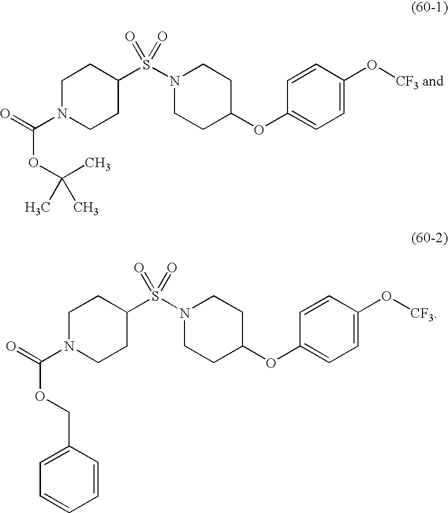

61. A process according to claim 52, wherein the hydroxamic acid compound corresponds in structure to Formula (61-1):

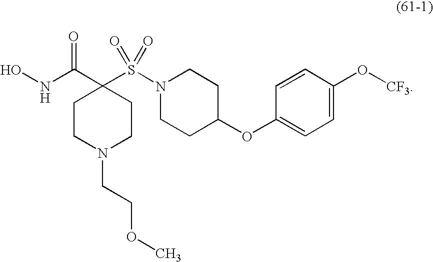

62. A process for making an unprotected piperidinyl compound, wherein:
the process comprises removing a nitrogen-protecting group (R^{x1}) from a piperidinyl nitrogen of a nitrogen-protected piperidinyl compound; and
the unprotected piperidinyl compound corresponds in structure to Formula (62-1): 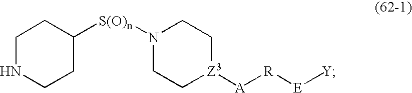
the nitrogen-protected piperidinyl compound corresponds in structure to Formula (62-2): 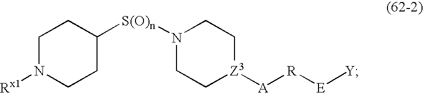
Z³ is selected from the group consisting of nitrogen and carbon bonded to hydrogen; and
each n is independently selected from the group consisting of zero, 1, and 2; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

63. A process according to claim 62, wherein the unprotected piperidinyl compound corresponds in structure to Formula (63-1):

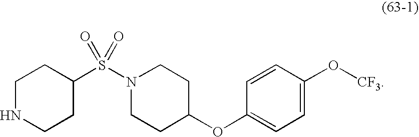

64. A process for making a hydroxamic acid compound or a salt thereof, wherein:
the process comprises contacting an unprotected piperidinyl compound with an N-alkylating agent; and
the hydroxamic acid compound corresponds in structure to Formula (64-1): 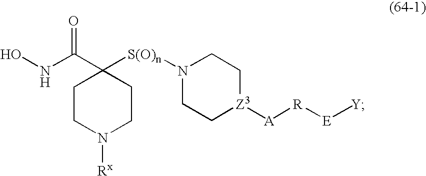
the unprotected piperidinyl compound corresponds in structure to Formula (64-2): 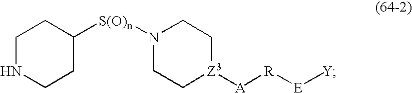
and
Z³ is selected from the group consisting of nitrogen and carbon bonded to hydrogen; and
each n is independently selected from the group consisting of zero, 1, and 2; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
R^{x} is alkyl optionally substituted with a substituent selected from the group consisting of R^{a}-oxy, R^{a}R^{a}-amino (wherein each R^{a} is other than hydrogen), carbocyclyl, and heterocyclyl, wherein:
any member of such group optionally is substituted with one or more substituents independently selected from the group consisting of halogen, cyano, carboxy, thiol, sulfo, nitro, nitroso, oxo, thioxo, imino, alkoxy, alkylthio, and alkoxyalkoxy, wherein:
any member of such group optionally is substituted with one or more substituents independently selected from the group consisting of halogen, hydroxy, and alkyl; and
each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

65. A process according to claim 64, wherein:
the hydroxamic acid compound corresponds in structure to a formula selected from the group consisting of: 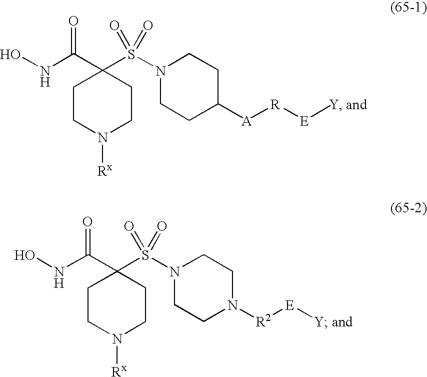
the unprotected piperidinyl compound corresponds in structure to a formula selected from the group consisting of: 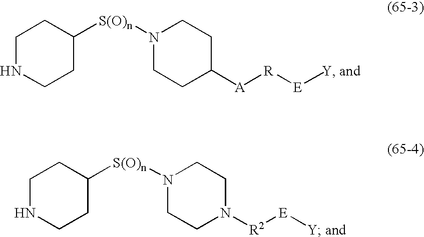
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

66. A process according to claim 65, wherein:
the N-alkylating agent is R^{x}-X³; and
X³ is halogen or corresponds in structure to a formula selected from the group consisting of:

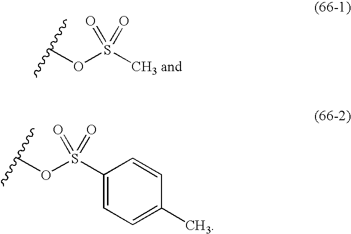

67. A process according to claim 66, wherein X³ is halogen.

68. A process according to claim 67, wherein contacting of the unprotected piperidinyl compound with the N-alkylating agent occurs at a temperature of greater than 30° C.

69. A process according to claim 67, wherein contacting of the unprotected piperidinyl compound with the N-alkylating agent occurs in the presence of base.

70. A process according to claim 69, wherein the base comprises potassium carbonate.

71. A process according to claim 67, wherein:
X³ is bromo, and
contacting of the unprotected piperidinyl compound with the N-alkylating agent occurs at a temperature of from about 50 to about 60° C.

72. A process according to claim 67, wherein:
X³ is chloro, and
contacting of the unprotected piperidinyl compound with the N-alkylating agent occurs at a temperature of greater than about 60° C.

73. A process according to claim 67, wherein the hydroxamic acid compound corresponds in structure to Formula (73-1):

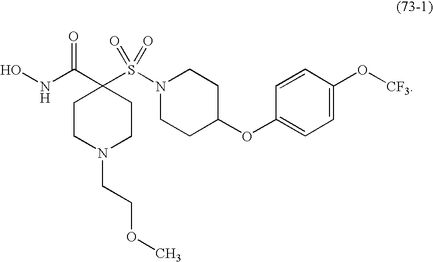

74. A process for making an alkylated piperidinyl compound or a salt thereof, wherein:
the process comprises contacting an unprotected piperidinyl compound with an N-alkylating agent; and
the alkylated piperidinyl compound corresponds in structure to Formula (74-1): 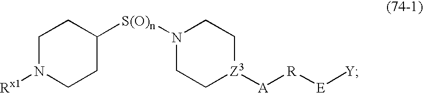
and
the unprotected piperidinyl compound corresponds in structure to Formula (74-2): 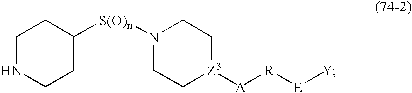
and
Z³ is selected from the group consisting of nitrogen and carbon bonded to hydrogen; and
each n is independently selected from the group consisting of zero, 1, and 2; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
R^{x} is alkyl optionally substituted with a substituent selected from the group consisting of R^{a}-oxy, R^{a}R^{a}-amino (wherein each R^{a} is other than hydrogen), carbocyclyl, and heterocyclyl, wherein:
any member of such group optionally is substituted on any atom capable of such substituent with one or more substituents independently selected from the group consisting of halogen, cyano, carboxy, thiol, sulfo, nitro, nitroso, oxo, thioxo, imino, alkoxy, alkylthio, and alkoxyalkoxy, wherein:
any member of such group optionally is substituted with one or more substituents independently selected from the group consisting of halogen, hydroxy, and alkyl; and
each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{b} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

75. A process according to claim 74, wherein the alkylated piperidinyl compound corresponds in structure to Formula (75-1):

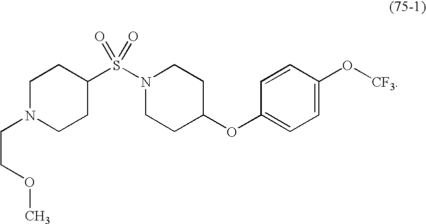

76. A process for making a hydroxamic acid compound or a salt thereof, wherein:
the process comprises contacting a sulfuramine compound with a base to form an anion, and contacting the anion with a carbon dioxide source; and
the hydroxamic acid compound corresponds in structure to Formula (76-1): 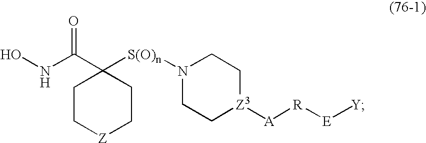
and
the sulfuramine compound corresponds in structure to Formula (76-2): 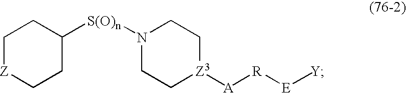
and
each n is independently selected from the group consisting of zero, 1, and 2; and
Z is selected from the group consisting of —O—, —S—, —S(O)—, —S(O)₂—, and —N(R^{x})—; and
Z³ is selected from the group consisting of nitrogen and carbon bonded to hydrogen; and
R^{x} is selected from the group consisting of hydrogen, alkyl, alkenyl, alkynyl, R^{a}-oxyalkyl, alkylsulfonyl, aminosulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
A is selected from the group consisting of —O—, —S—, —S(O)—, —S(O)₂—, —C(O)—, —NR^{b}—, —CO—N(R^{b}), —N—(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

77. A process according to claim 76, wherein:
the hydroxamic acid compound corresponds in structure to a formula selected from the group consisting of: 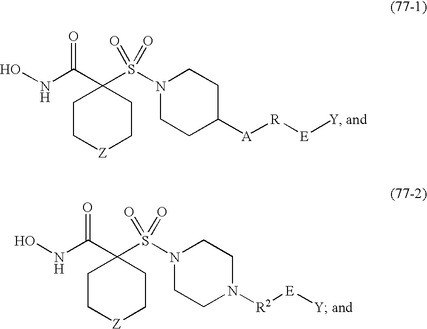
the sulfuramine compound corresponds in structure to a formula selected from the group consisting of: 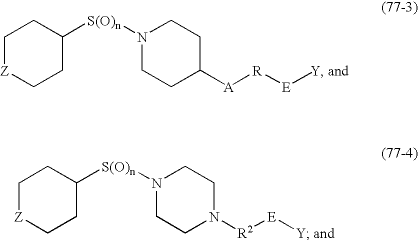
R² is selected from the group consisting of aryl and heteroaryl, wherein:
the aryl or heteroaryl optionally is substituted with one or more substituents independently selected from the group consisting of halogen, nitro, nitroso, hydroxy, alkyl, alkoxy, alkylthio, alkoxyalkyl, alkoxycarbonylalkyl, C₁—C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino.

78. A process according to claim 77, wherein the base comprises a strong, non-aqueous base.

79. A process according to claim 78, wherein the base comprises alkyllithium.

80. A process according to claim 79, wherein the base comprises butyllithium.

81. A process according to claim 78, wherein the base comprises lithium diisopropylamide.

82. A process according to claim 78, wherein contacting of the sulfuramine compound with the base occurs at a temperature of less than 20° C.

83. A process according to claim 78, wherein contacting of the anion with the carbon dioxide source occurs at a temperature of less than 20° C.

84. A process according to claim 78, wherein the source of carbon dioxide comprises methylchloroformate.

85. A process according to claim 78, wherein the source of carbon dioxide comprises CO₂ gas.

86. A process according to claim 85, wherein the sulfuramine compound corresponds in structure to Formula (86-1):

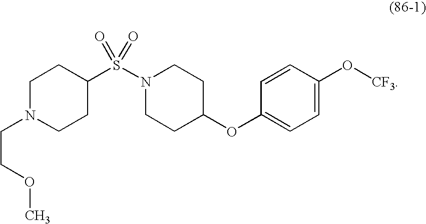

87. A process for making a carboxylic acid compound, wherein:
the process comprises contacting a sulfuramine compound with a base to form an anion, and contacting the anion with a carbon dioxide source; and
the carboxylic acid compound corresponds in structure to Formula (87-1): 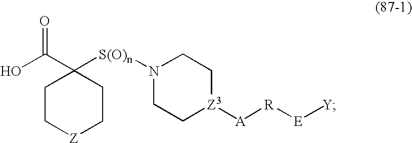
the sulfuramine compound corresponds in structure to Formula (87-2): 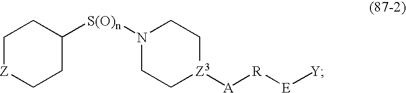
and
each n is independently selected from the group consisting of zero, 1, and 2; and
Z is selected from the group consisting of —O—, —S—, —S(O)—, —S(O)₂—, and —N(R^{x})—; and
Z³ is selected from the group consisting of nitrogen and carbon bonded to hydrogen; and
R^{x} is selected from the group consisting of hydrogen, alkyl, alkenyl, alkynyl, R^{a}-oxyalkyl, alkylsulfonyl, aminosulfonyl, R^{a}R^{a}-aminoalkyl, carbocyclyl, carbocyclylalkyl, carbocyclylsulfonyl, heterocyclyl, heterocyclylalkyl, and heterocyclylsulfonyl, wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkylthio, alkoxyalkyl, and alkoxyalkoxy, wherein:
any member of such group optionally is substituted with one or more substituents independently selected from the group consisting of halogen, hydroxy, and alkyl; and
A is selected from, the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

88. A process according to claim 87, wherein the carboxylic acid compound corresponds in structure to Formula (88-1):

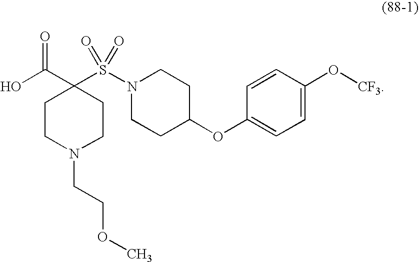

89. A compound or a salt thereof, wherein:
the compound corresponds in structure to Formula (89-1): 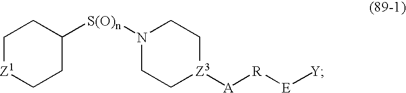
and
n is selected from the group consisting of zero, 1, and 2; and
Z¹ is selected from the group consisting of —N(H)—, X¹, and —N(R^{x2})—; and
Z³ is selected from the group consisting of nitrogen and carbon bonded to hydrogen; and
X¹ is selected from the group consisting of —O—, —S—, —S(O)—, —S(O)₂—, and —N(R^{x1})—; and
R^{x1} is a nitrogen-protecting group; and
R^{x2} is alkyl optionally substituted with a substituent selected from the group consisting of R^{a}-oxy, R^{a}R^{a}-amino (wherein each R^{a} is other than hydrogen), carbocyclyl, and heterocyclyl, wherein:
any member of such group optionally is substituted with one or more substituents independently selected from the group consisting of halogen, cyano, carboxy, thiol, sulfo, nitro, nitroso, oxo, thioxo, imino, alkoxy, alkylthio, and alkoxyalkoxy, wherein:
any member of such group optionally is substituted with one or more substituents independently selected from the group consisting of halogen, hydroxy, and alkyl; and
A is selected from the group consisting of —O—, —S—, —S(O)—, —S(O)₂—, —C(O)—, —NR^{b}—, —CO—N(R^{b}), —N(R^{b})—C(O)—, —C(O)—O—, —O—C(O)—, —O—C(O)—O—, —HC═CH—, —C≡C—, —N═N—, —C(S)—N(R^{b})—, —N(R^{b})—C(S)—, alkyl, alkoxy, oxyalkyl, alkylthio, thioalkyl, and a bond; and
R is selected from the group consisting of alkyl, alkenyl, alkynyl, alkoxyalkyl, carbocyclyl, heterocyclyl, carbocyclylalkyl, heterocyclylalkyl, carbocyclyloxyalkyl, heterocyclyloxyalkyl, carbocyclylthioalkyl, and heterocyclylthioalkyl, wherein:
any member of such group optionally is substituted with one or more substituents independently selected from the group consisting of halogen, nitro, nitroso, hydroxy, oxo, alkyl, alkoxy, alkylthio, alkoxyalkyl, alkoxycarbonylalkyl, C₁-C₂-alkylenedioxy, and alkoxycarbonyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
E is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
Y is selected from the group consisting of hydrogen, halogen, cyano, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, heterocyclylalkyl, and heterocyclylalkoxyalkyl, wherein:
any member of such group optionally is substituted with one or more independently selected R^{d} substituents; and
each R^{a} is independently selected from the group consisting of hydrogen, alkyl, alkoxyalkyl, bisalkoxyalkyl, alkylthioalkyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, and heterocyclylsulfonylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{b} is independently selected aryl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, alkyl, alkoxy, alkenyl, alkynyl, alkoxyalkyl, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino; and
each R^{g} is independently selected from the group consisting of hydrogen, alkyl, —O—R^{h}, carbocyclylalkyl, and heterocyclylalkyl; and
any member of such group optionally is substituted with one or more substituents independently selected from the group consisting of halogen, hydroxy, cyano, carboxy, thiol, sulfo, nitro, nitroso, oxo, thioxo, and imino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, and imino.

90. A compound or salt thereof according to claim 89, wherein Z is —N(H)—.

91. A compound or salt thereof according to claim 90, wherein the compound corresponds in structure to Formula (91-1):

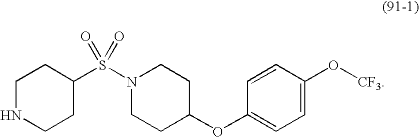

92. A compound or salt thereof according to claim 89, wherein Z¹ is X¹.

93. A compound or salt thereof according to claim 92, wherein:
X¹ is —N(R^{x1})—; and
R^{x1} is selected from the group consisting of alkoxyalkyl, alkoxycarbonyl, and arylalkoxycarbonyl.

94. A compound or salt thereof according to claim 93, wherein the compound corresponds is selected from the group consisting of: [chemical structure]
